(19) United States
(12) Patent Application Publication
(10) Pub. No.: US 2022/0290241 A1
(43) Pub. Date: Sep. 15, 2022

(54) PREDICTING EXTRAINTESTINAL MANIFESTATIONS OF INFLAMMATORY BOWEL DISEASE

(71) Applicant: Cedars-Sinai Medical Center, Los Angeles, CA (US)

(72) Inventors: Dermot MCGOVERN, Los Angeles, CA (US); Michelle KHROM, Northridge, CA (US); Talin HARITUNIANS, Los Angeles, CA (US)

(21) Appl. No.: 17/825,580

(22) Filed: May 26, 2022

Related U.S. Application Data
(63) Continuation of application No. PCT/US2020/062404, filed on Nov. 25, 2020.
(60) Provisional application No. 62/941,209, filed on Nov. 27, 2019.

(51) Int. Cl. C12Q 1/6883; A61K 45/06; A61P 37/06; A61K 31/573; C07K 16/24; C07K 16/28

(52) U.S. Cl. CPC C12Q 1/6883; A61K 45/06; A61P 37/06; A61K 31/573; C07K 16/241; C07K 16/2866; A61K 2039/505

(57) ABSTRACT

Provided are methods, systems, and kits for identifying a patient who has or has a likelihood of developing an extra-intestinal manifestation of inflammatory bowel disease.

### CROSS-REFERENCE

This application is a continuation of International Application No. PCT/US2020/062404 filed Nov. 25, 2020, which claims the benefit of U.S. Patent Application No. 62/941,209, filed Nov. 27, 2019, each of which is hereby incorporated by reference in its entirety.

### STATEMENT AS TO FEDERALLY SPONSORED RESEARCH

This invention was made with government support under Grant Nos. U01 DK062413 and P01 DK046763 awarded by the National Institutes of Health (NIH) and National Institute of Diabetes and Digestive Kidney Diseases (HIDDK). The government has certain rights in the invention.

### SEQUENCE LISTING

The instant application contains a Sequence Listing which has been submitted electronically in ASCII format and is hereby incorporated by reference in its entirety. Said ASCII copy, created on May 24, 2022, is named 56884-768_301_SL.txt and is 1,359,037 bytes in size.

### BACKGROUND

Inflammatory disease, fibrostenotic disease, and fibrotic disease pose a significant health burden worldwide due to the vast number of individuals affected, heterogeneous disease pathogenesis, and varied clinical manifestations. One such disease is inflammatory bowel disease (IBD), which has two common forms, Crohn's disease (CD) and ulcerative colitis (UC). IBD comprises chronic, relapsing inflammatory disorders of the gastrointestinal tract. Incidences of IBD are prevalent, affecting nearly three million individuals in the United States alone.

Extraintestinal manifestations (EIMs) of inflammatory bowel disease (IBD) may occur in both ulcerative colitis and Crohn's disease. EIMs may develop prior to the onset of colonic symptoms or after the onset of colonic symptoms. They commonly affect tissues including the skin, joints, eyes, or other organs. Some EIMs are associated with active intestinal inflammation and some EIMs occur independent of intestinal inflammation. Examples of EIMs include ankylosing spondylitis, sacroiliitis, erythema nodosum, pyoderma gangrenosum, psoriasis, a manifestation in the eye, and primary sclerosing cholangitis.

### SUMMARY

Provided herein are genotypes associated with and/or predictive of the likelihood that a patient has or will develop an extra-intestinal manifestation (EIM) of inflammatory bowel disease (IBD). In certain embodiments, genotypes described herein are also associated with IBD, such as Crohn's disease (CD). Further provided are methods for predicting the likelihood that a patient has or will develop an EIM based on the genotype of patient. Additional methods include treating a patient for IBD based on the patient's genotype. The patient may be diagnosed with, or suspected of having, IBD, such as CD. As an example treatment method, patients exhibiting a genotype associated with a particular EIM may be treated with an IBD therapeutic that may also prevent or treat the EIM. As another example, patients exhibiting a genotype associated with a particular EIM may be treated with a therapeutic that will reduce the likelihood of developing the EIM.

Non-limiting practical applications of the associations between the genotypes described herein and incidences of clinical and subclinical phenotypes in certain populations of individuals are provided herein. For example, some genotypes of the present disclosure can be used to predict a risk that a subject will develop ankylosing spondylitis, sacroiliitis, erythema nodosum, pyoderma gangrenosum, psoriasis, a manifestation in the eye, or primary sclerosing cholangitis, or a combination thereof. The genotypes are also useful to predict whether a patient diagnosed with some form of an inflammatory, fibrotic or fibrostenotic disease will develop a severe form of the disease, such as a subclinical phenotype thereof.

Aspects disclosed herein provide a method for treating a subject having an inflammatory bowel disease, the method comprising administering to the subject a therapeutic agent, provided the subject has been determined to have a genotype comprising one or more single nucleotide polymorphisms selected from the group comprising rs6905036, rs2844510, rs2516514, and rs17030062. In some embodiments, the subject has been determined to be at risk for developing ankylosing spondylitis and/or sacroiliitis.

Aspects disclosed herein provide a method for evaluating whether a subject having an inflammatory bowel disease will develop an extra-intestinal manifestation (EIM), the method comprising determining whether the subject has a genotype comprising one or more single nucleotide polymorphisms selected from the group comprising rs6905036, rs2844510, rs2516514, and rs17030062. In some embodiments, the EIM comprises ankylosing spondylitis and/or sacroiliitis. In some embodiments, the method comprises administering to the subject a therapeutic agent. In some embodiments, the therapeutic agent comprises an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof. In some embodiments, the therapeutic agent comprises etanercept, adalimumab, infliximab, cyclobenzaprine, a steroid, secukinumab, methotrexate, leflunomide, hydroxychloroquine, or sulfasalazine, or a combination thereof.

Aspects disclosed herein provide a method for treating a subject having an inflammatory bowel disease, the method comprising administering to the subject a therapeutic agent, provided the subject has been determined to have a genotype comprising one or more single nucleotide polymorphisms selected from the group comprising rs9276456, rs2858884, rs2858319, rs6917611, rs6930571, rs389419, rs76558762, rs7956721, rs887864, and rs9276424. In some embodiments, the subject has been determined to be at risk for developing primary sclerosing cholangitis. Aspects disclosed herein provide a method for evaluating whether a subject having an inflammatory bowel disease will develop an extra-intestinal manifestation (EIM), the method comprising determining whether the subject has a genotype comprising one or more single nucleotide polymorphisms selected from the group comprising rs9276456, rs2858884, rs2858319, rs6917611, rs6930571, rs389419, rs76558762, rs7956721, rs887864, and rs9276424. In some embodiments, the EIM comprises primary sclerosing cholangitis. In some embodiments, the method comprises administering to the subject a therapeutic agent. In some embodiments, the therapeutic agent comprises an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof. In some embodiments, the therapeutic agent comprises ursodeoxycholic acid (UDCA), antipruritic, cholestyramine, antibiotic, vitamin A, vitamin D, vitamin E, or vitamin K, or a combination thereof.

Aspects disclosed herein provide a method for treating a subject having an inflammatory bowel disease, the method comprising administering to the subject a therapeutic agent, provided the subject has been determined to have a genotype comprising one or more single nucleotide polymorphisms selected from the group comprising rs10056322, rs6461986, and rs13421864. In some embodiments, the subject has been determined to be at risk for developing arthritis. Aspects disclosed herein provide a method for evaluating whether a subject having an inflammatory bowel disease will develop an extra-intestinal manifestation (EIM), the method comprising determining whether the subject has a genotype comprising one or more single nucleotide polymorphisms selected from the group comprising rs10056322, rs6461986, and rs13421864. In some embodiments, the EIM comprises arthritis. In some embodiments, the arthritis is peripheral arthritis. In some embodiments, the method comprises administering to the subject a therapeutic agent. In some embodiments, the therapeutic agent comprises an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof. In some embodiments, the therapeutic agent comprises methotrexate, leflunomide, hydroxychloroquine, sulfasalazine, nonsteroidal anti-inflammatory drug (NSAID), TNF inhibitor, etanercept, infliximab, belimumab, rituximab, anakinra, tocilizumab, canakinumab, secukinumab, ustekinumab, ixekizumab, sarilumab, tofacitinib, abatacept, corticosteroid, prednisone, or cortisone, or a combination thereof.

Aspects disclosed herein provide a method for treating a subject having an inflammatory bowel disease, the method comprising administering to the subject a therapeutic agent, provided the subject has been determined to have a genotype comprising one or more single nucleotide polymorphisms selected from the group comprising rs4349859, rs4418214, rs3819299, rs2373969, rs4151651, and rs9366775. In some embodiments, the subject has been determined to be at risk for developing an extra-intestinal manifestation (EIM) of the eye. Aspects disclosed herein provide a method for evaluating whether a subject having an inflammatory bowel disease will develop an extra-intestinal manifestation (EIM), the method comprising determining whether the subject has a genotype comprising one or more single nucleotide polymorphisms selected from the group comprising rs4349859, rs4418214, rs3819299, rs2373969, rs4151651, and rs9366775. In some embodiments, the EIM comprises uveitis, iritis, episcleritis, scleritis, or undiagnosed ocular inflammation, or a combination thereof. In some embodiments, the method comprises administering to the subject a therapeutic agent. In some embodiments, the therapeutic agent comprises an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof. In some embodiments, the therapeutic agent comprises a steroid, methotrexate, mycophenolate, sulfasalazine, azathioprine, cyclosporine, adalimumab, infliximab, daclizumab, abatacept, or rituximab, or a combination thereof.

Aspects disclosed herein provide a method for treating a subject having an inflammatory bowel disease, the method comprising administering to the subject a therapeutic agent, provided the subject has been determined to have a genotype comprising one or more single nucleotide polymorphisms selected from the group comprising rs80079682, rs115994059, rs7297515, rs13166683, rs62376929, rs9305694, rs139009610, rs28732100, rs12199223, rs1265181, rs13417109, rs17026757, and rs117670930. In some embodiments, the subject has been determined to be at risk for developing erythema nodosum, pyoderma gangrenosum, or psoriasis, or a combination thereof. Aspects disclosed herein provide a method for evaluating whether a subject having an inflammatory bowel disease will develop an extra-intestinal manifestation (EIM), the method comprising determining whether the subject has a genotype comprising one or more single nucleotide polymorphisms selected from the group comprising rs80079682, rs115994059, rs7297515, rs13166683, rs62376929, rs9305694, rs139009610, rs28732100, rs12199223, rs1265181, rs13417109, rs17026757, and rs117670930. In some embodiments, the EIM comprises erythema nodosum, pyoderma gangrenosum, or psoriasis, or a combination thereof. In some embodiments, the method comprises administering to the subject a therapeutic agent. In some embodiments, the therapeutic agent comprises an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof. In some embodiments, the therapeutic agent comprises an anti-inflammatory drug, cortisone, colchicine, potassium iodine, steroid, hydroxychloroquine, cyclosporin A, or thalidomide, or a combination thereof. In some embodiments, the therapeutic agent comprises a corticosteroid, a ciclosporin, infliximab, canakinumab, clobetasol, mupirocin, gentamicin, tacrolimus, mycophenolate mofetil, or thalidomide, or a combination thereof. In some embodiments, the therapeutic agent comprises a steroid, retinoid, methotrexate, adalimumab, brodalumab, certolizumab pegol, etanercept, guselkumab, infliximab, ixekizumab, risankizumab-rzaa, secukinumab, tildrakizumab, ustekinumab, or apremilast, or a combination thereof.

Aspects disclosed herein provide a method for treating a subject having an inflammatory bowel disease, the method comprising administering to the subject a therapeutic agent, provided the subject has been determined to have a genotype comprising one or more single nucleotide polymorphisms selected from the group comprising rs4349859, rs4463302, rs4418214, rs2069835, rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, and rs9276424. In some embodiments, the subject has been determined to be at risk for developing an extra-intestinal manifestation (EIM). Aspects disclosed herein provide a method for evaluating whether a subject having an inflammatory bowel disease will develop an extra-intestinal manifestation (EIM), the method comprising determining whether the subject has a genotype comprising one or more single nucleotide polymorphisms selected from the group comprising rs4349859, rs4463302, rs4418214, rs2069835, rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, and rs9276424. In some embodiments, the EIM comprises ankylosing spondylitis, sacroiliitis, erythema nodosum, pyoderma gangrenosum, psoriasis, a manifestation in the eye, or primary sclerosing cholangitis, or a combination thereof. In some embodiments, the method comprises administering to the subject a therapeutic agent. In some embodiments, the therapeutic agent comprises an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof. In some embodiments, the therapeutic agent comprises an anti-inflammatory drug, cortisone, colchicine, potassium iodine, steroid, hydroxychloroquine, cyclosporin A, thalidomide, or a combination thereof. In some embodiments, the therapeutic agent comprises a corticosteroid, ciclosporin, infliximab, canakinumab, clobetasol, mupirocin, gentamicin, tacrolimus, mycophenolate mofetil, or thalidomide, or a combination thereof. In some embodiments, the therapeutic agent comprises a steroid, retinoid, methotrexate, adalimumab, brodalumab, certolizumab pegol, etanercept, guselkumab, infliximab, ixekizumab, risankizumab-rzaa, secukinumab, tildrakizumab, ustekinumab, or apremilast, or a combination thereof. In some embodiments, the therapeutic agent comprises a steroid, methotrexate, mycophenolate, sulfasalazine, azathioprine, cyclosporine, adalimumab, infliximab, daclizumab, abatacept, or rituximab, or a combination thereof. In some embodiments, the therapeutic agent comprises ursodeoxycholic acid (UDCA), antipruritic, cholestyramine, antibiotic, vitamin A, vitamin D, vitamin E, or vitamin K, or a combination thereof. In some embodiments, the therapeutic agent comprises etanercept, adalimumab, infliximab, cyclobenzaprine, steroid, secukinumab, methotrexate, leflunomide, hydroxychloroquine, or sulfasalazine, or a combination thereof.

Aspects disclosed herein provide a method for treating a subject having an inflammatory bowel disease, the method comprising administering to the subject a therapeutic agent, provided the subject has been determined to have a genotype comprising one or more single nucleotide polymorphisms selected from the group comprising rs61199332, rs7857730, rs497871, rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, rs9276424, rs10056322, rs6461986, rs13421864, rs80079682, rs6905036, rs4349859, rs2844510, rs9276456, rs4463302, rs4418214, and rs2069835. In some embodiments, the subject has been determined to be at risk for developing an extra-intestinal manifestation (EIM).

Aspects disclosed herein provide a method for evaluating whether a subject having an inflammatory bowel disease will develop an extra-intestinal manifestation (EIM), the method comprising determining whether the subject has a genotype comprising one or more single nucleotide polymorphisms selected from the group comprising rs61199332, rs7857730, rs497871, rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, rs9276424, rs10056322, rs6461986, rs13421864, rs80079682, rs6905036, rs4349859, rs2844510, rs9276456, rs4463302, rs4418214, and rs2069835. In some embodiments, the therapeutic agent comprises an anti-TL1A antibody, an anti-CD30L antibody, glucocorticosteroid, anti-TNF therapy, anti-a4-b7 therapy, anti-IL12p40 therapy, JAK inhibitor, thalidomide, or cyclophosphamide or a combination thereof. In some embodiments, the therapeutic agent comprises an anti-inflammatory drug, cortisone, colchicine, potassium iodine, steroid, hydroxychloroquine, cyclosporin A, thalidomide, or a combination thereof. In some embodiments, the therapeutic agent comprises a corticosteroid, ciclosporin, infliximab, canakinumab, clobetasol, mupirocin, gentamicin, tacrolimus, mycophenolate mofetil, or thalidomide, or a combination thereof. In some embodiments, the therapeutic agent comprises a steroid, retinoid, methotrexate, adalimumab, brodalumab, certolizumab pegol, guselkumab, infliximab, ixekizumab, risankizumab-rzaa, secukinumab, tildrakizumab, ustekinumab, or apremilast, or a combination thereof. In some embodiments, the therapeutic agent comprises a steroid, methotrexate, mycophenolate, sulfasalazine, azathioprine, cyclosporine, adalimumab, infliximab, daclizumab, abatacept, or rituximab, or a combination thereof. In some embodiments, the therapeutic agent comprises ursodeoxycholic acid (UDCA), antipruritic, cholestyramine, antibiotic, vitamin A, vitamin D, vitamin E, or vitamin K, or a combination thereof. In some embodiments, the therapeutic agent comprises methotrexate, leflunomide, hydroxychloroquine, sulfasalazine, a NSAID, TNF inhibitor, etanercept, infliximab, belimumab, rituximab, anakinra, tocilizumab, canakinumab, secukinumab, ustekinumab, ixekizumab sarilumab, tofacitinib, abatacept, corticosteroid, prednisone, cortisone, or a combination thereof. In some embodiments, the therapeutic agent comprises etanercept, adalimumab, infliximab, cyclobenzaprine, steroid, secukinumab, methotrexate, leflunomide hydroxychloroquine, or sulfasalazine, or a combination thereof. In some embodiments, the EIM comprises ankylosing spondylitis, sacroiliitis, erythema nodosum, pyoderma gangrenosum, psoriasis, a manifestation in the eye, primary sclerosing cholangitis, or peripheral arthritis, or a combination thereof. In some embodiments, the EIM comprises ankylosing spondylitis and sacroiliitis. In some embodiments, the EIM comprises erythema nodosum, pyoderma gangrenosum, and/or psoriasis. In some embodiments, the EIM comprises the manifestation in the eye. In some embodiments, the manifestation in the eye comprises uveitis, iritis, episcleritis, scleritis, or undiagnosed ocular inflammation, or a combination thereof. In some embodiments, the EIM comprises primary sclerosing cholangitis. In some embodiments, the EIM comprises peripheral arthritis. In some embodiments, the arthritis comprises large joint arthritis, small joint arthritis, extracolonic arthritis, Crohn's disease/ulcerative colitis non-specific joint inflammation, or IBD-associated arthralgia complication, or a combination thereof. In some embodiments, the EIM comprises ankylosing spondylitis, sacroiliitis, erythema nodosum, pyoderma gangrenosum, psoriasis, a manifestation in the eye, or primary sclerosing cholangitis, or a combination thereof. In some embodiments, the EIM comprises ankylosing spondylitis, sacroiliitis, erythema nodosum, pyoderma gangrenosum, psoriasis, eye, peripheral arthritis, or primary sclerosing cholangitis, or a combination thereof. In some embodiments, the subject comprises a serological marker selected from the group comprising anti-neutrophil cytoplasmic antibodies (ANCA), anti-Saccharomyces cerevisiae antibodies (ASCA), anti-outer member porin C (anti-OmpC), anti-Pseudomonas fluorescens bacterial sequence 12 (anti-I2), and anti-bacterial flagellin (CBir1). In some embodiments, the inflammatory bowel disease comprises Crohn's disease. In some embodiments, the subject has a Crohn's disease (CD) phenotype selected from the group comprising upper GI, colonic, small bowel, structuring, penetrating, structuring and penetrating, and perianal CD. In some embodiments, the subject has undergone surgery for treatment of the inflammatory bowel disease. In some embodiments, the inflammatory bowel disease comprises ulcerative colitis. In some embodiments, the subject is female. In some embodiments, the subject is male. In some embodiments, the subject is Jewish. In some embodiments, the subject is was diagnosed with inflammatory bowel disease before age 17. In some embodiments, the subject is was diagnosed with inflammatory bowel disease before age 10. In some embodiments, the subject is a smoker. In some embodiments, the method comprises determining whether the subject comprises the one or more single nucleotide polymorphisms.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the inventive concepts set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a workflow according to an embodiment of the present disclosure for processing a biological sample obtained from a subject to inform the selection of a therapeutic agent to treat a disease or a condition of the subject.

FIG. 2 shows a computer-implemented workflow according to an embodiment of the present disclosure for generating an electronic report to a user, such as a physician, comprising a EIM profile of a subject based on an analysis of genotype data from the subject.

FIG. 3 shows a computer system that is programmed or otherwise configured to implement methods provided herein.

FIG. 4 shows a computer-implemented workflow according to an embodiment of the present disclosure for producing a EIM profile.

FIG. 5 shows the numbers of subjects with an EIM in the CEDARS, SHARE, NIDDK and RISK (CD) cohorts.

FIG. 6 shows the total unique number of subjects with an EIM with Crohn's disease, ulcerative colitis, or ulcerative colitis and inflammatory bowel disease unknown.

FIG. 7 shows a principle component analysis of all cohorts analyzed for EIM risk.

FIGS. 8A-8C depicts the results of the models to predict sensitivity and specificity of primary sclerosing cholangitis in subjects with Crohn's disease.

### DETAILED DESCRIPTION

Provided herein are methods, systems, and kits for identifying a subject who may develop an extra-intestinal manifestation based on the subject's genotype. The subject may be a patient, who may be diagnosed with an inflammatory disease, a fibrostenotic disease, or a fibrotic disease, such as inflammatory bowel disease (IBD) or Crohn's disease (CD). The subject may not be a patient, but may be suspected of having the inflammatory disease, the fibrostenotic disease, or the fibrotic disease. The genotype may, in some cases, be useful for characterizing the inflammatory fibrostenotic, or fibrotic disease or condition, as mediated by TL1A. The subject, in some embodiments, is treated by administering the therapeutic agent (e.g., anti-TL1A antibody) to the subject, provided the genotype is detected. In some cases, identifying the subject as being suitable for treatment with the inhibitor of activity or expression is required in order to administer the inhibitor to the subject.

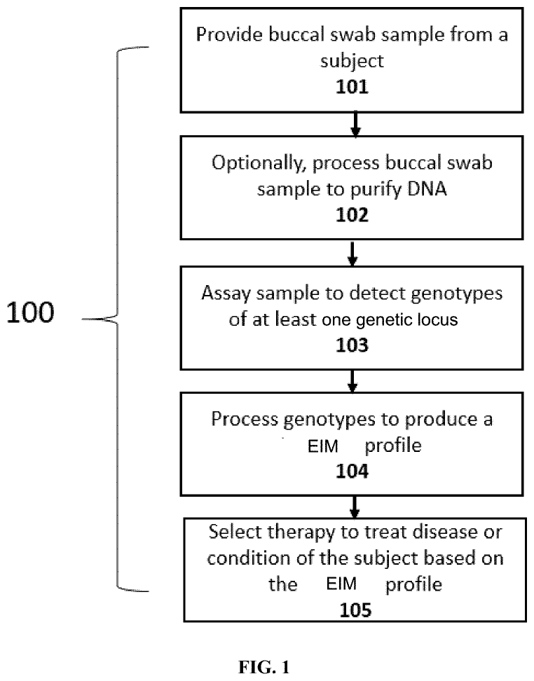

Referring to FIG. 1, the methods, systems and kits of the present disclosure involve, in some embodiments, the steps of providing a buccal swab sample from a subject 101 (although other biological samples and methods may be substituted), optionally purifying DNA from the sample by processing the sample 102, assaying the optionally processed sample to detect genotypes of at least one genetic locus in the sample 103, processing the genotypes to produce a EIM profile 104, and selecting a therapy to treat a disease or disorder of the subject based on the EIM profile 105.

The genotypes described herein are detected using suitable genotyping devices (e.g., array, sequencing). In some instances, a sample is obtained from the subject or patient indirectly or directly. In some instances, the sample may be obtained by the subject. In other instances, the sample may be obtained by a healthcare professional, such as a nurse or physician. The sample may be derived from virtually any biological fluid or tissue containing genetic information, such as blood.

The subject disclosed herein can be a mammal, such as for example a mouse, rat, guinea pig, rabbit, non-human primate, or farm animal. In some instances, the subject is human. In some instances, the subject is suffering from a symptom related to a disease or condition disclosed herein (e.g., abdominal pain, cramping, diarrhea, rectal bleeding, fever, weight loss, fatigue, loss of appetite, dehydration, and malnutrition, anemia, or ulcers).

In some embodiments, the subject is susceptible to, or is inflicted with, thiopurine toxicity, or a disease caused by thiopurine toxicity (such as pancreatitis or leukopenia). The subject may experience, or is suspected of experiencing, non-response or loss-of-response to a standard treatment (e.g., anti-TNF alpha therapy, anti-a4-b7 therapy (vedolizumab), anti-IL12p40 therapy (ustekinumab), Thalidomide, or Cytoxin).

The disease or condition disclosed herein may be an inflammatory disease, a fibrostenotic disease, or a fibrotic disease. In some instances, the disease or the condition is a TL1A-mediated disease or condition. The term, “TL1A-mediated disease or condition” refers to a disease or a condition pathology or pathogenesis that is driven, at least in part, by TL1A signaling. In some instances, the disease or the condition is immune-mediated disease or condition, such as those mediated by TL1A.

In some embodiments the disease or the condition is an inflammatory disease or disorder that is mediated, at least in part, by TL1A signaling. Non-limiting examples of inflammatory disease include, allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis, rheumatic disease, scleroderma, sinusitis, tuberculosis.

In some embodiments, the disease or the condition is an autoimmune disease that is mediated, at least in part, by TL1A signaling. Non-limiting examples of autoimmune disease or disorder include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

In some embodiments, the disease or the condition is a cancer that is mediated, at least in part, by TL1A signaling. Non-limiting examples of cancers include Adenoid Cystic Carcinoma, Adrenal Gland Cancer, Amyloidosis, Anal Cancer, Ataxia-Telangiectasia, Atypical Mole Syndrome, Basal Cell Carcinoma, Bile Duct Cancer, Birt Hogg Dube Syndrome, Bladder Cancer, Bone Cancer, Brain Tumor, Breast Cancer, Breast Cancer in Men, Carcinoid Tumor, Cervical Cancer, Colorectal Cancer, Ductal Carcinoma, Endometrial Cancer, Esophageal Cancer, Gastric Cancer, Gastrointestinal Stromal Tumor (GIST), HER2-Positive Breast Cancer, Islet Cell Tumor, Juvenile Polyposis Syndrome, Kidney Cancer, Laryngeal Cancer, Leukemia—Acute Lymphoblastic Leukemia, Leukemia—Acute Lymphocytic (ALL), Leukemia—Acute Myeloid AML, Leukemia—Adult, Leukemia—Childhood, Leukemia—Chronic Lymphocytic (CLL), Leukemia—Chronic Myeloid (CML), Liver Cancer, Lobular Carcinoma, Lung Cancer, Lung Cancer—Small Cell (SCLC), Lung Cancer—Non-small Cell (NSCLC), Lymphoma—Hodgkin's, Lymphoma—Non-Hodgkin's, Malignant Glioma, Melanoma, Meningioma, Multiple Myeloma, Myelodysplastic Syndrome (MDS), Nasopharyngeal Cancer, Neuroendocrine Tumor, Oral Cancer, Osteosarcoma, Ovarian Cancer, Pancreatic Cancer, Pancreatic Neuroendocrine Tumors, Parathyroid Cancer, Penile Cancer, Peritoneal Cancer, Peutz-Jeghers Syndrome, Pituitary Gland Tumor, Polycythemia Vera, Prostate Cancer, Renal Cell Carcinoma, Retinoblastoma, Salivary Gland Cancer, Sarcoma, Sarcoma—Kaposi, Skin Cancer, Small Intestine Cancer, Stomach Cancer, Testicular Cancer, Thymoma, Thyroid Cancer, Uterine (Endometrial) Cancer, Vaginal Cancer, and Wilms' Tumor.

In some embodiments, the disease or the condition is an inflammatory bowel disease, such as Crohn's disease (CD) or ulcerative colitis (UC). A subject may suffer from fibrosis, fibrostenosis, or a fibrotic disease, either isolated or in combination with an inflammatory disease. In some cases, the CD is severe CD. The severe CD may result from inflammation that has led to the formation of scar tissue in the intestinal wall (fibrostenosis) and/or swelling. In some cases, the severe CD is characterized by the presence of fibrotic and/or inflammatory strictures. The strictures may be determined by computed tomography enterography (CTE), and magnetic resonance imaging enterography (MRE). The disease or condition may be characterized as refractory, which in some cases, means the disease is resistant to a standard treatment (e.g., anti-TNFα therapy). Non-limiting examples of standard treatment include glucocorticosteriods, anti-TNF therapy, anti-a4-b7 therapy (vedolizumab), anti-IL12p40 therapy (ustekinumab), Thalidomide, and Cytoxin.

In some embodiments, the subject is at risk for an extra-intestinal manifestation (EIM). In some embodiments, the EIM comprises ankylosing spondylitis, sacroiliitis, erythema nodosum, pyoderma gangrenosum, psoriasis, a manifestation in the eye, or primary sclerosing cholangitis, or a combination thereof.

### Genotypes

Disclosed herein are genotypes that may be detected in a sample obtained from a subject by analyzing the genetic material in the sample. In some instances, the subject may be human. In some embodiments, the genetic material is obtained from a subject having a disease or condition disclosed herein. In some cases, the genetic material is obtained from blood, serum, plasma, sweat, hair, tears, urine, and other techniques known by one of skill in the art. In some cases, the genetic material is obtained from a biopsy, e.g., from the intestinal track of the subject.

The genotypes of the present disclosure comprise genetic material that is deoxyribonucleic acid (DNA). In some instances, the genotype comprises a denatured DNA molecule or fragment thereof. In some instances, the genotype comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented.

The genotypes disclosed herein comprise at least one polymorphism at a gene or genetic locus described herein. In some instances, the gene or genetic locus is selected from the group consisting of actin related protein 2 (ACTR2), DS cell adhesion molecule (DSCAM), interleukin 21 antisense RNA 1 (IL21-AS1), psoriasis susceptibility 1 candidate 1 (PSORSICI), psoriasis susceptibility 1 candidate 3 (PSORS1C3), dystrotelin (DYTN), interleukin 1 receptor like 2 (IL1RL2), centrosomal protein 128 (CEP128), B cell scaffold protein with ankyrin repeats 1 (BANK1), Long intergenic non-protein coding RNA 2825 (LINC02825), major histocompatibility complex class I B (HLA-B), complement factor B (CFB), histocompatibility complex class I C (HLA-C), mucin 19 (MUC19), C-type lectin domain containing 16A (CLEC16A), major histocompatibility complex class II DQ (HLA-DQA2), UDP glycosyltransferase family 3 member A1 (UGT3A1), homeobox A3 (HOXA3), SP140 nuclear body protein like (SP140L), or a combination thereof. In some instances, the gene or genetic locus comprises a gene or genetic locus provided in Table 1. The genotypes disclosed herein are, in some cases, a haplotype. In some instances, the genotype comprises a particular polymorphism, a polymorphism in linkage disequilibrium (LD) therewith, or a combination thereof. In some cases, LD is defined by an r² of at least or about 0.70, 0.75, 0.80, 0.85, 0.90, or 1.0. The genotypes disclosed herein can comprise at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more polymorphisms. In some embodiments, the genotypes disclosed herein comprise a combination of 3 polymorphisms, such as those provided in Table 1.

The polymorphisms described herein can be a single nucleotide polymorphism, or an indel (insertion/deletion). In some instances, the polymorphism is an insertion or a deletion of at least one nucleobase (e.g., an indel). In some instances, the genotype may comprise a copy number variation (CNV), which is a variation in a number of a nucleic acid sequence between individuals in a given population. In some instances, the CNV comprises at least or about two, three, four, five, six, seven, eight, nine, ten, twenty, thirty, forty or fifty nucleic acid molecules. In some instances, the genotype is heterozygous. In some instances, the genotype is homozygous.

Disclosed herein, in the following embodiments, are genotypes disclosed herein:
1. A genotype comprising at least one polymorphism at a gene or genetic locus.
2. The genotype of embodiment 1 comprising a polymorphism provided in Table 1.
3. The genotype of embodiments 1-2 that is heterozygous.
4. The genotype of embodiments 1-2 that is homozygous.
5. The genotype of embodiments 1-4, wherein the genotype comprises at least two polymorphisms.
6. The genotype of embodiments 1-4, wherein the genotype comprises at least three polymorphisms.
7. The genotype of embodiments 1-4, wherein the genotype comprises at least four polymorphisms.
8. The genotype of embodiment 1, comprising a polymorphism in linkage disequilibrium with a polymorphism provided in Table 1.
9. The genotype of embodiment 8, wherein LD is defined by (i) a D′ value of at least about 0.70, or (ii) a D′ value of 0 and an r² value of at least about 0.70.
10. The genotype of embodiment 8, wherein LD is defined by (i) a D′ value of at least about 0.80, or (ii) a D′ value of 0 and an r² value of at least about 0.80.
11. The genotype of embodiment 8, wherein LD is defined by (i) a D′ value of at least about 0.90, or (ii) a D′ value of 0 and an r² value of at least about 0.90.
12. The genotype of embodiment 8, wherein LD is defined by (i) a D′ value of at least about 0.95, or (ii) a D′ value of 0 and an r² value of at least about 0.95.
13. The genotype of embodiments 1-12, wherein the gene or genetic locus is selected from the group consisting of actin related protein 2 (ACTR2), DS cell adhesion molecule (DSCAM), interleukin 21 antisense RNA 1 (IL21-AS1), psoriasis susceptibility 1 candidate 1 (PSORSICI), psoriasis susceptibility 1 candidate 3 (PSORS1C3), dystrotelin (DYTN), interleukin 1 receptor like 2 (IL1RL2), centrosomal protein 128 (CEP128), B cell scaffold protein with ankyrin repeats 1 (BANK1), Long intergenic non-protein coding RNA 2825 (LINC02825), major histocompatibility complex class I B (HLA-B), complement factor B (CFB), histocompatibility complex class I C (HLA-C), mucin 19 (MUC19), C-type lectin domain containing 16A (CLEC16A), major histocompatibility complex class II DQ (HLA-DQA2), UDP glycosyltransferase family 3 member A1 (UGT3A1), homeobox A3 (HOXA3), SP140 nuclear body protein like (SP140L).
14. The genotype of embodiments 5-6, wherein the genotype comprises at least two polymorphisms selected from:
   a. rs6905036, rs2844510, rs2516514, and rs17030062;
   b. rs9276456, rs2858884, rs2858319, rs6917611, rs6930571, rs389419, rs76558762, rs7956721, rs887864, and rs9276424;
   c. rs10056322, rs6461986, and rs13421864;
   d. rs4349859, rs4418214, rs3819299, rs2373969, rs4151651, and rs9366775;
   e. rs80079682, rs115994059, rs7297515, rs13166683, rs62376929, rs9305694, rs139009610, rs28732100, rs12199223, rs1265181, rs13417109, rs17026757, and rs117670930;
   f. rs4349859, rs4463302, rs4418214, rs2069835, rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, and rs9276424; or
   g. rs61199332, rs7857730, rs497871, rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, rs9276424, rs10056322, rs6461986, rs13421864, rs80079682, rs6905036, rs4349859, rs2844510, rs9276456, rs4463302, rs4418214, and rs2069835.
15. The genotype of embodiments 1-14, wherein the genotype comprises a minor allele provided in Table 1 for at least one polymorphism.
16. The genotype of embodiments 1-15, wherein the genotype comprises a major allele provided in Table 1 for at least one polymorphism.

Aspects disclosed herein provide genotypes that are associated with, and therefore indicative of, a subject having or being susceptible to developing a particular disease or condition, or a subclinical phenotype thereof. In addition, the genotypes disclosed herein are associated with an extra-intestinal manifestation. Table 1 provides exemplary polymorphisms associated with, and therefore predictive of, an extra-intestinal manifestation. In some embodiments, the polymorphisms are associated with the likelihood of developing an EIM.

TABLE 1

| | | | | | SEQ |
|---|---|---|---|---|---|
| | | | Minor | Major | ID |
| rsID | Gene | | Allele | Allele | NO |
| rs2516514 | — | GGTAACTAGACCAGGCAGTGCCCTCCTTGTCAGTCTGCC | A, C | T | 2001 |
| | | TGTCTGCCTGG[T/A/C]TCCCTCTCAGAGTTTGTGTCTTCA | | | |
| | | CTCGGTTCATCATCCCCAGCCCCAGC | | | |
| rs17030062 | ACTR2 | TGTTAAGATACGAACTACCCATCTGAACGATTATAATTG | A | G | 2002 |
| | | GAAGACTTTGA[G/A]TGATGTGATTATTTGCCACTTTGCC | | | |
| | | ATGTTGTATGTCCAGTGTGTGTAGC | | | |
| rs9305694 | DSCAM | ACACAAGCATGCACACAAGTAGCTG[G]TAATCACATTG | A | G | 2003 |
| | | TCCTTAATTTGCAT | | | |
| rs139009610 | IL21- | CACACACACACACACACACACACACACACAAGCATGCA | C | A | 2004 |
| | AS1 | CACAAGTAGCTG[G/A]TAATCACATTGTCCTTAATTTGC | | | |
| | | ATCCAAAGAGGGTCAGCTGATTCTGAG | | | |
| rs28732100 | PSORS1C1 | TTACATGCTGGACATGGGCAAGATAGGAACTCAAGTTA | T | C | 2005 |
| | | CTTCCAGGTCTC[C/T]GTAAGTTTAGGACTGTGAAGAGG | | | |
| | | GCATCCTAATAGTCAAAAACATAAGTG | | | |
| rs12199223 | — | TCCTACCAGCAATAGCAGACATTCCTATTTTTTCCATAT | A | T | 2006 |
| | | TCTTGCCAGTG[T/A]TAAGACTTATCATATGTCTTTTTAA | | | |
| | | CTTTACCTGCTCTAGGTGATGTGTG | | | |
| rs1265181 | PSORS1C3 | TCTCTTTCCTTTGGCCTAGTTTGAGTGCCCAGCAGGTGT | C | G | 2007 |
| | | TTCAAGTCACT[G/C]ATTACGTATCTACTCTGCGAAAGTT | | | |
| | | GTTTGTGCAGCCTGTTTATCCTCTC | | | |
| rs13417109 | DYTN | TGAAAAACAGGTGGGAGAGAGTGAAGAGCATGATGAT | T | A | 2008 |
| | | GAATTCATTTTTT[A/T]AAAAATCTGTGCACTTTGCATTT | | | |
| | | TAATTTACAATAAACATACAGTATTAC | | | |
| rs17026757 | IL1RL2 | GCTTGGAAATTAGACAAAAGTAGAGAGGTCAGGTGGGC | C, T | A | 2009 |
| | | TAATTCCAGTTA[A/C/T]GATAGAAAAGTGAGTTGAATG | | | |
| | | GATAGAAAGTTCAGGTGGAAAAGTGTAAT | | | |
| rs117670930 | CEP128 | TCATTTGGTTACAAGGAAGTATTGAAAGTTGAGATGAG | C | T | 2010 |
| | | AAGAATAAATTA[T/C]GGAATGCAGCTCCCTGTCTAATA | | | |
| | | TTTGCATTATTATGTTTCAGACATACA | | | |
| rs115994059 | BANK1 | TTTTTTCTAGTGTCTGAAATGTGGTCTCAAAGGGAGGTG | T | G | 2011 |
| | | TAGAGAGGAGA[G/T]TAACAATGCCCCGTTGGTTGCAGT | | | |
| | | TAGAGGGGCAGACAAGTAAGAAGGTG | | | |
| rs7297515 | LINC02825 | TTTCCTGATCCTAAGCATCGAATATGGTTATGTAAGTAA | G | A | 2012 |
| | | CATCAGGAGAA[A/G]TTGGATGAAGAATATTTTGGAACT | | | |
| | | CTCTGTACTAATTTTTAGGCTGTTCT | | | |
| rs13166683 | — | CCTGTTTTACAGAGTAGTTGACCAAAGAAGAGCCTTAG | A, C | G | 2013 |
| | | GGAATAAATGAA[G/A/C]TAGTTCAAAAGGAAGTAGATT | | | |
| | | TTAATAAAAACCTAAATGAGTAGCATCAG | | | |
| rs62376929 | — | ATGAGGCACAACTTTAGTCCTTCCCCAAATACCTTGTCT | A | T | 2014 |
| | | GATCTGCCAAC[T/A]GAGACACCGCAGAGGGAAAGTGA | | | |
| | | TAGGGTGGCCAGTTCTGGGGCCTCACT | | | |
| rs4418214 | — | AAAGAAGAATAGCTCTCTCTCCTGTGAGAGAAGGGGCA | C | T | 2015 |
| | | CGCAAGTGGGAA[T/C]TCCAGCCCATGGCAGAGTGCACT | | | |
| | | GGATTTTATACACAGGCTGAAAGAGGT | | | |
| rs3819299 | HLA- | AAAACAGGACCTGGTCAGAGCCCGCAGGAGACGTGGG | G | T | 2016 |
| | B | ACAGGAGGAATTA[T/G]GGGGTGGGTGAGCTCCTCCACA | | | |
| | | CTCCCACCCCCACCACTTACACGCAGCC | | | |
| rs2373969 | — | CTTATAAGACTCTCCATGTTCTAGCCTCCCCAGCTACCT | A, C | G | 2017 |
| | | CCTGAATACTC[G/A/C]TGTGTTGTCACTCCCTTCTCTCTC | | | |
| | | ATACTCCATTTCATCACACACTCCAA | | | |
| rs4151651 | CFB | TGTCTTCCCTGACAGAGACCATAGAAGGAGTCGATGCT | A, T | G | 2018 |
| | | GAGGATGGGCAC[G/A/T]GCCCAGGTTTGAAGACAGAGA | | | |
| | | AGGGAGGCAGGGCAGGGAACTGGGGGAAA | | | |
| rs9366775 | HLA— | GCGGCGCCTCCCCAATGCAGACACGGCCCTTGGAGCCT | A, T | G | 2019 |
| | C | GAGACCCTGAGA[G/A/T]CCCCGCCCGGGACCTGGGACT | | | |
| | | TCGTCCTGATCCCTCTTCTCCTACACCAA | | | |
| rs2858884 | — | GCTCTGTTTTTAGGTGCATACACACTTAAGATTGTTTTG | C | A | 2020 |
| | | TGTCTTTGGAG[A/C]AATAACCCCTTATCATTAAATAAT | | | |
| | | ATCCCTCTTTATCCCTGGTAATATTC | | | |
| rs2858319 | — | TGTTCATTTTAATTGATGCTGAAAAAAGCATTTGATAAA | C, A | T | 2021 |
| | | ATTTAACATCA[T/A/C]TTCATGTTAAAAATTCTCAAAAA | | | |
| | | CTGGGGATATAAAGAACATACTTCAAC | | | |
| rs6917611 | — | TACACTCCAATGAGTGGTGTCAGCCAAAGTGTTTCATAG | C, T | G | 2022 |
| | | TACAGTGACAG[G/C/T]AGGATCTGTCCTTGTTTGAGTGT | | | |
| | | ACCAGCAGCAGTGGTAGTTGAAGCTGC | | | |
| rs6930571 | — | GAAGCTGCACTCACAGAGATATCCGGGCATGTCTCACA | T | G | 2023 |
| | | CTGGAAATAGGG[G/T]ACCCTTCCAAATATTGGGAGAAA | | | |
| | | GAAGGCAAAGAAAATCATACCGATATA | | | |
| rs389419 | — | CTCGTCTCCCCGCGCCTATGTCGCACGCTGGCCAGCGCC | T | C | 2024 |
| | | TCCTGGCTAAG[C/T]GGCCTCACCAACTCGGTTGCGCAA | | | |
| | | ACCGCGCTCCTGGCTGAGCGGCCGCT | | | |
| rs76558762 | — | ACATGGTCTTAATCAGTTTGAGGAAGAACTTTTCCCTTT | G | T | 2025 |
| | | GTTTTGTTTTG[T/G]TTTTGCTGAAACCTGTTTATTGTTGA | | | |
| | | TTGTTTTATCTTTTAATTTCAAAT | | | |
| rs7956721 | MUC19 | TCCCCCTTTCCTGGATTCTTCTGCTTTTCTGGGGAGATAA | C, T | G | 2026 |
| | | ACTAAGGACT[G/C/T]CTTTCATTTTAGCTGCTAAAGTCT | | | |
| | | TTTCTTGGTAGCAACTGGGAGCAGAT | | | |
| rs887864 | CLEC16A | CTTAAGCAACACTCTGAGAACAGTGTCCTGAGACATTC | A, C, | G | 2027 |
| | | AGGTCATTGCTC[G/A/C/T]GATGATGGCACTGTTTTGAG | T | | |
| | | GGGTAAGAAAGATATAGCTCTGGCTGCTTT | | | |
| rs9276424 | HLA- | GCCCGTCTCAACCTCCCAAAGTGCTGGGATTACAGGCG | A, G, | C | 2028 |
| | DQA2 | TGATCCACCGCG[C/A/G/T]CCGGCCTTAACTTTTAATGTA | T | | |
| | | GCCTGGATTGTATTTGTCTTTATACCAAT | | | |
| rs10056322 | UGT3A1 | AAGGGAAACTGATCTACCCTCTCTGAGGGCCTCTTTAGA | G | A | 2029 |
| | | AGATAACATTC[A/G]TGAAGAGGAGCTAGTAGTGTTCTA | | | |
| | | ATTCTGAAAGTCACTCCGGGTCTGTA | | | |
| rs6461986 | HOXA3 | CCTGCCATTGTATGGAGTTTGCTAACACCCACACCATAC | C, T | A | 2030 |
| | | ACTACAGACGC[A/C/T]AGAAACACCAAAAAGGCACATA | | | |
| | | ATTACCTGACAAAAGGTTAAAGCATCTC | | | |
| rs13421864. | SP140L | CACCCATCTTCTGCGTCGCTCACGCTGGGAGCTGTAGAC | A, G | C | 2031 |
| | | CGGAGCTGTTC[C/A/G]TATTCGGCCATCTTGGCTCCCGG | | | |
| | | AGCCTGCGTTTTTCTTTAATGGAAAGG | | | |

### Methods

### Methods of Detection

Methods disclosed herein for detecting a genotype in a sample from a subject comprise analyzing the genetic material in the sample to detect at least one of a presence, an absence, and a quantity of a nucleic acid sequence encompassing the genotype of interest. In some embodiments, the sample is assayed to measure a presence, absence or quantity of at least one, two, or three polymorphisms. In some embodiments, the sample is assayed to measure a presence, absence, or quantity of at least four polymorphisms. In some embodiments, the sample is assayed to measure a presence, absence, or quantity of at least five polymorphisms. In some embodiments, at least one, two, or three genotypes are detected, using the methods described herein.

In some cases, the nucleic acid sequence comprises DNA. In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises RNA. In some instances, the nucleic acid sequence comprises fragmented RNA. In some instances, the nucleic acid sequence comprises partially degraded RNA. In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Nucleic acid-based detection techniques that may be useful for the methods herein include quantitative polymerase chain reaction (qPCR), gel electrophoresis, immunochemistry, in situ hybridization such as fluorescent in situ hybridization (FISH), cytochemistry, and next generation sequencing. In some embodiments, the methods involve TaqMan™ qPCR, which involves a nucleic acid amplification reaction with a specific primer pair, and hybridization of the amplified nucleic acids with a hydrolysable probe specific to a target nucleic acid.

In some instances, the methods involve hybridization and/or amplification assays that include, but are not limited to, Southern or Northern analyses, polymerase chain reaction analyses, and probe arrays. Non-limiting amplification reactions include, but are not limited to, qPCR, self-sustained sequence replication, transcriptional amplification system, Q-Beta Replicase, rolling circle replication, or any other nucleic acid amplification known in the art. As discussed, reference to qPCR herein includes use of TaqMan™ methods. An additional exemplary hybridization assay includes the use of nucleic acid probes conjugated or otherwise immobilized on a bead, multi-well plate, or other substrate, wherein the nucleic acid probes are configured to hybridize with a target nucleic acid sequence of a genotype provided herein. A non-limiting method is one employed in Anal Chem. 2013 Feb. 5; 85(3):1932-9.

In some embodiments, detecting the presence or absence of a genotype comprises sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

In some instances, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some instances, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

Exemplary probes comprise a nucleic acid sequence of at least 10 contiguous nucleic acids provided in any one of SEQ ID NOS: 2001-2031 including the nucleobase indicated with a non-nucleobase letter (e.g., R, N, S), or a reverse complement thereof. In some instances, the probes may be used to detect the polymorphisms provided in Table 1, wherein the probe comprises a nucleic acid sequence of at least 10 contiguous nucleic acids provided in a corresponding SEQ ID NO or reverse complement thereof, the 10 contiguous nucleic acids comprising the “risk allele” also provided in Table 1 at a nucleoposition indicated with the non-nucleobase letter, or reverse complement thereof. In some embodiments, the probe comprises at least 70%, 80%, 85%, 90%, 91%, 92%, 93%, 94%, 95%, 96%, 97%, 98% or 99% sequence identity to any one of SEQ ID NOS: 2001-2031, or its reverse complement. In some instances, forward and reverse primers are used to amplify the target nucleic acid sequence. Forward and reverse primers may comprise a nucleic acid sequence flanking the risk allele provided in Table 1 corresponding to the nucleic acid sequence provided in any one of SEQ ID NOS: 2001-2031, or a reverse complement thereof.

Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i‘j’]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

In some instances, primers and/or probes described herein for detecting a target nucleic acid are used in an amplification reaction. In some instances, the amplification reaction is qPCR. An exemplary qPCR is a method employing a TaqMan™ assay. “Wt_Probe_Hex” and “Mut_Probe_FAM” mean “Wild type_probes_tagged with HEX reporter dye” and “Mut_probe_tagged with FAM reporter dye”, respectively. “+” stands for LNA bases (Locked nucleotides), which are analogues that are modified at 2′-O, 4′-C and form a bridge. This bridge results in restricted base pairing giving room to adjust the Tm as needed between the probes. Thus, +A, +T, +C or +G signify A, T, G or C bases are added on the modified backbone.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In some instances, a number of amplification cycles for detecting a target nucleic acid in an amplification assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles.

In one aspect, the methods provided herein for determining the presence, absence, and/or quantity of a nucleic acid sequence from a particular genotype comprise an amplification reaction such as qPCR. In an exemplary method, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol, or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification.

In the exemplary qPCR assay, the nucleic acid sample is combined with primers and probes specific for a target nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles.

In some embodiments, the target nucleic acid is at least 10 contiguous nucleic acid molecules of SEQ ID NO: 2001 comprising a non-reference allele at nucleoposition 51 within SEQ ID NO: 2001. In some embodiments, the target nucleic acid is at least 10 contiguous nucleic acid molecules of SEQ ID NO: 2001 comprising a “A”, “C” or a “G” allele at nucleoposition 51 within SEQ ID NO: 2001. In some embodiments, detecting the at least 10 contiguous nucleic acid molecules comprising a “A”, “C” or a “G” allele at nucleoposition 51 within SEQ ID NO: 2001 is sufficient to detect the polymorphism at rs2516514.

In some embodiments, the target nucleic acid is at least 10 contiguous nucleic acid molecules of SEQ ID NO: 2002 comprising a non-reference allele at nucleoposition 51 within SEQ ID NO: 2002. In some embodiments, the target nucleic acid is at least 10 contiguous nucleic acid molecules of SEQ ID NO: 2002 comprising a “A” or a “G” allele at nucleoposition 51 within SEQ ID NO: 2002. In some embodiments, detecting the at least 10 contiguous nucleic acid molecules comprising a “A” “or a “G” allele at nucleoposition 51 within SEQ ID NO: 2002 is sufficient to detect the polymorphism at rs17030062.

In some embodiments, the target nucleic acid is at least 10 contiguous nucleic acid molecules of SEQ ID NO: 2003 comprising a non-reference allele at nucleoposition 51 within SEQ ID NO: 2003. In some embodiments, the target nucleic acid is at least 10 contiguous nucleic acid molecules of SEQ ID NO: 2003 comprising a “A” or a “G” allele at nucleoposition 51 within SEQ ID NO: 2003. In some embodiments, detecting the at least 10 contiguous nucleic acid molecules comprising a “A” or a “G” allele at nucleoposition 51 within SEQ ID NO: 2003 is sufficient to detect the polymorphism at rs9305694.

In some embodiments, the target nucleic acid is at least 10 contiguous nucleic acid molecules of SEQ ID NO: 2004 comprising a non-reference allele at nucleoposition 51 within SEQ ID NO: 2004. In some embodiments, the target nucleic acid is at least 10 contiguous nucleic acid molecules of SEQ ID NO: 2004 comprising a “A” or a “C” allele at nucleoposition 51 within SEQ ID NO: 2004. In some embodiments, detecting the at least 10 contiguous nucleic acid molecules comprising a “A” or a “C” allele at nucleoposition 51 within SEQ ID NO: 2004 is sufficient to detect the polymorphism at rs139009610.

In some embodiments, the target nucleic acid is at least 10 contiguous nucleic acid molecules of SEQ ID NO: 2005 comprising a non-reference allele at nucleoposition 51 within SEQ ID NO: 2005. In some embodiments, the target nucleic acid is at least 10 contiguous nucleic acid molecules of SEQ ID NO: 2005 comprising a “A” or an “C” allele at nucleoposition 51 within SEQ ID NO: 2005. In some embodiments, detecting the at least 10 contiguous nucleic acid molecules comprising a “A” or an “C” allele at nucleoposition 51 within SEQ ID NO: 2005 is sufficient to detect the polymorphism at rs28732100.

In some embodiments, the target nucleic acid is at least 10 contiguous nucleic acid molecules of SEQ ID NO: 2006 comprising a non-reference allele at nucleoposition 51 within SEQ ID NO: 2006. In some embodiments, the target nucleic acid is at least 10 contiguous nucleic acid molecules of SEQ ID NO: 2006 comprising a “A” or an “T” allele at nucleoposition 51 within SEQ ID NO: 2006. In some embodiments, detecting the at least 10 contiguous nucleic acid molecules comprising a “A” or an “T” allele at nucleoposition 51 within SEQ ID NO: 2006 is sufficient to detect the polymorphism at rs12199223.

In some embodiments, the target nucleic acid is at least 10 contiguous nucleic acid molecules of SEQ ID NO: 2007 comprising a non-reference allele at nucleoposition 51 within SEQ ID NO: 2007. In some embodiments, the target nucleic acid is at least 10 contiguous nucleic acid molecules of SEQ ID NO: 2007 comprising a “C” or a “G” allele at nucleoposition 51 within SEQ ID NO: 2007. In some embodiments, detecting the at least 10 contiguous nucleic acid molecules comprising a “C” or a “G” allele at nucleoposition 51 within SEQ ID NO: 2007 is sufficient to detect the polymorphism at rs1265181.

In some embodiments, the target nucleic acid is at least 10 contiguous nucleic acid molecules of SEQ ID NO: 2008 comprising a non-reference allele at nucleoposition 51 within SEQ ID NO: 2008. In some embodiments, the target nucleic acid is at least 10 contiguous nucleic acid molecules of SEQ ID NO: 2008 comprising a “A” or an “T” allele at nucleoposition 51 within SEQ ID NO: 2008. In some embodiments, detecting the at least 10 contiguous nucleic acid molecules comprising a “A” or an “T” allele at nucleoposition 51 within SEQ ID NO: 2008 is sufficient to detect the polymorphism at rs13417109.

In some embodiments, the target nucleic acid is at least 10 contiguous nucleic acid molecules of SEQ ID NO: 2009 comprising a non-reference allele at nucleoposition 51 within SEQ ID NO: 2009. In some embodiments, the target nucleic acid is at least 10 contiguous nucleic acid molecules of SEQ ID NO: 2009 comprising a “A”, “C” or an “T” allele at nucleoposition 51 within SEQ ID NO: 2009. In some embodiments, detecting the at least 10 contiguous nucleic acid molecules comprising a “A”, “C” or an “T” allele at nucleoposition 51 within SEQ ID NO: 2009 is sufficient to detect the polymorphism at rs17026757.

In some embodiments, the target nucleic acid is at least 10 contiguous nucleic acid molecules of SEQ ID NO: 2010 comprising a non-reference allele at nucleoposition 51 within SEQ ID NO: 2010. In some embodiments, the target nucleic acid is at least 10 contiguous nucleic acid molecules of SEQ ID NO: 2010 comprising a C” or an “T” allele at nucleoposition 51 within SEQ ID NO: 2010. In some embodiments, detecting the at least 10 contiguous nucleic acid molecules comprising a “C” or an “T” allele at nucleoposition 51 within SEQ ID NO: 2010 is sufficient to detect the polymorphism at rs117670930.

In some embodiments, the target nucleic acid is at least 10 contiguous nucleic acid molecules of SEQ ID NO: 2011 comprising a non-reference allele at nucleoposition 51 within SEQ ID NO: 2011. In some embodiments, the target nucleic acid is at least 10 contiguous nucleic acid molecules of SEQ ID NO: 2011 comprising a “T” or a “G” allele at nucleoposition 51 within SEQ ID NO: 2011. In some embodiments, detecting the at least 10 contiguous nucleic acid molecules comprising a “T” or a “G” allele at nucleoposition 51 within SEQ ID NO: 2011 is sufficient to detect the polymorphism at rs115994059.

In some embodiments, the target nucleic acid is at least 10 contiguous nucleic acid molecules of SEQ ID NO: 2012 comprising a non-reference allele at nucleoposition 51 within SEQ ID NO: 2012. In some embodiments, the target nucleic acid is at least 10 contiguous nucleic acid molecules of SEQ ID NO: 2012 comprising a “A” or a “G” allele at nucleoposition 51 within SEQ ID NO: 2012. In some embodiments, detecting the at least 10 contiguous nucleic acid molecules comprising a “A” or a “G” allele at nucleoposition 51 within SEQ ID NO: 2012 is sufficient to detect the polymorphism at rs7297515.

In some embodiments, the target nucleic acid is at least 10 contiguous nucleic acid molecules of SEQ ID NO: 2013 comprising a non-reference allele at nucleoposition 51 within SEQ ID NO: 2013. In some embodiments, the target nucleic acid is at least 10 contiguous nucleic acid molecules of SEQ ID NO: 2013 comprising a “A”, “C” or a “G” allele at nucleoposition 51 within SEQ ID NO: 2013. In some embodiments, detecting the at least 10 contiguous nucleic acid molecules comprising a “A”, “C” or a “G” allele at nucleoposition 51 within SEQ ID NO: 2013 is sufficient to detect the polymorphism at rs9305694.

In some embodiments, the target nucleic acid is at least 10 contiguous nucleic acid molecules of SEQ ID NO: 2014 comprising a non-reference allele at nucleoposition 51 within SEQ ID NO: 2014. In some embodiments, the target nucleic acid is at least 10 contiguous nucleic acid molecules of SEQ ID NO: 2014 comprising a “A” or a “T” allele at nucleoposition 51 within SEQ ID NO: 2014. In some embodiments, detecting the at least 10 contiguous nucleic acid molecules comprising a “A” or a “T” allele at nucleoposition 51 within SEQ ID NO: 2014 is sufficient to detect the polymorphism at rs62376929.

In some embodiments, the target nucleic acid is at least 10 contiguous nucleic acid molecules of SEQ ID NO: 2015 comprising a non-reference allele at nucleoposition 51 within SEQ ID NO: 2015. In some embodiments, the target nucleic acid is at least 10 contiguous nucleic acid molecules of SEQ ID NO: 2015 comprising a “C” or an “T” allele at nucleoposition 51 within SEQ ID NO: 2015. In some embodiments, detecting the at least 10 contiguous nucleic acid molecules comprising a “C” or an “T” allele at nucleoposition 51 within SEQ ID NO: 2015 is sufficient to detect the polymorphism at rs4418214.

In some embodiments, the target nucleic acid is at least 10 contiguous nucleic acid molecules of SEQ ID NO: 2016 comprising a non-reference allele at nucleoposition 51 within SEQ ID NO: 2016. In some embodiments, the target nucleic acid is at least 10 contiguous nucleic acid molecules of SEQ ID NO: 2016 comprising a “G” or an “T” allele at nucleoposition 51 within SEQ ID NO: 2016. In some embodiments, detecting the at least 10 contiguous nucleic acid molecules comprising a “G” or an “T” allele at nucleoposition 51 within SEQ ID NO: 2016 is sufficient to detect the polymorphism at rs3819299.

In some embodiments, the target nucleic acid is at least 10 contiguous nucleic acid molecules of SEQ ID NO: 2017 comprising a non-reference allele at nucleoposition 51 within SEQ ID NO: 2017. In some embodiments, the target nucleic acid is at least 10 contiguous nucleic acid molecules of SEQ ID NO: 2017 comprising a “A”, “C” or a “G” allele at nucleoposition 51 within SEQ ID NO: 2017. In some embodiments, detecting the at least 10 contiguous nucleic acid molecules comprising a “A”, “C” or a “G” allele at nucleoposition 51 within SEQ ID NO: 2017 is sufficient to detect the polymorphism at rs2373969.

In some embodiments, the target nucleic acid is at least 10 contiguous nucleic acid molecules of SEQ ID NO: 2018 comprising a non-reference allele at nucleoposition 51 within SEQ ID NO: 2018. In some embodiments, the target nucleic acid is at least 10 contiguous nucleic acid molecules of SEQ ID NO: 2018 comprising a “A”, “T” or a “G” allele at nucleoposition 51 within SEQ ID NO: 2018. In some embodiments, detecting the at least 10 contiguous nucleic acid molecules comprising a “A”, “T” or a “G” allele at nucleoposition 51 within SEQ ID NO: 2018 is sufficient to detect the polymorphism at rs4151651.

In some embodiments, the target nucleic acid is at least 10 contiguous nucleic acid molecules of SEQ ID NO: 2019 comprising a non-reference allele at nucleoposition 51 within SEQ ID NO: 2019. In some embodiments, the target nucleic acid is at least 10 contiguous nucleic acid molecules of SEQ ID NO: 2019 comprising a “A”, “T” or a “G” allele at nucleoposition 51 within SEQ ID NO: 2019. In some embodiments, detecting the at least 10 contiguous nucleic acid molecules comprising a “A”, “T” or a “G” allele at nucleoposition 51 within SEQ ID NO: 2019 is sufficient to detect the polymorphism at rs9366775.

In some embodiments, the target nucleic acid is at least 10 contiguous nucleic acid molecules of SEQ ID NO: 2020 comprising a non-reference allele at nucleoposition 51 within SEQ ID NO: 2020. In some embodiments, the target nucleic acid is at least 10 contiguous nucleic acid molecules of SEQ ID NO: 2020 comprising a C” or an “A” allele at nucleoposition 51 within SEQ ID NO: 2020. In some embodiments, detecting the at least 10 contiguous nucleic acid molecules comprising a “C” or an “A” allele at nucleoposition 51 within SEQ ID NO: 2020 is sufficient to detect the polymorphism at rs2858884.

In some embodiments, the target nucleic acid is at least 10 contiguous nucleic acid molecules of SEQ ID NO: 2021 comprising a non-reference allele at nucleoposition 51 within SEQ ID NO: 2021. In some embodiments, the target nucleic acid is at least 10 contiguous nucleic acid molecules of SEQ ID NO: 2021 comprising a “C”, “T” or a “G” allele at nucleoposition 51 within SEQ ID NO: 2021. In some embodiments, detecting the at least 10 contiguous nucleic acid molecules comprising a “C”, “T” or a “G” allele at nucleoposition 51 within SEQ ID NO: 2021 is sufficient to detect the polymorphism at rs2858319.

In some embodiments, the target nucleic acid is at least 10 contiguous nucleic acid molecules of SEQ ID NO: 2022 comprising a non-reference allele at nucleoposition 51 within SEQ ID NO: 2022. In some embodiments, the target nucleic acid is at least 10 contiguous nucleic acid molecules of SEQ ID NO: 2022 comprising a “C”, “T” or a “G” allele at nucleoposition 51 within SEQ ID NO: 2022. In some embodiments, detecting the at least 10 contiguous nucleic acid molecules comprising a “C”, “T” or a “G” allele at nucleoposition 51 within SEQ ID NO: 2022 is sufficient to detect the polymorphism at rs6917611.

In some embodiments, the target nucleic acid is at least 10 contiguous nucleic acid molecules of SEQ ID NO: 2023 comprising a non-reference allele at nucleoposition 51 within SEQ ID NO: 2023. In some embodiments, the target nucleic acid is at least 10 contiguous nucleic acid molecules of SEQ ID NO: 2023 comprising a “T” or a “G” allele at nucleoposition 51 within SEQ ID NO: 2023. In some embodiments, detecting the at least 10 contiguous nucleic acid molecules comprising a “T” or a “G” allele at nucleoposition 51 within SEQ ID NO: 2023 is sufficient to detect the polymorphism at rs6930571.

In some embodiments, the target nucleic acid is at least 10 contiguous nucleic acid molecules of SEQ ID NO: 2024 comprising a non-reference allele at nucleoposition 51 within SEQ ID NO: 2024. In some embodiments, the target nucleic acid is at least 10 contiguous nucleic acid molecules of SEQ ID NO: 2024 comprising a “T” or a “C” allele at nucleoposition 51 within SEQ ID NO: 2024. In some embodiments, detecting the at least 10 contiguous nucleic acid molecules comprising a “T” or a “C” allele at nucleoposition 51 within SEQ ID NO: 2024 is sufficient to detect the polymorphism at rs389419.

In some embodiments, the target nucleic acid is at least 10 contiguous nucleic acid molecules of SEQ ID NO: 2025 comprising a non-reference allele at nucleoposition 51 within SEQ ID NO: 2025. In some embodiments, the target nucleic acid is at least 10 contiguous nucleic acid molecules of SEQ ID NO: 2025 comprising a “G” or an “T” allele at nucleoposition 51 within SEQ ID NO: 2025. In some embodiments, detecting the at least 10 contiguous nucleic acid molecules comprising a “G” or an “T” allele at nucleoposition 51 within SEQ ID NO: 2025 is sufficient to detect the polymorphism at rs76558762.

In some embodiments, the target nucleic acid is at least 10 contiguous nucleic acid molecules of SEQ ID NO: 2026 comprising a non-reference allele at nucleoposition 51 within SEQ ID NO: 2026. In some embodiments, the target nucleic acid is at least 10 contiguous nucleic acid molecules of SEQ ID NO: 2026 comprising a “C”, “T” or a “G” allele at nucleoposition 51 within SEQ ID NO: 2026. In some embodiments, detecting the at least 10 contiguous nucleic acid molecules comprising a “C”, “T” or a “G” allele at nucleoposition 51 within SEQ ID NO: 2026 is sufficient to detect the polymorphism at rs7956721.

In some embodiments, the target nucleic acid is at least 10 contiguous nucleic acid molecules of SEQ ID NO: 2027 comprising a non-reference allele at nucleoposition 51 within SEQ ID NO: 2027. In some embodiments, the target nucleic acid is at least 10 contiguous nucleic acid molecules of SEQ ID NO: 2027 comprising a “A”, “G”, “T” or a “C” allele at nucleoposition 51 within SEQ ID NO: 2027. In some embodiments, detecting the at least 10 contiguous nucleic acid molecules comprising a “A”, “G”, “T” or a “C” allele at nucleoposition 51 within SEQ ID NO: 2027 is sufficient to detect the polymorphism at rs887864.

In some embodiments, the target nucleic acid is at least 10 contiguous nucleic acid molecules of SEQ ID NO: 2028 comprising a non-reference allele at nucleoposition 51 within SEQ ID NO: 2028. In some embodiments, the target nucleic acid is at least 10 contiguous nucleic acid molecules of SEQ ID NO: 2028 comprising a “A”, “C”, “T” or a “G” allele at nucleoposition 51 within SEQ ID NO: 2028. In some embodiments, detecting the at least 10 contiguous nucleic acid molecules comprising a “A”, “C”, “T” or a “G” allele at nucleoposition 51 within SEQ ID NO: 2028 is sufficient to detect the polymorphism at rs9276424.

In some embodiments, the target nucleic acid is at least 10 contiguous nucleic acid molecules of SEQ ID NO: 2029 comprising a non-reference allele at nucleoposition 51 within SEQ ID NO: 2029. In some embodiments, the target nucleic acid is at least 10 contiguous nucleic acid molecules of SEQ ID NO: 2029 comprising a “G” or an “A” allele at nucleoposition 51 within SEQ ID NO: 2029. In some embodiments, detecting the at least 10 contiguous nucleic acid molecules comprising a “G” or an “A” allele at nucleoposition 51 within SEQ ID NO: 2029 is sufficient to detect the polymorphism at rs10056322.

In some embodiments, the target nucleic acid is at least 10 contiguous nucleic acid molecules of SEQ ID NO: 2030 comprising a non-reference allele at nucleoposition 51 within SEQ ID NO: 2030. In some embodiments, the target nucleic acid is at least 10 contiguous nucleic acid molecules of SEQ ID NO: 2030 comprising a “C”, “T” or an “A” allele at nucleoposition 51 within SEQ ID NO: 2030. In some embodiments, detecting the at least 10 contiguous nucleic acid molecules comprising a “C”, “T” or an “A” allele at nucleoposition 51 within SEQ ID NO: 2030 is sufficient to detect the polymorphism at rs6461986.

In some embodiments, the target nucleic acid is at least 10 contiguous nucleic acid molecules of SEQ ID NO: 2031 comprising a non-reference allele at nucleoposition 51 within SEQ ID NO: 2031. In some embodiments, the target nucleic acid is at least 10 contiguous nucleic acid molecules of SEQ ID NO: 2031 comprising a “A”, “G” or a “C” allele at nucleoposition 51 within SEQ ID NO: 2031. In some embodiments, detecting the at least 10 contiguous nucleic acid molecules comprising a “A”, “G” or a “C” allele at nucleoposition 51 within SEQ ID NO: 2031 is sufficient to detect the polymorphism at rs13421864.

In some embodiments, one target nucleic acid (e.g., a polymorphism) is detected with the methods disclosed herein. In some embodiments, at least 2, 3, 4, 5, 6, 7, 8, 9, or 10 target nucleic acids are detected. In some embodiments, the at least 2, 3, 4, 5, 6, 7, 8, 9, or 10 target nucleic acids are detected in a single multiplexed assay. In some embodiments, when 4 target nucleic acids are detected in a sample from subject, 4 unique 3-polymorphism combinations are measured. In a non-limiting example, a sample (e.g., blood or plasma) obtained from a subject is contacted by 4 primer pairs, each primer pair individually adapted to amplify rs6905036, rs2844510, rs2516514, and rs17030062, respectively. A positive, negative, or indeterminate profile may depend, at least in part, on which of the 3-polymorphism combinations is detected in the sample, and/or whether the genotype is heterozygous or homozygous for the polymorphism. In this example, assaying 4 polymorphism means a total of 4 unique 3-polymorphisms may be detected in the patient sample, which are rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, rs9276424, rs10056322, rs6461986, and rs13421864. Each polymorphism detected may be heterozygous or homozygous.

To practice the methods and systems provided herein, genetic material may be extracted from a sample obtained from a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification. In certain embodiments, RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland).

In some embodiments, methods of detecting a presence, absence, or level of a target protein (e.g., biomarker) in the sample obtained from the subject involve detecting protein activity or expression. In some embodiments, the target protein is TL1A, or a binding partner of TL1A such as Death Domain Receptor 3 (DcR3). A target protein may be detected by use of an antibody-based assay, where an antibody specific to the target protein is utilized. In some embodiments, antibody-based detection methods utilize an antibody that binds to any region of target protein. An exemplary method of analysis comprises performing an enzyme-linked immunosorbent assay (ELISA). The ELISA assay may be a sandwich ELISA or a direct ELISA. Another exemplary method of analysis comprises a single molecule array, e.g., Simoa. Other exemplary methods of detection include immunohistochemistry and lateral flow assay. Additional exemplary methods for detecting target protein include, but are not limited to, gel electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. Exemplary supports or carriers include glass, polystyrene, polypropylene, polyethylene, dextran, nylon, amylases, natural and modified celluloses, polyacrylamides, gabbros, and magnetite.

In some cases, a target protein may be detected by detecting binding between the target protein and a binding partner of the target protein. Non-limiting examples of binding partners to TL1A include DcR3, and Tumor necrosis factor receptor superfamily member 25 (TNR25). Exemplary methods of analysis of protein-protein binding comprise performing an assay in vivo or in vitro, or ex vivo. In some instances, the method of analysis comprises an assay such as a co-immunoprecipitation (co-IP), pull-down, crosslinking protein interaction analysis, labeled transfer protein interaction analysis, or Far-western blot analysis, FRET based assay, including, for example FRET-FLIM, a yeast two-hybrid assay, BiFC, or split luciferase assay.

Disclosed herein are methods of detecting a presence or a level of one or more serological markers in a sample obtained from a subject. In some embodiments, the one or more serological markers comprises anti-Saccharomyces cerevisiae antibody (ASCA), an anti-neutrophil cytoplasmic antibody (ANCA), antibody against E. coli outer membrane porin protein C (anti-OmpC), anti-chitin antibody, pANCA antibody, anti-I2 antibody, and anti-Cbirl flagellin antibody. In some embodiments, the antibodies comprises immunoglobulin A (IgA), immunoglobulin G (IgG), immunoglobulin E (IgE), or immunoglobulin M (IgM), immunoglobulin D (IgD), or a combination thereof. Any suitable method for detecting a target protein or biomarker disclosed herein may be used to detect a presence, absence, or level of a serological marker. In some embodiments, the presence or the level of the one or more serological markers is detected using an enzyme-linked immunosorbent assay (ELISA), a single molecule array (Simoa), immunohistochemistry, internal transcribed spacer (ITS) sequencing, or any combination thereof. In some embodiments, the ELISA is a fixed leukocyte ELISA. In some embodiments, the ELISA is a fixed neutrophil ELISA. A fixed leukocyte or neutrophil ELISA may be useful for the detection of certain serological markers, such as those described in Saxon et al., A distinct subset of antineutrophil cytoplasmic antibodies is associated with inflammatory bowel disease, J. Allergy Clin. Immuno. 86:2; 202-210 (August 1990). In some embodiments, ELISA units (EU) are used to measure positivity of a presence or level of a serological marker (e.g., seropositivity), which reflects a percentage of a standard or reference value. In some embodiments, the standard comprises pooled sera obtained from well-characterized patient population (e.g., diagnosed with the same disease or condition the subject has, or is suspected of having) reported as being seropositive for the serological marker of interest. In some embodiments, the control or reference value comprises 10, 20, 30, 40, 50, 60, 70, 80, 90, or 100 EU. In some instances, a quartile sum scores are calculated using, for example, the methods reported in Landers C J, Cohavy O, Misra R. et al., Selected loss of tolerance evidenced by Crohn's disease-associated immune responses to auto- and microbial antigens. Gastroenterology (2002)123:689-699.

### Extra-Intestinal Manifestations

Inflammatory Bowel Disease may cause symptoms outside of the gut. Symptoms of inflammatory bowel disease that occur outside of the gastrointestinal system are referred to as extra-intestinal manifestations (EIMs). 25%-40% of IBD patients experience EIMs. In some embodiments, the EIM may be present in a tissue selected from the group consisting of a joint, skin, bone, eye, kidney, or liver. In some embodiments, the EIM comprises ankylosing spondylitis, sacroiliitis, erythema nodosum, pyoderma gangrenosum, psoriasis, a manifestation in the eye, primary sclerosing cholangitis, or peripheral arthritis, or a combination thereof.

In some embodiments, the EIM comprises ankylosing spondylitis and sacroiliitis. In some embodiments, the therapeutic agent comprises an anti-TL1A antibody, anti-CD30L antibody, glucocorticosteroid, anti-TNF therapy, anti-a4-b7 therapy (e.g., vedolizumab), anti-IL12p40 therapy (e.g., ustekinumab), JAK inhibitor (e.g., tofacitinib), thalidomide, or cyclophosphamide (Cytoxan), or a combination thereof. In some embodiments, the therapeutic agent comprises etanercept (e.g., Enbrel), adalimumab (e.g., Humira), infliximab (e.g., Remicade), cyclobenzaprine, a steroid, secukinumab (e.g., Cosentyx), methotrexate, leflunomide (e.g., Arava), hydroxychloroquine, or sulfasalazine (e.g., Azulfidine), or a combination thereof. In some embodiments, the therapeutic agent comprises an anti-TL1A antibody as described herein. In some embodiments, the therapeutic agent comprises an anti-CD30L antibody as described herein. In some embodiments, the therapeutic agent comprises a glucocorticosteroid. In some embodiments, the therapeutic agent comprises anti-TNF therapy. In some embodiments, the therapeutic agent comprises anti-a4-b7 therapy. In some embodiments, the therapeutic agent comprises a JAK inhibitor. In some embodiments, the therapeutic agent comprises thalidomide. In some embodiments, the therapeutic agent comprises cyclophosphamide. In some embodiments, the therapeutic agent comprises adalimumab. In some embodiments, the therapeutic agent comprises etanercept. In some embodiments, the therapeutic agent comprises infliximab. In some embodiments, the therapeutic agent comprises cyclobenzaprine. In some embodiments, the therapeutic agent comprises a steroid. In some embodiments, the therapeutic agent comprises secukinumab. In some embodiments, the therapeutic agent comprises methotrexate. In some embodiments, the therapeutic agent comprises leflunomide. In some embodiments, the therapeutic agent comprises hydroxychloroquine. In some embodiments, the therapeutic agent comprises sulfasalazine.

EIMs may manifest in the skin. In some embodiments, the EIM comprises erythema nodosum. In some embodiments, the EIM comprises pyoderma gangrenosum. In some embodiments, the EIM comprises psoriasis. In some embodiments, the EIM comprises erythema nodosum, pyoderma gangrenosum, and/or psoriasis. In some embodiments, the EIM comprises erythema nodosum, pyoderma gangrenosum, and psoriasis. In some embodiments, the EIM comprises erythema nodosum and the therapeutic agent comprises an anti-inflammatory drug, cortisone, colchicine, potassium iodine, a steroid, hydroxychloroquine (Plaquenil), cyclosporin A (Sandimmune), thalidomide (Thalomid), or a combination thereof. In some embodiments, the EIM comprises pyoderma gangrenosum and the therapeutic agent comprises a corticosteroid, a ciclosporin, infliximab, canakinumab, clobetasol, mupirocin, gentamicin, tacrolimus, mycophenolate mofetil; mycophenolate mofetil, thalidomide; or a combination thereof. In some embodiments, the EIM comprises psoriasis and the therapeutic agent comprises (apremilast), a steroid, a retinoid, methotrexate, adalimumab (Humira), brodalumab (Siliq), certolizumab pegol (Cimzia) etanercept (Enbrel), guselkumab (Tremfya), infliximab (Remicade), ixekizumab (Taltz), risankizumab-rzaa (SKYRIZI), secukinumab (Cosentyx), tildrakizumab (Ilumya), ustekinumab (Stelara), apremilast (Otezla), or a combination thereof. In some embodiments, the therapeutic agent comprises an anti-TL1A antibody, anti-CD30L antibody, glucocorticosteroid, anti-TNF therapy, anti-a4-b7 therapy (e.g., vedolizumab), anti-IL12p40 therapy (e.g., ustekinumab), JAK inhibitor (e.g., tofacitinib), thalidomide, or cyclophosphamide (Cytoxan), or a combination thereof. In some embodiments, the therapeutic agent comprises an anti-inflammatory drug, cortisone, colchicine, potassium iodine, steroid, hydroxychloroquine, cyclosporin A (e.g., Sandimmune), or thalidomide, or a combination thereof. In some embodiments, the therapeutic agent comprises a corticosteroid, a ciclosporin, infliximab (e.g., Remicade), canakinumab (e.g., Ilaris), clobetasol, mupirocin, gentamicin, tacrolimus, mycophenolate mofetil, or thalidomide, or a combination thereof. In some embodiments, the therapeutic agent comprises a steroid, retinoid, methotrexate, adalimumab (e.g., Humira), brodalumab (e.g., Siliq), certolizumab pegol (e.g., Cimzia) etanercept (e.g., Enbrel), guselkumab (e.g., Tremfya), infliximab (e.g., Remicade), ixekizumab (e.g., Taltz), risankizumab-rzaa (e.g., SKYRIZI), secukinumab (e.g., Cosentyx), tildrakizumab (e.g., Ilumya), ustekinumab (e.g., Stelara), or apremilast (e.g., Otezla), or a combination thereof. In some embodiments, the therapeutic agent comprises an anti-TL1A antibody as described herein. In some embodiments, the therapeutic agent comprises an anti-CD30L antibody as described herein. In some embodiments, the therapeutic agent comprises a glucocorticosteroid. In some embodiments, the therapeutic agent comprises anti-TNF therapy. In some embodiments, the therapeutic agent comprises anti-a4-b7 therapy. In some embodiments, the therapeutic agent comprises a JAK inhibitor. In some embodiments, the therapeutic agent comprises thalidomide. In some embodiments, the therapeutic agent comprises cyclophosphamide. In some embodiments, the therapeutic agent comprises an anti-inflammatory drug. In some embodiments, the therapeutic agent comprises ciclosporin. In some embodiments, the therapeutic agent comprises ciclosporin. In some embodiments, the therapeutic agent comprises a steroid. In some embodiments, the therapeutic agent comprises adalimumab. In some embodiments, the therapeutic agent comprises canakinumab. In some embodiments, the therapeutic agent comprises brodalumab. In some embodiments, the therapeutic agent comprises certolizumab pegol. In some embodiments, the therapeutic agent comprises clobetasol. In some embodiments, the therapeutic agent comprises colchicine. In some embodiments, the therapeutic agent comprises gentamicin. In some embodiments, the therapeutic agent comprises cortisone. In some embodiments, the therapeutic agent comprises gentamicin. In some embodiments, the therapeutic agent comprises guselkumab. In some embodiments, the therapeutic agent comprises hydroxychloroquine. In some embodiments, the therapeutic agent comprises ixekizumab. In some embodiments, the therapeutic agent comprises methotrexate. In some embodiments, the therapeutic agent comprises mupirocin. In some embodiments, the therapeutic agent comprises mycophenolate mofetil. In some embodiments, the therapeutic agent comprises potassium iodine. In some embodiments, the therapeutic agent comprises retinoid. In some embodiments, the therapeutic agent comprises secukinumab. In some embodiments, the therapeutic agent comprises risankizumab-rzaa. In some embodiments, the therapeutic agent comprises a steroid. In some embodiments, the therapeutic agent comprises tacrolimus. In some embodiments, the therapeutic agent comprises thalidomide. In some embodiments, the therapeutic agent comprises tildrakizumab. In some embodiments, the therapeutic agent comprises ustekinumab. In some embodiments, the therapeutic agent comprises apremilast. In some embodiments, the therapeutic agent comprises etanercept. In some embodiments, the therapeutic agent comprises thalidomide.

In some embodiments, the EIM comprises the manifestation in the eye. In some embodiments, the manifestation in the eye comprises uveitis, iritis, episcleritis, scleritis, or undiagnosed ocular inflammation, or a combination thereof. In some embodiments, the therapeutic agent comprises an anti-TL1A antibody, anti-CD30L antibody, glucocorticosteroid, anti-TNF therapy, anti-a4-b7 therapy (e.g., vedolizumab), anti-IL12p40 therapy (e.g., ustekinumab), JAK inhibitor (e.g., tofacitinib), thalidomide, or cyclophosphamide (Cytoxan), or a combination thereof. In some embodiments, wherein the therapeutic agent comprises a steroid, methotrexate, mycophenolate, sulfasalazine (e.g., Azulfidine), azathioprine (e.g., Imuran), cyclosporine, adalimumab (e.g., Humira), infliximab (e.g., Remicade), daclizumab (e.g., Zinbryta), abatacept (e.g., Orencia), or rituximab (e.g., Rituxan), or a combination thereof. In some embodiments, the therapeutic agent comprises an anti-TL1A antibody as described herein. In some embodiments, the therapeutic agent comprises an anti-CD30L antibody as described herein. In some embodiments, the therapeutic agent comprises a glucocorticosteroid. In some embodiments, the therapeutic agent comprises anti-TNF therapy. In some embodiments, the therapeutic agent comprises anti-a4-b7 therapy. In some embodiments, the therapeutic agent comprises a JAK inhibitor. In some embodiments, the therapeutic agent comprises thalidomide. In some embodiments, the therapeutic agent comprises cyclophosphamide. In some embodiments, the therapeutic agent comprises a steroid. In some embodiments, the therapeutic agent comprises methotrexate. In some embodiments, the therapeutic agent comprises mycophenolate. In some embodiments, the therapeutic agent comprises sulfasalazine. In some embodiments, the therapeutic agent comprises azathioprine. In some embodiments, the therapeutic agent comprises cyclosporine. In some embodiments, the therapeutic agent comprises adalimumab. In some embodiments, the therapeutic agent comprises infliximab. In some embodiments, the therapeutic agent comprises daclizumab. In some embodiments, the therapeutic agent comprises abatacept. In some embodiments the therapeutic agent comprises rituximab.

Primary sclerosing cholangitis (PSC) is a long-term progressive disease of the liver and gallbladder characterized by inflammation and scarring of the bile ducts which normally allow bile to drain from the gallbladder. In some embodiments, the EIM comprises primary sclerosing cholangitis. In some embodiments, the therapeutic agent comprises an anti-TL1A antibody, anti-CD30L antibody, glucocorticosteroid, anti-TNF therapy, anti-a4-b7 therapy (e.g., vedolizumab), anti-IL12p40 therapy (e.g., ustekinumab), JAK inhibitor (e.g., tofacitinib), thalidomide, or cyclophosphamide (Cytoxan), or a combination thereof. In some embodiments, the therapeutic agent comprises ursodeoxycholic acid (UDCA), antipruritic, cholestyramine, antibiotic, vitamin A, vitamin D, vitamin E, or vitamin K, or a combination thereof. In some embodiments, the therapeutic agent comprises an anti-TL1A antibody as described herein. In some embodiments, the therapeutic agent comprises an anti-CD30L antibody as described herein. In some embodiments, the therapeutic agent comprises a glucocorticosteroid. In some embodiments, the therapeutic agent comprises anti-TNF therapy. In some embodiments, the therapeutic agent comprises anti-a4-b7 therapy. In some embodiments, the therapeutic agent comprises a JAK inhibitor. In some embodiments, the therapeutic agent comprises thalidomide. In some embodiments, the therapeutic agent comprises cyclophosphamide. In some embodiments, the therapeutic agent comprises UDCA. In some embodiments, the therapeutic agent comprises antipruritic. In some embodiments, the therapeutic agent comprises cholestryamine. In some embodiments, the therapeutic agent comprises an antibiotic. In some embodiments, the therapeutic agent comprises vitamin A. In some embodiments, the therapeutic agent comprises vitamin D. In some embodiments, the therapeutic agent comprises vitamin E. In some embodiments, the therapeutic agent comprises vitamin K.

In some embodiments, the EIM comprises peripheral arthritis. In some embodiments, the arthritis comprises large joint arthritis, small joint arthritis, extracolonic arthritis, Crohn's disease/ulcerative colitis non-specific joint inflammation, JBD-associated arthralgia complication, or a combination thereof. In some embodiments, the therapeutic agent comprises an anti-TL1A antibody, anti-CD30L antibody, glucocorticosteroid, anti-TNF therapy, anti-a4-b7 therapy (e.g., vedolizumab), anti-IL12p40 therapy (e.g., ustekinumab), JAK inhibitor (e.g., tofacitinib), thalidomide, or cyclophosphamide (Cytoxan), or a combination thereof. In some embodiments, the therapeutic agent comprises methotrexate, leflunomide (e.g., Arava), hydroxychloroquine, sulfasalazine (e.g., Azulfidine), nonsteroidal anti-inflammatory drug (NSAID), TNF inhibitor, etanercept (e.g., Enbrel), infliximab (e.g., Remicade), belimumab (e.g., Benlysta), rituximab (e.g., Rituxan), anakinra (e.g., Kineret), tocilizumab (e.g., Actemra), canakinumab (e.g., Ilaris), secukinumab (e.g., Cosentyx), ustekinumab (e.g., Stelara), ixekizumab (e.g., Taltz), sarilumab (e.g., Kevzara), tofacitinib (e.g., XELJANZ), abatacept (e.g., Orencia), corticosteroid, prednisone, or cortisone, or a combination thereof. In some embodiments, the therapeutic agent comprises an anti-TL1A antibody as described herein. In some embodiments, the therapeutic agent comprises an anti-CD30L antibody as described herein. In some embodiments, the therapeutic agent comprises a glucocorticosteroid. In some embodiments, the therapeutic agent comprises anti-TNF therapy. In some embodiments, the therapeutic agent comprises anti-a4-b7 therapy. In some embodiments, the therapeutic agent comprises a JAK inhibitor. In some embodiments, the therapeutic agent comprises thalidomide. In some embodiments, the therapeutic agent comprises cyclophosphamide. In some embodiments, the therapeutic agent comprises methotrexate. In some embodiments, the therapeutic agent comprises leflunomide. In some embodiments, the therapeutic agent comprises hydroxychloroquine. In some embodiments, the therapeutic agent comprises sulfasalazine. In some embodiments, the therapeutic agent comprises NSAID. In some embodiments, the therapeutic agent comprises TNF inhibitor. In some embodiments, the therapeutic agent comprises etanercept. In some embodiments, the therapeutic agent comprises infliximab. In some embodiments, the therapeutic agent comprises belimumab. In some embodiments, the therapeutic agent comprises rituximab. In some embodiments, the therapeutic agent comprises anakinra. In some embodiments, the therapeutic agent comprises tocilizumab. In some embodiments, the therapeutic agent comprises canakinumab. In some embodiments, the therapeutic agent comprises secukinumab. In some embodiments, the therapeutic agent comprises ixekizumab. In some embodiments, the therapeutic agent comprises sarilumab. In some embodiments, the therapeutic agent comprises ustekinumab. In some embodiments, the therapeutic agent comprises tofacitinib. In some embodiments, the therapeutic agent comprises abatacept. In some embodiments, the therapeutic agent comprises cortisone. In some embodiments, the therapeutic agent comprises a corticosteroid. In some embodiments, the therapeutic agent comprises prednisone.

In some embodiments, wherein the EIM comprises ankylosing spondylitis, sacroiliitis, erythema nodosum, pyoderma gangrenosum, psoriasis, a manifestation in the eye, or primary sclerosing cholangitis, or a combination thereof. In some embodiments, the EIM comprises ankylosing spondylitis, sacroiliitis, erythema nodosum, pyoderma gangrenosum, psoriasis, eye, and primary sclerosing cholangitis. In some embodiments, the EIM comprises ankylosing spondylitis, sacroiliitis, erythema nodosum, pyoderma gangrenosum, psoriasis, eye, peripheral arthritis, or primary sclerosing cholangitis, or a combination thereof. In some embodiments, the EIM comprises ankylosing spondylitis, sacroiliitis, erythema nodosum, pyoderma gangrenosum, psoriasis, eye, peripheral arthritis, and primary sclerosing cholangitis.

### Methods of Treatment

Disclosed herein are methods of treating a disease or condition, or a symptom of the disease or condition, in a subject, comprising administrating of therapeutic effective amount of one or more therapeutic agents to the subject. In some embodiments, the one or more therapeutic agents is administered to the subject alone (e.g., standalone therapy). In some embodiments, the one or more therapeutic agents is administered in combination with an additional agent. In some embodiments, the therapeutic agent is a first-line therapy for the disease or condition. In some embodiments, the therapeutic agent is a second-line, third-line, or fourth-line therapy, for the disease or condition.

A. Therapeutic Agents

In one aspect, provided herein are antibodies and antigen-binding fragments for treatment of IBD and/or an EIM of IBD. In some embodiments, an antibody comprises an antigen-binding fragment that refers to a portion of an antibody having antigenic determining variable regions of an antibody. Examples of antigen-binding fragments include, but are not limited to, Fab, Fab′, F(ab′)₂, and Fv fragments, linear antibodies, single chain antibodies, and multispecific antibodies formed from antibody fragments. In some embodiments, an antibody refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. In some embodiments, an antibody includes intact polyclonal antibodies, intact monoclonal antibodies, antibody fragments (such as Fab, Fab′, F(ab′)₂, and Fv fragments), single chain Fv (scFv) mutants, a CDR-grafted antibody, multispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc.

In some embodiments, a humanized antibody refers to forms of non-human (e.g., murine) antibodies having specific immunoglobulin chains, chimeric immunoglobulins, or fragments thereof that contain minimal non-human (e.g., murine) sequences. In a non-limiting example, a humanized antibody comprises less than about 40% non-human sequence in the variable region. In some cases, a humanized antibody comprises less than about 20% non-human sequence in a full-length antibody sequence. In a further non-limiting example, a humanized antibody comprises less than about 20% non-human sequence in the framework region of each of the heavy chain and light chain variable regions. For instance, the humanized antibody comprises less than about 20%, 19%, 18%, 17%, 16%, 15%, 14%, 13%, 12%, 11%, 10%, 9%, 8%, 7%, 6%, 5%, 4%, 3%, 2%, or 1% non-human sequence in the framework region of each of the heavy chain and light chain variable regions. As another example, the humanized antibody comprises about or less than about 15, 14, 13, 12, 11, 10, 9, 8, 7, 6, 5, 4, 3, 2, or 1 non-human sequences in the framework region of each of the heavy chain and light chain variable regions. In some cases, humanized antibodies are human immunoglobulins in which residues from the complementarity determining region (CDR) are replaced by residues from the CDR of a non-human species (e.g., mouse, rat, rabbit, hamster) that have the desired specificity, affinity, and capability. These humanized antibodies may contain one or more non-human species mutations, e.g., the heavy chain comprises about 1, 2, 3, 4, 5, 6, 7, 8, 9, 10, 11, 12, 13, 14, or 15 non-human species mutations in the framework region, and the light chain comprises about 1, 2, 3, 4, 5, 6, 7, 8, 9, 10, 11, 12, 13, 14, or 15 non-human species mutations in the framework region. The humanized heavy chain variable domain may comprise IGHV1-46*02 framework with no or fewer than about 10, 9, 8, 7, 6, 5, 4, 3, 2, or 1 amino acid mutations. The humanized light chain variable domain may comprise IGKV3-20 framework with no or fewer than about 10, 9, 8, 7, 6, 5, 4, 3, 2, or 1 amino acid mutations.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof.

In some embodiments, an antibody that specifically binds to a protein indicates that the antibody reacts or associates more frequently, more rapidly, with greater duration, with greater affinity, or with some combination of the above to the protein than with alternative substances, including unrelated proteins.

In some embodiments, the terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as fusion with another polypeptide and/or conjugation, e.g., with a labeling component. Also included within the definition are, for example, polypeptides containing one or more analogs of an amino acid (for example, unnatural amino acids, etc.), as well as other modifications known in the art.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.OD. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, the term “about” means within 10% of the stated amount. For instance, an antibody variable region comprising about 80% identity to a reference variable region may comprise 72% to 88% identity to the reference variable region.

TL1A Therapeutics

Aspects provided herein are methods of treating an inflammatory, fibrostenotic, or fibrotic disease or condition in a subject by administering a therapeutically effective amount of an inhibitor of TNF Superfamily Member 15 (TL1A) activity or expression to the subject, provided a genotype is detected in a sample obtained from the subject. In some embodiments, the inhibitor of TL1A activity or expression is effective to inhibit TL1A-DR3 binding. In some embodiments, the inhibitor of TL1A activity or expression comprises an allosteric modulator of TL1A. An allosteric modulator of TL1A may indirectly influence the effects TL1A on DR3, or TR6/DcR3 on TL1A or DR3. The inhibitor of TL1A activity or expression may be a direct inhibitor or indirect inhibitor. Non-limiting examples of an inhibitor of TL1A expression include RNA to protein TL1A translation inhibitors, antisense oligonucleotides targeting the TNFSF15 mRNA (such as miRNAs, or siRNA), epigenetic editing (such as targeting the DNA-binding domain of TNFSF15, or post-translational modifications of histone tails and/or DNA molecules). Non-limiting examples of an inhibitor of TL1A activity include antagonists to the TL1A receptors, (DR3 and TR6/DcR3), antagonists to TL1A antigen, and antagonists to gene expression products involved in TL1A mediated disease. Antagonists as disclosed herein, may include, but are not limited to, an anti-TL1A antibody, an anti-TL1A-binding antibody fragment, or a small molecule. The small molecule may be a small molecule that binds to TL1A or DR3. The anti-TL1A antibody may be monoclonal or polyclonal. The anti-TL1A antibody may be humanized or chimeric. The anti-TL1A antibody may be a fusion protein. The anti-TL1A antibody may be a blocking anti-TL1A antibody. A blocking antibody blocks binding between two proteins, e.g., a ligand and its receptor. Therefore, a TL1A blocking antibody includes an antibody that prevents binding of TL1A to DR3 and/or TR6/DcR3 receptors. In a non-limiting example, the TL1A blocking antibody binds to DR3. In another example, the TL1A blocking antibody binds to DcR3. In some cases, the TL1A antibody is an anti-TL1A antibody that specifically binds to TL1A.

In certain aspects, antibodies are described herein that specifically bind to TL1A (Entrez Gene: 9966; UniProtKB: 095150). In some embodiments, the antibodies specifically bind to soluble TL1A. In some embodiments, the antibodies specifically bind to membrane bound TL1A. In some embodiments, an anti-TL1A antibody is provided having a heavy chain comprising four heavy chain framework regions (HCFR) and three heavy chain complementarity-determining regions (HCDR): HCFR1, HCDR1, HCFR2, HCDR2, HCFR3, HCDR3, and HCFR4; and a light chain comprising four light chain framework regions (LCFR) and three light chain complementarity-determining regions (LCDR): LCFR1, LCDR1, LCFR2, LCDR2, LCFR3, LCDR3, and LCFR4. An anti-TL1A antibody may comprise any region provided herein, for example, as provided in Tables 2A-2H, the examples, and the sequences.

In certain embodiments, an anti-TL1A antibody comprises a HCDR1 as set forth by any one of SEQ ID NOS: 1 or 601-722. In certain embodiments, an anti-TL1A antibody comprises a HCDR2 as set forth by any one of SEQ ID NOS: 2-5 or 723-787. In certain embodiments, an anti-TL1A antibody comprises a HCDR3 as set forth by any one of SEQ ID NOS: 6-9 or 788-842. In certain embodiments, an anti-TL1A antibody comprises a LCDR1 as set forth by any one of SEQ ID NOS: 10 or 843-865. In certain embodiments, an anti-TL1A antibody comprises a LCDR2 as set forth by any one of SEQ ID NOS: 11 or 866-885. In certain embodiments, an anti-TL1A antibody comprises a LCDR3 as set forth by any one of SEQ ID NOS: 12-15 or 886-1101.

In one aspect, provided herein are anti-TL1A antibodies having a HCDR1 comprising any one of SEQ ID NOS: 1 or 601-708, a HCDR2 comprising any one of SEQ ID NOS: 2-5 or 723-774, a HCDR3 comprising any one of SEQ ID NOS: 6-9 or 788-828, a LCDR1 comprising any one of SEQ ID NOS: 10 or 843-852, a LCDR2 comprising any one of SEQ ID NOS: 11 or 866-873, and a LCDR3 comprising any one of SEQ ID NOS: 12-15 or 886-1089.

In one aspect, provided herein are anti-TL1A antibodies having a HCDR1 comprising SEQ ID NO: 709, a HCDR2 comprising SEQ ID NO: 775, a HCDR3 comprising SEQ ID NO: 829, a LCDR1 comprising SEQ ID NO: 853, a LCDR2 comprising SEQ ID NO: 874, and a LCDR3 comprising SEQ ID NO: 1090.

In one aspect, provided herein are anti-TL1A antibodies having a HCDR1 comprising SEQ ID NO: 710, a HCDR2 comprising SEQ ID NO: 776, a HCDR3 comprising SEQ ID NO: 830, a LCDR1 comprising SEQ ID NO: 854, a LCDR2 comprising SEQ ID NO: 875, and a LCDR3 comprising SEQ ID NO: 1091. In one aspect, provided herein are anti-TL1A antibodies having a HCDR1 comprising SEQ ID NO: 711 or 712, a HCDR2 comprising SEQ ID NO: 777 or 778, a HCDR3 comprising SEQ ID NO: 831 or 832, a LCDR1 comprising SEQ ID NO: 855, a LCDR2 comprising SEQ ID NO: 876, and a LCDR3 comprising SEQ ID NO: 1092.

In one aspect, provided herein are anti-TL1A antibodies having a HCDR1 comprising SEQ ID NO: 713, a HCDR2 comprising SEQ ID NO: 779, a HCDR3 comprising SEQ ID NO: 833, a LCDR1 comprising SEQ ID NO: 856, a LCDR2 comprising SEQ ID NO: 877, and a LCDR3 comprising SEQ ID NO: 1093.

In one aspect, provided herein are anti-TL1A antibodies having a HCDR1 comprising SEQ ID NO: 714, a HCDR2 comprising SEQ ID NO: 780, a HCDR3 comprising SEQ ID NO: 834, a LCDR1 comprising SEQ ID NO: 857, a LCDR2 comprising SEQ ID NO: 878, and a LCDR3 comprising SEQ ID NO: 1094.

In one aspect, provided herein are anti-TL1A antibodies having a HCDR1 comprising SEQ ID NO: 715 or 716, a HCDR2 comprising SEQ ID NO: 781, a HCDR3 comprising SEQ ID NO: 835 or 836, a LCDR1 comprising SEQ ID NO: 858 or 859, a LCDR2 comprising SEQ ID NO: 879, and a LCDR3 comprising SEQ ID NO: 1095.

In one aspect, provided herein are anti-TL1A antibodies having a HCDR1 comprising SEQ ID NO: 717, a HCDR2 comprising SEQ ID NO: 782, a HCDR3 comprising SEQ ID NO: 837, a LCDR1 comprising SEQ ID NO: 860, a LCDR2 comprising SEQ ID NO: 880, and a LCDR3 comprising SEQ ID NO: 1096.

In one aspect, provided herein are anti-TL1A antibodies having a HCDR1 comprising SEQ ID NO: 718, a HCDR2 comprising SEQ ID NO: 783, a HCDR3 comprising SEQ ID NO: 838, a LCDR1 comprising SEQ ID NO: 861, a LCDR2 comprising SEQ ID NO: 881, and a LCDR3 comprising SEQ ID NO: 1097.

In one aspect, provided herein are anti-TL1A antibodies having a HCDR1 comprising SEQ ID NO: 719, a HCDR2 comprising SEQ ID NO: 784, a HCDR3 comprising SEQ ID NO: 839, a LCDR1 comprising SEQ ID NO: 862, a LCDR2 comprising SEQ ID NO: 882, and a LCDR3 comprising SEQ ID NO: 1098.

In one aspect, provided herein are anti-TL1A antibodies having a HCDR1 comprising SEQ ID NO: 720, a HCDR2 comprising SEQ ID NO: 785, a HCDR3 comprising SEQ ID NO: 840, a LCDR1 comprising SEQ ID NO: 863, a LCDR2 comprising SEQ ID NO: 883, and a LCDR3 comprising SEQ ID NO: 1099.

In one aspect, provided herein are anti-TL1A antibodies having a HCDR1 comprising SEQ ID NO: 721, a HCDR2 comprising SEQ ID NO: 786, a HCDR3 comprising SEQ ID NO: 841, a LCDR1 comprising SEQ ID NO: 864, a LCDR2 comprising SEQ ID NO: 884, and a LCDR3 comprising SEQ ID NO: 1100.

In one aspect, provided herein are anti-TL1A antibodies having a HCDR1 comprising SEQ ID NO: 722, a HCDR2 comprising SEQ ID NO: 787, a HCDR3 comprising SEQ ID NO: 842, a LCDR1 comprising SEQ ID NO: 865, a LCDR2 comprising SEQ ID NO: 885, and a LCDR3 comprising SEQ ID NO: 1101.

In certain embodiments, an anti-TL1A antibody comprises a HCDR1, HCDR2, HCDR3, LCDR1, LCDR2, LCDR3 selected from Table 2A. In certain embodiments, an anti-TL1A antibody comprises the CDRs set forth in any one of the antibodies of Table 2B. In certain embodiments, an anti-TL1A antibody comprises the CDRs set forth in any one of the antibodies in Table 2C. In certain embodiments, an anti-TL1A antibody comprises the CDRs set forth in any one of the heavy chain variable regions in Table 2D. In certain embodiments, an anti-TL1A antibody comprises the CDRs set forth in any one of the light chain variable regions in Table 2E. The CDRs may be defined by the Kabat, Chothia, or IMGT method. In certain embodiments, an anti-TL1A antibody comprises the CDRs set forth in antibody M1 as shown in Table 2A. In certain embodiments, an anti-TL1A antibody comprises the CDRs set forth in antibody M2 as shown in Table 2A. In certain embodiments, an anti-TL1A antibody comprises the CDRs set forth in antibody M3 as shown in Table 2A. In certain embodiments, an anti-TL1A antibody comprises the CDRs set forth in antibody M4 as shown in Table 2A. In certain embodiments, an anti-TL1A antibody comprises the CDRs set forth in antibody M5 as shown in Table 2A. In certain embodiments, an anti-TL1A antibody comprises the CDRs set forth in antibody M6 as shown in Table 2A. In certain embodiments, an anti-TL1A antibody comprises the CDRs set forth in antibody M7 as shown in Table 2A. In certain embodiments, an anti-TL1A antibody comprises the CDRs set forth in antibody M8 as shown in Table 2A. In certain embodiments, an anti-TL1A antibody comprises the CDRs set forth in antibody M9 as shown in Table 2A. In certain embodiments, an anti-TL1A antibody comprises the CDRs set forth in antibody M10 as shown in Table 2A. In certain embodiments, an anti-TL1A antibody comprises the CDRs set forth in antibody M11 as shown in Table 2A. In certain embodiments, an anti-TL1A antibody comprises the CDRs set forth in antibody M12 as shown in Table 2A.

In certain embodiments, an anti-TL1A antibody comprises a P HCDR1 (any one of SEQ ID NOS: 1 or 601-708), a P HCDR2 (any one of SEQ ID NOS: 2-5 or 723-774), a P HCDR3 (any one of SEQ ID NOS: 6-9 or 788-828), a P LCDR1 (any one of SEQ ID NOS: 10 or 843-852), a P LCDR2 (any one of SEQ ID NOS: 11 or 866-873), and a P LCDR3 (any one of SEQ ID NOS: 12-15 or 886-1089) as shown in Table 2A.

In certain embodiments, an anti-TL1A antibody comprises the CDRs set forth in antibody A as shown in Table 2B. In certain embodiments, an anti-TL1A antibody comprises the CDRs set forth in antibody A2 as shown in Table 2B. In certain embodiments, an anti-TL1A antibody comprises the CDRs set forth in antibody B as shown in Table 2B. In certain embodiments, an anti-TL1A antibody comprises the CDRs set forth in antibody B2 as shown in Table 2B. In certain embodiments, an anti-TL1A antibody comprises the CDRs set forth in antibody C as shown in Table 2B. In certain embodiments, an anti-TL1A antibody comprises the CDRs set forth in antibody C2 as shown in Table 2B. In certain embodiments, an anti-TL1A antibody comprises the CDRs set forth in antibody D as shown in Table 2B. In certain embodiments, an anti-TL1A antibody comprises the CDRs set forth in antibody D2 as shown in Table 2B. In certain embodiments, an anti-TL1A antibody comprises the CDRs set forth in antibody E as shown in Table 2B. In certain embodiments, an anti-TL1A antibody comprises the CDRs set forth in antibody E2 as shown in Table 2B. In certain embodiments, an anti-TL1A antibody comprises the CDRs set forth in antibody F as shown in Table 2B. In certain embodiments, an anti-TL1A antibody comprises the CDRs set forth in antibody F2 as shown in Table 2B. In certain embodiments, an anti-TL1A antibody comprises the CDRs set forth in antibody G as shown in Table 2B. In certain embodiments, an anti-TL1A antibody comprises the CDRs set forth in antibody G2 as shown in Table 2B. In certain embodiments, an anti-TL1A antibody comprises the CDRs set forth in antibody H as shown in Table 2B. In certain embodiments, an anti-TL1A antibody comprises the CDRs set forth in antibody H2 as shown in Table 2B. In certain embodiments, an anti-TL1A antibody comprises the CDRs set forth in antibody I as shown in Table 2B. In certain embodiments, an anti-TL1A antibody comprises the CDRs set forth in antibody 12 as shown in Table 2B.

In one aspect, provided herein is an anti-TL1A antibody comprising a heavy chain variable region comprising an amino acid sequence at least about 85%, 90%, 91%, 92%, 93%, 94%, 95%, 96%, 97%, 98%, 99%, or 100% identical to any one of SEQ ID NOS: 101-135; and a light chain variable region at least about 85%, 90%, 91%, 92%, 93%, 94%, 95%, 96%, 97%, 98%, 99%, or 100% identical to any one of SEQ ID NOS: 201-206.

In one aspect, provided herein is an anti-TL1A antibody comprising a heavy chain variable region comprising an amino acid sequence at least about 85%, 90%, 91%, 92%, 93%, 94%, 95%, 96%, 97%, 98%, 99%, or 100% identical to any one of SEQ ID NOS: 1200-1263; and a light chain variable region at least about 85%, 90%, 91%, 92%, 93%, 94%, 95%, 96%, 97%, 98%, 99%, or 100% identical to any one of SEQ ID NOS: 1264-1300, 1304-1316.

Further provided herein is an antibody or antigen binding fragment thereof that binds to tumor necrosis factor-like protein 1A (TL1A), comprising a heavy chain variable domain comprising an amino acid sequence at least about 96% identical to SEQ ID NO: 104, and a light chain variable domain comprising an amino acid sequence at least about 97% identical to SEQ ID NO: 201. In some embodiments, the heavy chain variable domain comprises an amino acid sequence at least about 97% identical to SEQ ID NO: 104. In some embodiments, the heavy chain variable domain comprises an amino acid sequence at least about 98% identical to SEQ ID NO: 104. In some embodiments, the heavy chain variable domain comprises an amino acid sequence at least about 99% identical to SEQ ID NO: 104. In some embodiments, the heavy chain variable domain comprises SEQ ID NO: 104. In some embodiments, the light chain variable domain comprises an amino acid sequence at least about 98% identical to SEQ ID NO: 201. In some embodiments, the light chain variable domain comprises an amino acid sequence at least about 99% identical to SEQ ID NO: 201. In some embodiments, the light chain variable domain comprises SEQ ID NO: 201.

Tables 2D-2G provide exemplary framework and variable region sequences. In some embodiments, an anti-TL1A antibody comprises a HC FR1 of Table 2F (SEQ ID NO: 304), a HC FR2 of Table 2F (any one of SEQ ID NOS: 305, 313, 1317), a HC FR3 of Table 2F (any one of SEQ ID NOS: 306, 307, 314, 315, 1318-1323), a HC FR4 of Table 2F (SEQ ID NO: 308), a LC FR1 of Table 2F (SEQ ID NO: 309), a LC FR2 of Table 2F (SEQ ID NO: 310 or 1324), a LC FR3 of Table 2F (SEQ ID NO: 311), and a LC FR4 of Table 2F (SEQ ID NO: 312).

In certain embodiments, an anti-TL1A antibody comprises the framework regions set forth in any one of the antibodies in Table 2C, wherein the framework regions are defined by the Kabat, Chothia, or IMGT method. In certain embodiments, an anti-TL1A antibody comprises the heavy chain framework regions set forth in any one of the antibodies in Table 2D, and the light chain framework regions set forth in any one of the antibodies in Table 2E, wherein the framework regions are defined by the Kabat, Chothia, or IMGT method.

In some embodiments, an anti-TL1A antibody comprises a heavy chain framework comprising SEQ ID NO: 301 (X1VQLVQ SGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS). In some cases, X1 is Q. In some cases, X1=E. In some cases, X2=R. In some cases, X2=K. In some cases, X3=A. In some cases, X3=R. In some cases, X4=M. In some cases, X4=I. In some cases, X5=V. In some cases, X5=A. In some cases, X6=M. In some cases, X6=I. In some cases, X7=R. In some cases, X7=T. In some cases, X8=V. In some cases, X8=A. In some cases, X9=M. In some cases, X9=L. In some embodiments, X1 is at position 1 of IGHV1-46*02 as determined by Kabat numbering. In some embodiments, X2 is at position 45 of IGHV1-46*02 as determined by Kabat numbering. In some embodiments, X3 is at position 47 of IGHV1-46*02 as determined by Kabat numbering. In some embodiments, X4 is at position 55 of IGHV1-46*02 as determined by Kabat numbering. In some embodiments, X5 is at position 78 of IGHV1-46*02 as determined by Kabat numbering. In some embodiments, X6 is at position 80 of IGHV1-46*02 as determined by Kabat numbering. In some embodiments, X7 is at position 82 of IGHV1-46*02 as determined by Kabat numbering. In some embodiments, X8 is at position 89 of IGHV1-46*02 as determined by Kabat numbering. In some embodiments, X9 is at position 91 of IGHV1-46*02 as determined by Kabat numbering.

In one aspect, provided herein is a first embodiment of an anti-TL1A antibody comprising a heavy chain framework comprising IGHV1-46*02, or a variant thereof, wherein the variant comprises between about 1 and about 9 amino acid substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions from IGHV1-46*02 framework. Additional embodiments include: (2) The anti-TL1A of embodiment (1), wherein the heavy chain framework comprises SEQ ID NO: 301. (3) The anti-TL1A of embodiment 2, wherein X1=Q. (4) The anti-TL1A of embodiment 2, wherein X1=E. (5) The anti-TL1A of any one of embodiments 2-4, wherein X2=R. (6) The anti-TL1A of any one of embodiments 2-4, wherein X2=K. (7) The anti-TL1A of any one of embodiments 2-6, wherein X3=A. (8) The anti-TL1A of any one of embodiments 2-6, wherein X3=R. (9) The anti-TL1A of any one of embodiments 2-8, wherein X4=M. (10) The anti-TL1A of any one of embodiments 2-8, wherein X4=I. (11) The anti-TL1A of any one of embodiments 2-10, wherein X5=V. (12) The anti-TL1A of any one of embodiments 2-10, wherein X5=A. (13) The anti-TL1A of any one of embodiments 2-12, wherein X6=M. (14) The anti-TL1A of any one of embodiments 2-12, wherein X6=I. (15) The anti-TL1A of any one of embodiments 2-14, wherein X7=R. (16) The anti-TL1A of any one of embodiments 2-14, wherein X7=T. (17) The anti-TL1A of any one of embodiments 2-16, wherein X8=V. (18) The anti-TL1A of any one of embodiments 2-16, wherein X8=A. (19) The anti-TL1A of any one of embodiments 2-18, wherein X9=M. (20) The anti-TL1A of any one of embodiments 2-4, wherein X9=L. (21) The anti-TL1A of any one of embodiments 1-20, comprising antibody A. (22) The anti-TL1A of any one of embodiments 1-20, comprising antibody B. (23) The anti-TL1A of any one of embodiments 1-20, comprising antibody C. (24) The anti-TL1A of any one of embodiments 1-20, comprising antibody D. (25) The anti-TL1A of any one of embodiments 1-20, comprising antibody E. (26) The anti-TL1A of any one of embodiments 1-20, comprising antibody F. (27) The anti-TL1A of any one of embodiments 1-20, comprising antibody G or I. (28) The anti-TL1A of any one of embodiments 1-20, comprising antibody H. (29) The anti-TL1A of any one of embodiments 1-28, comprising a human IgG1 Fc region comprising: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, (jj) 373A, 373G, or 373S, (kk) 376E, 376 W, or 376Y, (ll) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. As used herein, any combination of a group, such as (a) to (uu), includes at least about two or more items from the group, e.g., any combination of a group of (a) to (uu) includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, and up to 47 or all of the members of the group. (30) The anti-TL1A of any one of embodiments 1-28, comprising a (i) human IgG4 Fc region or (ii) a human IgG4 Fc region comprising (a) S228P, (b) S228P and L235E, or (c) S228P, F234A, and L235A, per Kabat numbering. (31) The anti-TL1A of any one of embodiments 1-28, comprising a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG2σ). (32) The anti-TL1A of any one of embodiments 1-31, comprising a heavy chain Fc region comprising any one of SEQ ID NOS: 320-362. (33) The anti-TL1A of any one of embodiments 1-32, comprising a light chain constant region comprising SEQ ID NO: 319. (34) The anti-TL1A of any one of embodiments 1-33, comprising a light chain comprising a light chain framework comprising IGKV3-20*01, or a variant thereof, wherein the variant comprises between about 1 and about 2 substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions in the framework. (35) The anti-TL1A antibody of embodiment 34, wherein X10 is L. (36) The anti-TL1A antibody of embodiment 34, wherein X10 is P. (37) The anti-TL1A antibody of any one of embodiments 34-36, wherein X11 is L. (38) The anti-TL1A antibody of any one of embodiments 34-36, wherein X11 is W.

In some embodiments, an anti-TL1A antibody comprises a heavy chain framework comprising SEQ ID NO: 302 (X1VQLVQ SGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS). In some cases, X1 is Q. In some cases, X1=E. In some cases, X2=R. In some cases, X2=K. In some cases, X3=A. In some cases, X3=R. In some cases, X4=M. In some cases, X4=I. In some cases, X5=V. In some cases, X5=A. In some cases, X6=M. In some cases, X6=I. In some cases, X7=R. In some cases, X7=T. In some cases, X8=V. In some cases, X8=A. In some cases, X9=M. In some cases, X9=L. In some embodiments, X1 is at position 1 of IGHV1-46*02 as determined by Kabat numbering. In some embodiments, X2 is at position 45 of IGHV1-46*02 as determined by Kabat numbering. In some embodiments, X3 is at position 47 of IGHV1-46*02 as determined by Kabat numbering. In some embodiments, X4 is at position 55 of IGHV1-46*02 as determined by Kabat numbering. In some embodiments, X5 is at position 78 of IGHV1-46*02 as determined by Kabat numbering. In some embodiments, X6 is at position 80 of IGHV1-46*02 as determined by Kabat numbering. In some embodiments, X7 is at position 82 of IGHV1-46*02 as determined by Kabat numbering. In some embodiments, X8 is at position 89 of IGHV1-46*02 as determined by Kabat numbering. In some embodiments, X9 is at position 91 of IGHV1-46*02 as determined by Kabat numbering.

In one aspect, provided herein is another first embodiment of an anti-TL1A antibody comprising a heavy chain framework comprising IGHV1-46*02, or a variant thereof, wherein the variant comprises between about 1 and about 9 amino acid substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions from IGHV1-46*02 framework. Additional embodiments include: (2) The anti-TL1A of embodiment (1), wherein the heavy chain framework comprises SEQ ID NO: 302. (3) The anti-TL1A of embodiment 2, wherein X1=Q. (4) The anti-TL1A of embodiment 2, wherein X1=E. (5) The anti-TL1A of any one of embodiments 2-4, wherein X2=R. (6) The anti-TL1A of any one of embodiments 2-4, wherein X2=K. (7) The anti-TL1A of any one of embodiments 2-6, wherein X3=A. (8) The anti-TL1A of any one of embodiments 2-6, wherein X3=R. (9) The anti-TL1A of any one of embodiments 2-8, wherein X4=M. (10) The anti-TL1A of any one of embodiments 2-8, wherein X4=I. (11) The anti-TL1A of any one of embodiments 2-10, wherein X5=V. (12) The anti-TL1A of any one of embodiments 2-10, wherein X5=A. (13) The anti-TL1A of any one of embodiments 2-12, wherein X6=M. (14) The anti-TL1A of any one of embodiments 2-12, wherein X6=I. (15) The anti-TL1A of any one of embodiments 2-14, wherein X7=R. (16) The anti-TL1A of any one of embodiments 2-14, wherein X7=T. (17) The anti-TL1A of any one of embodiments 2-16, wherein X8=V. (18) The anti-TL1A of any one of embodiments 2-16, wherein X8=A. (19) The anti-TL1A of any one of embodiments 2-18, wherein X9=M. (20) The anti-TL1A of any one of embodiments 2-4, wherein X9=L. (21) The anti-TL1A of any one of embodiments 1-20, comprising antibody A. (22) The anti-TL1A of any one of embodiments 1-20, comprising antibody B. (23) The anti-TL1A of any one of embodiments 1-20, comprising antibody C. (24) The anti-TL1A of any one of embodiments 1-20, comprising antibody D. (25) The anti-TL1A of any one of embodiments 1-20, comprising antibody E. (26) The anti-TL1A of any one of embodiments 1-20, comprising antibody F. (27) The anti-TL1A of any one of embodiments 1-20, comprising antibody G or I. (28) The anti-TL1A of any one of embodiments 1-20, comprising antibody H. (29) The anti-TL1A of any one of embodiments 1-28, comprising a human IgG1 Fc region comprising: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 2921, (aa) 293S, (bb) 301 W, (cc) 304E, (dd) 311E, 311G, or 311S, (ee) 316F, (ff) 328V, (gg) 330R, (hh) 339E or 339L, (ii) 3431 or 343V, (jj) 373A, 373G, or 373S, (kk) 376E, 376 W, or 376Y, (ll) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. (30) The anti-TL1A of any one of embodiments 1-28, comprising a (i) human IgG4 Fc region or (ii) a human IgG4 Fc region comprising (a) S228P and L235E, or (b) S228P, F234A, and L235A, per Kabat numbering. (31) The anti-TL1A of any one of embodiments 1-28, comprising a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG2σ). (32) The anti-TL1A of any one of embodiments 1-31, comprising a heavy chain Fc region comprising any one of SEQ ID NOS: 320-362. (33) The anti-TL1A of any one of embodiments 1-32, comprising a light chain constant region comprising SEQ ID NO: 319. (34) The anti-TL1A of any one of embodiments 1-33, comprising a light chain comprising a light chain framework comprising IGKV3-20*01, or a variant thereof, wherein the variant comprises between about 1 and about 2 substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions in the framework. (35) The anti-TL1A antibody of embodiment 34, wherein X10 is L. (36) The anti-TL1A antibody of embodiment 34, wherein X10 is P. (37) The anti-TL1A antibody of any one of embodiments 34-36, wherein X1I is L. (38) The anti-TL1A antibody of any one of embodiments 34-36, wherein X1I is W.

In some embodiments, an anti-TL1A antibody comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK). In some cases, X10 is L. In some cases, X10 is P. In some cases, X1I is L. In some cases, X1I is W. In some embodiments, X10 is at position 54 of IGKV3-20*01 as determined by Kabat numbering. In some embodiments, X1I is at position 55 of IGKV3-20*01 as determined by Kabat numbering.

In some embodiments, an anti-TL1A antibody comprises a heavy chain framework comprising IGHV1-46*02. In some embodiments, an anti-TL1A antibody comprises a heavy chain framework comprising a variant of IGHV1-46*02 comprising between about 1 and about 20 amino acid substitutions from SEQ ID NO: 316. In some embodiments, an anti-TL1A antibody comprises a heavy chain framework comprising a variant of IGHV1-46*02 comprising between about 1 and about 9 amino acid substitutions from SEQ ID NO: 316. In some embodiments, an anti-TL1A antibody comprises a heavy chain framework comprising a variant of IGHV1-46*02 comprising about 1, 2, 3, 4, 5, 6, 7, 8, 9, 10, 11, 12, 13, 14, 15, 16, 17, 18, 19, or 20 amino acid substitutions from SEQ ID NO: 316 in the framework. In some cases, the heavy chain framework substitution comprises Q1E, as determined by Kabat numbering. In some cases, the heavy chain framework substitution comprises R45K, as determined by Kabat numbering. In some cases, the heavy chain framework substitution comprises A47R, as determined by Kabat numbering. In some cases, the heavy chain framework substitution comprises M55I, as determined by Kabat numbering. In some cases, the heavy chain framework substitution comprises V78A, as determined by Kabat numbering. In some cases, the heavy chain framework substitution comprises M80I, as determined by Kabat numbering. In some cases, the heavy chain framework substitution comprises R82T, as determined by Kabat numbering. In some cases, the heavy chain framework substitution comprises V89A, as determined by Kabat numbering. In some cases, the heavy chain framework substitution comprises M91L, as determined by Kabat numbering.

In some embodiments, an anti-TL1A antibody comprises a light chain framework comprising IGKV3-20*01. In some embodiments, an anti-TL1A antibody comprises a variant of IGKV3-20*01 comprising between about 1 and about 20 amino acid substitutions from SEQ ID NO: 317. In some embodiments, an anti-TL1A antibody comprises a variant of IGKV3-20*01 comprising about 1 amino acid substitution from SEQ ID NO: 317. In some embodiments, an anti-TL1A antibody comprises a light chain framework comprising a variant of IGKV3-20*01 comprising about 2 amino acid substitutions from SEQ ID NO: 317. In some embodiments, an anti-TL1A antibody comprises a light chain framework comprising a variant of IGKV3-20*01 comprising about 1, 2, 3, 4, 5, 6, 7, 8, 9, 10, 11, 12, 13, 14, 15, 16, 17, 18, 19, or 20 amino acid substitutions from SEQ ID NO: 317 in the framework. In some cases, the light chain framework substitution comprises Q1E, as determined by Kabat numbering. In some cases, the light chain framework substitution comprises R45K, as determined by Kabat numbering.

In some embodiments, an anti-TL1A antibody comprises a heavy chain FR1 as set forth by SEQ ID NO: 304. In some embodiments, an anti-TL1A antibody comprises a heavy chain FR2 as set forth by SEQ ID NO: 305. In some embodiments, an anti-TL1A antibody comprises a heavy chain FR2 as set forth by SEQ ID NO: 313. In some embodiments, an anti-TL1A antibody comprises a heavy chain FR2 as set forth by SEQ ID NO: 1317. In some embodiments, an anti-TL1A antibody comprises a heavy chain FR3 as set forth by SEQ ID NO: 306. In some embodiments, an anti-TL1A antibody comprises a heavy chain FR3 as set forth by SEQ ID NO: 307. In some embodiments, an anti-TL1A antibody comprises a heavy chain FR3 as set forth by SEQ ID NO: 314. In some embodiments, an anti-TL1A antibody comprises a heavy chain FR3 as set forth by SEQ ID NO: 315. In some embodiments, an anti-TL1A antibody comprises a heavy chain FR3 as set forth by SEQ ID NO: 1318. In some embodiments, an anti-TL1A antibody comprises a heavy chain FR3 as set forth by SEQ ID NO: 1319. In some embodiments, an anti-TL1A antibody comprises a heavy chain FR3 as set forth by SEQ ID NO: 1320. In some embodiments, an anti-TL1A antibody comprises a heavy chain FR3 as set forth by SEQ ID NO: 1321. In some embodiments, an anti-TL1A antibody comprises a heavy chain FR3 as set forth by SEQ ID NO: 1322. In some embodiments, an anti-TL1A antibody comprises a heavy chain FR3 as set forth by SEQ ID NO: 1323. In some embodiments, an anti-TL1A antibody comprises a heavy chain FR4 as set forth by SEQ ID NO: 308. In some embodiments, an anti-TL1A antibody comprises a light chain FR1 as set forth by SEQ ID NO: 309. In some embodiments, an anti-TL1A antibody comprises a light chain FR2 as set forth by SEQ ID NO: 310. In some embodiments, an anti-TL1A antibody comprises a light chain FR2 as set forth by SEQ ID NO: 1324. In some embodiments, an anti-TL1A antibody comprises a light chain FR3 as set forth by SEQ ID NO: 311. In some embodiments, an anti-TL1A antibody comprises a light chain FR3 as set forth by SEQ ID NO: 1325. In some embodiments, an anti-TL1A antibody comprises a light chain FR4 as set forth by SEQ ID NO: 312.

In some embodiments, one or more amino acid modifications may be introduced into the Fragment crystallizable (Fc) region of a human or humanized antibody, thereby generating an Fc region variant. An Fc region may comprise a C-terminal region of an immunoglobulin heavy chain that comprises a hinge region, CH2 domain, CH3 domain, or any combination thereof. As used herein, an Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution, addition, or deletion) at one or more amino acid positions. In an exemplary embodiment, the Fc region comprises any one of SEQ ID NOS: 320-362, 401-413, 501-515.

In some embodiments, antibodies of this disclosure have a reduced effector function as compared to a human IgG. Effector function refers to a biological event resulting from the interaction of an antibody Fc region with an Fc receptor or ligand. Non-limiting effector functions include C1q binding, complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation. In some cases, antibody-dependent cell-mediated cytotoxicity (ADCC) refers to a cell-mediated reaction in which nonspecific cytotoxic cells expressing Fc receptors (e.g., natural killer cells, neutrophils, macrophages) recognize bound antibody on a target cell, subsequently causing lysis of the target cell. In some cases, complement dependent cytotoxicity (CDC) refers to lysing of a target cells in the presence of complement, where the complement action pathway is initiated by the binding of C1q to antibody bound with the target.

Some Fc regions have a natural lack of effector function, and some Fc regions can comprise mutations that reduce effector functions. For instance, IgG4 has low ADCC and CDC activities and IgG2 has low ADCC activity.

The disclosure provides antibodies comprising Fc regions characterized by exhibiting ADCC that is reduced by at least about 30%, at least about 40%, at least about 50%, at least about 60%, at least about 70% or more as compared to an antibody comprising a non-variant Fc region, i.e., an antibody with the same sequence identity but for the substitution(s) that decrease ADCC (such as human IgG1, SEQ ID NO: 320). The disclosure provides antibodies comprising Fc regions characterized by exhibiting CDC that is reduced by at least about 30%, at least about 40%, at least about 50%, at least about 60%, at least about 70% or more as compared to an antibody comprising a non-variant Fc region, i.e., an antibody with the same sequence identity but for the substitution(s) that decrease CDC (such as human IgG1, SEQ ID NO: 320). In certain embodiments, the antibodies of this disclosure have reduced effector function as compared with human IgG1. Measurement of effector function may be performed as described in Example 3.

Non-limiting examples of Fc mutations in IgG1 that may reduce ADCC and/or CDC include substitutions at one or more of positions: 231, 232, 234, 235, 236, 237, 238, 239, 264, 265, 267, 269, 270, 297, 299, 318, 320, 322, 325, 327, 328, 329, 330, and 331 in IgG1, where the numbering system of the constant region is that of the EU index as set forth by Kabat. In certain embodiments, the antibodies of this disclosure have reduced effector function as compared with human IgG1.

In some embodiments, an antibody comprises an IgG1 Fc region comprising an N297A substitution, according to the Kabat numbering system. In some embodiments, an antibody comprises an IgG1 Fc region comprising an N297Q substitution, according to the Kabat numbering system. In some embodiments, an antibody comprises an IgG1 Fc region comprising an N297D substitution, according to the Kabat numbering system. In some embodiments, an antibody comprises an IgG1 Fc region comprising an D265A substitution, according to the Kabat numbering system. In some embodiments, an antibody comprises an IgG1 Fc region comprising an S228P substitution, according to the Kabat numbering system. In some embodiments, an antibody comprises an IgG1 Fc region comprising an L235A substitution, according to the Kabat numbering system. In some embodiments, an antibody comprises an IgG1 Fc region comprising an L237A substitution, according to the Kabat numbering system. In some embodiments, an antibody comprises an IgG1 Fc region comprising an L234A substitution, according to the Kabat numbering system. In some embodiments, an antibody comprises an IgG1 Fc region comprising an E233P substitution, according to the Kabat numbering system. In some embodiments, an antibody comprises an IgG1 Fc region comprising an L234V substitution, according to the Kabat numbering system. In some embodiments, an antibody comprises an IgG1 Fc region comprising an C236 deletion, according to the Kabat numbering system. In some embodiments, an antibody comprises an IgG1 Fc region comprising a P238A substitution, according to the Kabat numbering system. In some embodiments, an antibody comprises an IgG1 Fc region comprising an A327Q substitution, according to the Kabat numbering system. In some embodiments, an antibody comprises an IgG1 Fc region comprising a P329A substitution, according to the Kabat numbering system. In some embodiments, an antibody comprises an IgG1 Fc region comprising an P329G substitution, according to the Kabat numbering system. In some embodiments, an antibody comprises an IgG1 Fc region comprising an L235E substitution, according to the Kabat numbering system. In some embodiments, an antibody comprises an IgG1 Fc region comprising an P331S substitution, according to the Kabat numbering system. In some embodiments, an antibody comprises an IgG1 Fc region comprising an L234F substitution, according to the Kabat numbering system. In some embodiments, an antibody comprises an IgG1 Fc region comprising a 235G substitution, according to the Kabat numbering system. In some embodiments, an antibody comprises an IgG1 Fc region comprising an 235Q substitution, according to the Kabat numbering system. In some embodiments, an antibody comprises an IgG1 Fc region comprising an 235R substitution, according to the Kabat numbering system. In some embodiments, an antibody comprises an IgG1 Fc region comprising an 235S substitution, according to the Kabat numbering system. In some embodiments, an antibody comprises an IgG1 Fc region comprising an 236F substitution, according to the Kabat numbering system. In some embodiments, an antibody comprises an IgG1 Fc region comprising an 236R substitution, according to the Kabat numbering system. In some embodiments, an antibody comprises an IgG1 Fc region comprising an 237E substitution, according to the Kabat numbering system. In some embodiments, an antibody comprises an IgG1 Fc region comprising an 237K substitution, according to the Kabat numbering system. In some embodiments, an antibody comprises an IgG1 Fc region comprising an 237N substitution, according to the Kabat numbering system. In some embodiments, an antibody comprises an IgG1 Fc region comprising an 237R substitution, according to the Kabat numbering system. In some embodiments, an antibody comprises an IgG1 Fc region comprising an 238A substitution, according to the Kabat numbering system. In some embodiments, an antibody comprises an IgG1 Fc region comprising an 238E substitution, according to the Kabat numbering system. In some embodiments, an antibody comprises an IgG1 Fc region comprising an 238G substitution, according to the Kabat numbering system. In some embodiments, an antibody comprises an IgG1 Fc region comprising an 238H substitution, according to the Kabat numbering system. In some embodiments, an antibody comprises an IgG1 Fc region comprising an 238I substitution, according to the Kabat numbering system. In some embodiments, an antibody comprises an IgG1 Fc region comprising an 238V substitution, according to the Kabat numbering system. In some embodiments, an antibody comprises an IgG1 Fc region comprising an 238 W substitution, according to the Kabat numbering system. In some embodiments, an antibody comprises an IgG1 Fc region comprising an 238Y substitution, according to the Kabat numbering system. In some embodiments, an antibody comprises an IgG1 Fc region comprising an 248A substitution, according to the Kabat numbering system. In some embodiments, an antibody comprises an IgG1 Fc region comprising an 254D substitution, according to the Kabat numbering system. In some embodiments, an antibody comprises an IgG1 Fc region comprising an 254E substitution, according to the Kabat numbering system. In some embodiments, an antibody comprises an IgG1 Fc region comprising an 254G substitution, according to the Kabat numbering system. In some embodiments, an antibody comprises an IgG1 Fc region comprising an 254H substitution, according to the Kabat numbering system. In some embodiments, an antibody comprises an IgG1 Fc region comprising an 254I substitution, according to the Kabat numbering system. In some embodiments, an antibody comprises an IgG1 Fc region comprising an 254N substitution, according to the Kabat numbering system. In some embodiments, an antibody comprises an IgG1 Fc region comprising an 254P substitution, according to the Kabat numbering system. In some embodiments, an antibody comprises an IgG1 Fc region comprising an 254Q substitution, according to the Kabat numbering system. In some embodiments, an antibody comprises an IgG1 Fc region comprising an 254T substitution, according to the Kabat numbering system. In some embodiments, an antibody comprises an IgG1 Fc region comprising an 254V substitution, according to the Kabat numbering system. In some embodiments, an antibody comprises an IgG1 Fc region comprising an 255N substitution, according to the Kabat numbering system. In some embodiments, an antibody comprises an IgG1 Fc region comprising an 256H substitution, according to the Kabat numbering system. In some embodiments, an antibody comprises an IgG1 Fc region comprising an 256K substitution, according to the Kabat numbering system. In some embodiments, an antibody comprises an IgG1 Fc region comprising an 256R substitution, according to the Kabat numbering system. In some embodiments, an antibody comprises an IgG1 Fc region comprising an 256V substitution, according to the Kabat numbering system. In some embodiments, an antibody comprises an IgG1 Fc region comprising an 264S substitution, according to the Kabat numbering system. In some embodiments, an antibody comprises an IgG1 Fc region comprising an 265H substitution, according to the Kabat numbering system. In some embodiments, an antibody comprises an IgG1 Fc region comprising an 265K substitution, according to the Kabat numbering system. In some embodiments, an antibody comprises an IgG1 Fc region comprising an 265S substitution, according to the Kabat numbering system. In some embodiments, an antibody comprises an IgG1 Fc region comprising an 265Y substitution, according to the Kabat numbering system. In some embodiments, an antibody comprises an IgG1 Fc region comprising an 267G substitution, according to the Kabat numbering system. In some embodiments, an antibody comprises an IgG1 Fc region comprising an 267H substitution, according to the Kabat numbering system. In some embodiments, an antibody comprises an IgG1 Fc region comprising an 267I substitution, according to the Kabat numbering system. In some embodiments, an antibody comprises an IgG1 Fc region comprising an 267K substitution, according to the Kabat numbering system. In some embodiments, an antibody comprises an IgG1 Fc region comprising an 268K substitution, according to the Kabat numbering system. In some embodiments, an antibody comprises an IgG1 Fc region comprising an 269N substitution, according to the Kabat numbering system. In some embodiments, an antibody comprises an IgG1 Fc region comprising an 269Q substitution, according to the Kabat numbering system. In some embodiments, an antibody comprises an IgG1 Fc region comprising an 270A substitution, according to the Kabat numbering system. In some embodiments, an antibody comprises an IgG1 Fc region comprising an 270G substitution, according to the Kabat numbering system. In some embodiments, an antibody comprises an IgG1 Fc region comprising an 270M substitution, according to the Kabat numbering system. In some embodiments, an antibody comprises an IgG1 Fc region comprising an 270N substitution, according to the Kabat numbering system. In some embodiments, an antibody comprises an IgG1 Fc region comprising an 271T substitution, according to the Kabat numbering system. In some embodiments, an antibody comprises an IgG1 Fc region comprising an 272N substitution, according to the Kabat numbering system. In some embodiments, an antibody comprises an IgG1 Fc region comprising an 279F substitution, according to the Kabat numbering system. In some embodiments, an antibody comprises an IgG1 Fc region comprising an 279K substitution, according to the Kabat numbering system. In some embodiments, an antibody comprises an IgG1 Fc region comprising an 279L substitution, according to the Kabat numbering system. In some embodiments, an antibody comprises an IgG1 Fc region comprising an 292E substitution, according to the Kabat numbering system. In some embodiments, an antibody comprises an IgG1 Fc region comprising an 292F substitution, according to the Kabat numbering system. In some embodiments, an antibody comprises an IgG1 Fc region comprising an 292G substitution, according to the Kabat numbering system. In some embodiments, an antibody comprises an IgG1 Fc region comprising an 2921 substitution, according to the Kabat numbering system. In some embodiments, an antibody comprises an IgG1 Fc region comprising an 293S substitution, according to the Kabat numbering system. In some embodiments, an antibody comprises an IgG1 Fc region comprising an 301 W substitution, according to the Kabat numbering system. In some embodiments, an antibody comprises an IgG1 Fc region comprising an 304E substitution, according to the Kabat numbering system. In some embodiments, an antibody comprises an IgG1 Fc region comprising an 311E substitution, according to the Kabat numbering system. In some embodiments, an antibody comprises an IgG1 Fc region comprising an 311G substitution, according to the Kabat numbering system. In some embodiments, an antibody comprises an IgG1 Fc region comprising an 311S substitution, according to the Kabat numbering system. In some embodiments, an antibody comprises an IgG1 Fc region comprising an 316F substitution, according to the Kabat numbering system. In some embodiments, an antibody comprises an IgG1 Fc region comprising an 327T substitution, according to the Kabat numbering system. In some embodiments, an antibody comprises an IgG1 Fc region comprising an 328V substitution, according to the Kabat numbering system. In some embodiments, an antibody comprises an IgG1 Fc region comprising an 329Y substitution, according to the Kabat numbering system. In some embodiments, an antibody comprises an IgG1 Fc region comprising an 330R substitution, according to the Kabat numbering system. In some embodiments, an antibody comprises an IgG1 Fc region comprising an 339E substitution, according to the Kabat numbering system. In some embodiments, an antibody comprises an IgG1 Fc region comprising an 339L substitution, according to the Kabat numbering system. In some embodiments, an antibody comprises an IgG1 Fc region comprising an 3431 substitution, according to the Kabat numbering system. In some embodiments, an antibody comprises an IgG1 Fc region comprising an 343V substitution, according to the Kabat numbering system. In some embodiments, an antibody comprises an IgG1 Fc region comprising an 373A substitution, according to the Kabat numbering system. In some embodiments, an antibody comprises an IgG1 Fc region comprising an 373G substitution, according to the Kabat numbering system. In some embodiments, an antibody comprises an IgG1 Fc region comprising an 373S substitution, according to the Kabat numbering system. In some embodiments, an antibody comprises an IgG1 Fc region comprising an 376E substitution, according to the Kabat numbering system. In some embodiments, an antibody comprises an IgG1 Fc region comprising an 376 W substitution, according to the Kabat numbering system. In some embodiments, an antibody comprises an IgG1 Fc region comprising an 376Y substitution, according to the Kabat numbering system. In some embodiments, an antibody comprises an IgG1 Fc region comprising an 380D substitution, according to the Kabat numbering system. In some embodiments, an antibody comprises an IgG1 Fc region comprising an 382D substitution, according to the Kabat numbering system. In some embodiments, an antibody comprises an IgG1 Fc region comprising an 382P substitution, according to the Kabat numbering system. In some embodiments, an antibody comprises an IgG1 Fc region comprising an 385P substitution, according to the Kabat numbering system. In some embodiments, an antibody comprises an IgG1 Fc region comprising an 424H substitution, according to the Kabat numbering system. In some embodiments, an antibody comprises an IgG1 Fc region comprising an 424M substitution, according to the Kabat numbering system. In some embodiments, an antibody comprises an IgG1 Fc region comprising an 424V substitution, according to the Kabat numbering system. In some embodiments, an antibody comprises an IgG1 Fc region comprising an 4341 substitution, according to the Kabat numbering system. In some embodiments, an antibody comprises an IgG1 Fc region comprising an 438G substitution, according to the Kabat numbering system. In some embodiments, an antibody comprises an IgG1 Fc region comprising an 439E substitution, according to the Kabat numbering system. In some embodiments, an antibody comprises an IgG1 Fc region comprising an 439H substitution, according to the Kabat numbering system. In some embodiments, an antibody comprises an IgG1 Fc region comprising an 439Q substitution, according to the Kabat numbering system. In some embodiments, an antibody comprises an IgG1 Fc region comprising an 440A substitution, according to the Kabat numbering system. In some embodiments, an antibody comprises an IgG1 Fc region comprising an 440D substitution, according to the Kabat numbering system. In some embodiments, an antibody comprises an IgG1 Fc region comprising an 440E substitution, according to the Kabat numbering system. In some embodiments, an antibody comprises an IgG1 Fc region comprising an 440F substitution, according to the Kabat numbering system. In some embodiments, an antibody comprises an IgG1 Fc region comprising an 440M substitution, according to the Kabat numbering system. In some embodiments, an antibody comprises an IgG1 Fc region comprising an 440T Fc region substitution, according to the Kabat numbering system. In some embodiments, an antibody comprises an IgG1 Fc region comprising an 440V substitution, according to the Kabat numbering system.

In some embodiments, an antibody comprises a Fc region selected from the representative sequences disclosed in Table 2G. In some embodiments, an antibody comprises an IgG1 Fc region comprising E233P, according to the Kabat numbering system. In some embodiments, an antibody comprises an IgG4 Fc region comprising S228P and L235E. In some embodiments, an antibody comprises an IgG1 Fc region comprising L235E, according to the Kabat numbering system. In some embodiments, an antibody comprises an IgG1 Fc region comprising L234A and L235A, according to the Kabat numbering system. In some embodiments, an antibody comprises an IgG1 Fc region comprising L234A, L235A, and G237A, according to the Kabat numbering system. In some embodiments, an antibody comprises an IgG1 Fc region comprising L234A, L235A, P329G, according to the Kabat numbering system. In some embodiments, an antibody comprises an IgG1 Fc region comprising L234F, L235E, and P331S, according to the Kabat numbering system. In some embodiments, an antibody comprises an IgG1 Fc region comprising L234A, L235E, and G237A, according to the Kabat numbering system. In some embodiments, an antibody comprises an IgG1 Fc region comprising L234A, L235E, G237A, and P331S, according to the Kabat numbering system. In some embodiments, an antibody comprises an IgG1 Fc region comprising L234A, L235A, G237A, P238S, H268A, A330S, and P331S (IgG1σ), according to the Kabat numbering system. In some embodiments, an antibody comprises an IgG1 Fc region comprising L234A, L235A, and P329A, according to the Kabat numbering system. In some embodiments, an antibody comprises an IgG1 Fc region comprising G236R and L328R, according to the Kabat numbering system. In some embodiments, an antibody comprises an IgG1 Fc region comprising G237A, according to the Kabat numbering system. In some embodiments, an antibody comprises an IgG1 Fc region comprising F241A, according to the Kabat numbering system. In some embodiments, an antibody comprises an IgG1 Fc region comprising V264A, according to the Kabat numbering system. In some embodiments, an antibody comprises an IgG1 Fc region comprising D265A, according to the Kabat numbering system. In some embodiments, an antibody comprises an IgG1 Fc region comprising D265A and N297A, according to the Kabat numbering system. In some embodiments, an antibody comprises an IgG1 Fc region comprising D265A and N297G, according to the Kabat numbering system. In some embodiments, an antibody comprises an IgG1 Fc region comprising D270A, according to the Kabat numbering system. In some embodiments, an antibody comprises an IgG1 Fc region comprising N297A, according to the Kabat numbering system. In some embodiments, an antibody comprises an IgG1 Fc region comprising N297G, according to the Kabat numbering system. In some embodiments, an antibody comprises an IgG1 Fc region comprising N297D, according to the Kabat numbering system. In some embodiments, an antibody comprises an IgG1 Fc region comprising N297Q, according to the Kabat numbering system. In some embodiments, an antibody comprises an IgG1 Fc region comprising P329A, according to the Kabat numbering system. In some embodiments, an antibody comprises an IgG1 Fc region comprising P329G, according to the Kabat numbering system. In some embodiments, an antibody comprises an IgG1 Fc region comprising P329R, according to the Kabat numbering system. In some embodiments, an antibody comprises an IgG1 Fc region comprising A330L, according to the Kabat numbering system. In some embodiments, an antibody comprises an IgG1 Fc region comprising P331A, according to the Kabat numbering system. In some embodiments, an antibody comprises an IgG1 Fc region comprising P331S, according to the Kabat numbering system. In some embodiments, an antibody comprises an IgG2 Fc region. In some embodiments, an antibody comprises an IgG4 Fc region. In some embodiments, an antibody comprises an IgG4 Fc region comprising S228P, according to the Kabat numbering system. In some embodiments, an antibody comprises an IgG4 Fc region comprising S228P, F234A, and L235A, according to the Kabat numbering system. In some embodiments, an antibody comprises an IgG2-IgG4 cross-subclass (IgG2/G4) Fc region. In some embodiments, an antibody comprises an IgG2-IgG3 cross-subclass Fc region. In some embodiments, an antibody comprises an IgG2 Fc region comprising H268Q, V309L, A330S, and P331S, according to the Kabat numbering system. In some embodiments, an antibody comprises an IgG2 Fc region comprising V234A, G237A, P238S, H268A, V309L, A330S, and P331S, according to the Kabat numbering system. In some embodiments, an antibody comprises a Fc region comprising high mannose glycosylation.

In some embodiments, an antibody comprises an IgG4 Fc region comprising a S228P substitution, according to the Kabat numbering system. In some embodiments, an antibody comprises an IgG4 Fc region comprising an A330S substitution, according to the Kabat numbering system. In some embodiments, an antibody comprises an IgG4 Fc region comprising a P331S substitution, according to the Kabat numbering system.

In some embodiments, an antibody comprises an IgG2 Fc region comprising an A330S substitution, according to the Kabat numbering system. In some embodiments, an antibody comprises an IgG2 Fc region comprising an P331S substitution, according to the Kabat numbering system. In some embodiments, an antibody comprises an IgG2 Fc region comprising an 234A substitution, according to the Kabat numbering system. In some embodiments, an antibody comprises an IgG2 Fc region comprising an 237A substitution, according to the Kabat numbering system.

In certain embodiments, an anti-TL1A antibody described herein comprises a Fc region comprising a sequence from Table 2G. In certain embodiments, an anti-TL1A described herein comprises a Fc region as shown in Table 2H.

TABLE 2H
Exemplary Fc Mutations

| | Constant Region (SEQ ID NO) | | |
|---|---|---|---|
| Mutations | K_DL | R_EM | K_EM |
| Wild-type IgG1 | 320 | 321 | 322 |
| L235E | 323 | 324 | 325 |
| L234A, L235A | 326 | 327 | 328 |
| L234A, L235A, G237A | 329 | 330 | 331 |
| L234A, L235A, P329G | 332 | 333 | 334 |
| L234F, L235E, P331S | 335 | 336 | 337 |
| L234A, L235E, G237A | 338 | 339 | 340 |
| L234A, L235E, G237A, P331S | 341 | 342 | 343 |
| L234A, L235A, P329A | 344 | 345 | 346 |
| D265A | 347 | 348 | 349 |
| N297G | 350 | 351 | 352 |
| D265A, N297A | 353 | 354 | 355 |
| D265A, N297G | 356 | 357 | 358 |
| L235A, G237A | 359 | 360 | 361 |
| Wild-type IgG4 | 362 | | |

In some embodiments, the antibodies of this disclosure are variants that possess some but not all effector functions, which make it a desirable candidate for applications in which the half-life of the antibody in vivo is important yet certain effector functions (such as complement and ADCC) are unnecessary or deleterious.

In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. Measurement of effector function may be performed as described in Example 3.

In some embodiments, antibodies are tested for binding to Fc receptors and complement C1q by ELISA. In some embodiments, antibodies are tested for the ability to activate primary human immune cells in vitro, for example, by assessing their ability to induce expression of activation markers.

In some embodiments, assessment of ADCC activity of an anti-TL1A antibody comprises adding the antibody to target cells in combination with immune effector cells, which may be activated by the antigen antibody complexes resulting in cytolysis of the target cell. Cytolysis may be detected by the release of label (e.g. radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Wisecarver et al., 1985 79:277-282; Bruggemann et al., 1987, J Exp Med 166:1351-1361; Wilkinson et al., 2001, J Immunol Methods 258:183-191; Patel et al., 1995 J Immunol Methods 184:29-38. Alternatively, or additionally, ADCC activity of the antibody of interest may be assessed in vivo, e.g., in an animal model such as that disclosed in Clynes et al., 1998, PNAS USA 95:652-656.

In some embodiments, antibodies comprising a Fc region herein exhibit decreased ADCC activities as compared to an unmodified antibody (e.g., an antibody with human IgG1). In some embodiments, the antibodies herein exhibit ADCC activities that are at least 2-fold, or at least 3-fold, or at least 5-fold or at least 10-fold or at least 50-fold or at least 100-fold less than that of an unmodified antibody. In some embodiments, antibodies herein exhibit ADCC activities that are reduced by at least 10%, or at least 20%, or by at least 30%, or by at least 40%, or by at least 50%, or by at least 60%, or by at least 70%, or by at least 80%, or by at least 90%, or by at least 100%, or by at least 200%, or by at least 300%, or by at least 400%, or by at least 500% relative to an unmodified antibody. In certain embodiments, antibodies herein have no detectable ADCC activity. In certain embodiments, the reduction and/or abatement of ADCC activity may be attributed to the reduced affinity antibodies of the invention exhibit for Fc ligands and/or receptors.

In some embodiments, an assessment of complement activation, a CDC assay, may be performed as described in Gazzano-Santoro et al., 1996, J. Immunol. Methods, 202:163.

In some embodiments, antibodies comprising Fc regions described herein exhibit decreased affinities to C1q relative to an unmodified antibody (e.g., human IgG1). In some embodiments, antibodies herein exhibit affinities for C1q receptor that are at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than an unmodified antibody. In some embodiments, antibodies herein exhibit affinities for C1q that are at least 90%, at least 80%, at least 70%, at least 60%, at least 50%, at least 40%, at least 30%, at least 20%, at least 10%, or at least 5% less than an unmodified antibody. In some embodiments, antibodies herein exhibit affinities for C1q that are between about 100 nM to about 100 μM, or about 100 nM to about 10 μM, or about 100 nM to about 1 μM, or about 1 nM to about 100 μM, or about 10 nM to about 100 μM, or about 1 μM to about 100 μM, or about 10 μM to about 100 μM. In certain embodiments, antibodies herein exhibit affinities for C1q that are greater than 1 μM, greater than 5 μM, greater than 10 μM, greater than 25 μM, greater than 50 μM, or greater than 100 μM.

In some embodiments, antibodies comprising Fc regions described herein exhibit decreased CDC activities as compared to an unmodified antibody (e.g., human IgG1). In some embodiments, antibodies herein exhibit CDC activities that are at least 2-fold, or at least 3-fold, or at least 5-fold or at least 10-fold or at least 50-fold or at least 100-fold less than that of an unmodified antibody. In some embodiments, antibodies herein exhibit CDC activities that are reduced by at least 10%, or at least 20%, or by at least 30%, or by at least 40%, or by at least 50%, or by at least 60%, or by at least 70%, or by at least 80%, or by at least 90%, or by at least 100%, or by at least 200%, or by at least 300%, or by at least 400%, or by at least 500% relative to an unmodified antibody. In certain embodiments, antibodies herein exhibit no detectable CDC activities. In some embodiments, the reduction and/or ablatement of CDC activity may be attributed to the reduced affinity antibodies of the invention exhibit for Fc ligands and/or receptors.

Accordingly, further provided and described herein are anti-TL1A antibodies comprising a variant (e.g. harboring mutations) Fc region that reduce the cytotoxic response (e.g. ADCC or CDC) elicited by an anti-TL1A antibody.

In various embodiments, monoclonal antibodies are prepared using methods known in the art, such as, but not limited to the hybridoma method, where a host animal is immunized to elicit the production by lymphocytes of antibodies that will specifically bind to an immunizing antigen (Kohler and Milstein (1975) Nature 256:495). Hybridomas produce monoclonal antibodies directed specifically against a chosen antigen. The monoclonal antibodies are purified from the culture medium or ascites fluid by techniques known in the art, when propagated either in vitro or in vivo.

In some embodiments, monoclonal antibodies are made using recombinant DNA methods. The polynucleotides encoding a monoclonal antibody are isolated from mature B-cells or hybridoma cells. The isolated polynucleotides encoding the heavy and light chains are then cloned into suitable expression vectors, which when transfected into host cells (e.g., E. coli cells, simian COS cells, Chinese hamster ovary (CHO) cells, or myeloma cells) generate monoclonal antibodies. The polynucleotide(s) encoding a monoclonal antibody can further be modified in a number of different manners using recombinant DNA technology to generate alternative antibodies.

In various embodiments, a chimeric antibody, a molecule in which different portions are derived from different animal species, such as those having a variable region derived from a murine monoclonal antibody and a human immunoglobulin constant region (e.g., humanized antibodies) can be generated.

In some embodiments, the anti-TL1A monoclonal antibody is a humanized antibody, to reduce antigenicity and HAMA (human anti-mouse antibody) responses when administered to a human subject. Humanized antibodies can be produced using various techniques known in the art. For example, an antibody is humanized by (1) determining the nucleotide and predicted amino acid sequence of the starting antibody light and heavy variable domains; (2) designing the humanized antibody, e.g., deciding which antibody framework region to use during the humanizing process; (3) the actual humanizing methodologies/techniques; and (4) the transfection and expression of the humanized antibody. In various embodiments, a humanized antibody can be further optimized to decrease potential immunogenicity, while maintaining functional activity, for therapy in humans.

Humanized antibodies can also be made in transgenic mice containing human immunoglobulin loci that are capable, upon immunization, of producing the full repertoire of human antibodies in the absence of endogenous immunoglobulin production. A humanized antibody may also be obtained by a genetic engineering approach that enables production of affinity-matured human-like polyclonal antibodies in large animals.

A fully humanized antibody may be created by first designing a variable region amino acid sequence that contains non-human, e.g., rodent-derived CDRs, embedded in human-derived framework sequences. The non-human CDRs provide the desired specificity. Accordingly, in some cases these residues are included in the design of the reshaped variable region essentially unchanged. In some cases, modifications should therefore be restricted to a minimum and closely watched for changes in the specificity and affinity of the antibody. On the other hand, framework residues in theory can be derived from any human variable region. A human framework sequences should be chosen, which is equally suitable for creating a reshaped variable region and for retaining antibody affinity, in order to create a reshaped antibody which shows an acceptable or an even improved affinity. The human framework may be of germline origin, or may be derived from non-germline (e.g., mutated or affinity matured) sequences. Genetic engineering techniques well known to those in the art, for example, but not limited to, phage display of libraries of human antibodies, transgenic mice, human-human hybridoma, hybrid hybridoma, B cell immortalization and cloning, single-cell RT-PCR or HuRAb Technology, may be used to generate a humanized antibody with a hybrid DNA sequence containing a human framework and a non-human CDR.

In certain embodiments, the anti-TL1A antibody is a human antibody. Human antibodies can be directly prepared using various techniques known in the art. Immortalized human B lymphocytes immunized in vitro or isolated from an immunized individual that produce an antibody directed against a target antigen can be generated.

Chimeric, humanized and human antibodies may be produced by recombinant expression. Recombinant polynucleotide constructs typically include an expression control sequence operably linked to the coding sequences of antibody chains, including naturally associated or heterologous promoter regions. In certain embodiments, it may be desirable to generate amino acid sequence variants of these humanized antibodies, particularly where these improve the binding affinity or other biological properties of the antibody.

In certain embodiments, an antibody fragment is used to treat and/or ameliorate IBD. Various techniques are known for the production of antibody fragments. Generally, these fragments are derived via proteolytic digestion of intact antibodies (for example Morimoto et al., 1993, Journal of Biochemical and Biophysical Methods 24:107-117; Brennan et al., 1985, Science, 229:81). Fab, Fv, and scFv antibody fragments can all be expressed in and secreted from E. coli or other host cells, thus allowing the production of large amounts of these fragments. Other techniques for the production of antibody fragments will be apparent to the skilled practitioner.

According to the present disclosure, techniques can be adapted for the production of single-chain antibodies specific to TL1A. In addition, methods can be adapted for the construction of Fab expression libraries to allow rapid and effective identification of monoclonal Fab fragments with the desired specificity for TL1A, or derivatives, fragments, analogs or homologs thereof. Antibody fragments may be produced by techniques in the art including, but not limited to: (a) a F(ab′)₂ fragment produced by pepsin digestion of an antibody molecule; (b) a Fab fragment generated by reducing the disulfide bridges of an F(ab′)₂ fragment, (c) a Fab fragment generated by the treatment of the antibody molecule with papain and a reducing agent, and (d) Fv fragments.

Also provided herein are modified antibodies comprising any type of variable region that provides for the association of the antibody with TL1A. Those skilled in the art will appreciate that the modified antibodies may comprise antibodies (e.g., full-length antibodies or immunoreactive fragments thereof) in which at least a fraction of one or more of the constant region domains has been deleted or otherwise altered so as to provide desired biochemical characteristics such as decreasing TL1A. In certain embodiments, the variable regions in both the heavy and light chains are altered by at least partial replacement of one or more CDRs and, if necessary, by partial framework region replacement and sequence changing. In some embodiments, the replaced CDRs may be derived from an antibody of the same class, subclass, from an antibody of a different class, for instance, from an antibody from a different species and/or a combination thereof. In some embodiments, the constant region of the modified antibodies will comprise a human constant region. Modifications to the constant region compatible with this disclosure comprise additions, deletions or substitutions of one or more amino acids in one or more domains.

In various embodiments, the expression of an antibody or antigen-binding fragment thereof as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. In other embodiments, the antibody or antigen-fragment thereof as described herein may be transfected into the host.

In some embodiments, the expression vectors are transfected into the recipient cell line for the production of the chimeric, humanized, or composite human antibodies described herein. In various embodiments, mammalian cells can be useful as hosts for the production of antibody proteins, which can include, but are not limited to cells of fibroblast origin, such as Vero (ATCC CRL 81) or CHO-K1 (ATCC CRL 61) cells, HeLa cells and L cells. Exemplary eukaryotic cells that can be used to express polypeptides include, but are not limited to, COS cells, including COS 7 cells; 293 cells, including 293-6E cells; CHO cells, including CHO-S and DG44 cells; PER.C6™ cells (Crucell); and NSO cells. In some embodiments, a particular eukaryotic host cell is selected based on its ability to make desired post-translational modifications to the heavy chains and/or light chains.

A number of suitable host cell lines capable of secreting intact heterologous proteins have been developed in the art, and include, but are not limited to CHO cell lines, various COS cell lines, HeLa cells, L cells and multiple myeloma cell lines.

An expression vector carrying a chimeric, humanized, or composite human antibody construct, antibody or antigen-binding fragment thereof as described herein can be introduced into an appropriate host cell by any of a variety of suitable means, depending on the type of cellular host including, but not limited to transformation, transfection, lipofection, conjugation, electroporation, direct microinjection, and microprojectile bombardment, as known to one of ordinary skill in the art. Expression vectors for these cells can include expression control sequences, such as an origin of replication sites, a promoter, an enhancer and necessary processing information sites, such as ribosome binding sites, RNA splice sites, polyadenylation sites, and transcriptional terminator sequences.

In various embodiments, yeast can also be utilized as hosts for the production of the antibody molecules or peptides described herein. In various other embodiments, bacterial strains can also be utilized as hosts for the production of the antibody molecules or peptides described herein. Examples of bacterial strains include, but are not limited to E. coli, Bacillus species, enterobacteria, and various Pseudomonas species.

In some embodiments, one or more antibodies or antigen-binding fragments thereof as described herein can be produced in vivo in an animal that has been engineered (transgenic) or transfected with one or more nucleic acid molecules encoding the polypeptides, according to any suitable method. For production of transgenic animals, transgenes can be microinjected into fertilized oocytes, or can be incorporated into the genome of embryonic stem cells, and the nuclei of such cells transferred into enucleated oocytes. Once expressed, antibodies can be purified according to standard procedures of the art, including HPLC purification, column chromatography, gel electrophoresis and the like (see generally, Scopes, Protein Purification (Springer-Verlag, NY, 1982)).

Once expressed in the host, the whole antibodies, antibody-fragments (e.g., individual light and heavy chains), or other immunoglobulin forms of the present disclosure can be recovered and purified by known techniques, e.g., immunoabsorption or immunoaffinity chromatography, chromatographic methods such as HPLC (high performance liquid chromatography), ammonium sulfate precipitation, gel electrophoresis, or any combination of these. See generally, Scopes, PROTEIN PURIF. (Springer-Verlag, NY, 1982). Substantially pure immunoglobulins of at least about 90% to 95% homogeneity are advantageous, as are those with 98% to 99% or more homogeneity, particularly for pharmaceutical uses. Once purified, partially or to homogeneity as desired, a humanized or composite human antibody can then be used therapeutically or in developing and performing assay procedures, immunofluorescent stainings, etc. See generally, Vols. I & II Immunol. Meth. (Lefkovits & Pernis, eds., Acad. Press, NY, 1979 and 1981).

Various embodiments provide for a genetic construct comprising a nucleic acid encoding an anti-TL1A antibody or fragment provided herein. Genetic constructs of the antibody can be in the form of expression cassettes, which can be suitable for expression of the encoded anti-TL1A antibody or fragment. The genetic construct may be introduced into a host cell with or without being incorporated in a vector. For example, the genetic construct can be incorporated within a liposome or a virus particle. Alternatively, a purified nucleic acid molecule can be inserted directly into a host cell by methods known in the art. The genetic construct can be introduced directly into cells of a host subject by transfection, infection, electroporation, cell fusion, protoplast fusion, microinjection or ballistic bombardment.

Various embodiments provide a recombinant vector comprising the genetic construct of an antibody provided herein. The recombinant vector can be a plasmid, cosmid or phage. The recombinant vectors can include other functional elements; for example, a suitable promoter to initiate gene expression.

Various embodiments provide a host cell comprising a genetic construct and/or recombinant vector described herein.

Various host systems are also advantageously employed to express recombinant protein. Examples of suitable mammalian host cell lines include the COS-7 lines of monkey kidney cells, and other cell lines capable of expressing an appropriate vector including, for example, L cells, C127, 3T3, Chinese hamster ovary (CHO), HeLa and BHK cell lines. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine (SEQ ID NO: 1303), maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography. Recombinant protein produced in bacterial culture can be isolated.

One of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retain the ability to specifically bind the target antigen. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as He, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained.

Particular conservative substitutions include, for example; Ala into Gly or into Ser; Arg into Lys; Asn into Gin or into H is; Asp into Glu; Cys into Ser; Gin into Asn; Glu into Asp; Gly into Ala or into Pro; His into Asn or into Gin; lie into Leu or into Val; Leu into lie or into Val; Lys into Arg, into Gin or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu.

In some embodiments, the antibody and/or antigen-binding fragment thereof described herein can be a variant of a sequence described herein, e.g., a conservative substitution variant of an antibody polypeptide. In some embodiments, the variant is a conservatively modified variant. A variant may refer to a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity, e.g., antigen-specific binding activity for the relevant target polypeptide.

Alterations of the native amino acid sequence can be accomplished by any of a number of techniques known to one of skill in the art. Mutations can be introduced at particular loci or by oligonucleotide-directed site-specific mutagenesis procedures. Techniques for making such alterations are very well established and include, for example, those disclosed by Walder et al. (Gene 42: 133, 1986); Bauer et al. (Gene 37:73, 1985); Craik (BioTechniques, January 1985, 12-19); Smith et al. (Genetic Engineering: Principles and Methods, Plenum Press, 1981).

Nucleic acid molecules encoding amino acid sequence variants of antibodies are prepared by a variety of methods known in the art. These methods include, but are not limited to, preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including but not limited to, blunt-ended or staggered-ended termini for ligation and restriction enzyme digestion. Techniques for such manipulations are disclosed, e.g., by Maniatis et al., Molecular Cloning, Lab. Manual (Cold Spring Harbor Lab. Press, NY, 1982 and 1989), and can be used to construct nucleic acid sequences which encode a monoclonal antibody molecule or antigen-binding region.

In some embodiments, a nucleic acid encoding an antibody or antigen-binding fragment thereof as described herein is comprised by a vector. In some of the aspects described herein, a nucleic acid sequence encoding an antibody or antigen-binding fragment thereof as described herein, or any module thereof, is operably linked to a vector. The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding an antibody or antigen-binding portion thereof as described herein in place of non-essential viral genes. The vector and/or particle may be utilized for the purpose of transferring any nucleic acids into cells either in vitro or in vivo. Numerous forms of viral vectors are known in the art.

By “recombinant vector,” it is meant that the vector includes a heterologous nucleic acid sequence, or “transgene” that is capable of expression in vivo.

Non-limiting methods for determining whether an anti-TL1A antibody binds to the same region of a reference antibody are known in the art. An exemplary method comprises a competition assay. For instance, the method comprises determining whether a reference antibody can compete with binding between the reference antibody and the TL1A protein or portion thereof, or determining whether the reference antibody can compete with binding between the reference antibody and the TL1A protein or portion thereof. Exemplary methods include use of surface plasmon resonance to evaluate whether an anti-TL1A antibody can compete with the binding between TL1A and another anti-TL1A antibody. In some cases, surface plasmon resonance is utilized in the competition assay.

TABLE 2A
CDR amino acid sequences

| SEQ | | |
|---|---|---|
| ID | | |
| NO | Description | Sequence |
| 1 | P HCDR1 | GFDIQDTYMH |
| 601 | P HCDR1 | DTYMH |
| 602 | P HCDR1 | KYDIN |
| 603 | P HCDR1 | GFEIQDTYMH |
| 604 | P HCDR1 | GFDPQDTYMH |
| 605 | P HCDR1 | GFDVQDTYMH |
| 606 | P HCDR1 | GFDIGDTYMH |
| 607 | P HCDR1 | GFDISDTYMH |
| 608 | P HCDR1 | GFDIVDTYMH |
| 609 | P HCDR1 | GFDIQDAYMH |
| 610 | P HCDR1 | GFDIQDSYMH |
| 611 | P HCDR1 | GFDIQDTFMH |
| 612 | P HCDR1 | GFDIQDTYIH |
| 613 | P HCDR1 | GFDLQDTYMH |
| 614 | P HCDR1 | GFDIQDTYLH |
| 615 | P HCDR1 | GFDIGDTFIH |
| 616 | P HCDR1 | GFDIGDTFMH |
| 617 | P HCDR1 | GFDIGDTYIH |
| 618 | P HCDR1 | GFDIQDTFIH |
| 619 | P HCDR1 | GFDIQDTFMH |
| 620 | P HCDR1 | GFDIQDTYIH |
| 621 | P HCDR1 | GFDISDTFIH |
| 622 | P HCDR1 | GFDISDTFMH |
| 623 | P HCDR1 | GFDISDTYIH |
| 624 | P HCDR1 | GFDISDTYMH |
| 625 | P HCDR1 | GFDIVDTFIH |
| 626 | P HCDR1 | GFDIVDTFMH |
| 627 | P HCDR1 | GFDIVDTYIH |
| 628 | P HCDR1 | GFDPGDTFIH |
| 629 | P HCDR1 | GFDPGDTFMH |
| 630 | P HCDR1 | GFDPGDTYIH |
| 631 | P HCDR1 | GFDPGDTYMH |
| 632 | P HCDR1 | GFDPQDTFIH |
| 633 | P HCDR1 | GFDPQDTFMH |
| 634 | P HCDR1 | GFDPQDTYIH |
| 635 | P HCDR1 | GFDPQDTYMH |
| 636 | P HCDR1 | GFDPSDTFIH |
| 637 | P HCDR1 | GFDPSDTFMH |
| 638 | P HCDR1 | GFDPSDTYIH |
| 639 | P HCDR1 | GFDPSDTYMH |
| 640 | P HCDR1 | GFDPVDTFIH |
| 641 | P HCDR1 | GFDPVDTFMH |
| 642 | P HCDR1 | GFDPVDTYIH |
| 643 | P HCDR1 | GFDPVDTYMH |
| 644 | P HCDR1 | GFDVGDTFIH |
| 645 | P HCDR1 | GFDVGDTFMH |
| 646 | P HCDR1 | GFDVGDTYIH |
| 647 | P HCDR1 | GFDVGDTYMH |
| 648 | P HCDR1 | GFDVQDTFIH |
| 649 | P HCDR1 | GFDVQDTFMH |
| 650 | P HCDR1 | GFDVQDTYIH |
| 651 | P HCDR1 | GFDVSDTFIH |
| 652 | P HCDR1 | GFDVSDTFMH |
| 653 | P HCDR1 | GFDVSDTYIH |
| 654 | P HCDR1 | GFDVSDTYMH |
| 655 | P HCDR1 | GFDVVDTFIH |
| 656 | P HCDR1 | GFDVVDTFMH |
| 657 | P HCDR1 | GFDVVDTYIH |
| 658 | P HCDR1 | GFDVVDTYMH |
| 659 | P HCDR1 | GFEIGDTFIH |
| 660 | P HCDR1 | GFEIGDTFMH |
| 661 | P HCDR1 | GFEIGDTYIH |
| 662 | P HCDR1 | GFEIGDTYMH |
| 663 | P HCDR1 | GFEIQDTFIH |
| 664 | P HCDR1 | GFEIQDTFMH |
| 665 | P HCDR1 | GFEIQDTYIH |
| 666 | P HCDR1 | GFEIQDTYMH |
| 667 | P HCDR1 | GFEISDTFIH |
| 668 | P HCDR1 | GFEISDTFMH |
| 669 | P HCDR1 | GFEISDTYIH |
| 670 | P HCDR1 | GFEISDTYMH |
| 671 | P HCDR1 | GFEIVDTFIH |
| 672 | P HCDR1 | GFEIVDTFMH |
| 673 | P HCDR1 | GFEIVDTYIH |
| 674 | P HCDR1 | GFEIVDTYMH |
| 675 | P HCDR1 | GFEPGDTFIH |
| 676 | P HCDR1 | GFEPGDTFMH |
| 677 | P HCDR1 | GFEPGDTYIH |
| 678 | P HCDR1 | GFEPGDTYMH |
| 679 | P HCDR1 | GFEPQDTFIH |
| 680 | P HCDR1 | GFEPQDTFMH |
| 681 | P HCDR1 | GFEPQDTYIH |
| 682 | P HCDR1 | GFEPQDTYMH |
| 683 | P HCDR1 | GFEPSDTFIH |
| 684 | P HCDR1 | GFEPSDTFMH |
| 685 | P HCDR1 | GFEPSDTYIH |
| 686 | P HCDR1 | GFEPSDTYMH |
| 687 | P HCDR1 | GFEPVDTFIH |
| 688 | P HCDR1 | GFEPVDTFMH |
| 689 | P HCDR1 | GFEPVDTYIH |
| 690 | P HCDR1 | GFEPVDTYMH |
| 691 | P HCDR1 | GFEVGDTFIH |
| 692 | P HCDR1 | GFEVGDTFMH |
| 693 | P HCDR1 | GFEVGDTYIH |
| 694 | P HCDR1 | GFEVGDTYMH |
| 695 | P HCDR1 | GFEVQDTFIH |
| 696 | P HCDR1 | GFEVQDTFMH |
| 697 | P HCDR1 | GFEVQDTYIH |
| 698 | P HCDR1 | GFEVQDTYMH |
| 699 | P HCDR1 | GFEVSDTFIH |
| 700 | P HCDR1 | GFEVSDTFMH |
| 701 | P HCDR1 | GFEVSDTYIH |
| 702 | P HCDR1 | GFEVSDTYMH |
| 703 | P HCDR1 | GFEVVDTFIH |
| 704 | P HCDR1 | GFEVVDTFMH |
| 705 | P HCDR1 | GFEVVDTYIH |
| 706 | P HCDR1 | GFEVVDTYMH |
| 707 | P HCDR1 | GFX₁X₂X₃DX₄X₅X₆H |
| | | X1 = D or E |
| | | X2 = 1, L, P, or V |
| | | X3 = G, Q, S, or V |
| | | X4 = A, S, T |
| | | X5 = F or Y |
| | | X6 = I, L, or M |
| 708 | P HCDR1 | GFX₁X₂X₃DTX₄X₅H |
| | | X₁ = D, OR E |
| | | X₂ = I, P, OR V |
| | | X₃ = G, Q, S, OR V |
| | | X₄ = F, OR Y |
| | | X₅ = I, OR M |
| 709 | M1 HCDR1 | GFTFSSYW |
| 710 | M2 HCDR1 | GGSFTGFY |
| 711 | M3 HCDR1 | GY(X1)F(X2)(X3)YGIS; |
| | | X1 = P, S, D, Q, N; X2 = |
| | | T, R; X3 = N, T, Y, H |
| 712 | M3 HCDR1 | GYDFTYYGIS |
| 713 | M4 HCDR1 | SRSYYWG |
| 714 | MS HCDR1 | TSNMGVV |
| 715 | M6 HCDR1 | LYGMN |
| 716 | M6 HCDR1 | NYGMN |
| 717 | M7 HCDR1 | GYTFTSSWMH |
| 718 | M8 HCDR1 | GYTFTSYDIN |
| 719 | M9 HCDR1 | SYFWS |
| 720 | M10 HCDR1 | GYYWN |
| 721 | M11 HCDR1 | GFTFSTYG |
| 722 | M12 HCDR1 | TYGMS |
| 2 | P HCDR2 | RIDPASGHTKYDPKFQV |
| 3 | P HCDR2 | RIEPASGHIKYDPKFQG |
| 4 | P HCDR2 | RIDPASGHIKYDPKFQG |
| 5 | P HCDR2 | RIEPASGHIKYDPKFQV |
| 723 | P HCDR2 | WIFPGDGRTDYNEKFKG |
| 724 | P HCDR2 | RLDPASGHTK |
| 725 | P HCDR2 | RIEPASGHTK |
| 726 | P HCDR2 | RIDPESGHTK |
| 727 | P HCDR2 | RIDPASGHTK |
| 728 | P HCDR2 | RIDPAGGHTK |
| 729 | P HCDR2 | RIDPASAHTK |
| 730 | P HCDR2 | RIDPASGHIK |
| 731 | P HCDR2 | RIDPASGHLK |
| 732 | P HCDR2 | RIDPASGHVK |
| 733 | P HCDR2 | YDPKFQV |
| 734 | P HCDR2 | IDPKFQV |
| 735 | P HCDR2 | LDPKFQV |
| 736 | P HCDR2 | MDPKFQV |
| 737 | P HCDR2 | SDPKFQV |
| 738 | P HCDR2 | TDPKFQV |
| 739 | P HCDR2 | VDPKFQV |
| 740 | P HCDR2 | YIPKFQV |
| 741 | P HCDR2 | YNPKFQV |
| 742 | P HCDR2 | YRPKFQV |
| 743 | P HCDR2 | YSPKFQV |
| 744 | P HCDR2 | YDPKFRV |
| 745 | P HCDR2 | YDPKFQA |
| 746 | P HCDR2 | YDPKFQD |
| 747 | P HCDR2 | YDPKFQE |
| 748 | P HCDR2 | YDPKFQG |
| 749 | P HCDR2 | YDPKFQH |
| 750 | P HCDR2 | YDPKFQK |
| 751 | P HCDR2 | YDPKFQL |
| 752 | P HCDR2 | YDPKFQM |
| 753 | P HCDR2 | YDPKFQN |
| 754 | P HCDR2 | YDPKFQP |
| 755 | P HCDR2 | YDPKFQR |
| 756 | P HCDR2 | YDPKFQS |
| 757 | P HCDR2 | YDPKFQT |
| 758 | P HCDR2 | RIEPASGHIKYDPKFQG |
| 759 | P HCDR2 | RIEPASGHIKYSPKFQG |
| 760 | P HCDR2 | RIEPASGHVKYSPKFQV |
| 761 | P HCDR2 | RIEPASGHVKYDPKFQT |
| 762 | P HCDR2 | RIDPASGHIKYDPKFQK |
| 763 | P HCDR2 | RIDPASGHVKIDPKFQV |
| 764 | P HCDR2 | RIDPASGHLKYDPKFQV |
| 765 | P HCDR2 | RIDPASGHLKYDPKFQR |
| 766 | P HCDR2 | RIDPASGHLKYDPKFQN |
| 767 | P HCDR2 | RIEPASGHLKYDPKFQE |
| 768 | P HCDR2 | PASGH |
| 769 | P HCDR2 | RIDPASGHTKYDPKFQV |
| 770 | P HCDR2 | RIDPASGHLKYDPKFQG |
| 771 | P HCDR2 | RIDPASGHTKYDPKFQG |
| 772 | P HCDR2 | RIDPASGHSKYDPKFQV |
| 773 | P HCDR2 | RIDPASGHYKYDPKFQV |
| 774 | P HCDR2 | RX₁X₂PX₃X₄X₅HX₆KX₇X₈PKFX₉X₁₀ |
| | | X1 = I or L |
| | | X2 = D or E |
| | | X3 = A or E |
| | | X4 = G or S |
| | | X5 = A or G |
| | | X6 = I, L, T, or V |
| | | X7 = I, L, M, S, T, V, or Y |
| | | X8 = D, I, N, R, or S |
| | | X9 = Q or R |
| | | X10 = A, D, E, G, H, K, L, |
| | | M, N, P, R, S, T, or V |
| 775 | M1 HCDR2 | IKEDGSEK |
| 776 | M2 HCDR2 | INHRGNT |
| 777 | M3 HCDR2 | WIS(X1)YNG(X2)(X3)(X4)YA(X5) |
| | | (X6) (X7)QG; X1 = T, P, S, |
| | | A; X2 = N, G, V, K, A; X3 = |
| | | T, K; X4 = H, N; X5 = Q, R; |
| | | X6 = K, M; X7 = L, H |
| 778 | M3 HCDR2 | WISTYNGNTHYARMLQG |
| 779 | M4 HCDR2 | SIYYNGRTYYNPSLKS |
| 780 | MS HCDR2 | HILWDDREYSNPALKS |
| 781 | M6 HCDR2 | WINTYTGEPTYADDFKG |
| 782 | M7 HCDR2 | IHPNSGGT |
| 783 | M8 HCDR2 | WLNPNSGXTG; X = N, Y |
| 784 | M9 HCDR2 | YIYYSGNTKYNPSLKS |
| 785 | M10 HCDR2 | EINHAGNTNYNPSLKS |
| 786 | M11 HCDR2 | ISGTGRTT |
| 787 | M12 HCDR2 | WMNTYSGVTTYADDFKG |
| 6 | P HCDR3 | SGGLPDV |
| 7 | P HCDR3 | ARSGGLPDV |
| 8 | P HCDR3 | SGGLPDW |
| 9 | P HCDR3 | ARSGGLPDW |
| 788 | P HCDR3 | YGPAMDY |
| 789 | P HCDR3 | LGGLPDV |
| 790 | P HCDR3 | SAGLPDV |
| 791 | P HCDR3 | SGGAPDV |
| 792 | P HCDR3 | SGGMPDV |
| 793 | P HCDR3 | SGGLPEV |
| 794 | P HCDR3 | SGGLPDK |
| 795 | P HCDR3 | SGGLPDM |
| 796 | P HCDR3 | SGGLPDQ |
| 797 | P HCDR3 | SGGLPDR |
| 798 | P HCDR3 | SGGLPDS |
| 799 | P HCDR3 | SGGLPDT |
| 800 | P HCDR3 | SGGLPDH |
| 801 | P HCDR3 | SGGLPDF |
| 802 | P HCDR3 | SGGSPDV |
| 803 | P HCDR3 | ARSGGLPDM |
| 804 | P HCDR3 | ARSGGLPDK |
| 805 | P HCDR3 | SGGLPD |
| 806 | P HCDR3 | ARSGGLPDF |
| 807 | P HCDR3 | ARSGGLPDL |
| 808 | P HCDR3 | SGGLPDE |
| 809 | P HCDR3 | SGGLPDI |
| 810 | P HCDR3 | SGGLPDK |
| 811 | P HCDR3 | SGGLPDL |
| 812 | P HCDR3 | SGGLPDM |
| 813 | P HCDR3 | SGGLPDQ |
| 814 | P HCDR3 | SGGLPDT |
| 815 | P HCDR3 | SGGLPDW |
| 816 | P HCDR3 | SGGLPDY |
| 817 | P HCDR3 | SGGMPDE |
| 818 | P HCDR3 | SGGMPDI |
| 819 | P HCDR3 | SGGMPDK |
| 820 | P HCDR3 | SGGMPDL |
| 821 | P HCDR3 | SGGMPDM |
| 822 | P HCDR3 | SGGMPDQ |
| 823 | P HCDR3 | SGGMPDT |
| 824 | P HCDR3 | SGGMPDV |
| 825 | P HCDR3 | SGGMPDW |
| 826 | P HCDR3 | SGGMPDY |
| 827 | P HCDR3 | X₁X₂GX₃PX₄X₅ |
| | | X1 = L or S |
| | | X2 = A or G |
| | | X3 = A, L, or M |
| | | X4 = D or E |
| | | X5 = K, M, Q, R, S, T, V, |
| | | or W |
| 828 | P HCDR3 | SGGX₁PDX₂ |
| | | X₁ = L, OR M |
| | | X₂ = E, I, K, L, M, Q, T, |
| | | V, W, OR Y |
| 829 | M1 HCDR3 | AREDYDSYYKYGMDV |
| 830 | M2 HCDR3 | ASPFYDFWSGSDY |
| 831 | M3 HCDR3 | ENYYGSG(X1)(X2)R GGMD(X3); |
| | | X1 = S, A; X2 = Y, P; X3 = |
| | | V, A, G |
| 832 | M3 HCDR3 | ENYYGSGAYRGGMDV |
| 833 | M4 HCDR3 | EDYGDYGAFDI |
| 834 | MS HCDR3 | MSRNYYGSSYVMDY |
| 835 | M6 HCDR3 | DTAMDYAMAY |
| 836 | M6 HCDR3 | DYGKYGDYYAMDY |
| 837 | M7 HCDR3 | ARGDYYGYVSWFAY |
| 838 | M8 HCDR3 | EVPETAAFEY |
| 839 | M9 HCDR3 | ETGSYYGFDY |
| 840 | M10 HCDR3 | GYCRSTTCYFDY |
| 841 | M11 HCDR3 | TKERGDYYYGVFDY |
| 842 | M12 HCDR3 | EGYVFDDYYATDY |
| 10 | P LCDR1 | RASSSVSYMY |
| 843 | P LCDR1 | RSSQTIVHSNGDTYLD |
| 844 | P LCDR1 | GASSSVSYMY |
| 845 | P LCDR1 | WASSSVSYMY |
| 846 | P LCDR1 | RASSSVIYMY |
| 847 | P LCDR1 | RASSSVSFMY |
| 848 | P LCDR1 | RASSSVSYLY |
| 849 | P LCDR1 | RASSSVSYMR |
| 850 | P LCDR1 | GASSSVSYMY |
| 851 | P LCDR1 | ASSSVSYMY |
| 852 | P LCDR1 | X₁ASSSVX₂X₃X₄X₅ |
| | | X1 = G, R, or W |
| | | X2 = I or S |
| | | X3 = F or Y |
| | | X4 = L or M |
| | | X5 = R or Y |
| 853 | M1 LCDR1 | QSILYSSNNKNY |
| 854 | M2 LCDR1 | QSLVHSDGNTY |
| 855 | M3 LCDR1 | RASQSVSSYLA |
| 856 | M4 LCDR1 | RASQGISSALA |
| 857 | MS LCDR1 | SASSSVNYMH |
| 858 | M6 LCDR1 | KSSQNIVHSDGNTYLE |
| 859 | M6 LCDR1 | RSSQSIVHSNGNTYLD |
| 860 | M7 LCDR1 | QNINVL |
| 861 | M8 LCDR1 | TSSSSDIGA(X1)(X2)GV(X3); |
| | | X1 = G, A; X2 = L, S, Q; |
| | | X3 = H, L |
| 862 | M9 LCDR1 | RASQSINNYLN |
| 863 | M10 LCDR1 | RASQSVRSSYLA |
| 864 | M11 LCDR1 | QTISSW |
| 865 | M12 LCDR1 | RSSQNIVHSDGNTYLE |
| 11 | P LCDR2 | ATSNLAS |
| 866 | P LCDR2 | KVSNRFS |
| 867 | P LCDR2 | AKSNLAS |
| 868 | P LCDR2 | ATPNLAS |
| 869 | P LCDR2 | ATENLAS |
| 870 | P LCDR2 | ATSLLAS |
| 871 | P LCDR2 | ATSPLAS |
| 872 | P LCDR2 | ATSNLTS |
| 873 | P LCDR2 | AX₁X₂X₃LX₄S |
| | | X1 = K or T |
| | | X2 = E, P, or S |
| | | X3 = L, N, or P |
| | | X4 = A or T |
| 874 | M1 LCDR2 | WAS |
| 875 | M2 LCDR2 | KIS |
| 876 | M3 LCDR2 | DASNRAT |
| 877 | M4 LCDR2 | DASSLES |
| 878 | M5 LCDR2 | STSNLAS |
| 879 | M6 LCDR2 | KVSNRFS |
| 880 | M7 LCDR2 | KAS |
| 881 | M8 LCDR2 | GYYNRPS |
| 882 | M9 LCDR2 | AASSLQS |
| 883 | M10 LCDR2 | GASSRAT |
| 884 | M11 LCDR2 | AAS |
| 885 | M12 LCDR2 | KVSNRFS |
| 12 | P LCDR3 | QQWEGNPRT |
| 13 | P LCDR3 | QQWKGNPRT |
| 14 | P LCDR3 | QQWSGNPRT |
| 15 | P LCDR3 | QQWSRNPRT |
| 886 | P LCDR3 | FQGSHVPYT |
| 887 | P LCDR3 | HQWSGNPRT |
| 888 | P LCDR3 | NQWSGNPRT |
| 889 | P LCDR3 | SQWSGNPRT |
| 890 | P LCDR3 | QQSSGNPRT |
| 891 | P LCDR3 | QQWDGNPRT |
| 892 | P LCDR3 | QQWHGNPRT |
| 893 | P LCDR3 | QQWNGNPRT |
| 894 | P LCDR3 | QQWQGNPRT |
| 895 | P LCDR3 | QQWVGNPRT |
| 896 | P LCDR3 | QQWSANPRT |
| 897 | P LCDR3 | QQWSDNPRT |
| 898 | P LCDR3 | QQWSQNPRT |
| 899 | P LCDR3 | QQWSSNPRT |
| 900 | P LCDR3 | QQWSGNPRS |
| 901 | P LCDR3 | QQFSGNPRT |
| 902 | P LCDR3 | QQHSGNPRT |
| 903 | P LCDR3 | QQISGNPRT |
| 904 | P LCDR3 | QQPSGNPRT |
| 905 | P LCDR3 | QQRSGNPRT |
| 906 | P LCDR3 | QQYSGNPRT |
| 907 | P LCDR3 | QQWSGHPRT |
| 908 | P LCDR3 | QQWSGLPRT |
| 909 | P LCDR3 | QQWSGQPRT |
| 910 | P LCDR3 | QQWSGSPRT |
| 911 | P LCDR3 | QQWSGTPRT |
| 912 | P LCDR3 | QQWSGMPRT |
| 913 | P LCDR3 | QQWSGFPRT |
| 914 | P LCDR3 | QQWSGKPRT |
| 915 | P LCDR3 | QQWSGRPRT |
| 916 | P LCDR3 | QQWSGDPRT |
| 917 | P LCDR3 | QQWAGNPRT |
| 918 | P LCDR3 | QQWYGNPRT |
| 919 | P LCDR3 | QQWFGNPRT |
| 920 | P LCDR3 | QQWQGNPRT |
| 921 | P LCDR3 | GNPRT |
| 922 | P LCDR3 | SQWSGNPRT |
| 923 | P LCDR3 | QQWSGNPRS |
| 924 | P LCDR3 | QQWSGTPRT |
| 925 | P LCDR3 | QQWSGDPRT |
| 926 | P LCDR3 | QQWSGFPRT |
| 927 | P LCDR3 | QQWSGKPRT |
| 928 | P LCDR3 | QQWSGRPRT |
| 929 | P LCDR3 | QQWSGSPRT |
| 930 | P LCDR3 | QQWDADPRT |
| 931 | P LCDR3 | QQWDAFPRT |
| 932 | P LCDR3 | QQWDAKPRT |
| 933 | P LCDR3 | QQWDANPRT |
| 934 | P LCDR3 | QQWDARPRT |
| 935 | P LCDR3 | QQWDASPRT |
| 936 | P LCDR3 | QQWDATPRT |
| 937 | P LCDR3 | QQWDGDPRT |
| 938 | P LCDR3 | QQWDGFPRT |
| 939 | P LCDR3 | QQWDGKPRT |
| 940 | P LCDR3 | QQWDGNPRT |
| 941 | P LCDR3 | QQWDGRPRT |
| 942 | P LCDR3 | QQWDGSPRT |
| 943 | P LCDR3 | QQWDGTPRT |
| 944 | P LCDR3 | QQWEADPRT |
| 945 | P LCDR3 | QQWEAFPRT |
| 946 | P LCDR3 | QQWEAKPRT |
| 947 | P LCDR3 | QQWEANPRT |
| 948 | P LCDR3 | QQWEARPRT |
| 949 | P LCDR3 | QQWEASPRT |
| 950 | P LCDR3 | QQWEATPRT |
| 951 | P LCDR3 | QQWEGDPRT |
| 952 | P LCDR3 | QQWEGFPRT |
| 953 | P LCDR3 | QQWEGKPRT |
| 954 | P LCDR3 | QQWEGRPRT |
| 955 | P LCDR3 | QQWEGSPRT |
| 956 | P LCDR3 | QQWEGTPRT |
| 957 | P LCDR3 | QQWHADPRT |
| 958 | P LCDR3 | QQWHAFPRT |
| 959 | P LCDR3 | QQWHAKPRT |
| 960 | P LCDR3 | QQWHANPRT |
| 961 | P LCDR3 | QQWHARPRT |
| 962 | P LCDR3 | QQWHASPRT |
| 963 | P LCDR3 | QQWHATPRT |
| 964 | P LCDR3 | QQWHGDPRT |
| 965 | P LCDR3 | QQWHGFPRT |
| 966 | P LCDR3 | QQWHGKPRT |
| 967 | P LCDR3 | QQWHGNPRT |
| 968 | P LCDR3 | QQWHGRPRT |
| 969 | P LCDR3 | QQWHGSPRT |
| 970 | P LCDR3 | QQWHGTPRT |
| 971 | P LCDR3 | QQWNADPRT |
| 972 | P LCDR3 | QQWNAFPRT |
| 973 | P LCDR3 | QQWNAKPRT |
| 974 | P LCDR3 | QQWNANPRT |
| 975 | P LCDR3 | QQWNARPRT |
| 976 | P LCDR3 | QQWNASPRT |
| 977 | P LCDR3 | QQWNATPRT |
| 978 | P LCDR3 | QQWNGDPRT |
| 979 | P LCDR3 | QQWNGFPRT |
| 980 | P LCDR3 | QQWNGKPRT |
| 981 | P LCDR3 | QQWNGNPRT |
| 982 | P LCDR3 | QQWNGRPRT |
| 983 | P LCDR3 | QQWNGSPRT |
| 984 | P LCDR3 | QQWNGTPRT |
| 985 | P LCDR3 | QQWQADPRT |
| 986 | P LCDR3 | QQWQAFPRT |
| 987 | P LCDR3 | QQWQAKPRT |
| 988 | P LCDR3 | QQWQANPRT |
| 989 | P LCDR3 | QQWQARPRT |
| 990 | P LCDR3 | QQWQASPRT |
| 991 | P LCDR3 | QQWQATPRT |
| 992 | P LCDR3 | QQWQGDPRT |
| 993 | P LCDR3 | QQWQGFPRT |
| 994 | P LCDR3 | QQWQGKPRT |
| 995 | P LCDR3 | QQWQGRPRT |
| 996 | P LCDR3 | QQWQGSPRT |
| 997 | P LCDR3 | QQWQGTPRT |
| 998 | P LCDR3 | QQWSADPRT |
| 999 | P LCDR3 | QQWSAFPRT |
| 1000 | P LCDR3 | QQWSAKPRT |
| 1001 | P LCDR3 | QQWSANPRT |
| 1002 | P LCDR3 | QQWSARPRT |
| 1003 | P LCDR3 | QQWSASPRT |
| 1004 | P LCDR3 | QQWSATPRT |
| 1005 | P LCDR3 | NQWDAFPRT |
| 1006 | P LCDR3 | NQWDAKPRT |
| 1007 | P LCDR3 | NQWDANPRT |
| 1008 | P LCDR3 | NQWDARPRT |
| 1009 | P LCDR3 | NQWDASPRT |
| 1010 | P LCDR3 | NQWDATPRT |
| 1011 | P LCDR3 | NQWDGDPRT |
| 1012 | P LCDR3 | NQWDGFPRT |
| 1013 | P LCDR3 | NQWDGKPRT |
| 1014 | P LCDR3 | NQWDGNPRT |
| 1015 | P LCDR3 | NQWDGRPRT |
| 1016 | P LCDR3 | NQWDGSPRT |
| 1017 | P LCDR3 | NQWDGTPRT |
| 1018 | P LCDR3 | NQWEADPRT |
| 1019 | P LCDR3 | NQWEAFPRT |
| 1020 | P LCDR3 | NQWEAKPRT |
| 1021 | P LCDR3 | NQWEANPRT |
| 1022 | P LCDR3 | NQWEARPRT |
| 1023 | P LCDR3 | NQWEASPRT |
| 1024 | P LCDR3 | NQWEATPRT |
| 1025 | P LCDR3 | NQWEGDPRT |
| 1026 | P LCDR3 | NQWEGFPRT |
| 1027 | P LCDR3 | NQWEGKPRT |
| 1028 | P LCDR3 | NQWEGNPRT |
| 1029 | P LCDR3 | NQWEGRPRT |
| 1030 | P LCDR3 | NQWEGSPRT |
| 1031 | P LCDR3 | NQWEGTPRT |
| 1032 | P LCDR3 | NQWHADPRT |
| 1033 | P LCDR3 | NQWHAFPRT |
| 1034 | P LCDR3 | NQWHAKPRT |
| 1035 | P LCDR3 | NQWHANPRT |
| 1036 | P LCDR3 | NQWHARPRT |
| 1037 | P LCDR3 | NQWHASPRT |
| 1038 | P LCDR3 | NQWHATPRT |
| 1039 | P LCDR3 | NQWHGDPRT |
| 1040 | P LCDR3 | NQWHGFPRT |
| 1041 | P LCDR3 | NQWHGKPRT |
| 1042 | P LCDR3 | NQWHGNPRT |
| 1043 | P LCDR3 | NQWHGRPRT |
| 1044 | P LCDR3 | NQWHGSPRT |
| 1045 | P LCDR3 | NQWHGTPRT |
| 1046 | P LCDR3 | NQWNADPRT |
| 1047 | P LCDR3 | NQWNAFPRT |
| 1048 | P LCDR3 | NQWNAKPRT |
| 1049 | P LCDR3 | NQWNANPRT |
| 1050 | P LCDR3 | NQWNARPRT |
| 1051 | P LCDR3 | NQWNASPRT |
| 1052 | P LCDR3 | NQWNATPRT |
| 1053 | P LCDR3 | NQWNGDPRT |
| 1054 | P LCDR3 | NQWNGFPRT |
| 1055 | P LCDR3 | NQWNGKPRT |
| 1056 | P LCDR3 | NQWNGNPRT |
| 1057 | P LCDR3 | NQWNGRPRT |
| 1058 | P LCDR3 | NQWNGSPRT |
| 1059 | P LCDR3 | NQWNGTPRT |
| 1060 | P LCDR3 | NQWQADPRT |
| 1061 | P LCDR3 | NQWQAFPRT |
| 1062 | P LCDR3 | NQWQAKPRT |
| 1063 | P LCDR3 | NQWQANPRT |
| 1064 | P LCDR3 | NQWQARPRT |
| 1065 | P LCDR3 | NQWQASPRT |
| 1066 | P LCDR3 | NQWQATPRT |
| 1067 | P LCDR3 | NQWQGDPRT |
| 1068 | P LCDR3 | NQWQGFPRT |
| 1069 | P LCDR3 | NQWQGKPRT |
| 1070 | P LCDR3 | NQWQGNPRT |
| 1071 | P LCDR3 | NQWQGRPRT |
| 1072 | P LCDR3 | NQWQGSPRT |
| 1073 | P LCDR3 | NQWQGTPRT |
| 1074 | P LCDR3 | NQWSADPRT |
| 1075 | P LCDR3 | NQWSAFPRT |
| 1076 | P LCDR3 | NQWSAKPRT |
| 1077 | P LCDR3 | NQWSANPRT |
| 1078 | P LCDR3 | NQWSARPRT |
| 1079 | P LCDR3 | NQWSASPRT |
| 1080 | P LCDR3 | NQWSATPRT |
| 1081 | P LCDR3 | NQWSGDPRT |
| 1082 | P LCDR3 | NQWSGFPRT |
| 1083 | P LCDR3 | NQWSGKPRT |
| 1084 | P LCDR3 | NQWSGNPRT |
| 1085 | P LCDR3 | NQWSGRPRT |
| 1086 | P LCDR3 | NQWSGSPRT |
| 1087 | P LCDR3 | NQWSGTPRT |
| 1088 | P LCDR3 | X₁QWX₂X₃X₄PRT |
| | | X₁ = Q, OR N |
| | | X₂ = D, E, H, N, Q, OR S |
| | | X₃ = A, OR G |
| | | X₄ = D, F, K, N, R, S, OR T |
| 1089 | P LCDR3 | X₁QX₂X₃X₄X₅PRX₆ |
| | | X1 = H, N, Q, or S |
| | | X2 = F, H, I, P, R, S, W, or |
| | | Y |
| | | X3 = D, E, H, N, Q, S, or V |
| | | X4 = A, D, G, Q, or S |
| | | X5 = D, F, H, K, L, M, N, Q, |
| | | R, S, or T |
| | | X6 = T or S |
| 1090 | M1 LCDR3 | QQYYSTPFT |
| 1091 | M2 LCDR3 | MQATQFPLT |
| 1092 | M3 LCDR3 | QQRSNWPWT |
| 1093 | M4 LCDR3 | QQFNSYPLT |
| 1094 | M5 LCDR3 | HQWNNYGT |
| 1095 | M6 LCDR3 | FQGSHVPLT |
| 1096 | M7 LCDR3 | QQGQSYPYT |
| 1097 | M8 LCDR3 | QSXDGTLSAL; X = Y, W, F |
| 1098 | M9 LCDR3 | QQSYSTPRT |
| 1099 | M10 LCDR3 | QQYGSSPT |
| 1100 | M11 LCDR3 | QQYHRSWT |
| 1101 | M12 LCDR3 | FQGSHVPLT |

TABLE 2B
Select antibody CDRs

| | Heavy Chain | Light Chain |
|---|---|---|
| | CDR SEQ ID NOS | CDR SEQ ID NOS |
| Antibody | (CDR1, CDR2, CDR3) | (CDR1, CDR2, CDR3) |
| A | 1, 2, 6 | 10, 11, 12 |
| B | 1, 3, 8 | 10, 11, 12 |
| C | 1, 4, 8 | 10, 11, 12 |
| D | 1, 2, 6 | 10, 11, 13 |
| E | 1, 2, 6 | 10, 11, 14 |
| F | 1, 5, 8 | 10, 11, 12 |
| G | 1, 5, 8 | 10, 11, 13 |
| H | 1, 3, 8 | 10, 11, 13 |
| A2 | 1, 2, 7 | 10, 11, 12 |
| B2 | 1, 3, 9 | 10, 11, 12 |
| C2 | 1, 4, 9 | 10, 11, 12 |
| D2 | 1, 2, 7 | 10, 11, 13 |
| E2 | 1, 2, 7 | 10, 11, 14 |
| F2 | 1, 5, 9 | 10, 11, 12 |
| G2 | 1, 5, 9 | 10, 11, 13 |
| H2 | 1, 3, 9 | 10, 11, 13 |
| I | 1, 5, 8 | 10, 11, 15 |
| I2 | 1, 5, 9 | 10, 11, 15 |

TABLE 2C
Variable Regions of Certain anti-TL1A Antibodies
As used herein, reference to A(number), refers to an antibody of this
table. For instance, A15 used herein refers to A15 in Table 2C.

| | Heavy Chain Variable | Light Chain Variable |
|---|---|---|
| Antibody | Region SEQ ID NO | Region SEQ ID NOS |
| A15 | 108 | 203 |
| A29 | 108 | 205 |
| A30 | 108 | 204 |
| A31 | 136 | 205 |
| A32 | 137 | 205 |
| A33 | 137 | 202 |
| A34 | 107 | 208 |
| A35 | 138 | 208 |
| A36 | 139 | 208 |
| A37 | 140 | 208 |
| A38 | 141 | 208 |
| A39 | 142 | 208 |
| A40 | 143 | 208 |
| A41 | 115 | 208 |
| A42 | 144 | 208 |
| A43 | 145 | 208 |
| A44 | 146 | 208 |
| A45 | 120 | 208 |
| A46 | 147 | 208 |
| A47 | 148 | 208 |
| A48 | 108 | 210 |
| A49 | 108 | 211 |
| A50 | 108 | 212 |
| A51 | 108 | 213 |
| A52 | 108 | 214 |
| A53 | 146 | 208 |
| A54 | 149 | 208 |
| A55 | 109 | 208 |
| A56 | 108 | 215 |
| A57 | 150 | 202 |
| A58 | 125 | 202 |
| A59 | 117 | 202 |
| A60 | 151 | 202 |
| A61 | 152 | 202 |
| A62 | 153 | 202 |
| A63 | 154 | 202 |
| A64 | 121 | 202 |
| A65 | 128 | 202 |
| A66 | 155 | 202 |
| A67 | 122 | 202 |
| A68 | 123 | 202 |
| A69 | 156 | 202 |
| A70 | 157 | 202 |
| A71 | 158 | 202 |
| A72 | 131 | 202 |
| A73 | 157 | 205 |
| A74 | 158 | 205 |
| A75 | 131 | 205 |
| A76 | 159 | 202 |
| A77 | 160 | 202 |
| A78 | 124 | 202 |
| A79 | 107 | 208 |
| A81 | 139 | 208 |
| A82 | 140 | 208 |
| A83 | 144 | 208 |
| A85 | 136 | 209 |
| A86 | 136 | 216 |
| A87 | 136 | 217 |
| A88 | 136 | 218 |
| A89 | 136 | 219 |
| A90 | 136 | 220 |
| A91 | 133 | 202 |
| A92 | 161 | 202 |
| A93 | 162 | 202 |
| A94 | 124 | 202 |
| A95 | 131 | 205 |
| A96 | 128 | 205 |
| A97 | 121 | 202 |
| A98 | 122 | 202 |
| A99 | 123 | 202 |
| A100 | 107 | 204 |
| A101 | 140 | 204 |
| A102 | 115 | 204 |
| A103 | 120 | 204 |
| A104 | 139 | 204 |
| A105 | 143 | 204 |
| A107 | 108 | 202 |
| A108 | 156 | 205 |
| A109 | 133 | 205 |
| A110 | 125 | 205 |
| A111 | 150 | 205 |
| A112 | 117 | 205 |
| A113 | 124 | 205 |
| A114 | 121 | 205 |
| A115 | 122 | 205 |
| A116 | 123 | 205 |
| A117 | 151 | 205 |
| A118 | 153 | 205 |
| A119 | 159 | 205 |
| A120 | 154 | 205 |
| A121 | 163 | 204 |
| A122 | 113 | 204 |
| A123 | 112 | 204 |
| A124 | 164 | 204 |
| A125 | 105 | 204 |
| A126 | 114 | 204 |
| A127 | 118 | 204 |
| A128 | 111 | 204 |
| A129 | 110 | 204 |
| A130 | 121 | 205 |
| A132 | 128 | 206 |
| A133 | 121 | 206 |
| A134 | 122 | 206 |
| A135 | 133 | 206 |
| A136 | 125 | 206 |
| A137 | 121 | 207 |
| A138 | 122 | 207 |
| A139 | 110 | 207 |
| A140 | 110 | 202 |
| A141 | 111 | 207 |
| A142 | 111 | 202 |
| A143 | 136 | 202 |
| A144 | 111 | 204 |
| A145 | 133 | 201 |
| A146 | 125 | 201 |
| A147 | 117 | 201 |
| A148 | 121 | 201 |
| A149 | 122 | 201 |
| A150 | 128 | 201 |
| A151 | 124 | 201 |
| A152 | 131 | 201 |
| A153 | 133 | 205 |
| A154 | 125 | 205 |
| A155 | 121 | 205 |
| A156 | 122 | 205 |
| A157 | 104 | 204 |
| A158 | 101 | 204 |
| A159 | 119 | 204 |
| A160 | 102 | 204 |
| A161 | 165 | 204 |
| A162 | 106 | 204 |
| A163 | 166 | 204 |
| A164 | 167 | 204 |
| A165 | 139 | 205 |
| A166 | 146 | 205 |
| A167 | 120 | 205 |
| A168 | 147 | 205 |
| A169 | 126 | 205 |
| A170 | 135 | 205 |
| A171 | 168 | 205 |
| A172 | 130 | 205 |
| A173 | 127 | 205 |
| A174 | 132 | 205 |
| A175 | 126 | 201 |
| A176 | 135 | 201 |
| A177 | 168 | 201 |
| A178 | 130 | 201 |
| A179 | 127 | 201 |
| A180 | 132 | 201 |
| A181 | 107 | 202 |
| A182 | 138 | 202 |
| A183 | 140 | 202 |
| A184 | 145 | 202 |
| A185 | 147 | 202 |
| A186 | 144 | 202 |
| A187 | 120 | 202 |
| A188 | 115 | 202 |
| A189 | 146 | 202 |
| A190 | 141 | 202 |
| A191 | 142 | 202 |
| A192 | 143 | 202 |
| A193 | 109 | 205 |
| A194 | 103 | 205 |
| A195 | 169 | 205 |
| A196 | 129 | 205 |
| A197 | 116 | 205 |
| A198 | 134 | 205 |
| A199 | 109 | 201 |
| A200 | 103 | 201 |
| A201 | 169 | 201 |
| A202 | 129 | 201 |
| A203 | 116 | 201 |
| A204 | 134 | 201 |
| A205 | 109 | 202 |
| A206 | 103 | 202 |
| A207 | 169 | 202 |
| A208 | 129 | 202 |
| A209 | 116 | 202 |
| A210 | 134 | 202 |
| A211 | 108 | 201 |
| A212 | 107 | 201 |
| A213 | 106 | 201 |
| A214 | 111 | 201 |
| A215 | 110 | 201 |
| A216 | 112 | 201 |
| A217 | 101 | 201 |
| A218 | 119 | 201 |
| A219 | 104 | 201 |
| A220 | 102 | 205 |
| A221 | 105 | 201 |
| A222 | 114 | 201 |
| A223 | 103 | 202 |
| A224 | 116 | 201 |
| A500 | 301 | 303 |
| A501 | 302 | 303 |

TABLE 2D
Heavy chain variable region (VH) amino acid sequences

| SEQ | | |
|---|---|---|
| ID | | |
| NO | Description | Sequence |
| 101 | 217 VH, 158 VH | QVQLVQSGAEVKKPGASVKVSCKASGFDIQDTYMHWVKQRPGQGLEW |
| | | MGRIDPASGHTKYDPKFQVRVTITRDTSTSTVYMELSSLRSEDTAVYYC |
| | | ARSGGLPDVWGQGTTVTVSS |
| 102 | 220 VH, 160 VH | QVQLVQSGAEVKKPGASVKVSCKASGFDIQDTYMHWVKQRPGQGLEW |
| | | MGRIDPASGHTKYDPKFQVRVTMTRDTSTSTAYLELSSLRSEDTAVYYC |
| | | ARSGGLPDVWGQGTTVTVSS |
| 103 | 223 VH, 200 VH, | EVQLVQSGAEVKKPGASVKVSCKASGFDIQDTYMHWVRQRPGQGLEWI |
| | 194 VL, 206 VH | GRIDPASGHTKYDPKFQVRATITTDTSTSTAYLELSSLRSEDTAVYYCAR |
| | | SGGLPDVWGQGTTVTVSS |
| 104 | 219 VH, 157 VH | QVQLVQSGAEVKKPGASVKVSCKASGFDIQDTYMHWVKQRPGQGLEW |
| | | MGRIDPASGHTKYDPKFQVRVTITRDTSTSTVYLELSSLRSEDTAVYYCA |
| | | RSGGLPDVWGQGTTVTVSS |
| 105 | 221 VH, 125 VH | QVQLVQSGAEVKKPGASVKVSCKASGFDIQDTYMHWVKQRPGQGLEW |
| | | MGRIDPASGHTKYDPKFQVRATITRDTSTSTAYLELSSLRSEDTAVYYCA |
| | | RSGGLPDVWGQGTTVTVSS |
| 106 | 213 VH, 162 VH | QVQLVQSGAEVKKPGASVKVSCKASGFDIQDTYMHWVRQRPGQGLEW |
| | | MGRIDPASGHTKYDPKFQVRVTITTDTSTSTVYMELSSLRSEDTAVYYCA |
| | | RSGGLPDVWGQGTTVTVSS |
| 107 | 212 VH, 100 VH, | QVQLVQSGAEVKKPGASVKVSCKASGFDIQDTYMHWVRQRPGQGLEWI |
| | 181 VH, 34 VH, 79 | GRIDPASGHTKYDPKFQVRATITTDTSTSTAYLELSSLRSEDTAVYYCAR |
| | VH | SGGLPDVWGQGTTVTVSS |
| 108 | 107 VH, 211 VH, 15 | QVQLVQSGAEVKKPGASVKVSCKASGFDIQDTYMHWVKQRPGQGLEWI |
| | VH, 30 VH, 29 VH, | GRIDPASGHTKYDPKFQVRATITTDTSTSTAYLELSSLRSEDTAVYYCAR |
| | 48 VH, 49 VH, 50 | SGGLPDVWGQGTTVTVSS |
| | VH, 51 VH, 52 VH, | |
| | 56 VH | |
| 109 | 205 VH, 199 VH, 55 | EVQLVQSGAEVKKPGASVKVSCKASGFDIQDTYMHWVKQRPGQGLEWI |
| | VH, 193 VH | GRIDPASGHTKYDPKFQVRATITTDTSTSTAYLELSSLRSEDTAVYYCAR |
| | | SGGLPDVWGQGTTVTVSS |
| 110 | 129 VH, 139 VH, | QVQLVQSGAEVKKPGASVKVSCKASGFDIQDTYMHWVRQRPGQGLEW |
| | 140 VH, 215 VH | MGRIDPASGHTKYDPKFQVRVTITTDTSTSTAYLELSSLRSEDTAVYYCA |
| | | RSGGLPDVWGQGTTVTVSS |
| 111 | 214 VH, 128 VH, | QVQLVQSGAEVKKPGASVKVSCKASGFDIQDTYMHWVRQRPGQGLEW |
| | 141 VH, 142 VH, | MGRIDPASGHTKYDPKFQVRVTITRDTSTSTAYLELSSLRSEDTAVYYCA |
| | 144 VH | RSGGLPDVWGQGTTVTVSS |
| 112 | 216 VH, 123 VH | QVQLVQSGAEVKKPGASVKVSCKASGFDIQDTYMHWVRQRPGQGLEWI |
| | | GRIDPASGHTKYDPKFQVRVTITRDTSTSTAYLELSSLRSEDTAVYYCAR |
| | | SGGLPDVWGQGTTVTVSS |
| 113 | 122 VH | QVQLVQSGAEVKKPGASVKVSCKASGFDIQDTYMHWVRQRPGQGLEWI |
| | | GRIDPASGHTKYDPKFQVRATITRDTSTSTAYLELSSLRSEDTAVYYCAR |
| | | SGGLPDVWGQGTTVTVSS |
| 114 | 222 VH, 126 VH | QVQLVQSGAEVKKPGASVKVSCKASGFDIQDTYMHWVKQRPGQGLEW |
| | | MGRIDPASGHTKYDPKFQVRVTITRDTSTSTAYLELSSLRSEDTAVYYCA |
| | | RSGGLPDVWGQGTTVTVSS |
| 115 | 188 VH, 41 VH, 102 | QVQLVQSGAEVKKPGASVKVSCKASGFDIQDTYMHWVKQRPGQGLEWI |
| | VH | GRIDPASGHTKYDPKFQVRVTITTDTSTSTAYLELSSLRSEDTAVYYCAR |
| | | SGGLPDVWGQGTTVTVSS |
| 116 | 203 VH, 197 VH, | EVQLVQSGAEVKKPGASVKVSCKASGFDIQDTYMHWVKQAPGQGLEW |
| | 209 VH, 224 VH | MGRIEPASGHIKYDPKFQGRVTMTRDTSTSTVYMELSSLRSEDTAVYYC |
| | | ARSGGLPDWWGQGTTVTVSS |
| 117 | 147 VH, 112 VH, 59 | QVQLVQSGAEVKKPGASVKVSCKASGFDIQDTYMHWVKQRPGQGLEW |
| | VH | MGRIEPASGHIKYDPKFQGRVTMTRDTSTSTVYMELSSLRSEDTAVYYC |
| | | ARSGGLPDWWGQGTTVTVSS |
| 118 | 127 VH | QVQLVQSGAEVKKPGASVKVSCKASGFDIQDTYMHWVKQRPGQGLEW |
| | | MGRIDPASGHTKYDPKFQVRVTITTDTSTSTAYLELSSLRSEDTAVYYCA |
| | | RSGGLPDVWGQGTTVTVSS |
| 119 | 159 VH, 218 VH | QVQLVQSGAEVKKPGASVKVSCKASGFDIQDTYMHWVKQRPGQGLEW |
| | | MGRIDPASGHTKYDPKFQVRVTITRDTSTSTAYMELSSLRSEDTAVYYC |
| | | ARSGGLPDVWGQGTTVTVSS |
| 120 | 103 VH, 45 VH, 167 | QVQLVQSGAEVKKPGASVKVSCKASGFDIQDTYMHWVKQRPGQGLEWI |
| | VH, 187 VH | GRIDPASGHTKYDPKFQVRVTITRDTSTSTAYLELSSLRSEDTAVYYCAR |
| | | SGGLPDVWGQGTTVTVSS |
| 121 | 64 VH, 148 VH, 97 | QVQLVQSGAEVKKPGASVKVSCKASGFDIQDTYMHWVRQAPGQGLEW |
| | VH, 114 VH, 130 VH, | MGRIEPASGHIKYDPKFQVRVTMTRDTSTSTVYMELSSLRSEDTAVYYC |
| | 133 VH, 137 VH, | ARSGGLPDWWGQGTTVTVSS |
| | 155 VH | |
| 122 | 67 VH, 138 VH, 115 | QVQLVQSGAEVKKPGASVKVSCKASGFDIQDTYMHWVRQAPGQGLEW |
| | VH, 149 VH, 134 VH, | MGRIEPASGHIKYDPKFQVRATMTRDTSTSTVYMELSSLRSEDTAVYYC |
| | 98 VH, 156 VH | ARSGGLPDWWGQGTTVTVSS |
| 123 | 68 VH, 99 VH, 116 | QVQLVQSGAEVKKPGASVKVSCKASGFDIQDTYMHWVRQAPGQGLEW |
| | VH | MGRIEPASGHIKYDPKFQVRVTITRDTSTSTVYMELSSLRSEDTAVYYCA |
| | | RSGGLPDWWGQGTTVTVSS |
| 124 | 94 VH, 113 VH, 151 | QVQLVQSGAEVKKPGASVKVSCKASGFDIQDTYMHWVRQAPGQGLEW |
| | VH, 78 VH | MGRIEPASGHIKYDPKFQVRATITRDTSTSTVYMELSSLRSEDTAVYYCA |
| | | RSGGLPDWWGQGTTVTVSS |
| 125 | 110 VH, 58 VH, 136 | QVQLVQSGAEVKKPGASVKVSCKASGFDIQDTYMHWVKQAPGQGLEW |
| | VH, 146 VH, 154 VH | MGRIEPASGHIKYDPKFQGRVTMTRDTSTSTVYMELSSLRSEDTAVYYC |
| | | ARSGGLPDWWGQGTTVTVSS |
| 126 | 169 VH, 175 VH | QVQLVQSGAEVKKPGASVKVSCKASGFDIQDTYMHWVKQAPGQGLEW |
| | | MGRIDPASGHIKYDPKFQGRVTMTRDTSTSTVYMELSSLRSEDTAVYYC |
| | | ARSGGLPDWWGQGTTVTVSS |
| 127 | 173 VH, 179 VH | QVQLVQSGAEVKKPGASVKVSCKASGFDIQDTYMHWVKQAPGQGLEW |
| | | MGRIEPASGHIKYDPKFQGRATMTRDTSTSTVYMELSSLRSEDTAVYYC |
| | | ARSGGLPDWWGQGTTVTVSS |
| 128 | 96 VH, 132 VH, 65 | QVQLVQSGAEVKKPGASVKVSCKASGFDIQDTYMHWVRQAPGQGLEW |
| | VH, 150 VH | MGRIEPASGHIKYDPKFQGRATMTRDTSTSTVYMELSSLRSEDTAVYYC |
| | | ARSGGLPDWWGQGTTVTVSS |
| 129 | 196 VH, 202 VH, | EVQLVQSGAEVKKPGASVKVSCKASGFDIQDTYMHWVRQAPGQGLEW |
| | 208 VH | MGRIEPASGHIKYDPKFQGRATMTRDTSTSTVYMELSSLRSEDTAVYYC |
| | | ARSGGLPDWWGQGTTVTVSS |
| 130 | 172 VH, 178 VH | QVQLVQSGAEVKKPGASVKVSCKASGFDIQDTYMHWVRQAPGQGLEW |
| | | MGRIDPASGHIKYDPKFQGRATMTRDTSTSTVYMELSSLRSEDTAVYYC |
| | | ARSGGLPDWWGQGTTVTVSS |
| 131 | 75 VH, 72 VH, 95 | QVQLVQSGAEVKKPGASVKVSCKASGFDIQDTYMHWVRQAPGQGLEW |
| | VH, 152 VH | MGRIEPASGHIKYDPKFQGRATITTDTSTSTVYMELSSLRSEDTAVYYCA |
| | | RSGGLPDWWGQGTTVTVSS |
| 132 | 174 VH, 180 VH | QVQLVQSGAEVKKPGASVKVSCKASGFDIQDTYMHWVRQAPGQGLEW |
| | | MGRIEPASGHIKYDPKFQGRATMTRDTSTSTAYMELSSLRSEDTAVYYC |
| | | ARSGGLPDWWGQGTTVTVSS |
| 133 | 109 VH, 91 VH, 135 | QVQLVQSGAEVKKPGASVKVSCKASGFDIQDTYMHWVRQAPGQGLEW |
| | VH, 145 VH, 153 VH | MGRIEPASGHIKYDPKFQGRVTMTRDTSTSTAYMELSSLRSEDTAVYYC |
| | | ARSGGLPDWWGQGTTVTVSS |
| 134 | 198 VH, 204 VH, | EVQLVQSGAEVKKPGASVKVSCKASGFDIQDTYMHWVRQAPGQGLEW |
| | 210 VH | MGRIEPASGHIKYDPKFQGRVTMTRDTSTSTAYMELSSLRSEDTAVYYC |
| | | ARSGGLPDWWGQGTTVTVSS |
| 135 | 170 VH, 176 VH | QVQLVQSGAEVKKPGASVKVSCKASGFDIQDTYMHWVRQAPGQGLEW |
| | | MGRIDPASGHIKYDPKFQGRVTMTRDTSTSTAYMELSSLRSEDTAVYYC |
| | | ARSGGLPDWWGQGTTVTVSS |
| 136 | 31 VH, 85 VH, 86 | QVQLVQSGAEVKKPGASVKVSCKASGFDIQDTYMHWVRQAPGQGLEW |
| | VH, 87 VH, 88 VH, | MGRIEPASGHIKYDPKFQGRVTMTRDTSTSTVYMELSSLRSEDTAVYYC |
| | 89 VH, 90 VH, 143 | ARSGGLPDWWGQGTTVTVSS |
| | VH, clone 34 VH | |
| 137 | 32 VH, 33 VH | DVQLVQSGAEVKKPGASVKVSCKASGFDIQDTYMHWVRQAPGQGLEW |
| | | MGRIEPASGHIKYDPKFQGRVTMTRDTSTSTVYMELSSLRSEDTAVYYC |
| | | ARSGGLPDWWGQGTTVTVSS |
| 138 | 35 VH, 182 VH | QVQLVQSGAEVKKPGASVKVSCKASGFDIQDTYMHWVKQAPGQGLEWI |
| | | GRIDPASGHTKYDPKFQVRATITTDTSTSTAYLELSSLRSEDTAVYYCAR |
| | | SGGLPDVWGQGTTVTVSS |
| 139 | 36 VH, 81 VH, 104 | QVQLVQSGAEVKKPGASVKVSCKASGFDIQDTYMHWVRQAPGQGLEWI |
| | VH, 165 VH, | GRIDPASGHTKYDPKFQVRATITTDTSTSTAYLELSSLRSEDTAVYYCAR |
| | | SGGLPDVWGQGTTVTVSS |
| 140 | 37 VH, 82 VH, 101 | QVQLVQSGAEVKKPGASVKVSCKASGFDIQDTYMHWVKQRPGQGLEW |
| | VH, 183 VH | MGRIDPASGHTKYDPKFQVRATITTDTSTSTAYLELSSLRSEDTAVYYCA |
| | | RSGGLPDVWGQGTTVTVSS |
| 141 | 38 VH, 190 VH | QVQLVQSGAEVKKPGASVKVSCKASGFDIQDTYMHWVKQRPGQGLEWI |
| | | GRIDPASGHTKYDPKFQVRATITTDTSTSTVYLELSSLRSEDTAVYYCAR |
| | | SGGLPDVWGQGTTVTVSS |
| 142 | 39 VH, 191 VH | QVQLVQSGAEVKKPGASVKVSCKASGFDIQDTYMHWVKQRPGQGLEWI |
| | | GRIDPASGHTKYDPKFQVRATITTDTSTSTAYMELSSLRSEDTAVYYCAR |
| | | SGGLPDVWGQGTTVTVSS |
| 143 | 40 VH, 105 VH, 192 | QVQLVQSGAEVKKPGASVKVSCKASGFDIQDTYMHWVKQRPGQGLEWI |
| | VH | GRIDPASGHTKYDPKFQVRATITTDTSTSTVYMELSSLRSEDTAVYYCAR |
| | | SGGLPDVWGQGTTVTVSS |
| 144 | 42 VH, 83 VH, 186 | QVQLVQSGAEVKKPGASVKVSCKASGFDIQDTYMHWVKQRPGQGLEWI |
| | VH | GRIDPASGHTKYDPKFQVRATMTTDTSTSTAYLELSSLRSEDTAVYYCA |
| | | RSGGLPDVWGQGTTVTVSS |
| 145 | 43 VH, 184 VH | QVQLVQSGAEVKKPGASVKVSCKASGFDIQDTYMHWVKQRPGQGLEWI |
| | | GRIDPASGHTKYDPKFQVRATITRDTSTSTAYLELSSLRSEDTAVYYCAR |
| | | SGGLPDVWGQGTTVTVSS |
| 146 | 44 VH, 53 VH, 166 | QVQLVQSGAEVKKPGASVKVSCKASGFDIQDTYMHWVKQRPGQGLEWI |
| | VH, 189 VH | GRIDPASGHTKYDPKFQVRVTMTTDTSTSTAYLELSSLRSEDTAVYYCA |
| | | RSGGLPDVWGQGTTVTVSS |
| 147 | 46 VH, 168 VH, 185 | QVQLVQSGAEVKKPGASVKVSCKASGFDIQDTYMHWVKQRPGQGLEWI |
| | VH | GRIDPASGHTKYDPKFQVRATMTRDTSTSTAYLELSSLRSEDTAVYYCA |
| | | RSGGLPDVWGQGTTVTVSS |
| 148 | 47 VH | QVQLVQSGAEVKKPGASVKVSCKASGFDIQDTYMHWVKQRPGQGLEWI |
| | | GRIDPASGHTKYDPKFQVRVTMTRDTSTSTAYLELSSLRSEDTAVYYCA |
| | | RSGGLPDVWGQGTTVTVSS |
| 149 | 54 VH | DVQLVQSGAEVKKPGASVKVSCKASGFDIQDTYMHWVKQRPGQGLEWI |
| | | GRIDPASGHTKYDPKFQVRATITTDTSTSTAYLELSSLRSEDTAVYYCAR |
| | | SGGLPDVWGQGTTVTVSS |
| 150 | 57 VH, 111 VH | QVQLVQSGAEVKKPGASVKVSCKASGFDIQDTYMHWVRQRPGQGLEW |
| | | MGRIEPASGHIKYDPKFQGRVTMTRDTSTSTVYMELSSLRSEDTAVYYC |
| | | ARSGGLPDWWGQGTTVTVSS |
| 151 | 60 VH, 117 VH | QVQLVQSGAEVKKPGASVKVSCKASGFDIQDTYMHWVRQAPGQGLEW |
| | | MGRIDPASGHIKYDPKFQGRVTMTRDTSTSTVYMELSSLRSEDTAVYYC |
| | | ARSGGLPDWWGQGTTVTVSS |
| 152 | 61 VH | QVQLVQSGAEVKKPGASVKVSCKASGFDIQDTYMHWVRQAPGQGLEWI |
| | | GRIEPASGHIKYDPKFQGRVTMTRDTSTSTVYMELSSLRSEDTAVYYCAR |
| | | SGGLPDWWGQGTTVTVSS |
| 153 | 62 VH, 118 VH | QVQLVQSGAEVKKPGASVKVSCKASGFDIQDTYMHWVRQAPGQGLEWI |
| | | GRIDPASGHIKYDPKFQGRVTMTRDTSTSTVYMELSSLRSEDTAVYYCA |
| | | RSGGLPDWWGQGTTVTVSS |
| 154 | 63 VH, 120 VH | QVQLVQSGAEVKKPGASVKVSCKASGFDIQDTYMHWVRQAPGQGLEW |
| | | MGRIEPASGHVKYDPKFQGRVTMTRDTSTSTVYMELSSLRSEDTAVYYC |
| | | ARSGGLPDWWGQGTTVTVSS |
| 155 | 66 VH | QVQLVQSGAEVKKPGASVKVSCKASGFDIQDTYMHWVRQAPGQGLEW |
| | | MGRIEPASGHIKYDPKFQGRVTITRDTSTSTVYMELSSLRSEDTAVYYCA |
| | | RSGGLPDWWGQGTTVTVSS |
| 156 | 69 VH, 108 VH | EVQLVQSGAEVKKPGASVKVSCKASGFDIQDTYMHWVRQAPGQGLEW |
| | | MGRIEPASGHIKYDPKFQGRVTMTRDTSTSTVYMELSSLRSEDTAVYYC |
| | | ARSGGLPDWWGQGTTVTVSS |
| 157 | 70 VH, 73 VH | QVQLVQSGAEVKKPGASVKVSCKASGFDIQDTYMHWVRQAPGQGLEW |
| | | MGRIEPASGHIKYDPKFQGRVTMTTDTSTSTVYMELSSLRSEDTAVYYC |
| | | ARSGGLPDWWGQGTTVTVSS |
| 158 | 71 VH, 74 VH | QVQLVQSGAEVKKPGASVKVSCKASGFDIQDTYMHWVRQAPGQGLEW |
| | | MGRIEPASGHIKYDPKFQGRVTITTDTSTSTVYMELSSLRSEDTAVYYCA |
| | | RSGGLPDWWGQGTTVTVSS |
| 159 | 76 VH, 119 VH | QVQLVQSGAEVKKPGASVKVSCKASGFDIQDTYMHWVRQAPGQGLEW |
| | | MGRIEPASGHTKYDPKFQGRVTMTRDTSTSTVYMELSSLRSEDTAVYYC |
| | | ARSGGLPDWWGQGTTVTVSS |
| 160 | 77 VH | QVQLVQSGAEVKKPGASVKVSCKASGFDIQDTYMHWVRQAPGQGLEW |
| | | MGRIEPASGHIKYDPKFQGRATITRDTSTSTVYMELSSLRSEDTAVYYCA |
| | | RSGGLPDWWGQGTTVTVSS |
| 161 | 92 VH | QVQLVQSGAEVKKPGASVKVSCKASGFDIQDTYMHWVRQAPGQGLEW |
| | | MGRIEPASGHIKYDPKFQGRVTMTRDTSTSTVYLELSSLRSEDTAVYYCA |
| | | RSGGLPDWWGQGTTVTVSS |
| 162 | 93 VH | QVQLVQSGAEVKKPGASVKVSCKASGFDIQDTYMHWVRQAPGQGLEW |
| | | MGRIEPASGHIKYDPKFQGRVTMTRDTSTSTAYLELSSLRSEDTAVYYCA |
| | | RSGGLPDWWGQGTTVTVSS |
| 163 | 121 VH | QVQLVQSGAEVKKPGASVKVSCKASGFDIQDTYMHWVRQRPGQGLEW |
| | | MGRIDPASGHTKYDPKFQVRATITTDTSTSTAYLELSSLRSEDTAVYYCA |
| | | RSGGLPDVWGQGTTVTVSS |
| 164 | 124 VH | QVQLVQSGAEVKKPGASVKVSCKASGFDIQDTYMHWVRQRPGQGLEWI |
| | | GRIDPASGHTKYDPKFQVRVTITTDTSTSTAYLELSSLRSEDTAVYYCAR |
| | | SGGLPDVWGQGTTVTVSS |
| 165 | 161 VH | QVQLVQSGAEVKKPGASVKVSCKASGFDIQDTYMHWVRQRPGQGLEW |
| | | MGRIDPASGHTKYDPKFQVRVTITTDTSTSTVYLELSSLRSEDTAVYYCA |
| | | RSGGLPDVWGQGTTVTVSS |
| 166 | 163 VH | QVQLVQSGAEVKKPGASVKVSCKASGFDIQDTYMHWVRQRPGQGLEW |
| | | MGRIDPASGHTKYDPKFQVRVTITTDTSTSTAYMELSSLRSEDTAVYYCA |
| | | RSGGLPDVWGQGTTVTVSS |
| 167 | 164 VH | QVQLVQSGAEVKKPGASVKVSCKASGFDIQDTYMHWVRQRPGQGLEW |
| | | MGRIDPASGHTKYDPKFQVRVTMTTDTSTSTAYLELSSLRSEDTAVYYC |
| | | ARSGGLPDVWGQGTTVTVSS |
| 168 | 171 VH, 177 VH | QVQLVQSGAEVKKPGASVKVSCKASGFDIQDTYMHWVRQAPGQGLEW |
| | | MGRIDPASGHIKYDPKFQGRATITTDTSTSTVYMELSSLRSEDTAVYYCA |
| | | RSGGLPDWWGQGTTVTVSS |
| 169 | 195 VH, 201 VH, | EVQLVQSGAEVKKPGASVKVSCKASGFDIQDTYMHWVRQAPGQGLEW |
| | 207 VH | MGRIEPASGHIKYDPKFQGRATITTDTSTSTVYMELSSLRSEDTAVYYCA |
| | | RSGGLPDWWGQGTTVTVSS |
| 1200 | 5C3D11 VH | EVQLQQSGAELVKPGASVKLSCTASGFDIQDTYMHWVKQRPEQGLEWI |
| | | GRIDPASGHTKYDPKFQVKATITTDTSSNTAYLQLSSLTSEDTAVYYCSR |
| | | SGGLPDVWGAGTTVTVSS |
| 1201 | 9E12E5 VH | QVHLQQSGPELVKPGASVKLSCKASGYTFTKYDINWVRQRPEQGLEWIG |
| | | WIFPGDGRTDYNEKFKGKATLTTDKSSSTAYMEVSRLTSEDSAVYFCAR |
| | | YGPAMDYWGQGTSVTVAS |
| 1202 | AS12824 VH | QVQLVQSGAEVKKPGASVKVSCKASGFDICDTYMHWVKQRPGQGLEWI |
| | | GRIDPASGHTKYDPKFQVRATITTDTSTSTAYLELSSLRSEDTAVYYCAR |
| | | SGGLPDVWGQGTTVTVSS |
| 1203 | AS12823 VH | QVQLVQSGAEVKKPGASVKLSCKASGFDICDTYMHWVRQRPGQGLEWI |
| | | GRIDPASGHTKYDPKFQVRATMTTDTSTSTVYLELSSLRSEDTAVYYCA |
| | | RSGGLPDVWGQGTTVTVSS |
| 1204 | AS12819 VH | QVQLVQSGAEVVKPGASVKLSCKASGFDICDTYMHWVRQRPGQGLEW |
| | | MGRIDPASGHTKYDPKFQVRVTMTTDTSTSTVYLELSSLRSEDTAVYYC |
| | | ARSGGLPDVWGQGTTVTVSS |
| 1205 | AS12816 VH | QVQLVQSGAEVKKPGASVKVSCKASGFDICDTYMHWVKQRPGQGLEWI |
| | | GRIDPASGHTKYDPKFQVRATITRDTSTSTAYLELSSLRSEDTAVYYCSRS |
| | | GGLPDVWGQGTTVTVSS |
| 1206 | AS12825 VH | QVQLVQSGAEVKKPGASVKVSCKASGFDICDTYMHWVKQAPGQGLEW |
| | | MGRIDPASGHTKYDPKFQVRATMTTDTSTSTAYLELSSLRSEDTAVYYC |
| | | SRSGGLPDVWGQGTTVTVSS |
| 1207 | 12835 VH | QVQLVQSGAEVKKPGASVKLSCKASGFDIQDTYMHWVRQAPGQGLEW |
| | | MGRIDPASGHTKYDPKFQVRVTMTTDTSTSTVYMELSSLRSEDTAVYYC |
| | | SRSGGLPDVWGQGTTVTVSS |
| 1208 | 18-7 VH | QVQLVQSGAEVKKPGASVKLSCKASGFDIQDTYMHWVRQAPGQGLEW |
| | | MGRIDPASGHTKYDPKFQVRVTMTRDTSTSTVYMELSSLRSEDTAVYYC |
| | | SRSGGLPDVWGQGTTVTVSS |
| 1209 | 21-3 VH, L8 VH | QVQLVQSGAEVKKPGASVKVSCKASGFDIQDTYMHWVRQAPGQGLEW |
| | | MGRIDPASGHTKYDPKFQVRVTMTRDTSTSTVYMELSSLRSEDTAVYYC |
| | | ARSGGLPDVWGQGTTVTVSS |
| 1210 | 21-3 V102K VH | QVQLVQSGAEVKKPGASVKVSCKASGFDIQDTYMHWVRQAPGQGLEW |
| | | MGRIDPASGHTKYDPKFQVRVTMTRDTSTSTVYMELSSLRSEDTAVYYC |
| | | ARSGGLPDKWGQGTTVTVSS |
| 1211 | 21-3 V102M VH | QVQLVQSGAEVKKPGASVKVSCKASGFDIQDTYMHWVRQAPGQGLEW |
| | | MGRIDPASGHTKYDPKFQVRVTMTRDTSTSTVYMELSSLRSEDTAVYYC |
| | | ARSGGLPDMWGQGTTVTVSS |
| 1212 | 21-3 V102Q VH | QVQLVQSGAEVKKPGASVKVSCKASGFDIQDTYMHWVRQAPGQGLEW |
| | | MGRIDPASGHTKYDPKFQVRVTMTRDTSTSTVYMELSSLRSEDTAVYYC |
| | | ARSGGLPDQWGQGTTVTVSS |
| 1213 | 21-3 V102W VH | QVQLVQSGAEVKKPGASVKVSCKASGFDIQDTYMHWVRQAPGQGLEW |
| | | MGRIDPASGHTKYDPKFQVRVTMTRDTSTSTVYMELSSLRSEDTAVYYC |
| | | ARSGGLPDWWGQGTTVTVSS |
| 1214 | 21-3 CDRv VH | QVQLVQSGAEVKKPGASVKVSCKASGFX₁X₂X₃DTX₄X₅HWVRQAPGQGL |
| | | EWMGRIDPASGHTKYDPKFQVRVTMTRDTSTSTVYMELSSLRSEDTAV |
| | | YYCARSGGX₆PDX₇WGQGTTVTVSS |
| | | X₁ = D or E |
| | | X₂ = I, P, or V |
| | | X₃ = G, Q, S, or V |
| | | X₄ = F or Y |
| | | X₅ = I or M |
| | | X₆ = L or M |
| | | X₇ = E, I, K, L, M, Q T, V, W, or Y |
| 1215 | 21-3 CDRv VH | QVQLVQSGAEVKKPGASVKVSCKASGFX₁X₂X₃DTX₄X₅HWVRQAPGQGL |
| | | EWMGRIDPASGHTKYDPKFQVRVTMTRDTSTSTVYMELSSLRSEDTAV |
| | | YYCSRSGGX₆PDX₇WGQGTTVTVSS |
| | | X₁ = D or E |
| | | X₂ = I, P, or V |
| | | X₃ = G, Q, S, or V |
| | | X₄ = F or Y |
| | | X₅ = I or M |
| | | X₆ = L or M |
| | | X₇ = E, I, K, L, M, Q, T, V, W, or Y |
| 1216 | Clone 2 VH, clone 52 | QVQLVQSGAEVKKPGASVKVSCKASGFDIQDTYMEIWVRQAPG |
| | VH | QGLEWMGRIEPASGHIKYSPKFQGRVTMTRDTSTSTVYMELSSL |
| | | RSEDTAVYYCARSGGLPDWWGQGTTVTVSS |
| 1217 | Clone 46 VH | QVQLVQSGAEVKKPGASVKVSCKASGFDIQDTYMEIWVRQAPG |
| | | QGLEWMGRIEPASGHVKYSPKFQVRVTMTRDTSTSTVYMELSSL |
| | | RSEDTAVYYCARSGGLPDWWGQGTTVTVSS |
| 1218 | Clone 47 VH | QVQLVQSGAEVKKPGASVKVSCKASGFDIQDTYMEIWVRQAPG |
| | | QGLEWMGRIEPASGHVKYDPKFQTRVTMTRDTSTSTVYMELSSL |
| | | RSEDTAVYYCARSGGLPDWWGQGTTVTVSS |
| 1219 | Clone 14 VH | QVQLVQSGAEVKKPGASVKVSCKASGFDIQDTYMEIWVRQAPG |
| | | QGLEWMGRIDPASGHiKYDPKFQkRVTMTRDTSTSTVYMELSSL |
| | | RSEDTAVYYCARSGGLPDMWGQGTTVTVSS |
| 1220 | Clone 16L VH | QVQLVQSGAEVKKPGASVKVSCKASGFDIQDTYMEIWVRQAPG |
| | | QGLEWMGRIDPASGHvKiDPKFQVRVTMTRDTSTSTVYMELSSL |
| | | RSEDTAVYYCARSGGLPDMWGQGTTVTVSS |
| 1221 | Clone 17L VH | QVQLVQSGAEVKKPGASVKVSCKASGFDIQDTYMEIWVRQAPG |
| | | QGLEWMGRIDPASGHLKYDPKFQVRVTMTRDTSTSTVYMELSS |
| | | LRSEDTAVYYCARSGGLPDMWGQGTTVTVSS |
| 1222 | Clone 17L-1 VH | QVQLVQSGAEVKKPGASVKVSCKASGFDIQDTYMEIWVRQAPG |
| | | QGLEWMGRIDPASGHLKYDPKFQRRVTMTRDTSTSTVYMELSSL |
| | | RSEDTAVYYCARSGGLPDMWGQGTTVTVSS |
| 1223 | Clone 23 VH, clone | QVQLVQSGAEVKKPGASVKVSCKASGFDIQDTYMEIWVRQAPG |
| | A1 VH | QGLEWMGRIDPASGHLKYDPKFQNRVTMTRDTSTSTVYMELSS |
| | | LRSEDTAVYYCARSGGLPDKWGQGTTVTVSS |
| 1224 | Clone 53 VH, clone | QVQLVQSGAEVKKPGASVKVSCKASGFDIQDTYMEIWVRQAPG |
| | E1 VH | QGLEWMGRIEPASGHLKYDPKFQERVTMTRDTSTSTVYMELSSL |
| | | RSEDTAVYYCARSGGLPDKWGQGTTVTVSS |
| 1225 | Clone 3-17L V-A VH | QVQLVQSGAEVKKPGASVKVSCKASGFDIQDTYMEIWVRQAPG |
| | | QGLEWMGRIDPASGHLKYDPKFQGRVTITRDTSASTAYMELSSL |
| | | RSEDTAVYYCARSGGLPDMWGQGTTVTVSS |
| 1226 | Clone 3-17L VH | QVQLVQSGAEVKKPGASVKVSCKASGFDIQDTYMEIWVRQAPG |
| | | QGLEWMGRIDPASGHLKYDPKFQGRVTITRDTSASTVYMELSSL |
| | | RSEDTAVYYCARSGGLPDMWGQGTTVTVSS |
| 1227 | Clone L8mod VH | QVQLVQSGAEVKKPGASVKVSCKASGFDIQDTYMEIWVRQAPG |
| | | QGLEWMGRIDPASGHTKYDPKFQGRATITTDTSASTAYLQLSSL |
| | | RSEDTAVYYCARSGGLPDVWGQGTTVTVSS |
| 1228 | Clone X-V VH | QVQLVQSGAEVKKPGASVKVSCKASGFDIQDTYMEIWVRQAPG |
| | | QGLEWMGRIDPASGHTKYDPKFQVRATITTDTSASTAYLQLSSL |
| | | RSEDTAVYYCARSGGLPDFWGQGTTVTVSS |
| 1229 | Clone X VH, clone | QVQLVQSGAEVKKPGASVKVSCKASGFDIQDTYMEIWVRQAPG |
| | XL3-6 VH, clone | QGLEWMGRIDPASGHTKYDPKFQGRATITTDTSASTAYLQLSSL |
| | XL3-10 VH, clone | RSEDTAVYYCARSGGLPDFWGQGTTVTVSS |
| | XL3-15 VH, clone | |
| | L3-13 VH | |
| 1230 | Clone H3-1 VH | QVQLVQSGAEVKKPGASVKVSCKASGFDIQDTYMEIWVRQAPG |
| | | QGLEWMGRIDPASGHTKYDPKFQGRATITTDTSASTAYLQLSSL |
| | | RSEDTAVYYCARSGGLPDLWGQGTTVTVSS |
| 1231 | Clone H2-2 VH | QVQLVQSGAEVKKPGASVKVSCKASGFDIQDTYMEIWVRQAPG |
| | | QGLEWMGRIDPASGHSKYDPKFQVRATITTDTSASTAYLQLSSLR |
| | | SEDTAVYYCARSGGLPDFWGQGTTVTVSS |
| 1232 | Clone H2-5 VH | QVQLVQSGAEVKKPGASVKVSCKASGFDIQDTYMEIWVRQAPG |
| | | QGLEWMGRIDPASGHYKYDPKFQVRATITTDTSASTAYLQLSSL |
| | | RSEDTAVYYCARSGGLPDFWGQGTTVTVSS |
| 1233 | M1 VH | EVQLVESGGGLVQPGGSLRLSCAVSGFTFSSYWMSWVRQAPGKGLEWV |
| | | ANIKEDGSEKNYVDSVKGRFTLSSDNAKNSLYLQMNSLRAEDTAVYYCA |
| | | REDYDSYYKYGMDVWGQGTAVIVSS |
| 1234 | M2 VH | QVQLQQWGAGLLKPSETLSLTCAVYGGSFTGFYWSWIRQPPGKGLEWI |
| | | GEINHRGNTNYNPSLKSRVTMSVDTSKNQFSLNMISVTAADTAMYFCAS |
| | | PFYDFWSGSDYWGQGTLVTVSS |
| 1235 | M3 VH | QVQLVQSGAEVKKPGASVKVSCKASGYDFTYYGISWVRQAPGQGLEWM |
| | | GWISTYNGNTHYARMLQGRVTMTTDTSTRTAYMELRSLRSDDTAVYYC |
| | | ARENYYGSGAYRGGMDVWGQGTTVTVSS |
| 1236 | M3 VH + constant | QVQLVQSGAEVKKPGASVKVSCKASGYDFTYYGISWVRQAPGQGLEWM |
| | | GWISTYNGNTHYARMLQGRVTMTTDTSTRTAYMELRSLRSDDTAVYYC |
| | | ARENYYGSGAYRGGMDVWGQGTTVTVSSASTKGPSVFPLAPSSKSTSGG |
| | | TAALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPEAAGA |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHE |
| | | ALHNHYTQKSLSLSPG |
| 1237 | M4 VH | QLQLQESGPGLVKPSETLSLTCTVSGGSISSRSYYWGWIRQPPGKGLEWI |
| | | GSIYYNGRTYYNPSLKSRVTISVDTSKNQFSLKLSSVTAADTAVYYCARE |
| | | DYGDYGAFDIWGQGTMVTVSS |
| 1238 | M5 VH | QVTLKESGPALVKPTQTLTLTCTFSGFSLSTSNMGVVWIRQPPGKALEW |
| | | LAHILWDDREYSNPALKSRLTISKDTSKNQVVLTMTNMDPVDTATYYC |
| | | ARMSRNYYGSSYVMDYWGQGTLVTVSS |
| 1239 | M6 VH | QVQLVQSGSELKKPGASVKVSCKASGYTFTLYGMNWVRQAPGQGLEW |
| | | MGWINTYTGEPTYADDFKGRFVFSLDTSVSTAYLQISSLKAEDTAVYYC |
| | | ARDTAMDYAMAYWGQGTLVTVSS |
| 1240 | M6 VH | QVQLVQSGSELKKPGASVKVSCKASGYTFTLYGMNWVKQAPGKGLKW |
| | | MGWINTYTGEPTYADDFKGRFVFSLDTSVSTAYLQISSLKAEDTAVYFCA |
| | | RDTAMDYAMAYWGQGTLVTVSS |
| 1241 | M6 VH | QVQLVQSGSELKKPGASVKVSCKASGYTFTNYGMNWVRQAPGQGLEW |
| | | MGWINTYTGEPTYADDFKGRFVFSLDTSVSTAYLQISSLKAEDTAVYYC |
| | | ARDYGKYGDYYAMDYWGQGTLVTVSS |
| 1242 | M6 VH | QVQLVQSGSELKKPGASVKVSCKASGYTFTNYGMNWVRQAPGKGLKW |
| | | MGWINTYTGEPTYADDFKGRFVFSLDTSVSTAYLQISSLKAEDTAVYFCA |
| | | RDYGKYGDYYAMDYWGQGTLVTVSS |
| 1243 | M7 VH | QVQLQQPGSVLVRPGASVKVSCKASGYTFTSSWMHWAKQRPGQGLEWI |
| | | GEIHPNSGGTNYNEKFKGKATVDTSSSTAYVDLSSLTSEDSAVYYCARGD |
| | | YYGYVSWFAYWGQGTLVTVSS |
| 1244 | M7 VH | QVQLVQSGAEVKKPGASVKVSCKASGYTFTSSWMHWARQAPGQGLEW |
| | | IGEIHPNSGGTNYAQKFQGRATLTVDTSSSTAYMELSRLRSDDTAVYYCA |
| | | RGDYYGYVSWFAYWGQGTLVTVSS |
| 1245 | M7 VH | QVQLVQSGAEVKKPGASVKVSCKASGYTFTSSWMHWARQAPGQGLEW |
| | | IGEIHPNSGGTNYAQKFQGRATMTVDTSISTAYMELSRLRSDDTAVYYC |
| | | ARGDYYGYVSWFAYWGQGTLVTVSS |
| 1246 | M7 VH | QVQLVQSGAEVKKPGASVKVSCKASGYTFTSSWMHWARQAPGQGLEW |
| | | IGEIHPNSGGTNYAQKFQGRVTMTVDTSISTAYMELSRLRSDDTAVYYC |
| | | ARGDYYGYVSWFAYWGQGTLVTVSS |
| 1247 | M7 VH | QVQLVQSGAEVKKPGASVKVSCKASGYTFTSSWMHWARQAPGQGLEW |
| | | MGEIHPNSGGTNYAQKFQGRVTMTVDTSISTAYMELSRLRSDDTAVYYC |
| | | ARGDYYGYVSWFAYWGQGTLVTVSS |
| 1248 | M8 VH | QVQLVQSGAEVKKPGASVKVSCKASGYTFTSYDINWVRQAPGQGLEWM |
| | | GWLNPNSGNTGYAQKFQGRVTMTADRSTSTAYMELSSLRSEDTAVYYC |
| | | AREVPETAAFEYWGQGTLVTVSS |
| 1249 | M8 VH | QVQLVQSGAEVKKPGASVKVSCKASGYTFTSYDINWVRQAPGQGLEWM |
| | | GWLNPNSGNTGYAQKFQGRVTMTADRSTSTAYMELSSLRSEDTAVYYC |
| | | AREVPETAAFEYWGQGTLVTVSS |
| 1250 | M8 VH | QVQLVQSGAEVKKPGASVKVSCKASGYTFTSYDINWVRQAPGQGLEWM |
| | | GWLNPNSGYTGYAQKFQGRVTMTADRSTSTAYMELSSLRSEDTAVYYC |
| | | AREVPETAAFEYWGQGTLVTVSS |
| 1251 | M8 VH | QVQLVQSGAEVKKPGASVKVSCKASGYTFTSYDINWVRQAPGQGLEWM |
| | | GWLNPNSGNTGYAQKFQGRVTMTADRSTSTAYMELSSLRSEDTAVYYC |
| | | AREVPETAAFEYWGQGTLVTVSS |
| 1252 | M8 VH | QVQLVQSGAEVKKPGASVKVSCKASGYTFTSYDINWVRQAPGQGLEWM |
| | | GWLNPNSGNTGYAQKFQGRVTMTADRSTSTAYMELSSLRSEDTAVYYC |
| | | AREVPETAAFEYWGQGTLVTVSS |
| 1253 | M8 VH | QVQLVQSGAEVKKPGASVKVSCKASGYTFTSYDINWVRQAPGQGLEWM |
| | | GWLNPNSGNTGYAQKFQGRVTMTADRSTSTAYMELSSLRSEDTAVYYC |
| | | AREVPETAAFEYWGQGTLVTVSS |
| 1254 | M8 VH | QVQLVQSGAEVKKPGASVKVSCKASGYTFTSYDINWVRQAPGQGLEWM |
| | | GWLNPNSGNTGYAQKFQGRVTMTADRSTSTAYMELSSLRSEDTAVYYC |
| | | AREVPETAAFEYWGQGTLVTVSS |
| 1255 | M8 VH | QVQLVQSGAEVKKPGASVKVSCKASGYTFTSYDINWVRQAPGQGLEWM |
| | | GWLNPNSGYTGYAQKFQGRVTMTADRSTSTAYMELSSLRSEDTAVYYC |
| | | AREVPETAAFEYWGQGTLVTVSS |
| 1256 | M8 VH | QVQLVQSGAEVKKPGASVKVSCKASGYTFTSYDINWVRQAPGQGLEWM |
| | | GWLNPNSGYTGYAQKFQGRVTMTADRSTSTAYMELSSLRSEDTAVYYC |
| | | AREVPETAAFEYWGQGTLVTVSS |
| 1257 | M8 VH | QVQLVQSGAEVKKPGASVKVSCKASGYTFTSYDINWVRQAPGQGLEWM |
| | | GWLNPNSGYTGYAQKFQGRVTMTADRSTSTAYMELSSLRSEDTAVYYC |
| | | AREVPETAAFEYWGQGTLVTVSS |
| 1258 | M8 VH | QVQLVQSGAEVKKPGASVKVSCKASGYTFTSYDINWVRQAPGQGLEWM |
| | | GWLNPNSGYTGYAQKFQGRVTMTADRSTSTAYMELSSLRSEDTAVYYC |
| | | AREVPETAAFEYWGQGTLVTVSS |
| 1259 | M8 VH | QVQLVQSGAEVKKPGASVKVSCKASGYTFTSYDINWVRQAPGQGLEWM |
| | | GWLNPNSGYTGYAQKFQGRVTMTADRSTSTAYMELSSLRSEDTAVYYC |
| | | AREVPETAAFEYWGQGTLVTVSS |
| 1260 | M9 VH | QVQLQESGPGLVKPSETLSLTCTVSGGSISSYFWSWIRQPPGKGLEWIGYI |
| | | YYSGNTKYNPSLKSRVTISIDTSKNQFSLKLSSVTAADTAVYYCARETGSY |
| | | YGFDYWGQGTLVTVSS |
| 1261 | M10 VH | QVQLQQWGAGLLKPSETLSLTCAVHGGSFSGYYWNWIRQPPGKGLEWI |
| | | GEINHAGNTNYNPSLKSRVTISLDTSKNQFSLTLTSVTAADTAVYYCARG |
| | | YCRSTTCYFDYWGQGTLVTVSS |
| 1262 | M11 VH | EVQLLESGGGLVQPGKSLRLSCAVSGFTESTYGMNWVRQAPGK |
| | | GLEWVSSISGTGRTTYHADSVQGRFTVSRDNSKNILYLQMNSLR |
| | | ADDTAVYFCTKERGDYYYGVFDYWGQGTLVTVSS |
| 1263 | M12 VH | QIQLVQSGPELKKPGETVKISCKASGYTFTTYGMSWVKQAPGKG |
| | | LKWMGWMNTYSGVTTYADDFKGRFAFSLETSASTAYMQIDNL |
| | | KNEDTATYFCAREGYVFDDYYATDYWGQGTSVTVSS |
| 301 | Variable Heavy 1 | X1VQLVQSGAEVKKPGASVKVSCKAS[HCDR1]WVX2QX3PGQG |
| | | LEWX4G[HCDR2]RX5TX6TX7DTSTSTX8YX9ELSSLRSEDTAVY |
| | | YCAR[HCDR3]WGQGTTVTVSS |
| | | wherein each of X1-X11 is independently selected |
| | | from A, R, N, D, C, Q, E, G, H, I, L, K, M, F, P, |
| | | S, T, W, Y, or V |
| | | In some cases, X1 = Q or E |
| | | In some cases, X2 = R or K |
| | | In some cases, X3 = A or R |
| | | In some cases, X4 = M or I |
| | | In some cases, XS = V or A |
| | | In some cases, X6 = M or I |
| | | In some cases, X7 = R or T |
| | | In some cases, X8 = V or A |
| | | In some cases, X9 = M or L |
| 302 | Variable Heavy 2 | X1VQLVQSGAEVKKPGASVKVSCKAS[HCDR1]WVX2QX3PGQG |
| | | LEWX4G[HCDR2]RX5TX6TX7DTSTSTX8YX9ELSSLRSEDTAVY |
| | | YC[HCDR3]WGQGTTVTVSS |
| | | wherein each of X1-X11 is independently selected |
| | | from A, R, N, D, C, Q, E, G, H, I, L, K, M, F, P, |
| | | S, T, W, Y, or V |
| | | In some cases, X1 = Q or E |
| | | In some cases, X2 = R or K |
| | | In some cases, X3 = A or R |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |
| 1301 | Variable Heavy 3 | X1VQLVQSGAEVKKPGASVKVSCKAS[HCDR1]WVX2QRPGQGLEWX4G |
| | | [HCDR2]RX5TX6TX7DTSTSTX8YX9ELSSLRSEDTAVYYCAR[HCDR3] |
| | | WGQGTTVTVSS |
| | | wherein each of X1, X2, X4, XS, X6, X7, X8, X9, X10, |
| | | and X11 is independently selected from A, R, N, D, C, |
| | | Q, E, G, H, I, L, K, M, F, P, S, T, W, Y, or V |
| | | In some cases, X1 = Q or E |
| | | In some cases, X2 = R or K |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |
| 1302 | Variable Heavy 4 | X1VQLVQSGAEVKKPGASVKVSCKAS[HCDR1]WVX2QRPGQGLEWX4G |
| | | [HCDR2]RX5TX6TX7DTSTSTX8YX9ELSSLRSEDTAVYYC[HCDR3]WG |
| | | QGTTVTVSS |
| | | wherein each of X1, X2, X4, XS, X6, X7, X8, X9, X10, |
| | | and X11 is independently selected from A, R, N, D, C, |
| | | Q, E, G, H, I, L, K, M, F, P, S, T, W, Y, or V |
| | | In some cases, X1 = Q or E |
| | | In some cases, X2 = R or K |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |

TABLE 2E
Light chain variable region (VL) amino acid sequences

| SEQ | | |
|---|---|---|
| ID NO | Description | Sequence |
| 201 | 217 VL, | EIVLTQSPGTLSLSPGERATLSCRASSSVSYMYWYQQKPGQAPRPLIYA |
| | 219 VL, 221 VL, | TSNLASGIPDRFSGSGSGTDFTLTISRLEPEDFAVYYCQQWEGNPRTFG |
| | 200 VL, 213 VL, | GGTKLEIK |
| | 212 VL, 211 VL, | |
| | 199 VL, 214 VL, | |
| | 216 VL, 222 VL, | |
| | 203 VL, 147 VL, | |
| | 218 VL, 179 VL, | |
| | 148 VL, 149 VL, | |
| | 151 VL, 180 VL, 175 | |
| | VL, 178 VL, 145 VL, | |
| | 146 VL, 150 VL, 152 | |
| | VL, 176 VL, 177 VL, | |
| | 201 VL, 202 VL, 204 | |
| | VL, 215 VL, 224 VL | |
| 202 | 223 VL, 107 VL, | EIVLTQSPGTLSLSPGERATLSCRASSSVSYMYWYQQKPGQAPRLLIYA |
| | 205 VL, 181 VL, | TSNLASGIPDRFSGSGSGTDFTLTISRLEPEDFAVYYCQQWEGNPRTFG |
| | 188 VL, 64 VL, | GGTKLEIK |
| | 67 VL, 68 VL, | |
| | 94 VL, 33 VL, 57 | |
| | VL, 58 VL, 59 VL, | |
| | 60 VL, 61 VL, 62 | |
| | VL, 63 VL, 65 VL, | |
| | 66 VL, 69 VL, 70 | |
| | VL, 71 VL, 72 VL, | |
| | 76 VL, 77 VL, 78 | |
| | VL, 91 VL, 92 VL, | |
| | 93 VL, 97 VL, 98 | |
| | VL, 99 VL, 140 VL, | |
| | 142 VL, 143 VL, 182 | |
| | VL, 183 VL, 184 VL, | |
| | 185 VL, 186 VL, 187 | |
| | VL, 189 VL, 190 VL, | |
| | 191 VL, 192 VL, 206 | |
| | VL, 207 VL, 208 VL, | |
| | 209 VL, 210 VL, 18- | |
| | 7 S93E VL, clone 34 | |
| | VL, clone 2 VL, | |
| | clone 46 VL, clone | |
| | 47 VL, clone 23 VL, | |
| | clone 53 | |
| 203 | 15 VL, 18-7, L8, | EIVLTQSPGTLSLSPGERATLSCRASSSVSYMYWYQQKPGQAPRLLIYAT |
| | clone L8mod VL, | SNLASGIPDRFSGSGSGTDFTLTISRLEPEDFAVYYCQQWSGNPRTFGGG |
| | clone X-V VL, clone | TKLEIK |
| | X VL, clone H3-1 | |
| | VL, clone H2-2 VL, | |
| | clone H2-5 VL | |
| 204 | 30 VL, 100 VL, | EIVLTQSPGTLSLSPGERATLSCRASSSVSYMYWYQQKPGQAPRLLIYAT |
| | 129 VL, 122 VL, | SNLASGIPDRFSGSGSGTDFTLTISRLEPEDFAVYYCQQWKGNPRTFGGG |
| | 127 VL, 126 VL, | TKLEIK |
| | 160 VL, 157 VL, | |
| | 159 VL, 158 VL, | |
| | 125 VL, 103 VL, 101 | |
| | VL, 102 VL, 104 VL, | |
| | 105 VL, 121 VL, 123 | |
| | VL, 124 VL, 128 VL, | |
| | 144 VL, 161 VL, 162 | |
| | VL, 163 VL, 164 VL, | |
| | clone L3-13 VL | |
| 205 | 110 VL, 197 VL, | EIVLTQSPGTLSLSPGERATLSCRASSSVSYMYWYQQKPGQAPRP |
| | 112 VL, 169 VL, | WIYATSNLASGIPDRFSGSGSGTDFTLTISRLEPEDFAVYYCQQW |
| | 173 VL, 115 VL, | EGNPRTFGGGTKLEIK |
| | 113 VL, 96 VL, | |
| | 196 VL, 172 VL, | |
| | 75 VL, 174 VL, | |
| | 109 VL, 198 VL, | |
| | 170 VL, 29 VL, 31 | |
| | VL, 32 VL, 73 VL, | |
| | 74 VL, 95 VL, 108 | |
| | VL, 111 VL, 114 VL, | |
| | 116 VL, 117 VL, 118 | |
| | VL, 119 VL, 120 VL, | |
| | 130 VL, 153 VL, 154 | |
| | VL, 155 VL, 156 VL, | |
| | 165 VL, 166 VL, 167 | |
| | VL, 168 VL, 171 VL, | |
| | 193 VL, 194 VL, 195 | |
| | VL, 220 VL | |
| 206 | 134 VL, 132 VL, 133 | EIVLTQSPGTLSLSPGERATLSCRASSSVSYMYWYQQKPGQAPRP |
| | VL, 135 VL, 136 VL | LIYATSNLASGIPDRFSGSGSGTDFTLTISRLEPEDFAVYYCQQWK |
| | | GNPRTFGGGTKLEIK |
| 207 | 138 VL, 137 VL, 139 | EIVLTQSPGTLSLSPGERATLSCRASSSVSYMYWYQQKPGQAPRL |
| | VL, 141 VL, clone | LIYATSNLASGIPDRFSGSGSGTDFTLTISRLEPEDFAVYYCQQWS |
| | XL3-15 | VL RNPRTFGGGTKLEIK |
| 208 | 34 VL, 35 VL, 36 | EIVLTQSPGTLSASPGERATMSCRASSSVSYMYWYQQKPGQAPR |
| | VL, 37 VL, 38 VL, | PWIYATSNLASGVPDRFSGSGSGTDYTLTISRVEPEDFAVYYCQQ |
| | 39 VL, 40 VL, 41 | WSGNPRTFGGGTKLEIK |
| | VL, 42 VL, 43 VL, | |
| | 44 VL, 45 VL, 46 | |
| | VL, 47 VL, 53 VL, | |
| | 54 VL, 55 VL, 79 | |
| | VL, 81 VL, 82 VL, | |
| | 83 VL, AS12824 VL | |
| 209 | 85 VL | EIVLTQSPGTLSLSPGERATLSCRASSSVSYMYWYQQKPGQAPRL |
| | | LIYATSNLASGVPDRFSGSGSGTDFTLTISRLEPEDFAVYYCQQW |
| | | EGNPRTFGGGTKLEIK |
| 210 | 48 VL | EIVLTQSPGTLSASPGERATLSCRASSSVSYMYWYQQKPGQAPRP |
| | | WIYATSNLASGVPDRFSGSGSGTDYTLTISRVEPEDFAVYYCQQ |
| | | WSGNPRTFGGGTKLEIK |
| 211 | 49 VL | EIVLTQSPGTLSASPGERATMSCRASSSVSYMYWYQQKPGQAPR |
| | | LLIYATSNLASGVPDRFSGSGSGTDYTLTISRVEPEDFAVYYCQQ |
| | | WSGNPRTFGGGTKLEIK |
| 212 | 50 VL | EIVLTQSPGTLSASPGERATMSCRASSSVSYMYWYQQKPGQAPR |
| | | PWIYATSNLASGVPDRFSGSGSGTDFTLTISRVEPEDFAVYYCQQ |
| | | WSGNPRTFGGGTKLEIK |
| 213 | 51 VL | EIVLTQSPGTLSASPGERATMSCRASSSVSYMYWYQQKPGQAPR |
| | | PWIYATSNLASGVPDRFSGSGSGTDYTLTISRLEPEDFAVYYCQQ |
| | | WSGNPRTFGGGTKLEIK |
| 214 | 52 VL | EIVLTQSPGTLSASPGERATMSCRASSSVSYMYWYQQKPGQAPR |
| | | PWIYATSNLASGVPDRFSGSGSGTDFTLTISRLEPEDFAVYYCQQ |
| | | WSGNPRTFGGGTKLEIK |
| 215 | 56 VL | EIVLTQSPGTLSASPGERATMSCRASSSVSYMYWYQQKPGQAPR |
| | | PWIYATSNLASGIPDRFSGSGSGTDYTLTISRVEPEDFAVYYCQQ |
| | | WSGNPRTFGGGTKLEIK |
| 216 | 86 VL | EIVLTQSPGTLSLSPGERATLSCRASSSVSYMYWYQQKPGQAPRL |
| | | LIYATSNLASGIPDRFSGSGSGTDYTLTISRLEPEDFAVYYCQQWE |
| | | GNPRTFGGGTKLEIK |
| 217 | 87 VL | EIVLTQSPGTLSLSPGERATLSCRASSSVSYMYWYQQKPGQAPRL |
| | | LIYATSNLASGIPDRFSGSGSGTDFTLTISRVEPEDFAVYYCQQWE |
| | | GNPRTFGGGTKLEIK |
| 218 | 88 VL | EIVLTQSPGTLSLSPGERATLSCRASSSVSYMYWYQQKPGQAPRL |
| | | LIYATSNLASGIPDRFSGSGSGTDYTLTISRVEPEDFAVYYCQQWE |
| | | GNPRTFGGGTKLEIK |
| 219 | 89 VL | EIVLTQSPGTLSASPGERATLSCRASSSVSYMYWYQQKPGQAPRL |
| | | LIYATSNLASGIPDRFSGSGSGTDFTLTISRLEPEDFAVYYCQQWE |
| | | GNPRTFGGGTKLEIK |
| 220 | 90 VL | EIVLTQSPGTMSLSPGERATLSCRASSSVSYMYWYQQKPGQAPR |
| | | LLIYATSNLASGIPDRFSGSGSGTDFTLTISRLEPEDFAVYYCQQW |
| | | EGNPRTFGGGTKLEIK |
| 1264 | 5C3D11 VL | QIVLSQSPAILSASPGEKVTMTCRASSSVSYMYWYQQKPGSSPKP |
| | | WIYATSNLASGVPDRFSGSGSGTSYSLTISRVEAEDAATYYCQQ |
| | | WSGNPRTFGGGTKLEIK |
| 1265 | 9E12E5 VL | MKLPVRLLVLMFWIPASSSDVLMTQTPLSLPVSLGDQASISCRSS |
| | | QTIVHSNGDTYLDWFLQKPGQSPKLLIYKVSNRFSGVPDRFSGSG |
| | | SGTDFTLKISRVEAEDLGVYYCFQGSHVPYTFGGGTKLEIK |
| 1266 | AS12823 VL | EIVLTQSPGTLSLSPGERATMSCRASSSVSYMYWYQQKPGQAPR |
| | | PWIYATSNLASGIPDRFSGSGSGTDFTLTISRVEPEDFAVYYCQQ |
| | | WSGNPRTFGGGTKLEIK |
| 1267 | AS12819 VL | EIVLTQSPGTLSLSPGERVTMSCRASSSVSYMYWYQQKPGQAPR |
| | | PWIYATSNLASGVPDRFSGSGSGTDFTLTISRVEPEDFAVYYCQQ |
| | | WSGNPRTFGGGTKVEIK |
| 1268 | AS12816 VL | EIVLTQSPGTLSASPGERVTLSCRASSSVSYMYWYQQKPGQAPRP |
| | | WIYATSNLASGVPDRFSGSGSGTDFTLTISRLEPEDFAVYYCQQW |
| | | SGNPRTFGGGTKLEIK |
| 1269 | AS12825 VL | EIVLTQSPGTLSASPGERVTMSCRASSSVSYMYWYQQKPGQAPR |
| | | LLIYATSNLASGVPDRFSGSGSGTDFTLTISRVEPEDFAVYYCQQ |
| | | WSGNPRTFGGGTKLEIK |
| 1270 | 12835 VL | EIVLTQSPGTLSLSPGERVTMSCRASSSVSYMYWYQQKPGQAPR |
| | | PWIYATSNLASGVPDRFSGSGSGTDYTLTISRLEPEDFAVYYCQQ |
| | | WSGNPRTFGGGTKLEIK |
| 1271 | 21-3 VL | EIVLTQSPGTLSLSPGERATLSCRASSSVSYMYWYQQKPGQAPRL |
| | | LIYATSNLASGVPDRFSGSGSGTDYTLTISRLEPEDFAVYYCQQW |
| | | SGNPRTFGGGTKLEIK |
| 1272 | 18-7 592D VL | EIVLTQSPGTLSLSPGERATLSCRASSSVSYMYWYQQKPGQAPRL |
| | | LIYATSNLASGIPDRFSGSGSGTDFTLTISRLEPEDFAVYYCQQWD |
| | | GNPRTFGGGTKLEIK |
| 1273 | 18-7 592H VL | EIVLTQSPGTLSLSPGERATLSCRASSSVSYMYWYQQKPGQAPRL |
| | | LIYATSNLASGIPDRFSGSGSGTDFTLTISRLEPEDFAVYYCQQWH |
| | | GNPRTFGGGTKLEIK |
| 1274 | 18-7 S92N VL | EIVLTQSPGTLSLSPGERATLSCRASSSVSYMYWYQQKPGQAPRL |
| | | LIYATSNLASGIPDRFSGSGSGTDFTLTISRLEPEDFAVYYCQQWN |
| | | GNPRTFGGGTKLEIK |
| 1275 | 18-7 S92Q VL, clone | EIVLTQSPGTLSLSPGERATLSCRASSSVSYMYWYQQKPGQAPRL |
| | 14 VL, clone 16L | LIYATSNLASGIPDRFSGSGSGTDFTLTISRLEPEDFAVYYCQQWQ |
| | VL, clone 17L VL, | GNPRTFGGGTKLEIK |
| | clone 17L-1 VL, | |
| | clone 3-17L V-A VL, | |
| | clone 3-17L VL | |
| 1276 | 18-7 CDRv VL | EIVLTQSPGTLSLSPGERATLSCRASSSVSYMYWYQQKPGQAPRLLIYATS |
| | | NLASGIPDRFSGSGSGTDFTLTISRLEPEDFAVYYCX₁QWX₂X₃X₄PRTFGG |
| | | GTKLEIK |
| | | X₁ = Q or N |
| | | X₂ = D, E, H, N, Q, or S |
| | | X₃ = A or G |
| | | X₄ = D, F, K, N, R, S, or T |
| 1277 | Clone 52 VL, clone | EIVLTQSPGTLSLSPGERATLSCGASSSVSYMYWYQQKPGQAPRL |
| | A1 VL, clone E1 VL | LIYATSNLASGIPDRFSGSGSGTDFTLTISRLEPEDFAVYYCQQWE |
| | | GNPRTFGGGTKLEIK |
| 1278 | Clone XL3-6 VL | EIVLTQSPGTLSLSPGERATLSCRASSSVSYMYWYQQKPGQAPRL |
| | | LIYATSNLASGIPDRFSGSGSGTDFTLTISRLEPEDFAVYYCSQWS |
| | | GNPRTFGGGTKLEIK |
| 1279 | Clone XL3-10 VL | EIVLTQSPGTLSLSPGERATLSCRASSSVSYMYWYQQKPGQAPRL |
| | | LIYATSNLASGIPDRFSGSGSGTDFTLTISRLEPEDFAVYYCQQWS |
| | | GNPRSFGGGTKLEIK |
| 1280 | M1 VL | DIVMTQSPDSLAVSLGERATINCKSSQSILYSSNNKNYLAWYQQK |
| | | PGQPPKLLIYWASTRESGVPDRFSGSGSGTDFTLTISSLQAEDVS |
| | | VYYCQQYYSTPFTFGPGTKVDIK |
| 1281 | M2 VL | DIMLTQTPLTSPVTLGQPASISCKSSQSLVHSDGNTYLSWLQQRP |
| | | GQPPRLLFYKISNRFSGVPDRFSGSGAGTDFTLKISRVEAEDVGV |
| | | YYCMQATQFPLTFGGGTKVEIK |
| 1282 | M3 VL | EIVLTQSPATLSLSPGERATLSCRASQSVSSYLAWYQQKPGQAPR |
| | | LLIYDASNRATGIPARFSGSGSGTDFTLTISSLEPEDFAVYYCQQR |
| | | SNWPWTFGQGTKVEIK |
| 1283 | M3 VL | EIVLTQSPATLSLSPGERATLSCRASQSVSSYLAWYQQKPGQAPR |
| | | LLIYDASNRATGIPARFSGSGSGTDFTLTISSLEPEDFAVYYCQQR |
| | | SNWPWTFGQGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 1284 | M4 VL | AIQLTQSPSSLSASVGDRVTITCRASQGISSALAWYQQKPGKAPK |
| | | LLIYDASSLESGVPSRFSGSGSGTDFTLTISSLQPEDFATYYCQQFN |
| | | SYPLTFGGGTKVEIK |
| 1285 | M5 VL | DIQLTQSPSFLSASVGDRVTITCSASSSVNYMHWYQQKPGKAPKL |
| | | LIYSTSNLASGVPSRFSGSGSGTEFTLTISSLQPEDFATYYCHQWN |
| | | NYGTFGQGTKVEIKR |
| 1286 | M6 VL | DVVMTQSPLSLPVTLGQPASISCKSSQNIVHSDGNTYLEWFQQRP |
| | | GQSPRRLIYKVSNRFSGVPDRFSGSGSGTDFTLKISRVEAEDVGV |
| | | YYCFQGSHVPLTFGGGTKVEIKR |
| 1287 | M6 VL | DVVMTQSPLSLPVTLGQPASISCKSSQNIVHSDGNTYLEWFQQRP |
| | | GQSPRRLIYKVSNRFSGVPDRFSGSGSGTDFTLKISRVEAEDVGV |
| | | YYCFQGSHVPLTFGQGTKVEIKR |
| 1288 | M6 VL | DVVMTQTPLSLPVTPGEPASISCKSSQNIVHSDGNTYLEWYLQKP |
| | | GQSPQLLIYKVSNRFSGVPDRFSGSGSGTDFTLKISRVEAEDLGV |
| | | YYCFQGSHVPLTFGGGTKVEIKR |
| 1289 | M6 VL | DVVMTQTPLSLPVSLGDQASISCKSSQNIVHSDGNTYLEWYLQK |
| | | PGQSPKVLIYKVSNRFSGVPDRFSGSGSGTDFTLKISRVEAEDLGV |
| | | YYCFQGSHVPLTFGGGTKVEIKR |
| 1290 | M6 VL | DVVMTQSPLSLPVTLGQPASISCRSSQSIVHSNGNTYLDWFQQRP |
| | | GQSPRRLIYKVSNRFSGVPDRFSGSGSGTDFTLKISRVEAEDVGV |
| | | YYCFQGSHVPLTFGGGTKVEIKR |
| 1291 | M6 VL | DVVMTQSPLSLPVTLGQPASISCRSSQSIVHSNGNTYLDWFQQRP |
| | | GQSPRRLIYKVSNRFSGVPDRFSGSGSGTDFTLKISRVEAEDVGV |
| | | YYCFQGSHVPLTFGQGTKVEIKR |
| 1292 | M6 VL | DVVMTQTPLSLPVTPGEPASISCRSSQSIVHSNGNTYLDWYLQKP |
| | | GQSPQLLIYKVSNRFSGVPDRFSGSGSGTDFTLKISRVEAEDLGV |
| | | YYCFQGSHVPLTFGGGTKVEIKR |
| 1293 | M6 VL | DVVMTQTPLSLPVSLGDQASISCRSSQSIVHSNGNTYLDWYLQKP |
| | | GQSPKVLIYKVSNRFSGVPDRFSGSGSGTDFTLKINRVEAEDLGV |
| | | YFCFQGSHVPLTFGGGTKLEIKR |
| 1294 | M7 VL | DIQMNQSPSSLSASLGDTITITCHASQNINVLLSWYQQKPGNIPKL |
| | | LIYKASNLHTGVPSRFSGSGSGTGFTFTISSLQPEDIATYYCQQGQ |
| | | SYPYTFGGGTKLEIK |
| 1295 | M7 VL | DIQMTQSPSSLSASVGDRVTITCQASQDISNYLNWYQQKPGKAP |
| | | KLLIYDASNLETGVPSRFSGSGSGTDFTFTISSLQPEDIATYYCQQ |
| | | YDNLPYTFGQGTKLEIK |
| 1296 | M7 VL | DIQMTQSPSSLSASVGDRVTITCQASQNINVLLNWYQQKPGKAP |
| | | KLLIYKASNLHTGVPSRFSGSGSGTDFTFTISSLQPEDIATYYCQQ |
| | | GQSYPYTFGQGTKLEIK |
| 1297 | M7 VL | DIQMNQSPSSLSASVGDRVTITCQASQNINVLLSWYQQKPGKAP |
| | | KLLIYKASNLHTGVPSRFSGSGSGTDFTFTISSLQPEDIATYYCQQ |
| | | GQSYPYTFGQGTKLEIK |
| 1298 | M8 VL | QSVLTQPPSVSGAPGQRVTISCTSSSSDIGAXXGVXWYQQLPGTA |
| | | PKLLIEGYYNRPSGVPDRFSGSKSGTSASLTITGLLPEDEGDYYCQ |
| | | SXDGTLSALFGGGTKLTVLG |
| 1299 | M8 VL | QSVLTQPPSVSGAPGQRVTISCTSSSSDIGAGLGVHWYQQLPGTA |
| | | PKLLIEGYYNRPSGVPDRFSGSKSGTSASLTITGLLPEDEGDYYCQ |
| | | SWDGTLSALFGGGTKLTVLG |
| 1300 | M8 VL | QSVLTQPPSVSGAPGQRVTISCTSSSSDIGAGLGVHWYQQLPGTA |
| | | PKLLIEGYYNRPSGVPDRFSGSKSGTSASLTITGLLPEDEGDYYCQ |
| | | SYDGTLSALFGGGTKLTVLG |
| 1304 | M8 VL | QSVLTQPPSVSGAPGQRVTISCTSSSSDIGAALGVHWYQQLPGTA |
| | | PKLLIEGYYNRPSGVPDRFSGSKSGTSASLTITGLLPEDEGDYYCQ |
| | | SWDGTLSALFGGGTKLTVLG |
| 1305 | M8 VL | QSVLTQPPSVSGAPGQRVTISCTSSSSDIGAGSGVHWYQQLPGTA |
| | | PKLLIEGYYNRPSGVPDRFSGSKSGTSASLTITGLLPEDEGDYYCQ |
| | | SWDGTLSALFGGGTKLTVLG |
| 1306 | M8 VL | QSVLTQPPSVSGAPGQRVTISCTSSSSDIGAGQGVHWYQQLPGTA |
| | | PKLLIEGYYNRPSGVPDRFSGSKSGTSASLTITGLLPEDEGDYYCQ |
| | | SWDGTLSALFGGGTKLTVLG |
| 1307 | M8 VL | QSVLTQPPSVSGAPGQRVTISCTSSSSDIGAGLGVLWYQQLPGTA |
| | | PKLLIEGYYNRPSGVPDRFSGSKSGTSASLTITGLLPEDEGDYYCQ |
| | | SWDGTLSALFGGGTKLTVLG |
| 1308 | M8 VL | QSVLTQPPSVSGAPGQRVTISCTSSSSDIGAGLGVHWYQQLPGTA |
| | | PKLLIEGYYNRPSGVPDRFSGSKSGTSASLTITGLLPEDEGDYYCQ |
| | | SWDGTLSALFGGGTKLTVLG |
| 1309 | M8 VL | QSVLTQPPSVSGAPGQRVTISCTSSSSDIGAGSGVHWYQQLPGTA |
| | | PKLLIEGYYNRPSGVPDRFSGSKSGTSASLTITGLLPEDEGDYYCQ |
| | | SWDGTLSALFGGGTKLTVLG |
| 1310 | M8 VL | QSVLTQPPSVSGAPGQRVTISCTSSSSDIGAGQGVHWYQQLPGTA |
| | | PKLLIEGYYNRPSGVPDRFSGSKSGTSASLTITGLLPEDEGDYYCQ |
| | | SWDGTLSALFGGGTKLTVLG |
| 1311 | M8 VL | QSVLTQPPSVSGAPGQRVTISCTSSSSDIGAGLGVLWYQQLPGTA |
| | | PKLLIEGYYNRPSGVPDRFSGSKSGTSASLTITGLLPEDEGDYYCQ |
| | | SWDGTLSALFGGGTKLTVLG |
| 1312 | M8 VL | QSVLTQPPSVSGAPGQRVTISCTSSSSDIGAGLGVHWYQQLPGTA |
| | | PKLLIEGYYNRPSGVPDRFSGSKSGTSASLTITGLLPEDEGDYYCQ |
| | | SFDGTLSALFGGGTKLTVLG |
| 1313 | M9 VL | DIQMTQSPSSLSASVGDRVTITCRASQSINNYLNWYQQRPGKAPK |
| | | LLIYAASSLQSGVPSRFSGSGSGTDFTLTISSLQPGDFATYYCQQS |
| | | YSTPRTFGQGTKLEIK |
| 1314 | M10 VL | EIVLTQSPGTLSLSPGERATLSCRASQSVRSSYLAWYQQKPGQAP |
| | | RLLIYGASSRATGIPDRFSGSGSGTDFTLTISRLEPEDFAVYYCQQ |
| | | YGSSPTFGQGTRLEIK |
| 1315 | M11 VL | DIQMTQSPSTLSASVGDRVTITCRASQTISSWLAWYQQTPEKAPK |
| | | LLIYAASNLQSGVPSRFSGSGSGTEFTLTISSLQPDDFATYYCQQY |
| | | HRSWTFGQGTKVEIT |
| 1316 | M12 VL | DVLMTQTPLSLPVSLGDQASISCRSSQNIVHSDGNTYLEWYLQKP |
| | | GQSPKLLIYKVSNRFSGVPDRFSGSGSGTDFTLKISRVEAEDLGIY |
| | | YCFQGSHVPLTFGAGTKLELK |
| 303 | Variable Light | EIVLTQSPGTLSLSPGERATLSC[LCDR1]WYQQKPGQAPRX10X11 |
| | | IY[LCDR2]GIPDRFSGSGSGTDFTLTISRLEPEDFAVYYC[LCDR3]F |
| | | GGGTKLEIK |
| | | wherein each of X10 and X11 is independently selected |
| | | from A, R, N, D, C, Q, E, G, H, I, L, K, M, F, P, S, |
| | | T, W, Y, or V |
| | | In some cases, X10 = L or P |
| | | In some cases, X11 = L or W |

TABLE 2F
Additional sequences

| SEQ | | |
|---|---|---|
| ID NO | Description | Sequence |
| 304 | 219 HC FR1, | QVQLVQSGAEVKKPGASVKVSCKAS |
| | 212 HC FR1 | |
| 305 | 219 HC FR2 | WVKQRPGQGLEWMG |
| 313 | 212 HC FR2 | WVRQRPGQGLEWIG |
| 1317 | HC FR2 | WVRQAPGQGLEWMG |
| 306 | 219 HC FR3a | RVTITRDTSTSTVYLELSSLRSEDTAVYYCAR |
| 307 | 219 HC FR3b | RVTITRDTSTSTVYLELSSLRSEDTAVYYC |
| 314 | 212 HC FR3a | RATITTDTSTSTAYLELSSLRSEDTAVYYCAR |
| 315 | 212 HC FR3b | RATITTDTSTSTAYLELSSLRSEDTAVYYC |
| 1318 | HC FR3 | RVTMTRDTSTSTVYMELSSLRSEDTAVYYC |
| 1319 | HC FR3 | RATITTDTSASTAYLQLSSLRSEDTAVYYC |
| 1320 | HC FR3 | RVTITRDTSASTVYMELSSLRSEDTAVYYC |
| 1321 | HC FR3 | RVTITRDTSASTAYMELSSLRSEDTAVYYC |
| 1322 | HC FR3 | RVTMTRDTSTSTVYMELSSLRSEDTAVYYCSR |
| 1323 | HC FR3 | RVTMTRDTSTSTVYMELSSLRSEDTAVYYCAR |
| 308 | 219 HC FR4, | WGQGTTVTVSS |
| | 212 HC FR4 | |
| 309 | 219 LC FR1, | EIVLTQSPGTLSLSPGERATLSC |
| | 212 LC FR1 | |
| 310 | 219 LC FR2, | WYQQKPGQAPRPLIY |
| | 212 LC FR2 | |
| 1324 | LC FR2 | WYQQKPGQAPRLLIY |
| 311 | 219 LC FR3, | GIPDRFSGSGSGTDFTLTISRLEPEDFAVYYC |
| | 212 LC FR3 | |
| 1325 | LC FR3 | GVPDRFSGSGSGTDYTLTISRLEPEDFAVYYC |
| 312 | 219 LC FR4, | FGGGTKLEIK |
| | 212 LC FR4 | |
| 316 | IGHV1-46*02 | QVQLVQSGAEVKKPGASVKVSCKASGYTFNSYYMHWVRQAPG |
| | | QGLEWMGIINPSGGSTSYAQKFQGRVTMTRDTSTSTVYMELSSL |
| | | RSEDTAVYYCAR |
| 317 | IGKV3-20*01 | EIVLTQSPGTLSLSPGERATLSCRASQSVSSSYLAWYQQKPGQAP |
| | | RLLIYGASSRATGIPDRFSGSGSGTDFTLTISRLEPEDFAVYYCQQ |
| | | YGSSP |
| 319 | Light Chain | RTVAAPSVFIFPPSDEQLKSGTASVVCLLNNFYPREAKVQWKVDNALQS |
| | Constant | GNSQESVTEQDSKDSTYSLSSTLTLSKADYEKHKVYACEVTHQGLSSPVT |
| | | KSFNRGEC |

TABLE 2G
Fc and constant regions

SEQ ID NO: 320 IgG1 Constant
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 321 IgG1 Constant
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 322 IgG1 Constant
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 323 Fcl (L235E)
ASTKGPSVFPLAPSSKS1SGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELEGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 324 Fc2 (L235E)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELEGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 325 Fc3 (L235E)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELEGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 326 Fc4 (L234A, L235A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 327 Fc5 (L234A, L235A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 328 Fc6 (L234A, L235A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 329 Fc7 (L234A, L235A, G237A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPG
SEQ ID NO: 330 Fc8 (L234A, L235A, G237A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPG
SEQ ID NO: 331 Fc9 (L234A, L235A, G237A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPG
SEQ ID NO: 332 Fc10 (L234A, L235A, P329G)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALGAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 333 Fc11 (L234A, L235A, P329G)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALGAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 334 Fc12 (L234A, L235A, P329G)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALGAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 335 Fc13 (L234F, L235E, P331S)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEFEGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPASIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 336 Fc14 (L234F, L235E, P331S)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEFEGGPSVFLFPP
KPKDTLMISRTPEVTCVVVDVSHEDPEVKFNWYVDGVEVHNAKTKPREEQYNSTYRVVSVL
TVLHQDWLNGKEYKCKVSNKALPASIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 337 Fc15 (L234F, L235E, P331S)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEFEGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPASIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 338 Fc16 (L234A, L235E, G237A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAEGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPG
SEQ ID NO: 339 Fc17 (L234A, L235E, G237A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAEGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPG
SEQ ID NO: 340 Fc18 (L234A, L235E, G237A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAEGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPG
SEQ ID NO: 341 Fc19 (L234A, L235E, G237A, P331S)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAEGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPASIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPG
SEQ ID NO: 342 Fc20 (L234A, L235E, G237A, P331S)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAEGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPASIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPG
SEQ ID NO: 343 Fc21 (L234A, L235E, G237A, P331S)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAEGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPASIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPG
SEQ ID NO: 344 Fc22 (L234A, L235A, P329A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALAAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 345 Fc23 (L234A, L235A, P329A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALAAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 346 Fc24 (L234A, L235A, P329A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALAAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 347 Fc25 (D265A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVAVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 348 Fc26 (D265A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSVFLFP
PKPKDTLMISRTPEVTCVVVAVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 349 Fc27 (D265A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVAVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 350 Fc28 (N297G)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYGSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 351 Fc29 (N297G)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREEQYGSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 352 Fc30 (N297G)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYGSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 353 Fc31 (D265A, N297A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVAVSHEDPEVKFNWYVDGVEVHNAKTKPREEQYASTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 354 Fc32 (D265A, N297A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSVFLFP
PKPKDTLMISRTPEVTCVVVAVSHEDPEVKFNWYVDGVEVHNAKTKPREEQYASTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 355 Fc33 (D265A, N297A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVAVSHEDPEVKFNWYVDGVEVHNAKTKPREEQYASTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 356 Fc34 (D265A, N297G)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVAVSHEDPEVKFNWYVDGVEVHNAKTKPREEQYGSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 357 Fc35 (D265A, N297G)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSVFLFP
PKPKDTLMISRTPEVTCVVVAVSHEDPEVKFNWYVDGVEVHNAKTKPREEQYGSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 358 Fc36 (D265A, N297G)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVAVSHEDPEVKFNWYVDGVEVHNAKTKPREEQYGSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 359 Fc37 (L235A, G237A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELAGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 360 Fc38 (L235A, G237A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELAGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 361 Fc39 (L235A, G237A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELAGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 362 Fc40 (IgG4)
ASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPSCPAPEFLGGPSVFLFPPKPK
DTLMISRTPEVTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNVFSCSVMHEALH
NHYTQKSLSLSLGK
SEQ ID NO: 401 (L234A, L235A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 402 (L235E)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELEGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 403 (L234A, L235A, G237A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 404 (L234A, L235E, G237A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAEGAPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 405 (L234A, L235A, P329A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALAAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 406 (L234A, L235A, P329G)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALAAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 407 (P329A)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALAAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVMH
EALHNHYTQKSLSLSPGK
SEQ ID NO: 408 (L234E, L235F, P331S)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEEFGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPASIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 409 (D265A, N297G)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVAVSHEDPEVKFNWYVDGVEVHNAKTKPREEQYGSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 410 (N297G)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYGSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
SEQ ID NO: 411 (S228P)
ASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFLGGPSVFLFPPKPK
DTLMISRTPEVTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNVFSCSVMHEALH
NHYTQKSLSLSLGK
SEQ ID NO: 412 (S228P, L235E)
ASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFEGGPSVFLFPPKPK
DTLMISRTPEVTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNVFSCSVMHEALH
NHYTQKSLSLSLGK
SEQ ID NO: 413 (S228P, F234A, L235A)
ASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEAAGGPSVFLFPPKPK
DTLMISRTPEVTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNVFSCSVMHEALH
NHYTQKSLSLSLGK
SEQ ID NO: 501 (L234A, L235A)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHY
TQKSLSLSPGK
SEQ ID NO: 502 (L235E)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELEGGPSVFLFPPKPKDTL
MISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHY
TQKSLSLSPGK
SEQ ID NO: 503 (L234A, L235A, G237A)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGAPSVFLFPPKPKDTL
MISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHY
TQKSLSLSPGK
SEQ ID NO: 504 (L234A, L235E, G237A)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAEGAPSVFLFPPKPKDTL
MISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHY
TQKSLSLSPGK
SEQ ID NO: 505 (L234A, L235A, P329A)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQD
WLNGKEYKCKVSNKALAAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHY
TQKSLSLSPGK
SEQ ID NO: 506 (L234A, L235A, P329G)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQD
WLNGKEYKCKVSNKALAAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHY
TQKSLSLSPGK
SEQ ID NO: 507 (P329A)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNSTYRVVSVLTVLHQD
WLNGKEYKCKVSNKALAAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHY
TQKSLSLSPGK
SEQ ID NO: 508 (L234E, L235F, P33 1S)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEEFGGPSVFLFPPKPKDTL
MISRTPEVTCVVVDVSHEDPEVKFNWYVDGVEVHNAKTKPREEQYNSTYRVVSVLTVLHQD
WLNGKEYKCKVSNKALPASIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHY
TQKSLSLSPGK
SEQ ID NO: 509 (D265A, N297G)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTL
MISRTPEVTCVVVAVSHEDPEVKFNWYVDGVEVHNAKTKPREEQYGSTYRVVSVLTVLHQD
WLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHY
TQKSLSLSPGK
SEQ ID NO: 510 (N297G)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYGSTYRVVSVLTVLHQD
WLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHY
TQKSLSLSPGK
SEQ ID NO: 511 (S228P)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPCSRSTSESTAALGCLVKDYFPEPVTVSWNSGALTSGVHTFPAVLQSSGLYSLSSVVTV
PSSSLGTKTYTCNVDHKPSNTKVDKRVESKYGPPCPPCPAPEFLGGPSVFLFPPKPKDTLMISR
TPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMHEALHNHYTQKS
LSLSLGK
SEQ ID NO: 512 (S228P, L235E)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPCSRSTSESTAALGCLVKDYFPEPVTVSWNSGALTSGVHTFPAVLQSSGLYSLSSVVTV
PSSSLGTKTYTCNVDHKPSNTKVDKRVESKYGPPCPPCPAPEFEGGPSVFLFPPKPKDTLMISR
TPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMHEALHNHYTQKS
LSLSLGK
SEQ ID NO: 513 (S228P, F234A, L235A)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPCSRSTSESTAALGCLVKDYFPEPVTVSWNSGALTSGVHTFPAVLQSSGLYSLSSVVTV
PSSSLGTKTYTCNVDHKPSNTKVDKRVESKYGPPCPPCPAPEAAGGPSVFLFPPKPKDTLMISR
TPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMHEALHNHYTQKS
LSLSLGK
SEQ ID NO: 514
EIVLTQSPGTLSLSPGERATLSCRASSSVSYMYWYQQKPGQAPRPLIYATSNLASGIPDRFSGS
GSGTDFTLTISRLEPEDFAVYYCQQWEGNPRTFGGGTKLEIKRTVAAPSVFIFPPSDEQLKSGT
ASVVCLLNNFYPREAKVQWKVDNALQSGNSQESVTEQDSKDSTYSLSSTLTLSKADYEKHK
VYACEVTHQGLSSPVTKSFNRGEC
SEQ ID NO: 515 G2 constant domain
ASTKGPSVFPLAPCSRSTSESTAALGCLVKDYFPEPVTVSWNSGALTSGVHTFPAVLQSSGLYS
LSSVVTVPSSNFGTQTYTCNVDHKPSNTKVDKTVERKCCVECPPCPAPPVAGPSVFLFPPKPK
DTLMISRTPEVTCVVVDVSHEDPEVQFNWYVDGVEVHNAKTKPREEQFNSTFRVVSVLTVV
HQDWLNGKEYKCKVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGNVFSCSVMHEALH
NHYTQKSLSLSPGK

### Dosages and Routes of Administration

In general, methods disclosed herein comprise administering a therapeutic agent by oral administration. However, in some instances, methods comprise administering a therapeutic agent by intraperitoneal injection. In some instances, methods comprise administering a therapeutic agent in the form of an anal suppository. In some instances, methods comprise administering a therapeutic agent by intravenous (“i.v.”) administration. It is conceivable that one may also administer therapeutic agents disclosed herein by other routes, such as subcutaneous injection, intramuscular injection, intradermal injection, transdermal injection percutaneous administration, intranasal administration, intralymphatic injection, rectal administration intragastric administration, or any other suitable parenteral administration. In some embodiments, routes for local delivery closer to site of injury or inflammation are preferred over systemic routes. Routes, dosage, time points, and duration of administrating therapeutics may be adjusted. In some embodiments, administration of therapeutics is prior to, or after, onset of either, or both, acute and chronic symptoms of the disease or condition.

An effective dose and dosage of therapeutics to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition (e.g., reduced instances of diarrhea, rectal bleeding, weight loss, and size or number of intestinal lesions or strictures, reduced fibrosis or fibrogenesis, reduced fibrostenosis, reduced inflammation). In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic agent is not therapeutically effective or is not providing a sufficient alleviation of the disease or condition, or symptom of the disease or condition, then the dosage amount and/or route of administration may be changed, or an additional agent may be administered to the subject, along with the therapeutic agent. In some embodiments, as a patient is started on a regimen of a therapeutic agent, the patient is also weaned off (e.g., step-wise decrease in dose) a second treatment regimen.

Suitable dose and dosage administrated to a subject is determined by factors including, but no limited to, the particular therapeutic agent, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day. Non-limiting examples of effective dosages of for oral delivery of a therapeutic agent include between about 0.1 mg/kg and about 100 mg/kg of body weight per day, and preferably between about 0.5 mg/kg and about 50 mg/kg of body weight per day. In other instances, the oral delivery dosage of effective amount is about 1 mg/kg and about 10 mg/kg of body weight per day of active material. Non-limiting examples of effective dosages for intravenous administration of the therapeutic agent include at a rate between about 0.01 to 100 pmol/kg body weight/min. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the therapeutic agent used, the disease or condition to be treated, the mode of administration, the requirements of the individual subject, the severity of the disease or condition being treated, and the judgment of the practitioner.

In some embodiments, the administration of the therapeutic agent is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges may be adjusted based on subject's response to the treatment. Some routes of administration will require higher concentrations of effective amount of therapeutics than other routes.

In certain embodiments wherein the patient's condition does not improve, upon the doctor's discretion the administration of therapeutic agent is administered chronically, that is, for an extended period of time, including throughout the duration of the patient's life in order to ameliorate or otherwise control or limit the symptoms of the patient's disease or condition. In certain embodiments wherein a patient's status does improve, the dose of therapeutic agent being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the therapeutic agent described herein lies within a range of circulating concentrations that include the ED50 with minimal toxicity. In certain embodiments, the daily dosage range and/or the unit dosage amount varies within this range depending upon the dosage form employed and the route of administration utilized.

### Additional Therapeutic Agent

In some embodiments, a subject having, or suspected of having IBD and/or an EIM of IBD is treated with an additional therapeutic agent herein. In some embodiments, the additional therapeutic agent is used alone or in combination with another therapeutic agent. Therefore, “additional” does not limit the therapy to a therapy in addition to another therapy described herein, rather, an “additional” therapeutic agent may be used alone to treat the subject. The therapeutic agents may be administered together or sequentially. The combination therapies may be administered within the same day, or may be administered one or more days, weeks, months, or years apart. In some cases, a therapeutic agent provided herein is administered if the subject is determined to be non-responsive to a first line of therapy, e.g., such as TNF inhibitor. Such determination may be made by treatment with the first line therapy and monitoring of disease state and/or diagnostic determination that the subject would be non-responsive to the first line therapy.

In some embodiments, the additional therapeutic agent comprises an anti-TNF therapy, e.g., an anti-TNFα therapy. In some embodiments, the additional therapeutic agent comprises a second-line treatment to an anti-TNF therapy. In some embodiments, the additional therapeutic agent comprises an immunosuppressant, or a class of drugs that suppress, or reduce, the strength of the immune system. In some embodiments, the immunosuppressant is an antibody. Non-limiting examples of immunosuppressant therapeutic agents include STELARA® (ustekinumab) azathioprine (AZA), 6-mercaptopurine (6-MP), methotrexate, cyclosporin A. (CsA).

In some embodiments, the additional therapeutic agent comprises a selective anti-inflammatory drug, or a class of drugs that specifically target pro-inflammatory molecules in the body. In some embodiments, the anti-inflammatory drug comprises an antibody. In some embodiments, the anti-inflammatory drug comprises a small molecule. Non-limiting examples of anti-inflammatory drugs include ENTYVIO (vedolizumab), corticosteroids, aminosalicylates, mesalamine, balsalazide (Colazal) and olsalazine (Dipentum).

In some embodiments, the additional therapeutic agent comprises a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject. In some embodiments, the therapeutic agent comprises Cx601/Alofisel® (darvadstrocel).

In some embodiments, the additional therapeutic agent comprises a small molecule. The small molecule may be used to treat inflammatory diseases or conditions, or fibrostenonic or fibrotic disease. Non-limiting examples of small molecules include Otezla® (apremilast), alicaforsen, or ozanimod (RPC-1063).

The additional therapeutic agent may comprise an antimycotic agent. In some instances, the antimycotic agent comprises an active agent that inhibits growth of a fungus. In some instances, the antimycotic agent comprises an active agent that kills a fungus. In some embodiments, the antimycotic agent comprises polyene, an azole, an echinocandin, an flucytosine, an allylamine, a tolnaftate, or griseofulvin, or a combination thereof. In other embodiments, the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole. In some other embodiments, the polyene comprises amphotericin B, nystatin, or natamycin. In yet other embodiments, the echinocandin comprises caspofungin, anidulafungin, or micafungin. In various other embodiments, the allylamine comprises naftifine or terbinafine.

The additional therapeutic agent may comprise an agonist or an antagonist of therapeutic target. Non-limiting therapeutic targets include Mitogen-Activated Protein Kinase 4 (MAP4K4), Prostaglandin E Receptor 4 (PTGER4), Interleukin 18 Receptor 1 (IL18R1), Interleukin 18 Receptor Accessory Protein (IL18RAP), Adenylate Cyclase 7 (ADCY7), B Lymphoid Tyrosine Kinase (BLK), G Protein-Coupled Receptor 65 (GPR65), Sprouty Related EVH1 Domain Containing 2 (SPRED2), and Src Kinase Associated Phosphoprotein 2 (SKAP2). Non-limiting examples of MAP4K4 modulators include GNE-220 and PF-6260933. Non-limiting examples of PTGER4 modulators include grapiprant (CJ-023,423), ONO-AE3-208, GW627368X, AH23848, ONO-AE2-227, ONO-AE1-734, AGN205203, rivenprost (ONO-4819), CJ-023,423, and BGC20-1531. Exemplary modulators of PFKFB3 include, but are not limited to 3-(3-pyridinyl)-1-(4-pyridinyl)-2-propen-1-one (3PO), 1-(4-pyridinyl)-3-(2-quinolinyl)-2-propen-1-one (PFK15), 5-triazolo-2-arylpyridazinone, 125 1-(3-pyridinyl)-3-(2-quinolinyl)-2-propen-1-one (PQP), 126 5, 6, 7, 8-tetrahydroxy-2-(4-hydroxyphenyl) chrome-4-one (N4A), and 7, 8-dihydroxy-3-(4-hydroxyphenyl) chromen-4-one (YN1). Non-limiting modulators of ADCY7 include forskolin and colforsin daropate. Non-limiting examples of GPR65 modulators include BTB09089 (3-[(2,4-dichlorophenyl)methylsulfanyl]-1,6-dimethylpyridazino[4,5-e][1,3,4]thiadiazin-5-one), and ZINC62678696.

In some embodiments, the therapeutic agent comprises an agonist of Janus Kinase 1 (JAK1). Non-limiting examples of JAK1 inhibitors include Ruxolitinib (INCB018424), S-Ruxolitinib (INCB018424), Baricitinib (LY3009104, INCB028050), Filgotinib (GLPG0634), Momelotinib (CYT387), Cerdulatinib (PRT062070, PRT2070), LY2784544, NVP-BSK805, 2HCl, Tofacitinib (CP-690550, Tasocitinib), XL019, Pacritinib (SB1518), or ZM 39923 HCl.

The therapeutic agent targeting the above genes or gene expression products may be an antibody or antigen binding fragment thereof. The therapeutic agent may be a small molecule. The therapeutic agent may be a peptide or a protein. The therapeutic agent may be an agonist, or partial agonist. The therapeutic agent may be an allosteric modulator, such as a positive allosteric modulator (PAM). The therapeutic agent may be an antagonist, or partial antagonist. The therapeutic agent may be an inverse agonist. The therapeutic agent may be a negative allosteric modulator (NAM).

Disclosed herein are additional therapeutic agents comprising a modulator of CD30 ligand (CD30L) (Entrez Gene ID: 943). In some embodiments, the modulator of CD30L is an agonist or an antagonist of CD30L. In some instances, the antagonist of CD30L is an inhibitor of CD30L. In some embodiments, an inhibitor of CD30L specifically binds directly or indirectly to CD30L, CD30, or a molecule that interferes directly or indirectly with binding between CD30L and CD30. In some embodiments, as used herein, an inhibitor of CD30L comprises an agent that modulates at least one functional activity of CD30L, such as binding to CD30. Non-limiting examples of inhibitors of CD30L include agents that specifically bind to CD30L, including a polypeptide such as an anti-CD30L antibody or antigen binding fragment thereof, and a nucleic acid, e.g., an antisense construct, siRNA, and ribozyme. An antisense construct includes an expression plasmid that when transcribed in the cell produces RNA complementary to a portion of mRNA encoding CD30L, and an oligonucleotide that inhibits protein expression by hybridizing with the CD30L mRNA. In some embodiments the inhibitor of CD30L comprises a non-polypeptide or non-nucleic acid portion as an active agent that binds to and inhibits CD30L activity.

In some embodiments, an inhibitor of CD30L is a polypeptide that binds to CD30L and/or CD30. In some cases, the polypeptide is a CD30 polypeptide or a portion thereof, wherein the portion retains the ability to bind to CD30L. A portion of a CD30 polypeptide includes at least about 10, 15, 20, 25, 30, 35, 40, 45, or 50 amino acids that have at least about 85%, 90%, or 95% identity to human CD30 having SEQ ID NO: 53, or SEQ ID NO: 54, or a sequence of any CD30 protein-coding isoform (for e.g., P28908). For example, an inhibitor of CD30L comprises a CD30 polypeptide that comprises all or part of the extracellular region of human CD30. In some embodiments, the CD30 polypeptide comprises amino acids 19-390 of SEQ ID NO: 2018 or a binding fragment thereof, having at least about 85%, 90%, or 95% sequence identity to CD30. In some embodiments, the CD30 polypeptide is a homologue of mammalian CD30, e.g., the CD30 polypeptide inhibitor of CD30L is a viral CD30 polypeptide or fragment thereof. As a non-limiting example, the viral CD30 polypeptide comprises viral CD30 from a poxvirus, such as ectromelia virus or cowpox virus.

In a non-limiting example, the inhibitor is an anti-CD30L antibody or an anti-CD30 antibody. As used herein, an antibody includes an antigen-binding fragment of a full length antibody, e.g., a Fab or scFv. In some embodiments, the antibody binds to the extracellular domain of CD30L. In some embodiments, an anti-CD30L antibody comprises a heavy chain comprising three complementarity-determining regions: HCDR1, HCDR2, and HCDR3; and a light chain comprising three complementarity-determining regions: LCDR1, LCDR2, and LCDR3. In some embodiments, the anti-CD30L antibody comprises a HCDR1 comprising SEQ ID NO: 20100, a HCDR2 comprising SEQ ID NO: 20101, a HCDR3 comprising SEQ ID NO: 20102, a LCDR1 comprising SEQ ID NO: 20103, a LCDR2 comprising SEQ ID NO: 20104, and a LCDR3 comprising SEQ ID NO: 20105.

In some embodiments, the anti-CD30L antibody comprises a HCDR1 comprising SEQ ID NO: 20106, a HCDR2 comprising SEQ ID NO: 20107, a HCDR3 comprising SEQ ID NO: 20108, a LCDR1 comprising SEQ ID NO: 20109, a LCDR2 comprising SEQ ID NO: 20110, and a LCDR3 comprising SEQ ID NO: 20111.

In some embodiments, the anti-CD30L antibody comprises a HCDR1 comprising SEQ ID NO: 20112, a HCDR2 comprising SEQ ID NO: 20113, a HCDR3 comprising SEQ ID NO: 20114, a LCDR1 comprising SEQ ID NO: 20115, a LCDR2 comprising SEQ ID NO: 20116, and a LCDR3 comprising SEQ ID NO: 20117.

In some embodiments, the anti-CD30L antibody comprises a HCDR1 comprising SEQ ID NO: 20118, a HCDR2 comprising SEQ ID NO: 20119, a HCDR3 comprising SEQ ID NO: 20120, a LCDR1 comprising SEQ ID NO: 20121, a LCDR2 comprising SEQ ID NO: 20122, and a LCDR3 comprising SEQ ID NO: 20123.

In some embodiments, the anti-CD30L antibody comprises a HCDR1 comprising SEQ ID NO: 20124, a HCDR2 comprising SEQ ID NO: 20125, a HCDR3 comprising SEQ ID NO: 20126, a LCDR1 comprising SEQ ID NO: 20127, a LCDR2 comprising SEQ ID NO: 20128, and a LCDR3 comprising SEQ ID NO: 20129.

In some embodiments, the anti-CD30L antibody comprises a HCDR1 comprising SEQ ID NO: 20130, a HCDR2 comprising SEQ ID NO: 20131, a HCDR3 comprising SEQ ID NO: 20132, a LCDR1 comprising SEQ ID NO: 20133, a LCDR2 comprising SEQ ID NO: 20134, and a LCDR3 comprising SEQ ID NO: 20135.

In some cases, the anti-CD30L antibody comprises a heavy chain (HC) variable domain comprising SEQ ID NO: 20136 and a light chain (LC) variable domain comprising SEQ ID NO: 20137. In some cases, the anti-CD30L antibody comprises a heavy chain (HC) variable domain comprising SEQ ID NO: 20138 and a light chain (LC) variable domain comprising SEQ ID NO: 20139. In some cases, the anti-CD30L antibody comprises a heavy chain (HC) variable domain comprising SEQ ID NO: 20140 and a light chain (LC) variable domain comprising SEQ ID NO: 20141. In some cases, the anti-CD30L antibody comprises a heavy chain (HC) variable domain comprising SEQ ID NO: 20142 and a light chain (LC) variable domain comprising SEQ ID NO: 20143. In some cases, the anti-CD30L antibody comprises a heavy chain (HC) variable domain comprising SEQ ID NO: 20144 and a light chain (LC) variable domain comprising SEQ ID NO: 20145. In some cases, the anti-CD30L antibody comprises a heavy chain (HC) variable domain comprising SEQ ID NO: 20146 and a light chain (LC) variable domain comprising SEQ ID NO: 20154. In some cases, the anti-CD30L antibody comprises a heavy chain (HC) variable domain comprising SEQ ID NO: 20147 and a light chain (LC) variable domain comprising SEQ ID NO: 20154. In some cases, the anti-CD30L antibody comprises a heavy chain (HC) variable domain comprising SEQ ID NO: 20148 and a light chain (LC) variable domain comprising SEQ ID NO: 20154. In some cases, the anti-CD30L antibody comprises a heavy chain (HC) variable domain comprising SEQ ID NO: 20149 and a light chain (LC) variable domain comprising SEQ ID NO: 20154. In some cases, the anti-CD30L antibody comprises a heavy chain (HC) variable domain comprising SEQ ID NO: 20150 and a light chain (LC) variable domain comprising SEQ ID NO: 20154. In some cases, the anti-CD30L antibody comprises a heavy chain (HC) variable domain comprising SEQ TD NO: 20151 and a light chain (LC) variable domain comprising SEQ ID NO: 20154. In some cases, the anti-CD30L antibody comprises a heavy chain (HC) variable domain comprising SEQ TD NO: 20152 and a light chain (LC) variable domain comprising SEQ ID NO: 20154. In some cases, the anti-CD30L antibody comprises a heavy chain (HC) variable domain comprising SEQ TD NO: 20153 and a light chain (LC) variable domain comprising SEQ ID NO: 20154.

In some embodiments, the anti-CD30 antibody comprises a heavy chain variable region comprising SEQ TD NO: 55 and a light chain variable region comprising SEQ TD NO: 56. Non-limiting examples of anti-CD30 antibodies include MDX-60, Ber-H2, SGN-30 (cAC10), Ki-4.dgA, 2RS-3/A9, AFM13, and H22xKi-4.

In some embodiments, the anti-CD30 antibody comprises an antibody drug conjugate. As a non-limiting example, the antibody drug conjugate is brentuximab, an anti-CD3 antibody conjugated to monomethyl auristatin E.

TABLE 3
Exemplary CD30 Ligand Antibodies

| Antibody | SEQ ID NO | Sequence |
|---|---|---|
| HCDR1 A1 | 20100 | SYIWS |
| HCDR2 A1 | 20101 | RIYASGNTNYNPSLKS |
| HCDR3 A1 | 20102 | DYRVAGTYYYYYGLDV |
| LCDR1 A1 | 20103 | TGTSSDVGVYDYVS |
| LCDR2 A1 | 20104 | EVSNRPS |
| LCDR3 A1 | 20105 | SSYTSRSTWV |
| HCDR1 A2 | 20106 | SYYWT |
| HCDR2 A2 | 20107 | RIYTSGITNYNPSLKS |
| HCDR3 A2 | 20108 | ERVVGASRYYYYGVDV |
| LCDR1 A2 | 20109 | TGTSSDVGLYNYVS |
| LCDR2 A2 | 20110 | EVNNRPS |
| LCDR3 A2 | 20111 | SSYTSSSTWV |
| HCDR1 A3 | 20112 | SYYWT |
| HCDR2 A3 | 20113 | RIYTSGITNYNPSLKS |
| HCDR3 A3 | 20114 | ERVVGASRYYYYGVDV |
| LCDR1 A3 | 20115 | TGTSSDIGLYDYVS |
| LCDR2 A3 | 20116 | EVNNRPS |
| LCDR3 A3 | 20117 | SSYTSSSTWV |
| HCDR1 A4 | 20118 | SYSWS |
| HCDR2 A4 | 20119 | RTSTSGRNNYNPSLKS |
| HCDR3 A4 | 20120 | DFTIAARRYYYYGMDV |
| LCDR1 A4 | 20121 | TGTSSDIGLYNYVS |
| LCDR2 A4 | 20122 | EVINRPS |
| LCDR3 A4 | 20123 | SSYTSSSTWV |
| HCDR1 A5 | 20124 | NNYWS |
| HCDR2 A5 | 20125 | RVYSSGLTNYKPSLKS |
| HCDR3 A5 | 20126 | ERATVTTRYHYDGMDV |
| LCDR1 A5 | 20127 | TGSSSDIGTYNYVS |
| LCDR2 A5 | 20128 | EVNNRPS |
| LCDR3 A5 | 20129 | SSYSSSSTWV |
| HCDR1 A6 | 20130 | SYYWS |
| HCDR2 A6 | 20131 | RIFASGSTNYNPSLRS |
| HCDR3 A6 | 20132 | ERVGVQDYYHYSGMDV |
| LCDR1 A6 | 20133 | TGTSSDVGLYNYVS |
| LCDR2 A6 | 20134 | EVSKRPS |
| LCDR3 A6 | 20135 | SSYTSSSTWV |
| HC Var 1 | 20136 | QVQLQESGPGLVKPSETLSLTCTVSGGSISSYYWTWIR |
| | | QPAGKGLEWIGRIYTSGITNYNPSLKSRVTMSVDTSKN |
| | | QFSLKLSSVTAADTAVYYCARERVVGASRYYYYGVD |
| | | VWGQGTTVTVSS |
| LC Var 1 | 20137 | QSALTQPASVSGSPGQSITISCTGTSSDVGLYNYVSWY |
| | | QQHPDKAPKLMIFEVNNRPSGVSNRFSGSNSGNTASL |
| | | TISGLQAEDEADYYCSSYTSSSTWVFGGGTKLTVL |
| HC Var 2 | 20138 | QVQLQESGPGLVKPSETLSLTCTVSGGSISSYYWTWIR |
| | | QPAGKGLEWIGRIYTSGITNYNPSLKSRVTMSVDTSKN |
| | | QFSLKLSSVTAADTAVYYCARERVVGASRYYYYGVD |
| | | VWGQGTTVTVSS |
| LC Var 2 | 20139 | QSALTQPASVSGSPGQSITISCTGTSSDIGLYDYVSWYQ |
| | | QHPDRAPKLIIFEVNNRPSGVSYRFSGSNSGNTASLTIS |
| | | GLQAEDEADYYCSSYTSSSTWVFGGGTKLTVL |
| HC Var 3 | 20140 | QVQLQESGPGLVKPSETLSLTCTVSGGSISSYSWSWIR |
| | | QPAGKGLEWIGRTSTSGRNNYNPSLKSRVTMSVDTSK |
| | | NQFSLKLNSVTAADTAVYYCARDFTIAARRYYYYGM |
| | | DVWGQGTTVTVSS |
| LC Var 3 | 20141 | QSALTQPASVSGSPGQSITISCTGTSSDIGLYNYVSWYQ |
| | | QHPGKAPKLIIYEVINRPSGVSNRFSGSESGNTASLTIS |
| | | GLQAEDEANYYCSSYTSSSTWVFGGGTKLTVL |
| HC Var 4 | 20142 | QVQLQESGPRLVKPSETLSLTCTVSGGSITNNYWSWIR |
| | | QPAGKGLEWIGRVYSSGLTNYKPSLKSRVTMSVDTSK |
| | | NQFSLRLNSVTAADTAVYYCARERATVTTRYHYDGM |
| | | DVWGQGTSVTVSS |
| LC Var 4 | 20143 | QSALTQPASVSGSPGQSITISCTGSSSDIGTYNYVSWYQ |
| | | QYPGKAPELMIYEVNNRPSGVSDRFSGSTSGNTASLTI |
| | | SGLQANDEADYYCSSYSSSSTWVFGGGTKLTVL |
| HC Var 5 | 20144 | QVQLQESGPGLVKPSETLSLTCTVSGGSISSYYWSWIR |
| | | QPAGKGLEWIGRIFASGSTNYNPSLRSRVTMSRDTSKN |
| | | QFSLKLSSVTAADTAVYYCAKERVGVQDYYHYSGMD |
| | | VWGQGTTVTVSS |
| LC Var 5 | 20145 | QSALTQPASVSGSPGQSITISCTGTSSDVGLYNYVSWY |
| | | QQQPGKAPKLMIYEVSKRPSGVSNRFSGSTSGNTASLT |
| | | ISGLQADDEADYSCSSYTSSSTWVFGGGTKLTVL |
| HC Var 6 | 20146 | QVQLQESGPGLVKPSETLSLTCTVSGGSISSYIWSWIRQ |
| | | PAGKGLEWIGRIYASGNTNYNPSLKSRVTISVDTSKNQ |
| | | FSLKLSSMTAADTAVYYCARDYRVAGTYYYYYGLDV |
| | | WGQGTTVTVSS |
| HC Var 7 | 20147 | QVQLQESGPGLVKPSETLSLTCTVSGGSISSYIWSWIRQ |
| | | PAGKGLEWIGRIYASGNTNYNPSLKSRVTMSVDTSKN |
| | | QFSLKLSSMTAADTAVYYCARDYRVAGTYYYYYGLD |
| | | VWGQGTTVTVSS |
| HC Var 8 | 20148 | QVQLQESGPGLVKPSETLSLTCTVSGGSISSYIWSWIRQ |
| | | PAGKGLEWIGRIYASGNTNYNPSLKSRVTMSVDTSKN |
| | | QFSLKLSSVTAADTAVYYCARDYRVAGTYYYYYGLD |
| | | VWGQGTTVTVSS |
| HC Var 9 | 20149 | QVQLQESGPGLVKPSETLSLTCTVSGGSISSYIWSWIRQ |
| | | PAGKGLEWIGRIYASGQTNYNPSLKSRVTMSVDTSKN |
| | | QFSLKLSSMTAADTAVYYCARDYRVAGTYYYYYGLD |
| | | VWGQGTTVTVSS |
| HC Var 10 | 20150 | QVQLQESGPGLVKPSETLSLTCTVSGGSISSYIWSWIRQ |
| | | PAGKGLEWIGRIYASGQTNYNPSLKSRVTISVDTSKNQ |
| | | FSLKLSSVTAADTAVYYCARDYRVAGTYYYYYGLDV |
| | | WGQGTTVTVSS |
| HC Var 11 | 20151 | QVQLQESGPGLVKPSETLSLTCTVSGGSISSYIWSWIRQ |
| | | PAGKGLEWIGRIYASGNTNYNPSLKSRVTISVDTSKNQ |
| | | FSLKLSSVTAADTAVYYCARDYRVAGTYYYYYGLDV |
| | | WGQGTTVTVSS |
| HC Var 12 | 20152 | QVQLQESGPGLVKPSETLSLTCTVSGGSISSYIWSWIRQ |
| | | PAGKGLEWIGRIYASGQTNYNPSLKSRVTISVDTSKNQ |
| | | FSLKLSSMTAADTAVYYCARDYRVAGTYYYYYGLDV |
| | | WGQGTTVTVSS |
| HC Var 13 | 20153 | QVQLQESGPGLVKPSETLSLTCTVSGGSISSYIWSWIRQ |
| | | PAGKGLEWIGRIYASGQTNYNPSLKSRVTMSVDTSKN |
| | | QFSLKLSSVTAADTAVYYCARDYRVAGTYYYYYGLD |
| | | VWGQGTTVTVSS |
| LC Var 6-13 | 20154 | QSALTQPASVSGSPGQSITISCTGTSSDVGVYDYVSWY |
| | | QQHPGKAPKLMIYEVSNRPSGVSNRFSGSKSGNTASL |
| | | TISGLQTEDEADYYCSSYTSRSTWVFGGGTKLTVL |

### Pharmaceutical Composition

A pharmaceutical composition, as used herein, refers to a mixture of a therapeutic agent, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more therapeutic agent (e.g., one or more therapeutic agents and one or more additional agents) as discussed herein. In practicing the methods of treatment or use provided herein, therapeutically effective amounts of therapeutic agents described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the therapeutic agent used and other factors. The therapeutic agents can be used singly or in combination with one or more therapeutic agents as components of mixtures.

The pharmaceutical formulations described herein are administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a therapeutic agent are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a therapeutic agent as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In some embodiments, a therapeutic agent exists as a tautomer. All tautomers are included within the scope of the agents presented herein. As such, it is to be understood that a therapeutic agent or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound.

In some embodiments, a therapeutic agent exists as an enantiomer, diastereomer, or other stereoisomeric form. The agents disclosed herein include all enantiomeric, diastereomeric, and epimeric forms as well as mixtures thereof.

In some embodiments, therapeutic agents described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a therapeutic agent described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the therapeutic agent. In certain embodiments, a prodrug is enzymatically metabolized by one or more steps or processes to the biologically, pharmaceutically or therapeutically active form of the therapeutic agent.

Prodrug forms of the therapeutic agents, wherein the prodrug is metabolized in vivo to produce an agent as set forth herein are included within the scope of the claims. Prodrug forms of the herein described therapeutic agents, wherein the prodrug is metabolized in vivo to produce an agent as set forth herein are included within the scope of the claims. In some cases, some of the therapeutic agents described herein may be a prodrug for another derivative or active compound. In some embodiments described herein, hydrazones are metabolized in vivo to produce a therapeutic agent.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

The pharmaceutical compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, a therapeutic agent described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch.

Representative intranasal formulations are described in, for example, U.S. Pat. Nos. 4,476,116, 5,116,817 and 6,391,452. Formulations that include a therapeutic agent are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the therapeutic agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, pharmaceutical formulations of a therapeutic agent are in the form of a capsules, including push fit capsules made of gelatin, as well as soft, sealed capsules made of gelatin and a plasticizer, such as glycerol or sorbitol. The push fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration. In one aspect, solid oral dosage forms are prepared by mixing a therapeutic agent with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating can provide a delayed release of a therapeutic agent from the formulation. In other embodiments, the film coating aids in patient compliance (e.g., Opadry® coatings or sugar coating). Film coatings including Opadry® typically range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, IF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a therapeutic agent are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a therapeutic agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxypropylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a therapeutic agents and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a therapeutic agent is formulated to include one or more pharmaceutical excipients and flavors. Such a powder is prepared, for example, by mixing the therapeutic agent and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

In some embodiments, the pharmaceutical dosage forms are formulated to provide a controlled release of a therapeutic agent. Controlled release refers to the release of the therapeutic agent from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a therapeutic agent that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following: Shellac—this coating dissolves in media of pH>7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)—PVAP dissolves in pH>5, and it is much less permeable to water vapor and gastric fluids. Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of a therapeutic agent upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a therapeutic agent and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of a therapeutic agent, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium docusate, cholesterol, cholesterol esters, taurocholic acid, phosphatidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween @60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20@ and Tween 80@, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet®), malitol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

In some embodiments, a therapeutic agent is prepared as transdermal dosage form. In some embodiments, the transdermal formulations described herein include at least three components: (1) a therapeutic agent; (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of a therapeutic agent described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the therapeutic agents described herein can be accomplished by means of iontophoretic patches and the like. In one aspect, transdermal patches provide controlled delivery of a therapeutic agent. In one aspect, transdermal devices are in the form of a bandage comprising a backing member, a reservoir containing the therapeutic agent optionally with carriers, optionally a rate controlling barrier to deliver the therapeutic agent to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical therapeutic agents may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, a therapeutic agent described herein may be administered topically and can be formulated into a variety of topically administrable compositions, such as solutions, suspensions, lotions, gels, pastes, medicated sticks, balms, creams or ointments. Such pharmaceutical therapeutic agents can contain solubilizers, stabilizers, tonicity enhancing agents, buffers and preservatives.

B. Methods of Treatment

In certain embodiments, described herein are methods for evaluating an effect of a treatment described herein. In some instances, the treatment comprises administration with a therapeutic agent and optionally, one or more additional therapeutic agents. In some instances, the treatment is monitored by evaluating the quantity of TL1A in the subject prior to and/or after administration of a therapeutic agent.

### NUMBERED EMBODIMENTS

Non-limiting embodiments of the present disclosure include the following:
1. A method of treating a subject having or suspected of having at least one of an inflammatory, fibrostenotic, and fibrotic, disease or condition the method comprising:
   a) identifying the subject as being a carrier of a genotype comprising a polymorphism provided in Table 1 or Tables 10A-10E, or a polymorphism in linkage disequilibrium (LD) therewith; and
   b) administering to the subject a therapeutically effective amount of an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof.
2. The method of embodiment 1, provided that the inflammatory disease comprises Crohn's disease.
3. The method of embodiment 2, provided that the Crohn's disease comprises ileal, ileocolonic, or colonic Crohn's disease.
4. The method of embodiment 1, provided that the inflammatory disease comprises ulcerative colitis (UC).
5. The method of embodiment 4, provided that the UC is medically refractory UC.
6. The method of any of embodiments 1-5, wherein identifying the subject as being a carrier of the genotype of step (a) comprises:
   a) contacting a sample comprising genetic material from the subject with a nucleic acid sequence capable of hybridizing to at least 10 contiguous nucleobases comprising a risk allele located at nucleoposition 501 within any one of SEQ ID NOS: 2001-2031; and
   b) detecting binding between the nucleic acid sequence and the at least 10 contiguous nucleobases comprising the risk allele.
7. The method of embodiment 6, provided that the standard hybridization conditions comprise an annealing temperature between about 35° C. and about 65° C.
8. The method of embodiment 6 or embodiment 7, provided that the standard hybridization conditions are performed with a TaqMan master mix solution.
9. The method of any of embodiments 6-8, provided that the nucleic acid sequence is conjugated to a detectable molecule.
10. The method of embodiment 9, provided that the detectable molecule comprises a fluorophore.
11. The method of any of embodiments 6-10, provided that the nucleic acid sequence is conjugated to a quencher.
12. The method of any of embodiments 6-11, provided that the sample comprising genetic material from the subject is amplified genetic material obtained from a nucleic acid amplification assay.
13. The method of embodiment 12, provided that the nucleic acid amplification assay comprises amplification of DNA from the subject with a pair of primers capable of amplifying at least 15 contiguous nucleobases comprising the risk allele located at nucleoposition 51 within any one of SEQ ID NOS: 2001-2031, the pair of primers comprising a first primer and a second primer.
14. The method of embodiment 12, provided that the first primer comprises a nucleic acid sequence complimentary to at least 15 contiguous nucleobases upstream of the risk allele located at nucleobase 51 within any one of SEQ ID NOS: 2001-2031, and the second primer comprises a nucleic acid sequence complimentary to at least 15 contiguous nucleobases downstream of the risk allele located at nucleobase 51 within any one of SEQ ID NOS: 2001-2031.
15. The method of any of embodiments 1-14, provided that the subject has been determined to be a carrier of the genotype by a process comprising DNA sequencing.
16. The method of any of embodiments 1-15, provided that the subject further comprises soluble TL1A at a level greater than a control level derived from a non-diseased individual or population of non-diseased individuals.
17. The method of any of embodiments 1-16, provided that the subject is homozygous for the genotype.
18. The method of any of embodiments 1-17, wherein the genotype comprises at least two polymorphisms provided in Table 1 or Tables 10A-10E.
19. The method of any of embodiments 1-17, wherein the genotype comprises at least three polymorphisms provided in Table 1 or Tables 10A-10E.
20. The method of any of embodiments 1-19, wherein the genotype comprises a polymorphism selected from the group consisting of a “A”, or “T” allele at rs2516514 (SEQ ID NO:2001); a “A”, or “G” allele at rs17030062 (SEQ ID NO:2002); a “A”, or “G” allele at rs9305694 (SEQ ID NO:2003); a “C”, or “A” allele at rs139009610 (SEQ ID NO:2004); a “T”, or “C” allele at rs28732100 (SEQ ID NO:2005); a “A”, T”, or “T” allele at rs12199223 (SEQ ID NO:2006); a “C”, or “G” allele at rs1265181 (SEQ ID NO:2007); a “T”, or “A” allele at rs13417109 (SEQ ID NO:2008); a “C”, or “A” allele at rs17026757 (SEQ ID NO:2009); a “C”, C”, or “T” allele at rs117670930 (SEQ ID NO:2010); a “T”, or “G” allele at rs115994059 (SEQ ID NO:2011); a “G”, or “A” allele at rs7297515 (SEQ ID NO:2012); a “A”, or “G” allele at rs13166683 (SEQ ID NO:2013); a “A”, C”, or “T” allele at rs62376929 (SEQ ID NO:2014); a “C”, T”, or “T” allele at rs4418214 (SEQ ID NO:2015); a “G”, T”, or “T” allele at rs3819299 (SEQ ID NO:2016); a “A”, or “G” allele at rs2373969 (SEQ ID NO:2017); a “A”, A”, or “G” allele at rs4151651 (SEQ ID NO:2018); a “A”, T”, or “G” allele at rs9366775 (SEQ ID NO:2019); a “C”, or “A” allele at rs2858884 (SEQ ID NO:2020); a “C”, or “T” allele at rs2858319 (SEQ ID NO:2021); a “C”, or “G” allele at rs6917611 (SEQ ID NO:2022); a “T”, T”, or “G” allele at rs6930571 (SEQ ID NO:2023); a “T”, C”, T”, or “C” allele at rs389419 (SEQ ID NO:2024); a “G”, G”, T”, or “T” allele at rs76558762 (SEQ ID NO:2025); a “C”, or “G” allele at rs7956721 (SEQ ID NO:2026); a “A”, T”, or “G” allele at rs887864 (SEQ ID NO:2027); a “A”, G”, or “C” allele at rs9276424 (SEQ ID NO:2028); a “G”, or “A” allele at rs10056322 (SEQ ID NO:2029); a “C”, or “A” allele at rs6461986 (SEQ ID NO:2030); or a “A”, or “C” allele at rs13421864. (SEQ ID NO:2031).
21. The method of embodiments 1-20, wherein the therapeutic agent is an anti-TL1A antibody.
22. The method of embodiment 21, wherein the anti-TL1A antibody is selected from Table 2C.
23. The method of embodiment 21, wherein the anti-TL1A antibody comprises an amino acid sequence provided in any one of Tables 2A-2H.
24. The method of embodiment 21, wherein the anti-TL1A antibody binds to the same region of human TL1A as a reference antibody selected from Table 2C.
25. The method of embodiment 21, wherein the anti-TL1A antibody binds to the same region of human TL1A as a reference antibody, the reference antibody comprising an amino acid sequence provided in any one of Tables 2A-2H.
26. The method of embodiments 21-25, wherein the anti-TL1A antibody is a neutralizing TL1A antibody.
27. The method of embodiments 21-26, wherein the anti-TL1A antibody is an antagonist of TL1A.
28. A method of treating an inflammatory, fibrostenotic, and fibrotic, disease or condition in a subject comprising administering to the subject a therapeutically effective amount of a therapeutic agent, provided a presence of a genotype is detected in a sample obtained from the subject.
29. A method of treating an inflammatory, fibrostenotic, and fibrotic, disease or condition in a subject comprising:
   a) analyzing a sample obtained from a subject to detect a presence or an absence of a genotype;
   b) detect the presence of the genotype in the sample obtained from the subject;
   c) administering to the subject a therapeutically effective amount of a therapeutic agent.
30. The method of embodiment 28-29, provided that the inflammatory disease comprises Crohn's disease.
31. The method of embodiment 30, provided that the Crohn's disease comprises ileal, ileocolonic, or colonic Crohn's disease.
32. The method of embodiment 28-29, provided that the inflammatory disease is ulcerative colitis (UC).
33. The method of embodiment 32, provided that the UC is medically refractory UC.
34. The method of any of embodiments 29-33, wherein the presence of the genotype is detected in the sample obtained from the subject by:
   a) contacting the sample comprising genetic material from the subject with a nucleic acid sequence capable of hybridizing to at least 10 contiguous nucleobases comprising a risk allele located at nucleoposition 501 within any one of SEQ ID NOS: 2001-2031; and
   b) detecting binding between the nucleic acid sequence and the at least 10 contiguous nucleobases comprising the risk allele.
35. The method of embodiment 34, provided that the standard hybridization conditions comprise an annealing temperature between about 35° C. and about 65° C.
36. The method of embodiment 34 or embodiment 35, provided that the standard hybridization conditions are performed with a TaqMan master mix solution.
37. The method of any of embodiments 34-36, provided that the nucleic acid sequence is conjugated to a detectable molecule.
38. The method of embodiment 37, provided that the detectable molecule comprises a fluorophore.
39. The method of any of embodiments 34-38, provided that the nucleic acid sequence is conjugated to a quencher.
40. The method of any of embodiments 34-39, provided that the sample comprising genetic material from the subject is amplified genetic material obtained from a nucleic acid amplification assay.
41. The method of embodiment 40, provided that the nucleic acid amplification assay comprises amplification of DNA from the subject with a pair of primers capable of amplifying at least 15 contiguous nucleobases comprising the risk allele located at nucleoposition 501 within any one of SEQ ID NOS: 2001-2031, the pair of primers comprising a first primer and a second primer.
42. The method of embodiment 41, provided that the first primer comprises a nucleic acid sequence complimentary to at least 15 contiguous nucleobases upstream of the risk allele located at nucleobase 51 within any one of SEQ ID NOS: 2001-2031, and the second primer comprises a nucleic acid sequence complimentary to at least 15 contiguous nucleobases downstream of the risk allele located at nucleobase 51 within any one of SEQ ID NOS: 2001-2031.
43. The method of any of embodiments 29-42 the presence of the genotype is detected in the sample obtained from the subject by a process comprising DNA sequencing.
44. The method of any of embodiments 29-43, provided that the subject further comprises soluble TL1A at a level greater than a control level derived from a non-diseased individual or population of non-diseased individuals.
45. The method of any of embodiments 29-44, provided that the subject is homozygous for the genotype.
46. The method of any of embodiments 29-45, wherein the genotype comprises at least two polymorphisms provided in Table 1 or Tables 10A-10E.
47. The method of any of embodiments 29-46, wherein the genotype comprises at least three polymorphisms provided in Table 1 or Tables 10A-10E.
48. The method of any of embodiments 29-47, wherein the genotype comprises a polymorphism selected from the group consisting of a “A”, or “T” allele at rs2516514 (SEQ ID NO:2001); a “A”, or “G” allele at rs17030062 (SEQ ID NO:2002); a “A”, or “G” allele at rs9305694 (SEQ ID NO:2003); a “C”, or “A” allele at rs139009610 (SEQ ID NO:2004); a “T”, or “C” allele at rs28732100 (SEQ ID NO:2005); a “A”, T”, or “T” allele at rs12199223 (SEQ ID NO:2006); a “C”, or “G” allele at rs1265181 (SEQ ID NO:2007); a “T”, or “A” allele at rs13417109 (SEQ ID NO:2008); a “C”, or “A” allele at rs17026757 (SEQ ID NO:2009); a “C”, C”, or “T” allele at rs117670930 (SEQ ID NO:2010); a “T”, or “G” allele at rs115994059 (SEQ ID NO:2011); a “G”, or “A” allele at rs7297515 (SEQ ID NO:2012); a “A”, or “G” allele at rs13166683 (SEQ ID NO:2013); a “A”, C”, or “T” allele at rs62376929 (SEQ ID NO:2014); a “C”, T”, or “T” allele at rs4418214 (SEQ ID NO:2015); a “G”, T”, or “T” allele at rs3819299 (SEQ ID NO:2016); a “A”, or “G” allele at rs2373969 (SEQ ID NO:2017); a “A”, A”, or “G” allele at rs4151651 (SEQ ID NO:2018); a “A”, T”, or “G” allele at rs9366775 (SEQ ID NO:2019); a “C”, or “A” allele at rs2858884 (SEQ ID NO:2020); a “C”, or “T” allele at rs2858319 (SEQ ID NO:2021); a “C”, or “G” allele at rs6917611 (SEQ ID NO:2022); a “T”, T”, or “G” allele at rs6930571 (SEQ ID NO:2023); a “T”, C”, T”, or “C” allele at rs389419 (SEQ ID NO:2024); a “G”, G”, T”, or “T” allele at rs76558762 (SEQ ID NO:2025); a “C”, or “G” allele at rs7956721 (SEQ ID NO:2026); a “A”, T”, or “G” allele at rs887864 (SEQ ID NO:2027); a “A”, G”, or “C” allele at rs9276424 (SEQ ID NO:2028); a “G”, or “A” allele at rs10056322 (SEQ ID NO:2029); a “C”, or “A” allele at rs6461986 (SEQ ID NO:2030); or a “A”, or “C” allele at rs13421864. (SEQ ID NO:2031).
49. The method of embodiments 29-48, wherein the therapeutic agent is an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof.
50. The method of embodiment 49, wherein the therapeutic agent is an anti-TL1A antibody.
51. The method of embodiment 50, wherein the anti-TL1A antibody is selected from Table 2C.
52. The method of embodiment 50, wherein the anti-TL1A antibody comprises an amino acid sequence provided in any one of Tables 2A-2H.
53. The method of embodiment 50, wherein the anti-TL1A antibody binds to the same region of human TL1A as a reference antibody selected from Table 2C.
54. The method of embodiment 50, wherein the anti-TL1A antibody binds to the same region of human TL1A as a reference antibody, the reference antibody comprising an amino acid sequence provided in any one of Tables 2A-2H.
55. The method of embodiments 50-54, wherein the anti-TL1A antibody is a neutralizing TL1A antibody.
56. The method of embodiments 50-55, wherein the anti-TL1A antibody is an antagonist of TL1A.
57. A method of characterizing at least one of an inflammatory, fibrostenotic, and fibrotic, disease or condition of a subject, the method comprising assaying genetic material from the subject to identify the presence or absence of a genotype comprising a polymorphism provided in Table 1 or Tables 10A-10E.
58. The method of embodiment 57, further comprising assigning a more favorable prognosis to treatment with a therapeutic agent when the genotype is present.
59. The method of embodiment 57, further comprising assigning a less favorable prognosis to with a therapeutic agent when the genotype is absent.
60. The method of embodiment 57, further comprising assigning the subject to treatment with a therapeutic agent when the genotype is present.
61. The method of embodiment 57, further comprising prescribing to the subject a therapeutic agent when the genotype is present.
62. The method of embodiment 57, further comprising administering to the subject an inhibitor of anti-CD30 ligand activity or expression when the genotype is present.
63. The method of any of embodiments 58-62, provided that the therapeutic agent is an anti-TL1A antibody or antigen-binding fragment thereof.
64. The method of any of embodiments 57-63, provided that assaying comprises amplifying from the genetic material comprising at least 15 contiguous nucleobases including a risk allele located at nucleoposition 501 within any one of SEQ ID NOS: 2001-2031 using a pair of primers comprising a first primer and a second primer.
65. The method of any of embodiment 64, provided that the first primer comprises a nucleic acid sequence complimentary to at least 15 contiguous nucleobases upstream of the risk allele located at nucleobase 51 within any one of SEQ ID NOS: 2001-2031, and the second primer comprises a nucleic acid sequence complimentary to at least 15 contiguous nucleobases downstream of the risk allele located at nucleobase 501 within any one of SEQ ID NOS: 2001-2031.
66. The method of any of embodiments 57-65, provided that assaying comprises hybridizing to the genetic material a nucleic acid comprising any one of SEQ ID NOS: 2001-2031.
67. The method of embodiment 66, provided that the nucleic acid sequence is conjugated to a detectable molecule.
68. The method of embodiment 67, provided that the detectable molecule comprises a fluorophore.
69. The method of any of embodiments 66-68, provided that the nucleic acid sequence is conjugated to a quencher.
70. The method of any of embodiments 57-65, provided that assaying comprises DNA sequencing.
71. The method of any of embodiments 57-70, further comprising measuring the level of TL1A in the subject.
72. The method of any of embodiments 57-71, provided that the subject is homozygous for the genotype.
73. The method of embodiments 57-72, wherein the genotype comprises at least two polymorphisms provided in Table 1 or Tables 10A-10E.
74. The method of any of embodiments 57-73, wherein the genotype comprises at least three polymorphisms provided in Table 1 or Tables 10A-10E
75. The method of any one of embodiments 57-74, wherein the genotype comprises at least one polymorphism comprising a non-reference allele.
76. The method of any of embodiments 57-75, further comprising characterizing the at least one of the inflammatory, the fibrostenotic, and the fibrotic, disease or condition as Crohn's disease (CD) provided the genotype is present.
77. The method of embodiment 76, provided that the CD comprises ileal, ileocolonic, or colonic CD.
78. The method of any of embodiments 57-77, further comprising characterizing the at least one of the inflammatory, the fibrostenotic, and the fibrotic, disease or condition as a ulcerative colitis (UC), provided the genotype is present.
79. The method of embodiment 78, provided that the fibrotic disease is medically refractory UC.
80. The method of embodiments 57-79, comprising treating the subject with a therapeutic agent comprising an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof.
81. The method of embodiment 80, wherein the therapeutic agent is an anti-TL1A antibody.
82. The method of embodiment 81, wherein the anti-TL1A antibody is selected from Table 2C.
83. The method of embodiment 81, wherein the anti-TL1A antibody comprises an amino acid sequence provided in any one of Tables 2A-2H.
84. The method of embodiment 81, wherein the anti-TL1A antibody binds to the same region of human TL1A as a reference antibody selected from Table 2C.
85. The method of embodiment 81, wherein the anti-TL1A antibody binds to the same region of human TL1A as a reference antibody, the reference antibody comprising an amino acid sequence provided in any one of Tables 2A-2H.
86. The method of embodiments 80-85, wherein the anti-TL1A antibody is a neutralizing TL1A antibody.
87. The method of embodiments 80-85, wherein the anti-TL1A antibody is an antagonist of TL1A.
88. A method for detecting a genotype of interest in a subject comprising at least one of an inflammatory, a fibrostenotic, and a fibrotic, disease or condition, the method comprising:
   (a) contacting genetic material from the subject with a composition sufficiently complementary to and capable of hybridizing to the genotype of interest, the composition comprising:
      (i) a detectably labeled oligonucleotide probe comprising at least 10 contiguous nucleobases provided in any one of SEQ ID NOS: 2001-2031,
      (ii) a detectably labeled oligonucleotide probe comprising at least 10 contiguous nucleobases provided in any one of SEQ ID NOS: 2001-2031,
      (iii) a detectably labeled oligonucleotide probe comprising at least 10 contiguous nucleobases provided in any one of SEQ ID NOS: 2001-2031,
      (iv) a detectably labeled oligonucleotide probe comprising a nucleic acid sequence that differs from a probe selected from the group consisting of (i)-(iii) by up to three nucleobases, provided the detectably labeled oligonucleotide probe of (iv) hybridizes to the genotype of interest,
      (v) a detectably labeled oligonucleotide probe comprising a nucleic acid sequence complementary to a probe selected from the group consisting of (i)-(iv), or
      (vi) a combination of probes selected from the group consisting of (i)-(v),
      wherein the detectably labeled oligonucleotide probe of (i), (ii), and (iii) are different,
   (b) detecting the presence or absence of hybridization of the genetic material with the composition using the detectably labeled probe, whereby hybridization of the genetic material with the composition is indicative of the presence of the genotype of interest in the subject.
89. The method of embodiment 88, provided that the presence of the genotype of interest is indicative of the subject comprising elevated levels of TL1A.
90. The method of embodiment 88 or embodiment 89, provided that the inflammatory disease comprises Crohn's disease (CD).
91. The method of embodiment 88, provided that the CD comprises ileal, ileocolonic, or colonic CD.
92. The method of any of embodiments 88-91, provided that the inflammatory disease is ulcerative colitis (UC).
93. A method of treating the at least one of an inflammatory disease, a fibrostenotic disease, in the subject of any one of embodiments 88-92, the method comprising:
   a) administering to the subject of any of embodiments 88-92 a therapeutically effective amount of a therapeutic agent, provided that the subject comprises the genotype of interest.
94. The method of embodiment 93, wherein the therapeutic agent is an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof.
95. The method of embodiment 94, provided that the therapeutic agent comprises an anti-TL1A ligand antibody or antigen binding fragment thereof.
96. A composition comprising at least 10 but less than 50 contiguous nucleobase residues of any one of SEQ ID NOS: 2001-2031 or its complement, wherein the contiguous nucleobase residues comprise the nucleobase at position 501 of the any one of SEQ ID NOS: 2001-2031, and wherein the contiguous nucleobase residues are connected to a detectable molecule.
97. The composition of embodiment 96, provided that the detectable molecule is a fluorophore.
98. The composition of embodiments 96-97, wherein the contiguous nucleobase residues comprise the nucleobase at position 501 of any one of SEQ ID NOS: 2001-2031.
99. The composition of embodiments 96-98, provided that the contiguous nucleobase residues are connected to a quencher.
100. A kit comprising the composition of any of embodiments 96-99, and a primer pair capable of amplifying at least 15 contiguous nucleic acid molecules of any one of SEQ ID NOS: 2001-2031, the at least 15 contiguous nucleic acid molecules comprising the nucleic acid located at position 501 of any one of SEQ ID NOS: 2001-2031.
101. A method comprising contacting DNA from a subject with the composition of any of embodiments 96-99 or the kit of any of embodiment 100 under conditions configured to hybridize the composition to the DNA if the DNA comprises a sequence complementary to the composition.
102. A method comprising treating the subject of embodiment 101 with a therapeutic agent, provided that the DNA from the subject comprises the sequence complementary to the composition.
103. The method of embodiment 102, wherein the therapeutic agent is an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof.
104. The method of embodiment 103, wherein the therapeutic agent is an anti-TL1A antibody.
105. A method of identifying a risk of developing a TL1A mediated disease or condition comprising at least one of an inflammatory, a fibrostenotic, and a fibrotic, disease or condition in a subject, the method comprising:
   a) assaying a sample obtained from the subject to identify the presence of a genotype comprising a polymorphism provided in Table 1 or Tables 10A-10E, or a polymorphism in linkage disequilibrium (LD) therewith; and
   b) identifying the risk of developing at least one of an inflammatory, a fibrostenotic, and a fibrotic, disease or condition in the subject, provided the presence of the genotype is identified in step (a).
106. A method of selecting a subject for treatment, the method comprising:
   a) assaying a sample obtained from the subject to identify the presence of a genotype comprising a polymorphism provided in Table 1 or Tables 10A-10E, or a polymorphism in linkage disequilibrium (LD) therewith; and
   b) selecting the subject for treatment with a therapeutic agent, provided the presence of the genotype is identified in step (a).
107. The method of any of embodiments 105-106, provided that the subject is homozygous for the genotype.
108. The method of any of embodiments 105-107, wherein the genotype comprises at least two polymorphisms provided in Table 1 or Tables 10A-10E.
109. The method of any of embodiments 105-108, wherein the genotype comprises at least three polymorphisms provided in Table 1 or Tables 10A-10E.
110. The method of any of embodiments 105-109, wherein the genotype comprises at least one polymorphism comprising a non-reference allele.
111. The method of embodiments 105-110, further comprising treating the subject by administering to the subject a therapeutically effective amount of a therapeutic agent.
112. The method of embodiment 111, wherein the therapeutic agent is an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof.
113. The method of embodiment 111, wherein the therapeutic agent is an anti-TL1A antibody.
114. The method of embodiment 113, wherein the anti-TL1A antibody is selected from Table 2C.
115. The method of embodiment 113, wherein the anti-TL1A antibody comprises an amino acid sequence provided in any one of Tables 2A-2H.
116. The method of embodiment 113, wherein the anti-TL1A antibody binds to the same region of human TL1A as a reference antibody selected from Table 2C.
117. The method of embodiment 113, wherein the anti-TL1A antibody binds to the same region of human TL1A as a reference antibody, the reference antibody comprising an amino acid sequence provided in any one of Tables 2A-2H.
118. The method of embodiments 113-117, wherein the anti-TL1A antibody is a neutralizing TL1A antibody.
119. The method of embodiments 113-117, wherein the anti-TL1A antibody is an antagonist of TL1A.
120. The methods of any previous embodiment, comprising administering a therapeutically effective amount of an additional therapeutic agent to the subject.
121. The method of embodiment 120, wherein the additional therapeutic agent is a modulator of Receptor Interacting Serine/Threonine Kinase 2 (RIPK2).
122. The method of embodiment 120, wherein the additional therapeutic agent is a modulator of G Protein-Coupled Receptor 35 (GPR35).
123. The method of embodiment 120, wherein the additional therapeutic agent is a modulator of CD30 ligand (CD30L) 124. The method of embodiment 123, wherein the modulator of CD30L comprises an amino acid sequence provided in Table 3.
125. The method of embodiment 120, wherein the additional therapeutic agent comprises an anti-TL1A antibody, an anti-CD30L antibody, glucocorticosteroid, anti-TNF therapy, anti-a4-b7 therapy, anti-IL12p40 therapy, JAK inhibitor, thalidomide, or cyclophosphamide or a combination thereof.
126. The method of embodiment 120, wherein the additional therapeutic agent comprises an anti-inflammatory drug, cortisone, colchicine, potassium iodine, steroid, hydroxychloroquine, cyclosporin A, thalidomide, or a combination thereof.
127. The method of embodiment 120, wherein the additional therapeutic agent comprises a corticosteroid, ciclosporin, infliximab, canakinumab, clobetasol, mupirocin, gentamicin, tacrolimus, mycophenolate mofetil, or thalidomide, or a combination thereof.
128. The method of embodiment 120, wherein the additional therapeutic agent comprises a steroid, retinoid, methotrexate, adalimumab, brodalumab, certolizumab pegol, guselkumab, infliximab, ixekizumab, risankizumab-rzaa, secukinumab, tildrakizumab, ustekinumab, or apremilast, or a combination thereof.
129. The method of embodiment 120, wherein the additional therapeutic agent comprises a steroid, methotrexate, mycophenolate, sulfasalazine, azathioprine, cyclosporine, adalimumab, infliximab, daclizumab, abatacept, or rituximab, or a combination thereof.
130. The method of embodiment 120, wherein the additional therapeutic agent comprises ursodeoxycholic acid (UDCA), antipruritic, cholestyramine, antibiotic, vitamin A, vitamin D, vitamin E, or vitamin K, or a combination thereof.
131. The method of embodiment 120, wherein the additional therapeutic agent comprises methotrexate, leflunomide, hydroxychloroquine, sulfasalazine, a NSAID, TNF inhibitor, etanercept, infliximab, belimumab, rituximab, anakinra, tocilizumab, canakinumab, secukinumab, ustekinumab, ixekizumab sarilumab, tofacitinib, abatacept, corticosteroid, prednisone, cortisone, or a combination thereof.
132. The method of embodiment 120, wherein the additional therapeutic agent comprises etanercept, adalimumab, infliximab, cyclobenzaprine, steroid, secukinumab, methotrexate, leflunomide hydroxychloroquine, or sulfasalazine, or a combination thereof.
133. The method of any previous embodiment, wherein the subject has or is suspected of having an EIM of IBD.
134. The method of any previous embodiment, wherein the subject is at risk for developing an EIM of IBD.
135. The method of embodiment 133 or 134, wherein the EIM comprises ankylosing spondylitis and sacroiliitis.
136. The method of embodiment 133 or 134, wherein the EIM comprises erythema nodosum, pyoderma gangrenosum, and/or psoriasis.
137. The method of embodiment 133 or 134, wherein the EIM comprises the manifestation in the eye.
138. The method of embodiment 137, wherein the manifestation in the eye comprises uveitis, iritis, episcleritis, scleritis, or undiagnosed ocular inflammation, or a combination thereof.
139. The method of embodiment 133 or 134, wherein the EIM comprises primary sclerosing cholangitis.
140. The method of embodiment 133 or 134, wherein the EIM comprises peripheral arthritis.
141. The method of embodiment 133 or 134, wherein the arthritis comprises large joint arthritis, small joint arthritis, extracolonic arthritis, Crohn's disease/ulcerative colitis non-specific joint inflammation, or IBD-associated arthralgia complication, or a combination thereof.
142. The method of embodiment 133 or 134, wherein the EIM comprises ankylosing spondylitis, sacroiliitis, erythema nodosum, pyoderma gangrenosum, psoriasis, a manifestation in the eye, or primary sclerosing cholangitis, or a combination thereof.
143. The method of embodiment 133 or 134, wherein the EIM comprises ankylosing spondylitis, sacroiliitis, erythema nodosum, pyoderma gangrenosum, psoriasis, eye, peripheral arthritis, or primary sclerosing cholangitis, or a combination thereof.
144. The method of any previous embodiment, wherein the subject comprises a serological marker selected from the group comprising anti-neutrophil cytoplasmic antibodies (ANCA), anti-Saccharomyces cerevisiae antibodies (ASCA), anti-outer member porin C (anti-OmpC), anti-Pseudomonas fluorescens bacterial sequence 12 (anti-I2), and anti-bacterial flagellin (CBir1).
145. The method of any previous embodiment, wherein the subject has or is suspected of having an inflammatory bowel disease.
146. The method of embodiment 145, wherein the inflammatory bowel disease comprises Crohn's disease.
147. The method of embodiment 146, wherein the subject has a Crohn's disease (CD) phenotype selected from the group comprising upper GI, colonic, small bowel, structuring, penetrating, structuring and penetrating, and perianal CD.
148. The method of embodiment 145, wherein the inflammatory bowel disease comprises ulcerative colitis.
149. The method of any previous embodiment, wherein the subject has undergone surgery for treatment of the inflammatory bowel disease.
150. The method of any previous embodiment, wherein the subject is female.
151. The method of any one of embodiments 1-149, wherein the subject is male.
152. The method of any previous embodiment, wherein the subject is Jewish.
153. The method of any previous embodiment, wherein the subject is was diagnosed with inflammatory bowel disease before age 17.
154. The method of any previous embodiment, wherein the subject is was diagnosed with inflammatory bowel disease before age 10.
155. The method of any previous embodiment, wherein the subject is a smoker.
156. The method of any previous embodiment, further comprising determining whether the subject comprises the one or more single nucleotide polymorphisms.

### Kits and Compositions

### Compositions

Disclosed herein are compositions useful for the detection of a genotype or biomarker in a sample obtained from a subject according to the methods described herein. Aspects disclosed herein provide compositions comprises a polynucleotide sequence comprising at least 10 but less than 50 contiguous nucleotides of any one of SEQ ID NOS: 2001-2031, or reverse complements thereof, wherein the contiguous polynucleotide sequence comprises a detectable molecule. In some embodiments, the polynucleotide sequence comprises the nucleobase at a nucleoposition indicated by the non-nucleic acid letter (e.g., S, R, V) in any one of SEQ ID NOS: 2001-2031. In various embodiments, the detectable molecule comprises a fluorophore. In other embodiments, the polynucleotide sequences further comprise a quencher.

Also disclosed herein are compositions comprising an antibody or antigen-binding fragment that specifically binds to a target protein described herein (e.g., TL1A) wherein the antibody or antigen-binding fragment comprises a detectable molecule. In various embodiments, the antibody comprises a monoclonal antibody, a chimeric antibody, a CDR-grafted antibody, a Fab, a Fab′, a F(ab′)₂, a Fv, a disulfide linked Fv, a scFv, a single domain antibody, a diabody, a multispecific antibody, a dual specific antibody, an anti-idiotypic antibody, or a bispecific antibody. In some embodiments, the antibody or antigen-binding fragment comprises an IgG antibody, an IgM antibody, and/or an IgE antibody. In some embodiments, the detectable molecule comprises a fluorophore. In some embodiments, the antibody or antigen-binding fragment is conjugated to a paramagnetic particle (e.g., bead).

### Kits

Disclosed herein, are kits useful for to detect the genotypes and/or biomarkers disclosed herein. In some embodiments, the kits disclosed herein may be used to diagnose and/or treat a disease or condition in a subject; or select a patient for treatment and/or monitor a treatment disclosed herein. In some embodiments, the kit comprises the compositions described herein, which can be used to perform the methods described herein. Kits comprise an assemblage of materials or components, including at least one of the compositions. Thus, in some embodiments the kit contains a composition including of the pharmaceutical composition, for the treatment of IBD. In other embodiments, the kits contains all of the components necessary and/or sufficient to perform an assay for detecting and measuring IBD markers, including all controls, directions for performing assays, and any necessary software for analysis and presentation of results.

In some instances, the kits described herein comprise components for detecting the presence, absence, and/or quantity of a target nucleic acid and/or protein described herein. In some embodiments, the kit further comprises components for detecting the presence, absence, and/or quantity of a serological marker described herein. In some embodiments, the kit comprises the compositions (e.g., primers, probes, antibodies) described herein. The disclosure provides kits suitable for assays such as enzyme-linked immunosorbent assay (ELISA), single-molecular array (Simoa), PCR, and qPCR. The exact nature of the components configured in the kit depends on its intended purpose.

In some embodiments, the kits described herein are configured for the purpose of treating and/or characterizing a disease or condition (e.g., Crohn's disease), or subclinical phenotype thereof (e.g., stricturing, penetrating, or stricturing and penetrating disease phenotypes) in a subject. In some embodiments, the kits described herein are configured for the purpose of identifying a subject suitable for treatment with a therapeutic agent (e.g., anti-TL1A antibody). In some embodiments, the kit is configured particularly for the purpose of treating mammalian subjects. In some embodiments, the kit is configured particularly for the purpose of treating human subjects. In further embodiments, the kit is configured for veterinary applications, treating subjects such as, but not limited to, farm animals, domestic animals, and laboratory animals. In some embodiments, the kit is configured to select a subject for a therapeutic agent, such as those disclosed herein. In some embodiments, the kit is configured to select a subject for treatment with a therapeutic agent disclosed herein. An exemplary therapeutic agent is an anti-TL1A antibody.

Instructions for use may be included in the kit. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### Systems

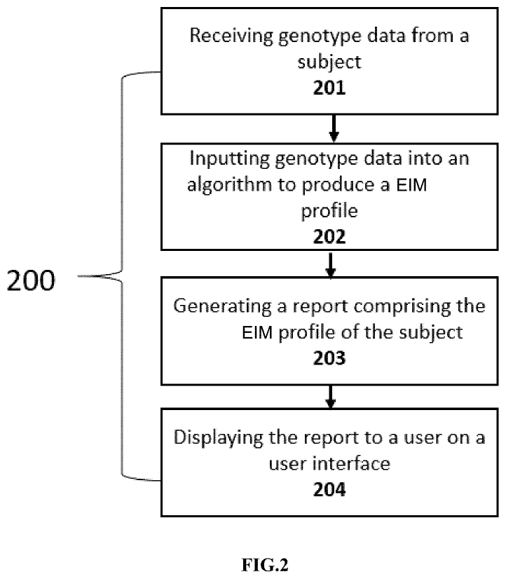

Disclosed herein are systems for identifying a subject as being suitable for treatment with a therapeutic agent (e.g., anti-TL1A antibody). In some embodiments, the systems described herein comprise kits and compositions for detecting the genotypes described herein in a biological sample of a subject. The system may comprise a computer system for implementing one or more methods of the disclosure, such as for example, receiving genotype data of a subject 201, inputting the genotype data into an algorithm to produce a EIM profile 202, and generating a report comprising the EIM profile of the subject 203, and displaying the report to a user on a graphical user interface 204, as shown in FIG. 2. A “EIM profile” as used herein refers to a profile of expression of one or more genotypes described herein in a subject that is detected in a biological sample obtained from the subject. In some embodiments, a EIM profile comprises a likelihood that a patient has or will develop an EIM.

### Computer Systems

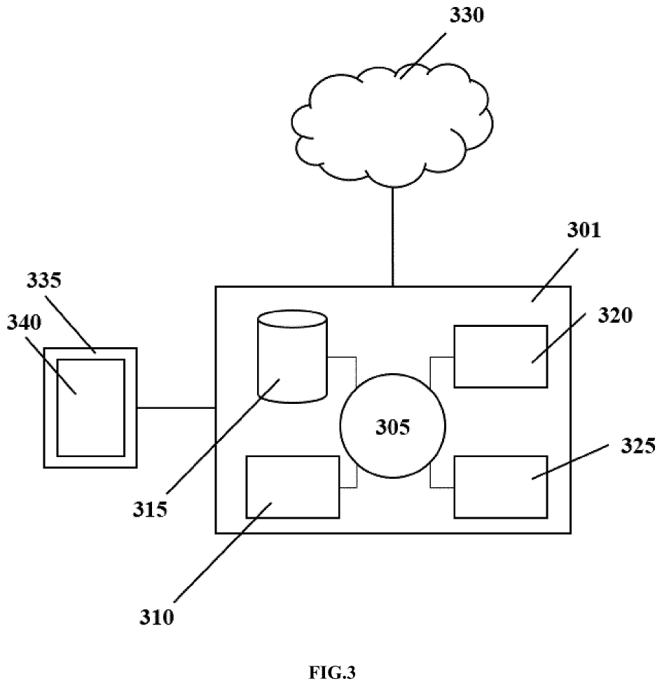

FIG. 3 shows a computer system 301 that is programmed or otherwise configured to generate a EIM profile for a subject in need thereof. The computer system 301 can regulate various aspects of producing the EIM profile (e.g., receiving genotype data, generating a report with the EIM profile of the biological sample, and displaying the report to a user), of the present disclosure, such as, for example, by including permissions or encryption of genotype data and/or EIM profile of the subject to ensure patient privacy.

The computer system 301 can be an electronic device of a user or a computer system that is remotely located with respect to the electronic device. The electronic device can be a mobile electronic device, such as a mobile electronic device belonging to a physician.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, a report comprising the EIM profile of the subject or other relevant clinical information for purposes of informing a selection of a therapeutic agent (e.g., anti-TL1A antibody) to treat a disease or condition of the subject described herein. Examples of UI's include, without limitation, a graphical user interface (GUI) and web-based user interface.

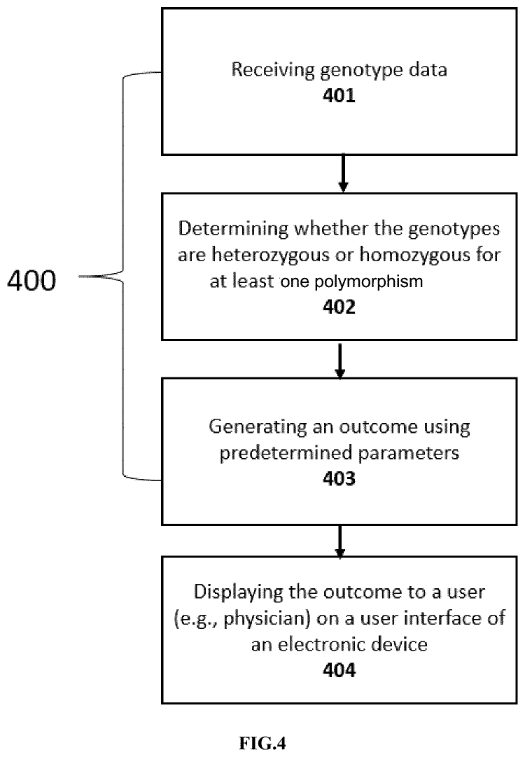

Methods and systems of the present disclosure can be implemented by way of one or more algorithms. An algorithm can be implemented by way of software upon execution by the central processing unit 305. The algorithm can, for example, perform: (a) receiving genotype data of a subject 401, (b) determining whether the genotypes are heterozygous or homozygous for at least one polymorphisms 402, (c) generating an outcome using predetermined parameters 403, and (d) displaying the outcome to a user (e.g., physician) on a user interface of an electronic device 404, as shown in FIG. 4. In some embodiments, the outcome is positive, negative or indeterminate. In some embodiments, the predetermined parameters are genotype combinations known to be predictive of a therapeutic response to a treatment, such as with a therapeutic agent.

### Web Application

In some embodiments, the computer system comprises software for a web application. In light of the disclosure provided herein, those of skill in the art will recognize that a web application may utilize one or more software frameworks and one or more database systems. A web application, for example, is created upon a software framework such as Microsoft® NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PUP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, the computer system comprises software for a mobile application. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, the computer system comprises software a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB .NET, or combinations thereof. Compilation may be often performed, at least in part, to create an executable program. In some instances, a computer program includes one or more executable complied applications.

### Web Browser Plug-In

In some embodiments, the computer system comprises software that comprises a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called microbrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### Databases

The medium, method, and system disclosed herein comprise one or more databases, or use of the same. In view of the disclosure provided herein, those of skill in the art will recognize that many databases are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

### Data Transmission

The subject matter described herein, including methods for producing a EIM profile are configured to be performed in one or more facilities at one or more locations. Facility locations are not limited by country and include any country or territory. In some instances, one or more steps are performed in a different country than another step of the method. In some instances, one or more steps for obtaining a sample are performed in a different country than one or more steps for detecting the presence or absence of a genotype in a biological sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a subject, e.g., processed cellular material. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the stratification of a subject, and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy (e.g., anti-TL1A therapy), or the like, in the same or different location or country.

### Business Methods Utilizing a Computer

The methods described herein may utilize one or more computers. The computer may be used for managing customer and biological sample information such as sample or customer tracking, database management, analyzing molecular profiling data, analyzing cytological data, storing data, billing, marketing, reporting results, storing results, or a combination thereof. The computer may include a monitor or other user interface for displaying data, results, billing information, marketing information (e.g. demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to an individual, a health care provider (e.g., physician) or a health care manager. In one embodiment, a computer-readable medium includes a medium suitable for transmission of a result of an analysis of a biological sample, such as exosome bio-signatures. The medium can include a result regarding an exosome bio-signature of a subject, wherein such a result is derived using the methods described herein.

The entity obtaining a report with the EIM profile may enter biological sample information into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### Exemplary Embodiments

Among the exemplary embodiments are:
1. A computer system for evaluating a sample from a subject, the system comprising:
   a) a central computing environment;
   b) an input device operatively connected to said central computing environment, wherein said input device is configured to receive a presence or absence of a genotype that correlates with a disease state in the sample;
   c) a trained algorithm executed by said central computing environment, wherein the trained algorithm is configured to use the presence or absence of the genotype to classify said sample as at least one of (i) a disease or normal sample, and (ii) a likelihood that a subject has or will develop an EIM; and
   d) an output device operatively connected to said central computing environment, wherein said output device is configured to provide information on the classification to a user.
2. The computer system of embodiment 1, wherein the disease state comprises at least one of an inflammatory, a fibrostenotic, and a fibrotic, disease or condition.
3. The computer system of embodiment 1 or embodiment 2, wherein the disease state is a TL1A mediated disease state selected from the group consisting of inflammatory bowel disease (IBD), Crohn's disease (CD), obstructive CD, ulcerative colitis (UC), intestinal fibrosis, intestinal fibrostenosis, rheumatoid arthritis, and primary sclerosing cholangitis.
4. The computer system of any previous embodiment, wherein the sample comprises whole blood, plasma, serum, or tissue.
5. The computer system of any previous embodiment, wherein the genotype comprises at least one polymorphism selected from Table 1 or Tables 10A-10E, a polymorphism in linkage disequilibrium (LD) therewith, and any combination thereof.
6. The computer system of any previous embodiment, wherein the genotype comprises at least one polymorphism comprising a non-reference allele.
7. The computer system of any previous embodiment, wherein the genotype comprises at least two polymorphisms provided in Table 1 or Tables 10A-10E.
8. The computer system of any previous embodiment, wherein the genotype comprises at least three polymorphisms provided in Table 1 or Tables 10A-10E.
9. The computer system of any previous embodiment, further comprising the genotype is homozygous.
10. The computer system of embodiment 5-9, where LD is defined by an r² value of at least 0.80, 0.85, 0.90, 0.95, or 1.0.
11. The computer system of any previous embodiment, wherein the genotype is associated with a risk that a subject has, or will develop, the disease state by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰.
12. The computer system of any previous embodiment, wherein said output device provides a report summarizing said information on said classification.
13. The computer system of any previous embodiment, wherein said report comprises a recommendation for treatment of said disease state.
14. The computer system of embodiment 13, wherein the treatment comprises administration of a therapeutic agent.
15. The computer system of embodiment 14, wherein the therapeutic agent comprises an antibody or antigen-binding fragment, peptide, or small molecule.
16. The computer system of any preceding embodiment, wherein said genotype is determined with an assay comprising polymerase chain reaction (PCR), quantitative reverse-transcription PCR (qPCR), automated sequencing, genotype array, or a combination thereof.
17. Use of a composition comprising one or more binding agents for generating a report that classifies a sample from a subject as at least one of (i) a disease or non-disease state and (ii) a likelihood that a patient has or will develop an EIM, wherein the one or more binding agents specifically bind to a risk allele provided in Table 1 corresponding to a polymorphism provided in Table 1, their compliment, a polymorphism in linkage disequilibrium therewith, and any combination thereof.
18. The use of embodiment 17, wherein generating the report further comprises:
   a) providing the sample from the subject;
   b) assaying the sample from the subject for detecting the presence of the risk allele corresponding to the polymorphism provided in Table 1;
   c) generating the report based on the result of step (b); and
   d) determining whether said subject has or is likely to develop an EIM based on the results of step (b).
19. The use of embodiment 17 or 18, wherein the disease state comprises at least one of an inflammatory, a fibrostenotic, and a fibrotic, disease or condition.
20. The use of embodiment 17-19, wherein the disease state is a TL1A-mediated disease state selected from the group consisting of inflammatory bowel disease (IBD), Crohn's disease (CD), obstructive CD, ulcerative colitis (UC), intestinal fibrosis, intestinal fibrostenosis, and primary sclerosing cholangitis.
21. The use of any of embodiments 17-20, wherein the sample comprises whole blood, plasma, serum, or tissue.
22. The use of embodiment 18, wherein assaying the sample from the subject for detecting the presence of the risk allele corresponding to the polymorphism provided in Table 1 of step (b) comprises:
   a) contacting the sample with the one or more binding agents that specifically bind to at least 10 contiguous nucleobases that includes the risk allele provided in any one of SEQ ID NOS: 2001-2031; and
   b) determining whether the sample specifically binds to said one or more binding agents, wherein binding of the sample to the one or more binding agents indicates the presence of the polymorphism in the subject.
23. The use of embodiment 18, wherein assaying the sample from the subject for detecting the presence of the risk allele corresponding to the polymorphism provided in Table 1 of step (b) comprises sequencing of the sample.
24. The use of embodiment 18, wherein assaying the sample from the subject for detecting the presence of the one or more polymorphisms of step (b) comprises quantifying the amount of DNA comprising the risk allele.
25. The use of embodiment 24, wherein the quantifying comprises PCR.
26. The use of embodiment 25, wherein the PCR comprises real-time PCR.
27. The use of embodiment 24, wherein the quantifying comprises hybridization.
28. A composition comprising one or more binding agents that specifically bind to a risk allele corresponding to a polymorphism provided in Table 1, wherein the one or more binding agents are selected to classify a sample as at least one of (i) a disease or non-disease or a disease state and (ii) a likelihood that a patient has or will develop an EIM.
29. The composition of embodiment 28, wherein the one or more binding agents comprise oligonucleotides.
30. The composition of embodiment 29, wherein the oligonucleotides comprise RNA or DNA.
31. The composition of embodiment 29, wherein the one or more binding agents comprise aptamers, antibodies, peptide nucleic acids, or pyranosyl RNA.
32. A kit for detecting at least one of an inflammatory, a fibrostenotic, and a fibrotic, disease or condition in a subject, the kit comprising:
   a) at least one binding agent that specifically binds to at least 10 contiguous nucleic acid molecules provided in any one of SEQ ID NOS: 2001-2031 including a corresponding risk allele provided in Table 1, or their complement, wherein the at least one binding agent is selected to detect at least one of (i) a disease or non-disease state and (ii) likelihood that a patient has or will develop an EIM; and
   b) reagents for detecting binding of said at least one binding agent to a DNA sample from a subject.
33. The kit of embodiment 32, wherein the at least one binding agent comprises at least one oligonucleotide.
34. The kit of embodiment 32, wherein the at least one binding agent comprises at least one aptamer, antibody, peptide nucleic acid, or pyranosyl RNA.
35. The kit of embodiment 32-34, wherein the at least one binding agent is labelled with a detectable label.
36. The kit of embodiment 32-35 wherein the at least one binding agent is immobilized to a surface.
37. A system for generating a report that classifies a sample a disease or non-disease of a disease state, comprising:
   a) a computer system that:
      i. generates a molecular profile of a DNA sample based upon the presence of at least one polymorphism, or their complement; and
      ii. generates a report that classifies the sample based on said molecular profile; and
   b) a computer screen that displays said report.
38. The system of embodiment 37, wherein the presence of the at least one polymorphism is based on the result of an assay of said DNA sample, which result is entered into a database.
39. The system of embodiment 37-38, further comprising an input for said result.
40. The system of claim 37-39, wherein the at least one polymorphism is selected from Table 1.
41. The system of claim 37-41, wherein the at least one polymorphism comprises a non-reference allele.
42. The system of claim 41, wherein the at least one polymorphism is two polymorphisms.
43. The system of claim 41, wherein the at least one polymorphism is three polymorphisms.
44. Use of a composition comprising an inhibitor of TL1A for treating a subject, provided the subject is a carrier of a genotype comprising a polymorphism provided in Table 1 or Tables 10A-10E.
45. The use of embodiment 44, wherein the therapeutic agent comprises an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof.
46. The use of embodiment 44, wherein the therapeutic agent is an anti-TL1A antibody.
47. The use of embodiment 46, wherein the anti-TL1A antibody is selected from Table 2C.
48. The use of embodiment 46, wherein the anti-TL1A antibody comprises an amino acid sequence provided in any one of Tables 2A-2H.
49. The use of embodiment 46, wherein the anti-TL1A antibody binds to the same region of human TL1A as a reference antibody selected from Table 2C.
50. The use of embodiment 46, wherein the anti-TL1A antibody binds to the same region of human TL1A as a reference antibody, the reference antibody comprising an amino acid sequence provided in any one of Tables 2A-2H.
51. The use of embodiments 46-50, wherein the anti-TL1A antibody is a neutralizing TL1A antibody.
52. The use of embodiments 46-50, wherein the anti-TL1A antibody is an antagonist of TL1A.
53. The use of embodiments 44-52, wherein the genotype comprises at least two polymorphisms provided in Table 1 or Tables 10A-10E.
54. The use of embodiments 44-53, wherein the genotype comprises at least three polymorphisms provided in Table 1 or Tables 10A-10E.
55. The method of use of embodiments 44-55, wherein the genotype comprises at least one polymorphism comprising a non-reference allele.
56. The computer system of embodiments 1-16, wherein said trained algorithm is configured to classify said sample as likely to have or develop an EIM with a positive predictive value of at least or about 50%, 55%, 60%, 65%, 70%, 75%, 80%, 85%, 90%, 91%, 92%, 93%, 94% 95%, 96%, 97%, 98%, 99%, or 100%.
57. The computer system of embodiments 1-16, wherein said trained algorithm is configured to classify said sample as likely to have or develop an EIM with a specificity of at least or about 50%, 55%, 60%, 65%, 70%, 75%, 80%, 85%, 90%, 91%, 92%, 93%, 94% 95%, 96%, 97%, 98%, 99%, or 100%.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215(3):403-10; Nucleic Acids Res. 1997 Sep. 1; 25(17):3389-402). Percent homology of sequences can be determined using the most recent version of BLAST, as of the filing date of this application. Percent identity of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level. An increase can be an absolute amount (e.g., level of protein expression), or a rate of production (e.g., rate of protein expression between two points in time).

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease. Other examples of “decrease” include a decrease of at least 2-fold, at least 5-fold, at least 10-fold, at least 20-fold, at least 50-fold, at least 100-fold, at least 1000-fold or more as compared to a reference level. A decrease can be an absolute amount (e.g., level of protein expression), or a rate of production (e.g., rate of protein expression between two points in time).

The terms “subject” encompass mammals. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In some instances, a human subject is a “patient,” which as used herein, refers to a subject who may be diagnosed with a disease or condition disclosed herein.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. A “genetic locus” referred to herein, is a particular location within a gene.

The term, “genotype” as disclosed herein, refers to the chemical composition of polynucleotide sequences within the genome of an individual. In some embodiments, the genotype comprises a single nucleotide polymorphism (SNP) or and indel (insertion or deletion, of a nucleobase within a polynucleotide sequence). In some embodiments, a genotype for a particular SNP, or indel is heterozygous. In some embodiments, a genotype for a particular SNP, or indel is homozygous.

A “polymorphism” as used herein refers to an aberration in (e.g., a mutation), or of (e.g., insertion/deletion), a nucleic acid sequence, as compared to the nucleic acid sequence in a reference population. In some embodiments, the polymorphism is common in the reference population. In some embodiments, the polymorphism is rare in the reference population. In some embodiments, the polymorphism is a single nucleotide polymorphism.

The term, “single nucleotide polymorphism” or SNP as disclosed herein, refers to a variation in a single nucleotide within a polynucleotide sequence. The term should not be interpreted as placing a restriction on a frequency of the SNP in a given population. The variation of an SNP may have multiple different forms. A single form of an SNP is referred to as an “allele.” An SNP can be mono-, bi-, tri, or tetra-allelic. A SNP may include a “risk allele,” a “protective allele,” or neither. By way of example, a reference polynucleotide sequence reading 5′ to 3′ is TTACG. A SNP at allele position 3 (of 5′-TTACG-3′) comprise a substitution of the reference allele, “A” to a non-reference allele, “C.” If the “C” allele of the SNP is associated with an increased probability of developing a phenotypic trait, the allele is considered a “risk” allele. However, the same SNP may also comprise a substitution of the “A” allele to a “T” allele at position 3. If the T allele of the SNP is associated with a decreased probability of developing a phenotypic trait, the allele is considered a “protective” allele. The SNP may be observed in at least 1% of a given population. In some embodiments, the SNP is represented by an “rs” number, which refers to the accession of reference cluster of one more submitted SNPs in the dbSNP bioinformatics database as of the filing date of this patent application, and which is included within a sequence that comprises the total number of nucleobases from 5′ to 3′. In some embodiments, a SNP may be further defined by the position of the SNP (nucleobase) within the dbSNP sequence, the position of which is always with reference to 5′ length of the sequence plus 1. In some embodiments, a SNP is defined as the genomic position in a reference genome and the allele change (e.g. chromosome 7 at position 234,123,567 from G allele to A allele in the reference human genome build 37). In some embodiments, the SNV is defined as the genomic position identified with [brackets] or an “N” in a sequence disclosed herein.

The term, “indel,” as disclosed herein, refers to an insertion, or a deletion, of a nucleobase within a polynucleotide sequence. An indel can be mono-, bi-, tri, or tetra-allelic. An indel may be “risk,” a “protective,” or neither, for a phenotypic trait. In some embodiments, the indel is represented by an “rs” number, which refers to the accession of reference cluster of one more submitted indels in the dbSNP bioinformatics database as of the filing date of this patent application, and which is included in a sequence that comprises the total number of nucleobases from 5′ to 3′. In some embodiments, an indel may be further defined by the position of the insertion/deletion within the dbSNP sequence, the position of which is always with reference to the 5′ length of the sequence plus 1. In some embodiments, an indel is defined as the genomic position in a reference genome and the allele change. In some embodiments, the indel is defined as the genomic position identified with [brackets] or an “N” in a sequence disclosed herein.

“Haplotype” as used herein, encompasses a group of one or more genotypes, which tend to be inherited together in a reference population. In some embodiments, a haplotype comprises particular polymorphism or another polymorphism in linkage disequilibrium (LD) therewith.

“Linkage disequilibrium,” or “LD,” as used herein refers to the non-random association of alleles or indels in different gene loci in a given population. LD may be defined by a D′ value corresponding to the difference between an observed and expected allele or indel frequencies in the population (D=Pab−PaPb), which is scaled by the theoretical maximum value of D. LD may be defined by an r² value corresponding to the difference between an observed and expected unit of risk frequencies in the population (D=Pab−PaPb), which is scaled by the individual frequencies of the different loci. In some embodiments, D′ comprises at least 0.20. In some embodiments, r² comprises at least 0.70.

The term “medically refractory,” or “refractory,” as used herein, refers to the failure of a standard treatment to induce remission of a disease. In some embodiments, the disease comprises an inflammatory disease disclosed herein. A non-limiting example of refractory inflammatory disease includes refractory Crohn's disease, and refractory ulcerative colitis (e.g., mrUC). Non-limiting examples of standard treatment include glucocorticosteriods, anti-TNF therapy, anti-a4-b7 therapy (vedolizumab), anti-IL12p40 therapy (ustekinumab), Thalidomide, and Cytoxin.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

The term “inflammatory bowel disease” or “IBD” as used herein refers to gastrointestinal disorders of the gastrointestinal tract. Non-limiting examples of IBD include, Crohn's disease (CD), ulcerative colitis (UC), indeterminate colitis (IC), microscopic colitis, diversion colitis, Behcet's disease, and other inconclusive forms of IBD. In some instances, IBD comprises fibrosis, fibrostenosis, stricturing and/or penetrating disease, obstructive disease, or a disease that is refractory (e.g., mrUC, refractory CD), perianal CD, or other complicated forms of IBD.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples.

The term “biomarker” comprises a measurable substance in a subject whose presence, level, or activity, is indicative of a phenomenon (e.g., phenotypic expression or activity; disease, condition, subclinical phenotype of a disease or condition, infection; or environmental stimuli). In some embodiments, a biomarker comprises a gene, gene expression product (e.g., RNA or protein), or a cell-type (e.g., immune cell).

The term “serological marker,” as used herein refers to a type of biomarker representing an antigenic response in a subject that may be detected in the serum of the subject. In some embodiments, a serological comprises an antibody against various fungal antigens. Non-limiting examples of a serological marker comprise anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globasa antibody, anti-Cladosporium albicans antibody, anti-laminaribiose antibody (ALCA), anti-chitobioside antibody (ACCA), anti-laminarin antibody, anti-chitin antibody, pANCA antibody, anit-I2 antibody, and anti-Cbirl flagellin antibody.

The term “microbiome” and its variation used herein describe the populations and interactions of the bacteria, fungi, protists, and virus that align the gastrointestinal tract of a subject. A subject afflicted with IBD may possess presence, absence, excess, diminished, or a combination thereof of a microbiome s compared to a healthy subject.

The terms “non-response,” or “loss-of-response,” as used herein, refer to phenomena in which a subject or a patient does not respond to the induction of a standard treatment (e.g., anti-TNF therapy), or experiences a loss of response to the standard treatment after a successful induction of the therapy. The induction of the standard treatment may include 1, 2, 3, 4, or 5, doses of the therapy. A “successful induction” of the therapy may be an initial therapeutic response or benefit provided by the therapy. The loss of response may be characterized by a reappearance of symptoms consistent with a flare after a successful induction of the therapy.

The section headings used herein are for organizational purposes only and are not to be construed as limiting the subject matter described.

### Examples

The following examples are included for illustrative purposes only and are not intended to limit the scope of the invention.

### Example 1: Mega-Analysis Reveals Novel Genetic Associations With Extraintestinal Manifestations in Ibd

Genotype data from ImmunoChip array was available for 11,913 unrelated Caucasian IBD cases and 8,059 non-IBD controls from three cohorts: Cedars-Sinai IBD Research Institute (CS-IBD; n=12,886), NIDDK IBD Genetics Consortium (IBDGC; n=3551), and SHARE Consortium (n=3535). Standard SNP and sample QC measures were applied. Clinical charts were reviewed to verify EIMs. We performed mega case-control regression analyses of IBD patients comparing cases with at least one EIM (n=884) and cases without EIMs (n=11,029), adjusting for population substructure. For the CS-IBD cohort, we also investigated the HLA region with logistic regression of classical HLA alleles, imputed to 2-digit resolution with HIBAG, for MHC class I and II genes.

Results: We identified EIM associations at established IBD susceptibility loci, as well as putative novel loci. Known IBD locus HLA-B reached genome-wide (GW) significance for “EIM-4” (ankylosing spondylitis (AS), erythema nodosum (EN), pyoderma (PY), and uveitis/iritis (UVIR)) and AS (r56905036 P_{EIM4}=2.9E-08 OR_{EIM4}=1.7; P_{As}=1.1E-17 OR_{As}=2.8). GW association was also observed at MICA/HCP5 with EIM-4 and AS (r52844510 P_{EIM4}=1.9E-08 OR_{EIM4}=1.6; P_{AS}=1.8E-15 OR_{As}=2.5) and reached suggestive significance with UVIR (P_{UVIR}=4.0E-06 OR_{UVIR}=2.0). Novel associations include GW significance with METTL24 (rs7745186 P_{AS}=1.2E-08 OR_{As}=0.54) and CACNA 1C (rs 11614966 P_{AS}=1.7E-09 OR_{As}=0.40) with AS; as well as PLD5 with EN and suggestive significance with EIM-4 (r512757496 P_{EN}=4.5E-08 OR_{EN}=0.26; P_{EIM4}=5.7E-07 OR_{EIM4}=0.49). Analyses of imputed HLA alleles demonstrated association with AS at known alleles HLA-B*27 (P=9.0E-09 OR=2.7) and HLA-C*02 (P=2.8E-04 OR=2.0), and psoriasis at HLA-C*06 (P=6.3E-04 OR=1.5). Putative novel association was identified for psoriasis at HLA-DPB1* 10 (P=3.4E-05 OR=2.8) and HLA-DPB1*14 (P=5.3E-04 OR=2.0).

Conclusion: Genetics underlying EIMs in IBD is currently not as well understood as the genetics of IBD susceptibility. We have identified genetic variants and HLA alleles across several loci demonstrating association with EIM in IBD that highlight novel candidate loci for further investigation. Additional analyses, including IBD-disease phenotype associations, gene-based tests and pathway analyses, are in progress.

### Example 2: Analysis of Extraintestinal Manifestation Risk in Subjects with Inflammatory Bowel Disease

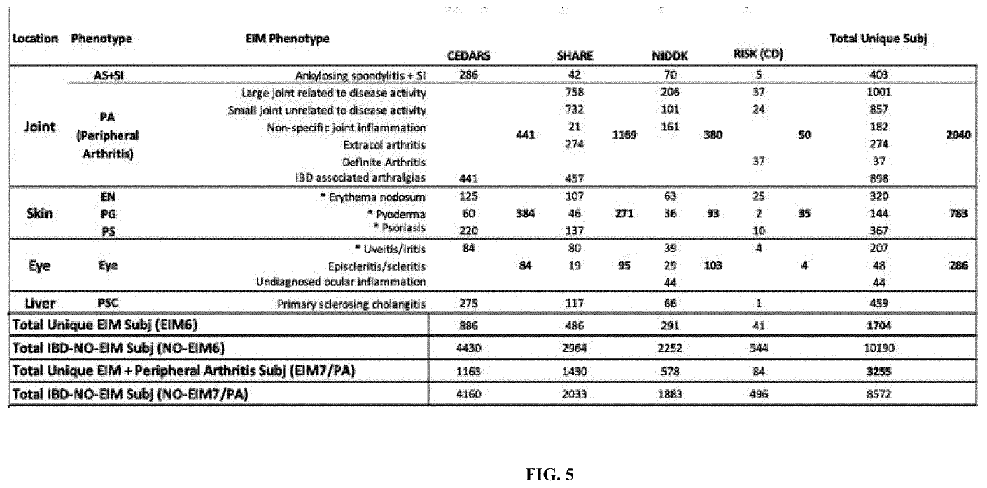

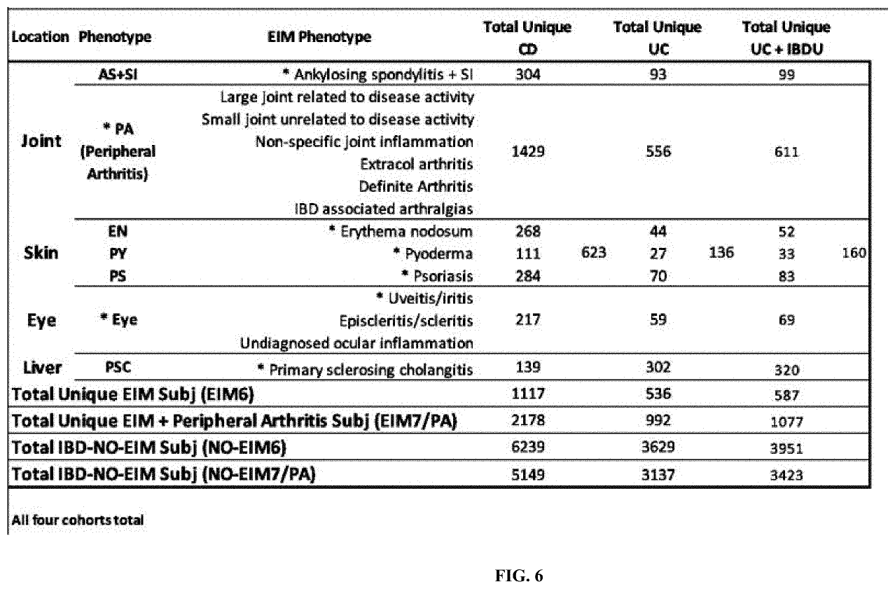

Data was analyzed from patient samples collected from four different cohorts. The four cohorts tested were CSMC (CEDARS), SHARE (Sinai Helmsley Alliance for Research Excellence with 7 recruiting sites), NIDDK (IBD Genetics Consortium with 6 GRCs since 2002), and RISK (pediatric CD inception cohort of newly diagnosed, treatment naïve patients across 28 sites). These cohorts are depicted in FIGS. 5-6 and Table 4.

TABLE 4
Study subjects

| | Pre-QC (N) | | | | |
|---|---|---|---|---|---|
| | IBD Cases | | | Healthy | Pre-QC |
| Cohort | EIM (+) | EIM (−) | Total | Controls | Total |
| CSMC | 1348 | 5303 | 6651 | 5127 | 11,778 |
| SHARE | 1798 | 2625 | 4423 | — | 4423 |
| NIDDK | 580 | 2059 | 2639 | 924 | 3563 |
| RISK | 87 | 558 | 645 | 1637 | 2282 |
| Total | 3813 | 10,545 | 14,358 | 7688 | 22,046 |
| | Post-QC (N) | | | | |
| | IBD Cases | | | Healthy | Post-QC |
| Cohort | EIM (+) | EIM (−) | Total | Controls | Total |
| CSMC | 1163 | 4195 | 5358 | 4,923 | 10,281 |
| SHARE | 1430 | 2066 | 3496 | — | 3496 |
| NIDDK | 578 | 2032 | 2610 | 920 | 3530 |
| RISK | 84 | 535 | 619 | 1629 | 2248 |
| Total | 3255 | 8828 | 12,083 | 7,472 | 19,555 |

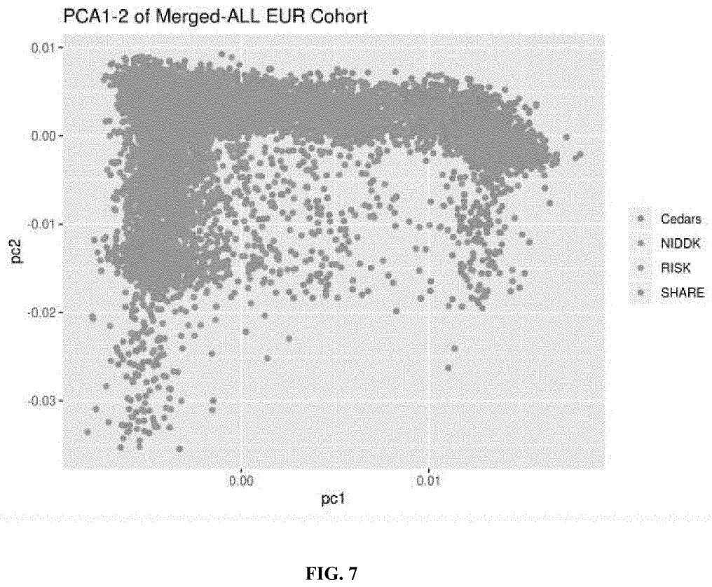

The data was analyzed through multiple comparisons. Logistic regressions were performed between the 5 patient cohorts and the cohort batch, within case analyses (e.g. IBD cases with EIM versus cases without EIM). SNPs were excluded under quality control if the MAF was less than 1%, the deviation from HWE_{controls} had a p1<e-5), a SNP missingness of more than 10% or an AF difference of more than 10% (cohort, gnomAD). Minimal genomic inflation was observed (λGC˜1.02-1.07). FIG. 7 depicts a principle component analysis of the 2 cohorts.

After quality control, the percentage of patients with EIMs is listed in Table 5-6. SKIN-3 describes the percent of patients with erythema nodosum, pyoderma gangernosum, and psoriasis. EIMs in the eye include uveitis, iritis, episcleritis, scleritis, and other eye manifestations. EIM-6 describes the numbers of patients with ankylosing spondylitis and sacroiliitis, erythema nodosum, pyoderma gangernosum, psoriasis, EIMs in the eye, or primary sclerosing cholangitis. EIM-7 describes the numbers of patients with ankylosing spondylitis and sacroiliitis, erythema nodosum, pyoderma gangernosum, psoriasis, EIMs in the eye, primary sclerosing cholangitis, or peripheral arthritis. Peripheral arthritis includes Large & small joint arthritis, Extracolonic arthritis, Arthritis, CD/UC non-specific joint inflammation, or TBD-associated arthralgia complications.

TABLE 5
Phenotypes available after quality control

Post-QC

| | EIM (+) | EIM (−) |
|---|---|---|
| Ankylosing spondylitis & | 403 | 11,624 |
| Sacroiliitis | | |
| SKIN-3 * | 783 | 11,149 |
| Erythema nodosum | 320 | 11,712 |
| Pyoderma gangrenosum | 144 | 11,895 |
| Psoriasis | 367 | 8999 |
| Eye | 286 | 11,738 |
| Primary sclerosing cholangitis | 459 | 11,573 |
| Peripheral Arthritis | 2040 | 9874 |
| EIM-6 ^{#} | 1704 | 10,190 |
| EIM-7 ⁺ | 3255 | 8572 |

TABLE 6
EIMs broken down by cohort

| | CSMC IBD | | SHARE IBD | |
|---|---|---|---|---|
| | Subjects (Post-QC) | | Subjects (Post-QC) | |
| | EIM (+) | EIM (−) | EIM (+) | EIM (−) |
| Ankylosing spondylitis & | 286 | 5072 | 42 | 3453 |
| Sacroiliitis | | | | |
| SKIN-3 * | 384 | 4925 | 271 | 3179 |
| Erythema nodosum | 125 | 5233 | 107 | 3388 |
| Pyoderma gangrenosum | 60 | 5298 | 46 | 3450 |
| Psoriasis | 220 | 5083 | 137 | 3309 |
| Eye | 84 | 5274 | 95 | 3400 |
| Primary sclerosing cholangitis | 275 | 5083 | 117 | 3379 |
| Peripheral Arthritis | 441 | 4917 | 1169 | 2327 |
| EIM-6 ^{#} | 886 | 4430 | 486 | 2964 |
| EIM-7 ⁺ | 1163 | 4160 | 1430 | 2033 |
| | NIDDK IBD | | RISK IBD | |
| | Subjects (Post-QC) | | Subjects (Post-QC) | |
| | EIM (+) | EIM (−) | EIM (+) | EIM (−) |
| Ankylosing spondylitis & | 70 | 2517 | 5 | 582 |
| Sacroiliitis | | | | |
| SKIN-3 * | 93 | 2495 | 35 | 550 |
| Erythema nodosum | 63 | 2530 | 25 | 561 |
| Pyoderma gangrenosum | 36 | 2562 | 2 | 585 |
| Psoriasis | — | — | 10 | 607 |
| Eye | 103 | 2482 | 4 | 582 |
| Primary sclerosing cholangitis | 66 | 2525 | 1 | 586 |
| Peripheral Arthritis | 380 | 2100 | 50 | 530 |
| EIM-6 ^{#} | 291 | 2252 | 41 | 544 |
| EIM-7 ⁺ | 578 | 1883 | 84 | 496 |

The demographics of the patients across the study cohorts are listed in Table 7. Across the study cohort, about 15% of subjects with CD and about 13% of subjects with UC had any EIM. There was a predominancy of EIMs in CD, except for primary sclerosing cholangitis. Female subjects had heater levels of EIM, with the excepts of ankylosing spondylitis and primary sclerosing cholangitis. The mean age of diagnosis was 26 years of age. The majority of subjects had no family history of IBD and were not current smokers. The majority of subjects with CD had ileocolonic disease, inflammatory disease and no perianal complications. The majority of the subjects with UC had extensive disease.

TABLE 7
Cohort demographics

| | EIM-6 (+) | | EIM-6 (−) | |
|---|---|---|---|---|
| | (n = 1704) | | (n = 10,190) | |
| CD | 1129 | (15.3) | 6259 | (84.7) |
| UC | 528 | (12.7) | 3619 | (87.3) |
| IBDU | 47 | (13.1) | 312 | (86.9) |
| Sex (Female) | 919 | (53.9) | 4949 | (48.6) |
| Age at diagnosis (yrs) (mean (SD)) | 26.97 | (13.47) | 26.87 | (14.34) |
| Age at diagnosis (yrs) | | | | |
| A1 (<17) | 393 | (23.1) | 2414 | (23.7) |
| A2 (17-40) | 992 | (58.2) | 5169 | (50.7) |
| A3 (>40) | 266 | (15.6) | 1561 | (15.3) |
| Missing | 53 | (3.1) | 1046 | (10.3) |
| Family history of IBD | | | | |
| Yes | 453 | (26.6) | 2250 | (22.1) |
| No | 1207 | (70.8) | 6722 | (66.0) |
| Missing | 44 | (2.6) | 1218 | (12.0) |
| Current smoking | | | | |
| Yes | 282 | (16.5) | 1331 | (13.1) |
| No | 1394 | (81.8) | 7987 | (78.4) |
| Missing | 28 | (1.6) | 872 | (8.6) |
| Race (self-declared) | | | | |
| White | 1660 | (97.4) | 9853 | (96.7) |
| Black | 0 | (0.0) | 5 | (0.0) |
| Asian | 6 | (0.4) | 35 | (0.3) |
| Other | 36 | (2.1) | 195 | (1.9) |
| Missing | 2 | (0.1) | 102 | (1.0) |
| Jewish (self-reported) | | | | |
| Yes | 496 | (29.2) | 2546 | (25) |
| No | 1192 | (70.0) | 7511 | (73.7) |
| Missing | 16 | (0.9) | 133 | (1.3) |
| Hispanic (self-reported) | | | | |
| Yes | 36 | (2.1) | 232 | (2.3) |
| No | 1657 | (97.2) | 9849 | (96.7) |
| Missing | 11 | (0.6) | 109 | (1.1) |
| Disease location (CD) | | | | |
| Ileal (L1) | 165 | (14.6) | 1311 | (20.9) |
| Colorectal (L2) | 212 | (18.8) | 1022 | (16.3) |
| Ileocolonic (L3) | 709 | (62.8) | 3308 | (52.9) |
| Missing | 43 | (3.8) | 618 | (9.9) |
| Upper GI (L4) | 51 | (4.5) | 687 | (11.0) |
| Isolated small bowel disease (L1) | 165 | | 1311 | |
| Any colonic disease (L2 + L3) | 921 | | 4330 | |
| Inflammatory (B1) | 521 | (46.1) | 2907 | (46.4) |
| Stricturing (B2) | 262 | (23.2) | 1387 | (22.2) |
| Penetrating (B3) | 323 | (28.6) | 1442 | (23.0) |
| Missing | 23 | (2.0) | 523 | (8.4) |
| Perianal (CD) | | | | |
| Yes | 355 | (31.4) | 1477 | (23.6) |
| No | 737 | (65.3) | 4126 | (65.9) |
| Missing | 37 | (3.3) | 656 | (10.5) |
| Disease extent (UC/IBDU) | | | | |
| Proctitis (E1) | 19 | (3.3) | 273 | (6.9) |
| Left-sided [distal] (E2) | 92 | (16.0) | 998 | (25.4) |
| Extensive [pancolitis] (E3) | 403 | (70.1) | 1930 | (49.1) |
| Missing | 61 | (10.6) | 730 | (18.6) |
| IBD-related surgery | | | | |
| Yes | 827 | (48.5) | 3047 | (29.9) |
| No | 582 | (34.2) | 4097 | (40.2) |
| Missing | 295 | (17.3) | 3046 | (29.9) |

The clinical associations of the SNPs are shown in Table 8. This analysis is shown for EIM-6 as both a multivariate and univariate analysis. The association of EIM-6 with gender was maintained in both the univariate and multivariate analysis.

TABLE 8
Clinical associations

| | Univariate: EIM-6 | | | | | | |
|---|---|---|---|---|---|---|---|
| | | | Lower | Upper | | | |
| | | | CI | CI | Multivariate: EIM-6 | | |
| | Pval | OR | (95%) | (95%) | CD | UC | IBD |
| CD (Yes) | 1.66E−05 | 1.272 | 1.140 | 1.419 | | | |
| UC_IBDU (Yes) | 1.67E−05 | 0.786 | 0.705 | 0.877 | | | |
| Sex (Female) | 9.00E−05 | 1.229 | 1.109 | 1.363 | 6.74E−08 | | 1.13E−04 |
| Hispanic (Yes) | 0.33 | 0.838 | 0.587 | 1.195 | | | |
| Jewish (Yes) | 0.17 | 1.088 | 0.964 | 1.228 | | | |
| Age at Diagnosis | 0.43 | 0.999 | 0.995 | 1.002 | | | |
| Age at Diagnosis: < 17 | 0.44 | 0.950 | 0.834 | 1.083 | | | |
| years (Yes) | | | | | | | |
| Age at Diagnosis: < 10 | 0.54 | 0.926 | 0.726 | 1.182 | | | |
| years (Yes) | | | | | | | |
| Current Smoking (Yes) | 0.20 | 1.098 | 0.951 | 1.268 | | | |
| Family History (Yes) | 0.04 | 1.131 | 1.004 | 1.275 | | | |
| CD: Upper GI (Yes) | 0.84 | 1.021 | 0.834 | 1.249 | | | |
| CD: Any Colonic (L2L3 vs L1) | 9.78E−09 | 1.686 | 1.410 | 2.015 | 4.22E−09 | | |
| CD: Any Small Bowel | 0.38 | 0.929 | 0.787 | 1.097 | | | |
| (L1L3 vs L2) | | | | | | | |
| CD: Stricturing (B2 vs B1) | 0.66 | 0.963 | 0.818 | 1.135 | | | |
| CD: Penetrating (B3 vs B1) | 0.16 | 1.117 | 0.956 | 1.305 | | | |
| CD: Stricturing, | 0.57 | 1.039 | 0.911 | 1.186 | | | |
| Penetrating (B2B3 vs B1) | | | | | | | |
| CD: Penetrating vs | 0.11 | 1.158 | 0.967 | 1.387 | | | |
| Stricturing (B3 vs B2) | | | | | | | |
| CD: Perianal (Yes) | 6.60E−03 | 0.197 | 0.055 | 0.340 | | | |
| UC: Left-sided, | 4.08E−04 | 2.361 | 1.466 | 3.803 | | 0.01 | |
| Extensive (E2E3 vs E1) | | | | | | | |
| Surgery All (Yes) | 3.63E−19 | 1.715 | 1.524 | 1.931 | | | 8.45E−18 |
| Surgery CD (Yes) | 3.95E−04 | 1.307 | 1.127 | 1.516 | 1.32E−03 | | |
| Surgery UC (Yes) | 1.16E−20 | 2.714 | 2.200 | 3.348 | | 9.06E−16 | |

A multivariate analysis was run on the different categories. Table 9 shows the p values and odds ratios (parenthesis) of the different comparisons. An increased risk of EMs was associated with CD, although UC was associated with PSC. An increased risk of EMs was associated with female subjects, except male subjects has an increased risk of AS and PSC. An increased risk of EIMs was associated with being Jewish. An increased risk of EIMs was associated with an increased age of diagnosis for AS, GA and EIM7 and a degreased age of diagnosis for EN. An increased risk of EIMs was associated with smoking—for patients with ulcerative colitis, an increased risk of PS and SKIN3 was associated with smoking, for patients with CD, an increased risk of PA and EIM7 was associated with smoking. An increased with of EIM4s was associated with colonic disease location and extensive disease in UC (EIM6, EIM7, PSC). An increased risk of EIMs was associated with surgery, except for PS and eye-associated manifestations. For instance, a physician can make a treatment plan based on if a patient has an EIM or a risk of developing an EIM. In one instance, if a patient has a risk of developing psoriasis, then a physician would not treat with vedolizumab, but could treat with anti-TNF, ustekinumab, or tofacitinib.

TABLE 9
Clinical associations

| * ≤ 0.05 | | | | | | | | |
|---|---|---|---|---|---|---|---|---|
| ** ≤ 0.01 | | | | | | | | |
| *** ≤ 0.001 | Multivariate (OR) | | | | | | | |
| **** ≤ 0.0001 | EIM-6 | AS | EN | PG | PS | SKIN-3 | EYE | PSC |
| CD (Yes) | **** | **** | **** | *** | **** | **** | **** | **** |
| | (1.27) | (2.14) | (3.43) | (2.13) | (2.05) | (2.55) | (2.18) | (0.28) |
| UC_IBDU (yes) | **** | **** | **** | *** | **** | **** | **** | **** |
| | (0.79) | (0.47) | (0.29) | (0.47) | (0.9) | (0.39) | (0.46) | (3.61) |
| Sex (Female) | CD | CD * | CD | CD * | CD ** | CD | CD | CD * |
| | **** | (0.68) | **** | (1.56) | (1.53) | **** | **** | (0.54) |
| | (1.53) | | (4.34) | | | (2.53) | (1.76) | |
| | | | UC ** | UC * | | UC ** | UC * | |
| | | | (2.46) | (2.61) | | (1.81) | (1.66) | |
| Hispanic (Yes) | | | | | | | | |
| Jewish (Yes) | | | | | UC * | UC * | | |
| | | | | | (1.82) | (1.55) | | |
| Age at Diagnosis | | CD* | CD* | | | | | |
| | | (1.02) | (0.98) | | | | | |
| Age at Diagnosis: < 17 | | | | | | | | |
| years (Yes) | | | | | | | | |
| Age at Diagnosis: < 10 | | | | | | | | |
| years (Yes) | | | | | | | | |
| Current Smoking | | | | | UC * | UC ** | | |
| (Yes) | | | | | (1.78) | (1.96) | | |
| Family History (Yes) | | | | | | | | |
| CD: Upper GI (Yes) | | | | | | | | |
| CD: Any Colonic | **** | | * | ** | | ** | | ** |
| (L2L3 vs L1) | (1.92) | | (1.80) | (6.94) | | (2.60) | | (8.0) |
| CD: Any Small | | | | ** | | | | |
| Bowel (L1L3 vs L2) | | | | (0.46) | | | | |
| CD: Stricturing | | | | | | | | |
| (B2 vs B1) | | | | | | | | |
| CD: Penetrating | | | | | | | | |
| (B3 vs B1) | | | | | | | | |
| CD: Stricturing, | | | | | | | | **** |
| Penetrating (B2B3 vs B1) | | | | | | | | (0.27) |
| CD: Penetrating | | | | | | | | |
| vs Stricturing (B3 vs B2) | | | | | | | | |
| CD: Perianal (Yes) | | | **** | | | **** | | |
| UC: Left-sided, | * | | | | | | | ** |
| Extensive (E2E3 | (2.07) | | | | | | | (3.76) |
| vs E1) | | | | | | | | |
| Surgery | CD ** | | (IBD | CD | | (IBD | | CD |
| | (1.29) | | ****) | **** | | ****) | | **** |
| | | | | (3.30) | | | | (3.71) |
| | UC | UC | | | | | | UC |
| | **** | *** | | | | | | **** |
| | (2.51) | (2.61) | | | | | | (3.52) |

| | Multivariate (OR) | | |
|---|---|---|---|
| | PA | EIM-7 | >=2 any EIM-6 |
| CD (Yes) | **** (1.58) | **** (1.40) | **** (2.19) |
| UC_IBDU (Yes) | **** (0.63) | **** (0.72) | **** (0.46) |
| Sex (Female) | CD **** (1.69) | CD **** (1.53) | CD *** (1.86) |
| | UC **** (1.51) | UC * (1.20) | |
| Hispanic (Yes) | | | |
| Jewish (Yes) | | | |
| Age at Diagnosis | CD/UC **** | CD/UC *** | |
| | (1.02) | (1.01) | |
| Age at Diagnosis: < 17 years (Yes) | | | |
| Age at Diagnosis: < 10 years (Yes) | | | |
| Current Smoking (Yes) | CD **** (1.66) | CD ** (1.38) | |
| Family History (Yes) | CD/UC * | CD/UC */** | |
| | (1.2/1.31) | (1.21/1.33) | |
| CD: Upper GI (Yes) | | | |
| CD: Any Colonic (L2L3 vs L1) | **** (1.51) | **** (1.54) | *** (2.38) |
| CD: Any Small Bowel (L1L3 vs L2) | | | |
| CD: Stricturing (B2 vs B1) | | | |
| CD: Penetrating (B3 vs B1) | | | |
| CD: Stricturing, Penetrating | | | |
| (B2B3 vs B1) | | | |
| CD: Penetrating vs Stricturing | | | |
| (B3 vs B2) | | | |
| CD: Perianal (Yes) | | | |
| UC: Left-sided, Extensive (E2E3 vs E1) | | * (1.46) | |
| Surgery | CD **** (1.34) | CD **** | CD ** (1.61) |
| | | (1.47) | |
| | UC **** (1.59) | UC **** | UC *** (3.49) |
| | | (2.12) | |

Serological associations were also compared with the risk of EIMs. Specifically, Anti-Neutrophil Cytoplasmic Antibodies (ANCA), Anti-Saccharomyces Cerevisiae Antibodies (IgA, IgG ASCA), Anti-Outer membrane porin C (anti-OmpC), Anti-Pseudomonas fluorescens bacterial sequence 12 (anti-2), and Anti-Bacterial Flagellin (CBir1) were analyzed. In the Cedars cohort, 940 subjects had EIM4s and 2896 subjects did not. In the RISK cohort, 76 subjects has EIM4s and 474 did not. Serological associations are shown in Table 9A-9B.

TABLE 9A
Serological associations

| | | | | | |
|---|---|---|---|---|---|
| | | | | 95% CI | 95% CI |
| EIM_Pheno | Serology | Pval | OR | (lower) | (upper) |

CD

| | | | | | |
|---|---|---|---|---|---|
| AS | CBir1_pos | 2.73E−03 | 1.559 | 1.166 | 2.084 |
| EN | OmpC_level | 1.51E−02 | 1.008 | 1.002 | 1.015 |
| PY | ANCA_pos | 1.07E−02 | 2.245 | 1.206 | 4.179 |
| PY | ANCA_level | 4.05E−02 | 1.008 | 1.000 | 1.016 |
| PY | CBir1_pos | 2.79E−02 | 0.483 | 0.253 | 0.924 |
| PY | IgG_ASCA_pos | 4.62E−02 | 0.456 | 0.211 | 0.987 |
| PY | IgG_ASCAlevel | 3.47E−02 | 0.988 | 0.977 | 0.999 |
| PY | ASCA_pos | 2.58E−02 | 0.465 | 0.237 | 0.912 |
| PS | OmpC_pos | 2.19E−02 | 1.536 | 1.064 | 2.216 |
| PS | I2_pos | 4.15E−02 | 1.509 | 1.016 | 2.241 |
| SKIN3 | OmpC_level | 1.67E−02 | 1.006 | 1.001 | 1.011 |
| SKIN3 | OmpC_pos | 1.41E−02 | 1.427 | 1.074 | 1.895 |
| SKIN3 | I2_pos | 4.05E−02 | 1.369 | 1.014 | 1.850 |
| PSC | ANCA_pos | 3.37E−08 | 3.334 | 2.174 | 5.111 |
| PSC | ANCA_level | 7.27E−14 | 1.017 | 1.012 | 1.021 |
| PSC | CBir1_pos | 1.19E−03 | 0.466 | 0.293 | 0.739 |
| PSC | CBir1_level | 3.67E−03 | 0.988 | 0.979 | 0.996 |
| PSC | IgA_ASCA_pos | 4.50E−03 | 0.443 | 0.253 | 0.777 |
| PSC | IgA_ASCAlevel | 1.17E−03 | 0.981 | 0.970 | 0.993 |
| PSC | IgG_ASCA_pos | 1.29E−05 | 0.214 | 0.107 | 0.428 |
| PSC | IgG_ASCAlevel | 1.48E−05 | 0.978 | 0.968 | 0.988 |
| PSC | ASCA_pos | 8.96E−06 | 0.312 | 0.186 | 0.521 |
| PA | OmpC_pos | 1.39E−02 | 1.385 | 1.069 | 1.796 |
| PA | I2_pos | 1.89E−02 | 1.393 | 1.056 | 1.838 |
| PA | I2_level | 4.37E−02 | 1.003 | 1.000 | 1.006 |
| AS | CBir1_pos | 2.73E−03 | 1.559 | 1.166 | 2.084 |
| EN | OmpC_level | 1.51E−02 | 1.008 | 1.002 | 1.015 |
| PY | ANCA_pos | 1.07E−02 | 2.245 | 1.206 | 4.179 |
| PY | ANCA_level | 4.05E−02 | 1.008 | 1.000 | 1.016 |
| PY | CBir1_pos | 2.79E−02 | 0.483 | 0.253 | 0.924 |
| PY | IgG_ASCA_pos | 4.62E−02 | 0.456 | 0.211 | 0.987 |
| PY | IgG_ASCAlevel | 3.47E−02 | 0.988 | 0.977 | 0.999 |
| PY | ASCA_pos | 2.58E−02 | 0.465 | 0.237 | 0.912 |
| PS | OmpC_pos | 2.19E−02 | 1.536 | 1.064 | 2.216 |
| PS | I2_pos | 4.15E−02 | 1.509 | 1.016 | 2.241 |
| SKIN3 | OmpC_level | 1.67E−02 | 1.006 | 1.001 | 1.011 |
| SKIN3 | OmpC_pos | 1.41E−02 | 1.427 | 1.074 | 1.895 |
| SKIN3 | I2_pos | 4.05E−02 | 1.369 | 1.014 | 1.850 |
| PSC | ANCA_pos | 3.37E−08 | 3.334 | 2.174 | 5.111 |
| PSC | ANCA_level | 7.27E−14 | 1.017 | 1.012 | 1.021 |
| PSC | CBir1_pos | 1.19E−03 | 0.466 | 0.293 | 0.739 |
| PSC | CBir1_level | 3.67E−03 | 0.988 | 0.979 | 0.996 |
| PSC | IgA_ASCA_pos | 4.50E−03 | 0.443 | 0.253 | 0.777 |
| PSC | IgA_ASCAlevel | 1.17E−03 | 0.981 | 0.970 | 0.993 |
| PSC | IgG_ASCA_pos | 1.29E−05 | 0.214 | 0.107 | 0.428 |
| PSC | IgG_ASCAlevel | 1.48E−05 | 0.978 | 0.968 | 0.988 |
| PSC | ASCA_pos | 8.96E−06 | 0.312 | 0.186 | 0.521 |
| PA | OmpC_pos | 1.39E−02 | 1.385 | 1.069 | 1.796 |
| PA | I2_pos | 1.89E−02 | 1.393 | 1.056 | 1.838 |
| PA | I2_level | 4.37E−02 | 1.003 | 1.000 | 1.006 |
| EIM6 | ANCA_level | 6.30E−03 | 1.004 | 1.001 | 1.007 |
| EIM6 | I2_pos | 6.30E−03 | 1.359 | 1.091 | 1.694 |
| EIM6 | I2_level | 1.48E−02 | 1.003 | 1.001 | 1.005 |
| EIM6 | OmpC_pos | 3.45E−02 | 1.264 | 1.017 | 1.571 |
| EIM6 | OmpC_level | 1.74E−02 | 1.005 | 1.001 | 1.009 |
| EIM6 | IgG_ASCA_pos | 3.98E−03 | 0.733 | 0.594 | 0.906 |
| EIM6 | IgG_ASCA_level | 1.36E−03 | 0.996 | 0.993 | 0.998 |
| EIM6 | ASCA_pos | 1.14E−02 | 0.777 | 0.639 | 0.945 |
| EIM7 | ANCA_level | 4.33E−02 | 1.003 | 1.000 | 1.006 |
| EIM7 | I2_pos | 1.01E−04 | 1.476 | 1.213 | 1.796 |
| EIM7 | I2_level | 6.14E−04 | 1.004 | 1.002 | 1.006 |
| EIM7 | OmpC_pos | 4.81E−03 | 1.322 | 1.089 | 1.604 |
| EIM7 | OmpC_level | 1.01E−03 | 1.006 | 1.003 | 1.010 |
| EIM7 | IgG_ASCA_pos | 3.15E−03 | 0.759 | 0.631 | 0.911 |
| EIM7 | IgG_ASCA_level | 2.64E−03 | 0.996 | 0.994 | 0.999 |
| EIM7 | ASCA_pos | 2.07E−02 | 0.818 | 0.690 | 0.970 |
| >=2 any | ANCA_level | 4.26E−03 | 1.008 | 1.003 | 1.014 |
| EIM-6 | | | | | |
| >=2 any | IgG_ASCA_level | 3.45E−02 | 0.993 | 0.986 | 0.999 |
| EIM-6 | | | | | |

Cedars

| | | | | | |
|---|---|---|---|---|---|
| EIM6 | QSS | 0.08 | 1.034 | 0.996 | 1.074 |
| >=2 any | QSS | 0.50 | 0.973 | 0.900 | 1.053 |
| EIM-6 | | | | | |
| AS | QSS | 1.90E−02 | 1.067 | 1.011 | 1.126 |
| EN | QSS | 2.73E−02 | 1.104 | 1.011 | 1.205 |
| PY | QSS | 1.48E−02 | 0.856 | 0.755 | 0.970 |
| PS | QSS | 0.12 | 1.057 | 0.986 | 1.132 |
| SKIN3 | QSS | 0.23 | 1.032 | 0.980 | 1.087 |
| EYE | QSS | 0.62 | 0.977 | 0.892 | 1.070 |
| PSC | QSS | 8.00E−03 | 0.896 | 0.827 | 0.972 |
| PA | QSS | 3.75E−02 | 1.052 | 1.003 | 1.104 |
| EIM7 | QSS | 1.85E−03 | 1.055 | 1.020 | 1.092 |

TABLE 9B
UC serological associations

| | | | | | |
|---|---|---|---|---|---|
| | | | | 95% CI | 95% CI |
| EIM_Pheno | Serology | Pval | OR | (lower) | (upper) |

UC

| | | | | | |
|---|---|---|---|---|---|
| EN | OmpC_level | 4.43E−02 | 3.238 | 1.031 | 10.174 |
| PA | ASCA_pos | 2.53E−02 | 0.447 | 0.221 | 0.905 |
| PSC | CBir1_pos | 4.37E−02 | 1.559 | 1.013 | 2.401 |
| PSC | CBir1_level | 2.99E−03 | 1.008 | 1.003 | 1.014 |
| PSC | I2_level | 2.11E−02 | 1.007 | 1.001 | 1.013 |
| EN | OmpC_level | 4.43E−02 | 3.238 | 1.031 | 10.174 |
| PA | ASCA_pos | 2.53E−02 | 0.447 | 0.221 | 0.905 |
| PSC | CBir1_pos | 4.37E−02 | 1.559 | 1.013 | 2.401 |
| PSC | CBir1_level | 2.99E−03 | 1.008 | 1.003 | 1.014 |
| PSC | I2_level | 2.11E−02 | 1.007 | 1.001 | 1.013 |
| EN | OmpC_level | 4.43E−02 | 3.238 | 1.031 | 10.174 |
| PA | ASCA_pos | 2.53E−02 | 0.447 | 0.221 | 0.905 |
| PSC | CBir1_pos | 4.37E−02 | 1.559 | 1.013 | 2.401 |
| PSC | CBir1_level | 2.99E−03 | 1.008 | 1.003 | 1.014 |
| PSC | I2_level | 2.11E−02 | 1.007 | 1.001 | 1.013 |
| EIM6 | CBir1_level | 2.60E−03 | 1.008 | 1.003 | 1.013 |
| EIM7 | CBir1_level | 1.62E−02 | 1.006 | 1.001 | 1.011 |
| >=2 any | I2_pos | 2.18E−02 | 2.528 | 1.144 | 5.584 |
| EIM-6 | | | | | |

Cedars

| | | | | | |
|---|---|---|---|---|---|
| EIM6 | 7.35E−04 | QSS | 1.118 | 1.048 | 1.192 |
| >=2 any | 1.70E−02 | QSS | 1.206 | 1.034 | 1.406 |
| EIM-6 | | | | | |
| AS | 0.07 | QSS | 1.115 | 0.992 | 1.252 |
| EN | 0.14 | QSS | 1.204 | 0.943 | 1.538 |
| PY | 0.22 | QSS | 1.241 | 0.876 | 1.758 |
| PS | 0.88 | QSS | 1.013 | 0.856 | 1.199 |
| SKIN3 | 0.13 | QSS | 1.106 | 0.970 | 1.261 |
| EYE | 0.06 | QSS | 1.295 | 0.990 | 1.695 |
| PSC | 6.80E−03 | QSS | 1.111 | 1.029 | 1.198 |
| PA | 0.05 | QSS | 1.111 | 0.999 | 1.235 |
| EIM7 | 4.15E−04 | QSS | 1.117 | 1.051 | 1.188 |

A genome wide association analysis was performed to identify relevant loci. The following associations were identified are shown in Tables 10A-10E.

TABLE 10A
Genomic associations

| | | | | | GW | | | | | |
|---|---|---|---|---|---|---|---|---|---|---|
| | | | | | SNPs | | | | | |
| | | | hg19 | Candidate | (LD | Effect | | Beta | 95% | MAF |
| Phenotype | CHR | SNP | (Mb) | Gene(s) | r2 > 0.5) | Allele | Pval | (OR) | CI | (%) |
| SKIN-3 | 5 | rs80079682 | 173.29 | BOD1, CPEB4 | | A | 2.71E−08 | 0.418 | 0.271- | 15.7 |
| | | | | | | | | (1.52) | 0.565 | |
| AS-SI | 6 | rs6905036 | 31.27 | HLA-C, HLA-B | 3 | G | 1.36E−15 | 0.916 | 0.691- | 6.3 |
| | | | | | | | | (2.5) | 1.140 | |
| EYE | 6 | rs4349859 | 31.37 | HLA-B, MICA | | A | 1.99E−08 | 1.283 | 0.835- | 4.2 |
| | | | | | | | | (3.61) | 1.731 | |
| EIM-6 | 6 | rs4349859 | 31.37 | HLA-B, MICA | | A | 8.35E−09 | 0.788 | 0.520- | 4.2 |
| | | | | | | | | (2.2) | 1.056 | |
| AS-SI | 6 | rs2844510 | 31.41 | MICA, HCP5 | | A | 9.89E−13 | 0.745 | 0.541- | 10.1 |
| | | | | | | | | (2.11) | 0.950 | |
| PSC | 6 | rs9276456 | 32.72 | HLA- | 36 | G | 2.71E−10 | 1.014 | 0.699- | 18.1 |
| | | | | DQA2, HLA- | | | | (2.76) | 1.328 | |
| | | | | DQB2, | | | | | | |
| | | | | MIR3135B | | | | | | |

TABLE 10B
Genetic associations

| | | | | | Multiple | | | | | | |
|---|---|---|---|---|---|---|---|---|---|---|---|
| | | | | | assoc | | | | | | |
| | | | hg19 | Candidate | (LD | Effect | | | | | |
| Phenotype | CHR | SNP | (Mb) | Gene(s) | r2 > 0.5) | Allele | Pval | Beta | L95 | U95 | MAF |
| EIM6 | 6 | rs4463302 | 31.34 | HLA-B; MICA | * | G | 1.50E−07 | 0.595 | 0.373 | 0.817 | 6.42% |
| EIM6 | 6 | rs4418214 | 31.39 | MICA; HCP5 | | G | 1.03E−07 | 0.569 | 0.359 | 0.779 | 8.07% |
| EIM6 | 7 | rs2069835 | 22.77 | IL6 | | G | 6.14E−06 | 0.290 | 0.164 | 0.416 | 7.36% |
| EIM7 | 8 | rs61199332 | 11.43 | BLK; LINC28 | | A | 9.13E−06 | −0.442 | −0.638 | −0.247 | 2.56% |
| EIM7 | 9 | rs7857730 | 5.08 | JAK2 | * | C | 2.71E−06 | 0.195 | 0.113 | 0.276 | 46.33% |
| EIM7 | 11 | rs497871 | 114.35 | REXO2; | | G | 7.94E−06 | 0.203 | 0.114 | 0.292 | 11.16% |
| | | | | NXPE1 | | | | | | | |

TABLE 10C
Genetic associations

| Phenotype | HLA 2-digit | Pval | OR |
|---|---|---|---|
| AS | HLA_B_27 | 1.66E−08 | 2.74 |
| AS | HLA_C_2 | 2.99E−04 | 2.01 |
| PS | HLA_DPB1_10 | 4.03E−05 | 3.01 |
| PS | HLA_DRB1_14 | 1.98E−04 | 2.40 |

TABLE 10D
Additional Genetic associations

| | | | | | Multiple | | | | | | |
|---|---|---|---|---|---|---|---|---|---|---|---|
| | | | | | assoc | | | | | | |
| | | | hg19 | Candidate | (LD | Effect | | | | | |
| PHENO | CHR | SNP | (Mb) | Gene(s) | r2 > 0.5) | Allele | Pval | Beta | L95 | U95 | MAF |
| AS | 6 | rs2516514 | 31.45 | HCG26; MICB | | G | 2.15 | 0.457 | 0.284 | 0.630 | 21.39% |
| | | | | | | | E−07 | | | | |
| AS | 2 | rs17030062 | 65.46 | ACTR2 | | A | 8.76 | 0.892 | 0.499 | 1.285 | 1.39% |
| | | | | | | | E−06 | | | | |
| EN | 21 | rs9305694 | 41.54 | DSCAM | | A | 1.36 | 0.928 | 0.552 | 1.305 | 7.08% |
| | | | | | | | E−06 | | | | |
| PG | 4 | rs139009610 | 123.57 | IL21-AS1 | | C | 8.04 | 1.275 | 0.715 | 1.834 | 2.93% |
| | | | | | | | E−06 | | | | |
| PS | 6 | rs28732100 | 31.10 | PSORS1C1 | * | A | 4.24 | 1.043 | 0.639 | 1.447 | 4.87% |
| | | | | | | | E−07 | | | | |
| PS | 6 | rs12199223 | 31.24 | HLA-C; HLA-B | * | T | 8.69 | 0.561 | 0.338 | 0.785 | 9.87% |
| | | | | | | | E−07 | | | | |
| PS | 6 | rs1265181 | 31.16 | PSORS1C3; HCG27 | * | G | 2.51 | 0.538 | 0.314 | 0.763 | 20.20% |
| | | | | | | | E−06 | | | | |
| PS | 2 | rs13417109 | 207.52 | DYTN | * | T | 3.19 | 1.099 | 0.636 | 1.561 | 1.12% |
| | | | | | | | E−06 | | | | |
| PS | 2 | rs17026757 | 102.81 | IL1RL2 | * | C | 3.96 | 0.535 | 0.308 | 0.762 | 8.26% |
| | | | | | | | E−06 | | | | |
| PS | 14 | rs117670930 | 81.11 | CEP128 | | G | 4.44 | 0.847 | 0.485 | 1.209 | 2.30% |
| | | | | | | | E−06 | | | | |
| SKIN3 | 4 | rs115994059 | 102.89 | BANK1 | * | A | 3.05 | 0.574 | 0.333 | 0.815 | 3.12% |
| | | | | | | | E−06 | | | | |
| SKIN3 | 12 | rs7297515 | 126.90 | LINC939; | * | G | 4.04 | 1.033 | 0.594 | 1.473 | 1.91% |
| | | | | LOC1128554 | | | E−06 | | | | |
| SKIN3 | 5 | rs13166683 | 120.92 | PRR16; FTMT | | A | 5.20 | 0.625 | 0.356 | 0.894 | 6.56% |
| | | | | | | | E−06 | | | | |
| SKIN3 | 5 | rs62376929* | 173.29 | BOD1; CPEB4 | * | A | 8.62 | 0.293 | 0.164 | 0.422 | 15.80% |
| | | | | | | | E−06 | | | | |
| EYE | 6 | rs4418214 | 31.39 | MICA; HCP5 | | G | 5.56 | 1.007 | 0.612 | 1.401 | 8.07% |
| | | | | | | | E−07 | | | | |
| EYE | 6 | rs3819299 | 31.32 | HLA-B | * | C | 5.03 | 0.945 | 0.539 | 1.350 | 5.07% |
| | | | | | | | E−06 | | | | |
| EYE | 2 | rs2373969 | 41.21 | SLC8A1; Mir_584 | | G | 5.04 | 0.502 | 0.287 | 0.718 | 17.01% |
| | | | | | | | E−06 | | | | |
| EYE | 6 | rs4151651 | 31.92 | CFB | * | A | 8.77 | 0.700 | 0.391 | 1.009 | 4.42% |
| | | | | | | | E−06 | | | | |
| EYE | 6 | rs9366775 | 31.24 | HLA-C | | A | 9.29 | 0.941 | 0.525 | 1.356 | 3.47% |
| | | | | | | | E−06 | | | | |
| PSC | 6 | rs2858884 ** | 32.70 | HLA- | * | C | 5.72 | 0.858 | 0.548 | 1.168 | 22.49% |
| | | | | DQB1; HLA- | | | E−08 | | | | |
| | | | | DQA2 | | | | | | | |
| PSC | 6 | rs2858319 | 32.66 | HLA- | * | A | 2.11 | 0.807 | 0.502 | 1.111 | 35.02% |
| | | | | DQB1; HLA- | | | E−07 | | | | |
| | | | | DQA2 | | | | | | | |
| PSC | 6 | rs6917611 | 32.77 | HLA- | | G | 4.25 | 0.753 | 0.461 | 1.044 | 44.41% |
| | | | | DQB2; HLA-DOB | | | E−07 | | | | |
| PSC | 6 | rs6930571 | 32.38 | BTNL2; HLA- | * | A | 4.64 | 0.726 | 0.444 | 1.009 | 13.73% |
| | | | | DRA | | | E−07 | | | | |
| PSC | 6 | rs389419 | 29.52 | LINC115; UBD | * | A | 2.38 | −1.473 | −2.085 | −0.861 | 7.41% |
| | | | | | | | E−06 | | | | |
| PSC | 17 | rs76558762 | 26.05 | LGALS9; NOS2 | | C | 4.37 | 1.168 | 0.670 | 1.667 | 1.78% |
| | | | | | | | E−06 | | | | |
| PSC | 12 | rs7956721 | 40.79 | MUC19 | * | A | 5.63 | 0.321 | 0.182 | 0.459 | 30.06% |
| | | | | | | | E−06 | | | | |
| PSC | 16 | rs887864 | 11.16 | CLEC16A | * | G | 5.75 | −0.338 | −0.483 | −0.192 | 36.79% |
| | | | | | | | E−06 | | | | |
| PSC | 6 | rs9276424 | 32.71 | HLA-DQA2 | * | G | 8.25 | 0.662 | 0.371 | 0.954 | 38.17% |
| | | | | | | | E−06 | | | | |
| PA | 5 | rs10056322 | 35.99 | UGT3A1 | | G | 1.05 | −1.287 | −1.804 | −0.771 | 1.88% |
| | | | | | | | E−06 | | | | |
| PA | 7 | rs6461986 | 27.16 | HOXA3 | | T | 2.16 | 0.637 | 0.374 | 0.901 | 1.77% |
| | | | | | | | E−06 | | | | |
| PA | 2 | rs13421864 | 231.21 | SP14L | | C | 9.34 | 0.471 | 0.263 | 0.680 | 3.17% |
| | | | | | | | E−06 | | | | |

TABLE 10E
ChX Genetic associations

| | | | | | | | | | | | | Sex- | |
|---|---|---|---|---|---|---|---|---|---|---|---|---|---|
| | | | | | | | | | | | | Stratified | |
| | | | | | | | | XCI_{complete} | | XCI_{escape} | | F_{comb} | S_{comb} |
| SNP | Gene | BP | MAF | F-MAF | M-MAF | Phenotype | A1 | Pval | Beta | Pval | Beta | Pval | Pval |
| 1 kg_X_ | GAB3 | 153.94 | 0.022 | 0.033 | 0.002 | PA | A | 1.69 | −0.744 | 1.0 | −0.765 | 2.9 | 6.0 |
| 153591526 | | | | | | | | E−07 | | 3E−07 | | 0E−07 | 3E−05 |
| rs7877788 | ARSF | 2.98 | 0.036 | 0.035 | 0.038 | PA | G | 7.51 | −0.382 | 3.5 | −0.523 | 1.2 | 3.8 |
| | | | | | | | | E−05 | | 2E−05 | | 9E−04 | 8E−05 |

### Example 3: Modeling Primary Sclerosing Cholangitis in Crohn's Disease

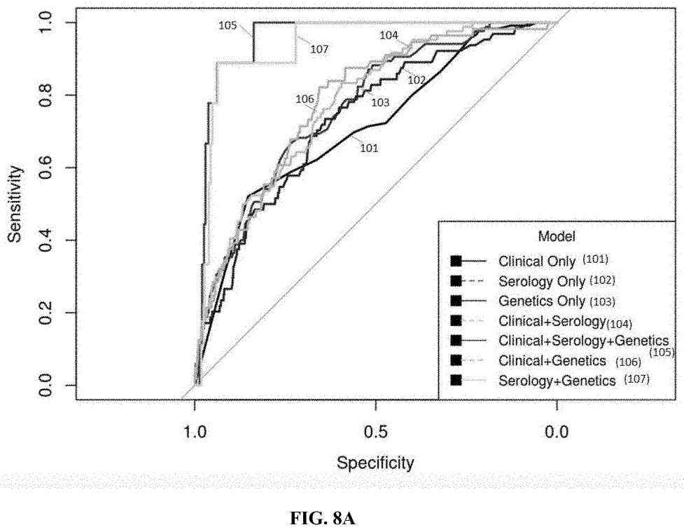

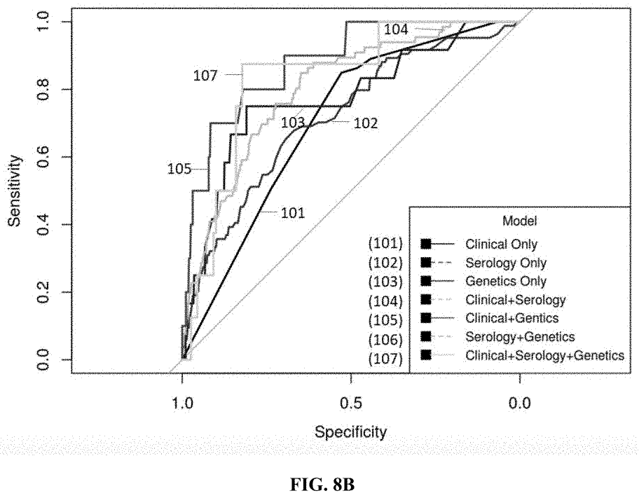

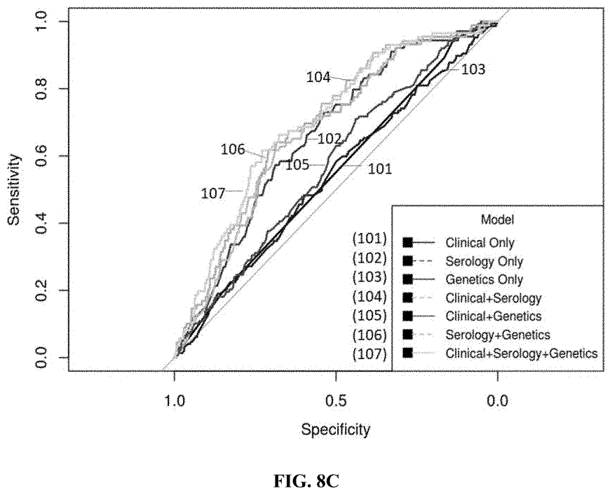

A model is generated to predict PSC in CD. The model variables include sex, any colonic disease, surgery, ANCA level ASCA positivity and 7 SNPS with a p value of less than 1×10⁻⁵. The results of the model are shown in FIGS. 8A-8C and Tables 11A-11C. Model A is depicted in FIG. 8A Model B, as depicted in FIG. 8B shows the results using only the updated Cedars dataset. Model C, depicted in FIG. 8C is a null model using only the updated Cedars dataset. The models tested a comparison of clinical data only (101), serology only (102), genetics only (103), a combination of clinical and serology data (104), a combination of clinical, serology, and genetic markers (105), a combination of clinical and genetic markers (106) and a combination of serology and genetic markers (107).

TABLE 11A
Efficacy of the tested model in FIG. 8A

| | AUC | 95% CI |
|---|---|---|
| Clinical Only | 0.7108 | 0.6622-0.7593 |
| Serology Only | 0.7636 | 0.7157-0.8114 |
| Genetics Only | 0.7319 | 0.6721-0.7918 |
| Clinical + Serology | 0.7764 | 0.7316-0.8213 |
| Clinical + Genetics | 0.7796 | 0.7237-0.8355 |
| Serology + Genetics | 0.9353 | 0.8807-0.9899 |
| Clinical + Serology + Genetics | 0.9554 | 0.9231-0.9878 |

TABLE 11B
Efficacy of the tested model in FIG. 8B

| | AUC | 95% CI |
|---|---|---|
| Clinical Only | 0.704 | 0.6552-0.7528 |
| Serology Only | 0.7168 | 0.6598-0.7739 |
| Genetics Only | 0.7726 | 0.6188-0.9264 |
| Clinical + Serology | 0.8018 | 0.751-0.8525 |
| Clinical + Genetics | 0.8795 | 0.7804-0.9786 |
| Serology + Genetics | 0.8324 | 0.7067-0.9581 |
| Clinical + Serology + Genetics | 0.909 | 0.8452-0.9728 |

TABLE 11C
Efficacy of the “null model” in FIG. 8C

| | AUC | 95% CI |
|---|---|---|
| Clinical Only B (multivar) | 0.5466 | 0.5088-0.5845 |
| Serology Only B (multivar) | 0.6611 | 0.6089-0.7134 |
| Genetics Only 2 (reading/writing) | 0.5382 | 0.4911-0.5852 |
| Clinical + Serology B (multivar) | 0.683 | 0.6322-0.7337 |
| Clinical B + Genetics 2 | 0.574 | 0.5289-0.6192 |
| Serology B + Genetics 2 | 0.6798 | 0.627-0.7327 |
| Clinical B + Serology B + Genetics 2 | 0.7018 | 0.6493-0.7544 |

### Example 4: Phase I Clinical Trial

A phase 1 clinical trial is performed to evaluate the safety, tolerability, pharmacokinetics and pharmacodynamics of a therapeutic agent on subjects having Crohn's disease (CD).

Single ascending dose (SAD) arms: Subjects in each group (subjects are grouped based on the presence of a genotype comprising at least one, two, or three polymorphism(s) selected from Table 1 and subjects without the presence of the genotype) receive either a single dose of the antibody or a placebo. Exemplary doses are 1, 3, 10, 30, 100, 300, 600 and 800 mg of antibody. Safety monitoring and PK assessments are performed for a predetermined time. Based on evaluation of the PK data, and if the antibody is deemed to be well tolerated, dose escalation occurs, either within the same groups or a further group of healthy subjects. Dose escalation continues until the maximum dose has been attained unless predefined maximum exposure is reached or intolerable side effects become apparent.

Multiple ascending dose (MAD) arms: Subjects in each group (subjects are grouped based on the same criteria as above) receive multiple doses of the antibody or a placebo. The dose levels and dosing intervals are selected as those that are predicted to be safe from the SAD data. Dose levels and dosing frequency are chosen to achieve therapeutic drug levels within the systemic circulation that are maintained at steady state for several days to allow appropriate safety parameters to be monitored. Samples are collected and analyzed to determination PK profiles.

Inclusion Criteria: Healthy subjects of non-childbearing potential between the ages of 18 and 55 years. Healthy is defined as no clinically relevant abnormalities identified by a detailed medical history, full physical examination, including blood pressure and pulse rate measurement, 12 lead ECG and clinical laboratory tests. Female subjects of non-childbearing potential must meet at least one of the following criteria: (1) achieved postmenopausal status, defined as: cessation of regular menses for at least 12 consecutive months with no alternative pathological or physiological cause; and have a serum follicle stimulating hormone (FSH) level within the laboratory's reference range for postmenopausal females; (2) have undergone a documented hysterectomy and/or bilateral oophorectomy; (3) have medically confirmed ovarian failure. All other female subjects (including females with tubal ligations and females that do NOT have a documented hysterectomy, bilateral oophorectomy and/or ovarian failure) will be considered to be of childbearing potential. Body Mass Index (BMI) of 17.5 to 30.5 kg/m2; and a total body weight >50 kg (110 lbs). Evidence of a personally signed and dated informed consent document indicating that the subject (or a legal representative) has been informed of all pertinent aspects of the study.

Two groups of CD patients are selected: patients having the genotype described herein, and patients without the genotype. For example, the genotype may comprise rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, rs9276424, rs10056322, rs6461986, and rs13421864.

Exclusion Criteria: Evidence or history of clinically significant hematological, renal, endocrine, pulmonary, gastrointestinal, cardiovascular, hepatic, psychiatric, neurologic, or allergic disease (including drug allergies, but excluding untreated, asymptomatic, seasonal allergies at time of dosing). Subjects with a history of or current positive results for any of the following serological tests: Hepatitis B surface antigen (HBsAg), Hepatitis B core antibody (HBcAb), anti-Hepatitis C antibody (HCV Ab) or human immunodeficiency virus (HIV). Subjects with a history of allergic or anaphylactic reaction to a therapeutic drug. Treatment with an investigational drug within 30 days (or as determined by the local requirement, whichever is longer) or 5 half-lives or 180 days for biologics preceding the first dose of study medication. Pregnant females; breastfeeding females; and females of childbearing potential.

Primary Outcome Measures: Incidence of dose limiting or intolerability treatment related adverse events (AEs) [Time Frame: 12 weeks]. Incidence, severity and causal relationship of treatment emergent AEs (TEAEs) and withdrawals due to treatment emergent adverse events [Time Frame: 12 weeks]. Incidence and magnitude of abnormal laboratory findings [Time Frame: 12 weeks]. Abnormal and clinically relevant changes in vital signs, blood pressure (BP) and electrocardiogram (ECG) parameters [Time Frame: 12 weeks]. Extra-intestinal manifestations are also measured.

Secondary Outcome Measures: Single Ascending Dose: Maximum Observed Plasma Concentration (Cmax) [Time Frame: 12 weeks]. Single Ascending Dose: Time to Reach Maximum Observed Plasma Concentration (Tmax) [Time Frame: 12 weeks]. Single Ascending Dose: Area under the plasma concentration-time profile from time zero to 14 days (AUC14 days) [Time Frame: 12 weeks]. Single Ascending Dose: Area under the plasma concentration-time profile from time zero extrapolated to infinite time (AUCinf) [Time Frame: 12 weeks]. Single Ascending Dose: Area under the plasma concentration-time profile from time zero to the time of last quantifiable concentration (AUClast) [Time Frame: 12 weeks]. Single Ascending Dose: Dose normalized maximum plasma concentration (Cmax[dn]) [Time Frame: 12 weeks]. Single Ascending Dose: Dose normalized area under the plasma concentration-time profile from time zero extrapolated to infinite time (AUCinf[dn]) [Time Frame: 12 weeks]. Single Ascending Dose: Dose normalized area under the plasma concentration-time profile from time zero to the time of last quantifiable concentration (AUClast[dn]) [Time Frame: 12 weeks]. Single Ascending Dose: Plasma Decay Half-Life (t½) [Time Frame: 12 weeks]. Plasma decay half-life is the time measured for the plasma concentration to decrease by one half. Single Ascending Dose: Mean residence time (MRT) [Time Frame: 12 weeks]. Single Ascending Dose: Volume of Distribution at Steady State (Vss) [Time Frame: 6 weeks]. Volume of distribution is defined as the theoretical volume in which the total amount of drug would need to be uniformly distributed to produce the desired blood concentration of a drug. Steady state volume of distribution (Vss) is the apparent volume of distribution at steady-state. Single Ascending Dose: Systemic Clearance (CL) [Time Frame: 6]. CL is a quantitative measure of the rate at which a drug substance is removed from the body.

Multiple Ascending Dose First Dose: Maximum Observed Plasma Concentration (Cmax) [Time Frame: 12 weeks]. Multiple Ascending Dose First Dose: Time to Reach Maximum Observed Plasma Concentration (Tmax) [Time Frame: 12 weeks]. Multiple Ascending Dose First Dose: Area under the plasma concentration-time profile from time zero to time τ, the dosing interval where r=2 weeks (AUCτ) [Time Frame: 12 weeks]. Multiple Ascending Dose First Dose: Dose normalized maximum plasma concentration (Cmax[dn]) [Time Frame: 12 weeks]. Multiple Ascending Dose First Dose: Dose normalized Area under the plasma concentration-time profile from time zero to time τ, the dosing interval where i=2 weeks (AUCτ [dn]) [Time Frame: 12 weeks]. Plasma Decay Half-Life (t½) [Time Frame: 12 weeks]. Plasma decay half-life is the time measured for the plasma concentration to decrease by one half. Multiple Ascending Dose First Dose: Mean residence time (MRT) [Time Frame: 12 weeks]. Apparent Volume of Distribution (Vz/F) [Time Frame: 12 weeks]. Volume of distribution is defined as the theoretical volume in which the total amount of drug would need to be uniformly distributed to produce the desired plasma concentration of a drug. Apparent volume of distribution after oral dose (Vz/F) is influenced by the fraction absorbed. Multiple Ascending Dose First Dose: Volume of Distribution at Steady State (Vss) [Time Frame: 12 weeks]. Volume of distribution is defined as the theoretical volume in which the total amount of drug would need to be uniformly distributed to produce the desired blood concentration of a drug. Steady state volume of distribution (Vss) is the apparent volume of distribution at steady-state. Multiple Ascending Dose First Dose: Apparent Oral Clearance (CL/F) [Time Frame: 12 weeks]. Clearance of a drug is a measure of the rate at which a drug is metabolized or eliminated by normal biological processes. Clearance obtained after oral dose (apparent oral clearance) is influenced by the fraction of the dose absorbed. Clearance is estimated from population pharmacokinetic (PK) modeling. Drug clearance is a quantitative measure of the rate at which a drug substance is removed from the blood. Multiple Ascending Dose First Dose: Systemic Clearance (CL) [Time Frame: 12 weeks]. CL is a quantitative measure of the rate at which a drug substance is removed from the body.

Multiple Ascending Dose Multiple Dose: Maximum Observed Plasma Concentration (Cmax) [Time Frame: 12 weeks]. Multiple Ascending Dose Multiple Dose: Time to Reach Maximum Observed Plasma Concentration (Tmax) [Time Frame: 12 weeks]. Multiple Ascending Dose Multiple Dose: Area under the plasma concentration-time profile from time zero to time τ, the dosing interval where r=2 weeks (AUCτ) [Time Frame: 12 weeks]. Multiple Ascending Dose Multiple Dose: Dose normalized maximum plasma concentration (Cmax[dn]) [Time Frame: 12 weeks]. Multiple Ascending Dose Multiple Dose: Dose normalized Area under the plasma concentration-time profile from time zero to time τ, the dosing interval where r=2 weeks (AUCτ [dn]) [Time Frame: 12 weeks]. Multiple Ascending Dose Multiple Dose: Plasma Decay Half-Life (t½) [Time Frame: 12 weeks]. Plasma decay half-life is the time measured for the plasma concentration to decrease by one half. Multiple Ascending Dose Multiple Dose: Apparent Volume of Distribution (Vz/F) [Time Frame: 12 weeks]. Volume of distribution is defined as the theoretical volume in which the total amount of drug would need to be uniformly distributed to produce the desired plasma concentration of a drug. Apparent volume of distribution after oral dose (Vz/F) is influenced by the fraction absorbed. Multiple Ascending Dose Multiple Dose: Volume of Distribution at Steady State (Vss) [Time Frame: 12 weeks]. Volume of distribution is defined as the theoretical volume in which the total amount of drug would need to be uniformly distributed to produce the desired blood concentration of a drug. Steady state volume of distribution (Vss) is the apparent volume of distribution at steady-state.

Multiple Ascending Dose Multiple Dose: Apparent Oral Clearance (CL/F) [Time Frame: 12 weeks]. Clearance of a drug is a measure of the rate at which a drug is metabolized or eliminated by normal biological processes. Clearance obtained after oral dose (apparent oral clearance) is influenced by the fraction of the dose absorbed. Clearance was estimated from population pharmacokinetic (PK) modeling. Drug clearance is a quantitative measure of the rate at which a drug substance is removed from the blood. Multiple Ascending Dose Multiple Dose: Systemic Clearance (CL) [Time Frame: 12 weeks]. CL is a quantitative measure of the rate at which a drug substance is removed from the body. Multiple Ascending Dose Multiple Dose: Minimum Observed Plasma Trough Concentration (Cmin) [Time Frame: 12 weeks]. Multiple Ascending Dose Multiple Dose: Average concentration at steady state (Cav) [Time Frame: 12 weeks]. Multiple Ascending Dose Multiple Dose: Observed accumulation ratio (Rac) [Time Frame: 12 weeks]. Multiple Ascending Dose Multiple Dose: Peak to trough fluctuation (PTF) [Time Frame: 12 weeks]. Multiple Ascending Dose Additional Parameter: estimate of bioavailability (F) for subcutaneous administration at the corresponding intravenous dose [Time Frame: 12 weeks]. Immunogenicity for both Single Ascending Dose and Multiple Ascending Dose: Development of anti-drug antibodies (ADA) [Time Frame: 12 weeks].

### Example 5: Phase 1B Clinical Trial

A phase 1b open label clinical trial is performed to evaluate efficacy of a therapeutic agent antibody on subjects having CD.

Arms: 10 patients positive for genotypes comprising at least one polymorphism(s) provided in Table 1 are administered the antibody. 10 patients negative for the genotype are administered the therapeutic agent. Patients are monitored in real-time. Central ready of endoscopy and biopsy is employed, with readers blinded to point of time of treatment and endpoints. Patients are also monitored for extra-intestinal manifestations.

For example, the genotypes may comprise rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, rs9276424, rs10056322, rs6461986, and rs13421864.

Inclusion Criteria: Two groups of patients are selected: subject with the genotype described herein, and patients without the genotype.

Primary Outcome Measures: Simple Endoscopic Score for Crohn's Disease (SESCD), Crohn's Disease Activity Index (CDAI), and Patient Reported Outcome (PRO). If risk either positive group shows 50% reduction from baseline, a Phase 2a clinical trial is performed.

Inclusion Criteria: PRO entry criteria: Abdominal pain score of 2 or more and/or stool frequency score of 4 or more. Primary outcome would be pain core of 0 or 1 and stool frequency score of 3 or less with no worsening from baseline. Endoscopy entry criteria: SESCD ileum only entry at score of 4 and 6 if colon is involved. Primary endoscopic outcome is 40-50% delta of mean SESCD.

### Example 6: Phase 2A Clinical Trial

A phase 2a clinical trial is performed to evaluate the efficacy of a therapeutic in patients with CD. Optionally, the patients are positive for a genotype comprising at least one, two, or at least three, polymorphism(s) provided in Table 1. Patients are also monitored for development of an extra-intestinal manifestation.

Arms: 40 patients per arm (antibody and placebo arms) are treated with antibody or placebo for 12 weeks. An interim analysis is performed after 20 patients from each group are treated at the highest dose to look for a 40-50% delta between placebo and treated group in primary outcome (50% reduction from baseline in SESCD, CDAI, and PRO).

Primary Outcome Measures: Simple Endoscopic Score for Crohn's Disease (SESCD), Crohn's Disease Activity Index (CDAI), and Patient Reported Outcome (PRO).

Inclusion Criteria: PRO entry criteria: Abdominal pain score of 2 or more and/or stool frequency score of 4 or more. Primary outcome would be pain core of 0 or 1 and stool frequency score of 3 or less with no worsening from baseline. Endoscopy entry criteria: SESCD ileum only entry at score of 4 and 6 if colon is involved. Primary endoscopic outcome is 40-50% delta of mean SESCD.

### Example 7: Treating Crohn's Disease (CD) in a Subject

CD is treated in a subject, by first, determining the genotypes of the subject. Optionally, the subject is, or is susceptible to, non-response to the induction of certain therapies such as anti-TNF, steroids, or immunomodulators, or loses response to such therapies after a period of time. A sample of whole blood is obtained from the subject. An assay is performed on the sample obtained from the subject to detect a presence of a genotype comprising at least one polymorphism(s) provided in Table 1. Patients are also monitored for the development of an extra-intestinal manifestation.

At least one, two, or three polymorphisms comprising rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, rs9276424, rs10056322, rs6461986, and rs13421864, or any of the above combinations in which a polymorphism is substituted with a proxy polymorphism, are detected in the sample by Illumina ImmunoArray or polymerase chain reaction (PCR) under standard hybridization conditions. Proxy polymorphisms are identified using linkage disequilibrium with a D′1 value of at least 0.8, or an r² value of at least 0.85. In some cases, the proxy polymorphism is additionally selected based on an independent association between the polymorphism and a relevant clinical phenotype of CD (e.g., stricturing and penetrating disease) In this example, one or more primer pairs described herein and/or nucleic acid probes comprising nucleic acid sequences capable of hybridizing the nucleic acid sequences, or their reverse compliments, provided in SEQ ID NOS: 2001-2031, are used.

A EIM profile is generated that correlates the presence or absence of the genotypes with a positive, negative or indeterminate result for a therapeutic response to treatment with a therapeutic agent with a positive predictive value and specificity of at least or about 70%.

The EIM profile of the subject is positive. Based on the EIM profile of the CD patient, a doctor determines that the subject is suitable for treatment with the therapeutic agent. A therapeutically effective amount of a therapeutic agent is administered to the subject with the positive EIM profile.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed:

1. A method for treating a subject having an inflammatory bowel disease, the method comprising administering to the subject a therapeutic agent, provided the subject has been determined to have a genotype comprising one or more single nucleotide polymorphisms selected from the group comprising rs6905036, rs2844510, rs2516514, and rs17030062.

2. The method of claim 1, wherein the subject has been determined to be at risk for developing ankylosing spondylitis and/or sacroiliitis.

3. A method for evaluating whether a subject having an inflammatory bowel disease will develop an extra-intestinal manifestation (EIM), the method comprising determining whether the subject has a genotype comprising one or more single nucleotide polymorphisms selected from the group comprising rs6905036, rs2844510, rs2516514, and rs17030062.

4. The method of claim 3, wherein the EIM comprises ankylosing spondylitis and/or sacroiliitis.

5. The method of claim 3 or claim 4, further comprising administering to the subject a therapeutic agent.

6. The method of any one of claim 1, 2, or 5, wherein the therapeutic agent comprises an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof.

7. The method of any one of claim 1, 2, 5, or 6, wherein the therapeutic agent comprises etanercept, adalimumab, infliximab, cyclobenzaprine, a steroid, secukinumab, methotrexate, leflunomide, hydroxychloroquine, or sulfasalazine, or a combination thereof.

8. A method for treating a subject having an inflammatory bowel disease, the method comprising administering to the subject a therapeutic agent, provided the subject has been determined to have a genotype comprising one or more single nucleotide polymorphisms selected from the group comprising rs9276456, rs2858884, rs2858319, rs6917611, rs6930571, rs389419, rs76558762, rs7956721, rs887864, and rs9276424.

9. The method of claim 8, wherein the subject has been determined to be at risk for developing primary sclerosing cholangitis.

10. A method for evaluating whether a subject having an inflammatory bowel disease will develop an extra-intestinal manifestation (EIM), the method comprising determining whether the subject has a genotype comprising one or more single nucleotide polymorphisms selected from the group comprising rs9276456, rs2858884, rs2858319, rs6917611, rs6930571, rs389419, rs76558762, rs7956721, rs887864, and rs9276424.

11. The method of claim 10, wherein the EIM comprises primary sclerosing cholangitis.

12. The method of claim 10 or claim 11, further comprising administering to the subject a therapeutic agent.

13. The method of any one of claim 8, 9, or 12, wherein the therapeutic agent comprises an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof.

14. The method of any one of the claim 8, 9, 12, or 13, wherein the therapeutic agent comprises ursodeoxycholic acid (UDCA), antipruritic, cholestyramine, antibiotic, vitamin A, vitamin D, vitamin E, or vitamin K, or a combination thereof.

15. A method for treating a subject having an inflammatory bowel disease, the method comprising administering to the subject a therapeutic agent, provided the subject has been determined to have a genotype comprising one or more single nucleotide polymorphisms selected from the group comprising rs10056322, rs6461986, and rs13421864.

16. The method of claim 15, wherein the subject has been determined to be at risk for developing arthritis.

17. A method for evaluating whether a subject having an inflammatory bowel disease will develop an extra-intestinal manifestation (EIM), the method comprising determining whether the subject has a genotype comprising one or more single nucleotide polymorphisms selected from the group comprising rs10056322, rs6461986, and rs13421864.

18. The method of claim 17, wherein the EIM comprises arthritis.

19. The method of claim 16 or 18, wherein the arthritis is peripheral arthritis.

20. The method of any one of claim 17-19, further comprising administering to the subject a therapeutic agent.

21. The method of any one of claim 15, 16, or 20, wherein the therapeutic agent comprises an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof.

22. The method of any one of claim 15, 16, 20, or 21, wherein the therapeutic agent comprises methotrexate, leflunomide, hydroxychloroquine, sulfasalazine, nonsteroidal anti-inflammatory drug (NSAID), TNF inhibitor, etanercept, infliximab, belimumab, rituximab, anakinra, tocilizumab, canakinumab, secukinumab, ustekinumab, ixekizumab, sarilumab, tofacitinib, abatacept, corticosteroid, prednisone, or cortisone, or a combination thereof.

23. A method for treating a subject having an inflammatory bowel disease, the method comprising administering to the subject a therapeutic agent, provided the subject has been determined to have a genotype comprising one or more single nucleotide polymorphisms selected from the group comprising rs4349859, rs4418214, rs3819299, rs2373969, rs4151651, and rs9366775.

24. The method of claim 23, wherein the subject has been determined to be at risk for developing an extra-intestinal manifestation (EIM) of the eye.

25. A method for evaluating whether a subject having an inflammatory bowel disease will develop an extra-intestinal manifestation (EIM), the method comprising determining whether the subject has a genotype comprising one or more single nucleotide polymorphisms selected from the group comprising rs4349859, rs4418214, rs3819299, rs2373969, rs4151651, and rs9366775.

26. The method of claim 24 or claim 25, wherein the EIM comprises uveitis, iritis, episcleritis, scleritis, or undiagnosed ocular inflammation, or a combination thereof.

27. The method of claim 25 or claim 26, further comprising administering to the subject a therapeutic agent.

28. The method of any one of claim 23, 24, or 27, wherein the therapeutic agent comprises an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof.

29. The method of any one of the claim 23, 24, 27, or 28, wherein the therapeutic agent comprises a steroid, methotrexate, mycophenolate, sulfasalazine, azathioprine, cyclosporine, adalimumab, infliximab, daclizumab, abatacept, or rituximab, or a combination thereof.

30. A method for treating a subject having an inflammatory bowel disease, the method comprising administering to the subject a therapeutic agent, provided the subject has been determined to have a genotype comprising one or more single nucleotide polymorphisms selected from the group comprising rs80079682, rs115994059, rs7297515, rs13166683, rs62376929, rs9305694, rs139009610, rs28732100, rs12199223, rs1265181, rs13417109, rs17026757, and rs117670930.

31. The method of claim 30, wherein the subject has been determined to be at risk for developing erythema nodosum, pyoderma gangrenosum, or psoriasis, or a combination thereof.

32. A method for evaluating whether a subject having an inflammatory bowel disease will develop an extra-intestinal manifestation (EIM), the method comprising determining whether the subject has a genotype comprising one or more single nucleotide polymorphisms selected from the group comprising rs80079682, rs115994059, rs7297515, rs13166683, rs62376929, rs9305694, rs139009610, rs28732100, rs12199223, rs1265181, rs13417109, rs17026757, and rs117670930.

33. The method of claim 32, wherein the EIM comprises erythema nodosum, pyoderma gangrenosum, or psoriasis, or a combination thereof.

34. The method of claim 32 or claim 33, further comprising administering to the subject a therapeutic agent.

35. The method of any one of claim 30, 31, or 34, wherein the therapeutic agent comprises an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof.

36. The method of any one of claim 30, 31, 34, or 35, wherein the therapeutic agent comprises an anti-inflammatory drug, cortisone, colchicine, potassium iodine, steroid, hydroxychloroquine, cyclosporin A, or thalidomide, or a combination thereof.

37. The method of any one of the claim 30, 31, or 34-36, wherein the therapeutic agent comprises a corticosteroid, a ciclosporin, infliximab, canakinumab, clobetasol, mupirocin, gentamicin, tacrolimus, mycophenolate mofetil, or thalidomide, or a combination thereof.

38. The method of any one of the claim 30, 31, or 34-37, wherein the therapeutic agent comprises a steroid, retinoid, methotrexate, adalimumab, brodalumab, certolizumab pegol, etanercept, guselkumab, infliximab, ixekizumab, risankizumab-rzaa, secukinumab, tildrakizumab, ustekinumab, or apremilast, or a combination thereof.

39. A method for treating a subject having an inflammatory bowel disease, the method comprising administering to the subject a therapeutic agent, provided the subject has been determined to have a genotype comprising one or more single nucleotide polymorphisms selected from the group comprising rs4349859, rs4463302, rs4418214, rs2069835, rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, and rs9276424.

40. The method of claim 39, wherein the subject has been determined to be at risk for developing an extra-intestinal manifestation (EIM).

41. A method for evaluating whether a subject having an inflammatory bowel disease will develop an extra-intestinal manifestation (EIM), the method comprising determining whether the subject has a genotype comprising one or more single nucleotide polymorphisms selected from the group comprising rs4349859, rs4463302, rs4418214, rs2069835, rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, and rs9276424.

42. The method of claim 40 or claim 41, wherein the EIM comprises ankylosing spondylitis, sacroiliitis, erythema nodosum, pyoderma gangrenosum, psoriasis, a manifestation in the eye, or primary sclerosing cholangitis, or a combination thereof.

43. The method of claim 41 or claim 42, further comprising administering to the subject a therapeutic agent.

44. The method of any one of claim 39, 40, or 43, wherein the therapeutic agent comprises an anti-TL1A antibody, anti-CD30L antibody, glucocorticosteroid, anti-TNF therapy, anti-a4-b7 therapy, anti-IL12p40 therapy, JAK inhibitor, thalidomide, or cyclophosphamide, or a combination thereof.

45. The method of any one of claim 39, 40, 43, or 44, wherein the therapeutic agent comprises an anti-inflammatory drug, cortisone, colchicine, potassium iodine, steroid, hydroxychloroquine, cyclosporin A, thalidomide, or a combination thereof.

46. The method of any one of the claim 39, 40, or 43-45, wherein the therapeutic agent comprises a corticosteroid, ciclosporin, infliximab, canakinumab, clobetasol, mupirocin, gentamicin, tacrolimus, mycophenolate mofetil, or thalidomide, or a combination thereof.

47. The method of any one of the claim 39, 40, or 43-46, wherein the therapeutic agent comprises a steroid, retinoid, methotrexate, adalimumab, brodalumab, certolizumab pegol, etanercept, guselkumab, infliximab, ixekizumab, risankizumab-rzaa, secukinumab, tildrakizumab, ustekinumab, or apremilast, or a combination thereof.

48. The method of any one of the claim 39, 40, or 43-47, wherein the therapeutic agent comprises a steroid, methotrexate, mycophenolate, sulfasalazine, azathioprine, cyclosporine, adalimumab, infliximab, daclizumab, abatacept, or rituximab, or a combination thereof.

49. The method of any one of the claim 39, 40, or 43-48, wherein the therapeutic agent comprises ursodeoxycholic acid (UDCA), antipruritic, cholestyramine, antibiotic, vitamin A, vitamin D, vitamin E, or vitamin K, or a combination thereof.

50. The method of any one of the claim 39, 40, or 43-49, wherein the therapeutic agent comprises etanercept, adalimumab, infliximab, cyclobenzaprine, steroid, secukinumab, methotrexate, leflunomide, hydroxychloroquine, or sulfasalazine, or a combination thereof.

51. A method for treating a subject having an inflammatory bowel disease, the method comprising administering to the subject a therapeutic agent, provided the subject has been determined to have a genotype comprising one or more single nucleotide polymorphisms selected from the group comprising rs61199332, rs7857730, rs497871, rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, rs9276424, rs10056322, rs6461986, rs13421864, rs80079682, rs6905036, rs4349859, rs2844510, rs9276456, rs4463302, rs4418214, and rs2069835.

52. The method of claim 51, wherein the subject has been determined to be at risk for developing an extra-intestinal manifestation (EIM).

53. A method for evaluating whether a subject having an inflammatory bowel disease will develop an extra-intestinal manifestation (EIM), the method comprising determining whether the subject has a genotype comprising one or more single nucleotide polymorphisms selected from the group comprising rs61199332, rs7857730, rs497871, rs2516514, rs17030062, rs9305694, rs139009610, rs28732100, rs12199223, rs1265181, rs13417109, rs17026757, rs117670930, rs115994059, rs7297515, rs13166683, rs62376929, rs4418214, rs3819299, rs2373969, rs4151651, rs9366775, rs2858884, rs2858319, rs6917611, rs6930571, rs389419, rs76558762, rs7956721, rs887864, rs9276424, rs10056322, rs6461986, rs13421864, rs80079682, rs6905036, rs4349859, rs2844510, rs9276456, rs4463302, rs4418214, and rs2069835.

54. The method of claim 53, further comprising administering to the subject a therapeutic agent.

55. The method of any one of claim 51, 52, or 54, wherein the therapeutic agent comprises an anti-TL1A antibody, an anti-CD30L antibody, glucocorticosteroid, anti-TNF therapy, anti-a4-b7 therapy, anti-IL12p40 therapy, JAK inhibitor, thalidomide, or cyclophosphamide or a combination thereof.

56. The method of any one of claim 51, 52, 54, or 55, wherein the therapeutic agent comprises an anti-inflammatory drug, cortisone, colchicine, potassium iodine, steroid, hydroxychloroquine, cyclosporin A, thalidomide, or a combination thereof.

57. The method of any one of the claim 51, 52, or 54-56, wherein the therapeutic agent comprises a corticosteroid, ciclosporin, infliximab, canakinumab, clobetasol, mupirocin, gentamicin, tacrolimus, mycophenolate mofetil, or thalidomide, or a combination thereof.

58. The method of any one of the claim 51, 52, or 54-57, wherein the therapeutic agent comprises a steroid, retinoid, methotrexate, adalimumab, brodalumab, certolizumab pegol, guselkumab, infliximab, ixekizumab, risankizumab-rzaa, secukinumab, tildrakizumab, ustekinumab, or apremilast, or a combination thereof.

59. The method of any one of the claim 51, 52, or 54-58, wherein the therapeutic agent comprises a steroid, methotrexate, mycophenolate, sulfasalazine, azathioprine, cyclosporine, adalimumab, infliximab, daclizumab, abatacept, or rituximab, or a combination thereof.

60. The method of any one of the claim 51, 52, or 54-59, wherein the therapeutic agent comprises ursodeoxycholic acid (UDCA), antipruritic, cholestyramine, antibiotic, vitamin A, vitamin D, vitamin E, or vitamin K, or a combination thereof.

61. The method of any one of the claim 51, 52, or 54-60, wherein the therapeutic agent comprises methotrexate, leflunomide, hydroxychloroquine, sulfasalazine, a NSAID, TNF inhibitor, etanercept, infliximab, belimumab, rituximab, anakinra, tocilizumab, canakinumab, secukinumab, ustekinumab, ixekizumab sarilumab, tofacitinib, abatacept, corticosteroid, prednisone, cortisone, or a combination thereof.

62. The method of any one of the claim 51, 52, or 54-61, wherein the therapeutic agent comprises etanercept, adalimumab, infliximab, cyclobenzaprine, steroid, secukinumab, methotrexate, leflunomide hydroxychloroquine, or sulfasalazine, or a combination thereof.

63. The method of any one of claim 52-62, wherein the EIM comprises ankylosing spondylitis, sacroiliitis, erythema nodosum, pyoderma gangrenosum, psoriasis, a manifestation in the eye, primary sclerosing cholangitis, or peripheral arthritis, or a combination thereof.

64. The method of claim 63, wherein the EIM comprises ankylosing spondylitis and sacroiliitis.

65. The method of claim 63, wherein the EIM comprises erythema nodosum, pyoderma gangrenosum, and/or psoriasis.

66. The method of claim 63, wherein the EIM comprises the manifestation in the eye.

67. The method of claim 66, wherein the manifestation in the eye comprises uveitis, iritis, episcleritis, scleritis, or undiagnosed ocular inflammation, or a combination thereof.

68. The method of claim 63, wherein the EIM comprises primary sclerosing cholangitis.

69. The method of claim 63, wherein the EIM comprises peripheral arthritis.

70. The method of claim 63, wherein the arthritis comprises large joint arthritis, small joint arthritis, extracolonic arthritis, Crohn's disease/ulcerative colitis non-specific joint inflammation, or TBD-associated arthralgia complication, or a combination thereof.

71. The method of claim 63, wherein the EIM comprises ankylosing spondylitis, sacroiliitis, erythema nodosum, pyoderma gangrenosum, psoriasis, a manifestation in the eye, or primary sclerosing cholangitis, or a combination thereof.

72. The method of claim 63, wherein the EIM comprises ankylosing spondylitis, sacroiliitis, erythema nodosum, pyoderma gangrenosum, psoriasis, eye, peripheral arthritis, or primary sclerosing cholangitis, or a combination thereof.

73. The method of any one of claim 1-72, wherein the subject comprises a serological marker selected from the group comprising anti-neutrophil cytoplasmic antibodies (ANCA), anti-Saccharomyces cerevisiae antibodies (ASCA), anti-outer member porin C (anti-OmpC), anti-Pseudomonas fluorescens bacterial sequence 12 (anti-I2), and anti-bacterial flagellin (CBir1).

74. The method of any one of claim 1-73, wherein the inflammatory bowel disease comprises Crohn's disease.

75. The method of claim 74, wherein the subject has a Crohn's disease (CD) phenotype selected from the group comprising upper GI, colonic, small bowel, structuring, penetrating, structuring and penetrating, and perianal CD.

76. The method of any one of claim 1-75, wherein the subject has undergone surgery for treatment of the inflammatory bowel disease.

77. The method of any one of claim 1-76, wherein the inflammatory bowel disease comprises ulcerative colitis.

78. The method of any one of claim 1-77, wherein the subject is female.

79. The method of any one of claim 1-77, wherein the subject is male.

80. The method of any one of claim 1-79, wherein the subject is Jewish.

81. The method of any one of claim 1-80, wherein the subject is was diagnosed with inflammatory bowel disease before age 17.

82. The method of any one of claim 1-80, wherein the subject is was diagnosed with inflammatory bowel disease before age 10.

83. The method of any one of claim 1-82, wherein the subject is a smoker.

84. The method of any one of claim 1-83, further comprising determining whether the subject comprises the one or more single nucleotide polymorphisms.
